(19) United States
(12) Patent Application Publication
(10) Pub. No.: US 2023/0136702 A1
(43) Pub. Date: May 4, 2023

(54) Inhibitors of Human Herpesviruses

(71) Applicant: The Governors of the University of Alberta, Edmonton (CA)

(72) Inventors: James Nieman, Sherwood Park (CA); Mostofa Hena, Edmonton (CA); Bing Bai, Edmonton (CA); Appan Srinivas Kandadai, Edmonton (CA); Alexandr Belovodskiy, Edmonton (CA)

(21) Appl. No.: 17/800,516

(22) PCT Filed: Mar. 2, 2021
(86) PCT No.: PCT/CA2021/050275

§ 371 Date: Aug. 17, 2022

Related U.S. Application Data
(60) Provisional application No. 62/984,456, filed on Mar. 3, 2020.

(51) Int. Cl. A61K 31/4985; C07D 471/04; C07D 519/00; A61K 31/4375; A61K 31/522; A61K 31/662; A61K 31/517; A61P 31/22

(52) U.S. Cl. CPC A61K 31/4985; C07D 471/04; C07D 519/00; A61K 31/4375; A61K 31/522; A61K 31/662; A61K 31/517; A61P 31/22

(57) ABSTRACT

Provided herein are compounds, compositions and methods for inhibition of herpesviruses. In some cases, the subject compounds inhibit a herpesvirus in a cell. Also provided are compounds, compositions and methods for treating a herpesvirus in an individual. In some cases, the methods include administering to an individual a therapeutically effective amount of a subject compound to treat the individual for the herpesvirus. In certain embodiments, the compounds disclosed herein are cytomegalovirus (CMV) inhibitors. In certain embodiments, the compounds disclosed herein are human cytomegalovirus (CMV) inhibitors.

### CROSS-REFERENCE TO RELATED APPLICATIONS

This application claims the benefit of priority to U.S. Provisional Application No. 62/984,456, filed Mar. 3, 2020, the disclosure of which is incorporated herein by reference in its entirety.

### INTRODUCTION

Herpesviruses routinely infect humans. Herpesviruses include, herpes simplex virus types 1 and 2, varicella-zoster virus, cytomegalovirus, Epstein-Barr virus, human herpesvirus 6 (variants A and B), human herpesvirus 7, and Kaposi's sarcoma virus or human herpesvirus 8. A simian virus, B virus, occasionally infects humans. All herpesviruses can establish latent infection within specific tissues.

Herpesviruses are divided into three groups: The α herpesviruses, herpes simplex virus types 1 and 2, and varicella-zoster virus; β herpesviruses, cytomegalovirus, and human herpesviruses 6 and 7; and γ herpesviruses, Epstein-Barr virus and human herpesvirus 8.

Cytomegalovirus (CMV) is associated with widespread morbidity and mortality. Infection with CMV is common, and although CMV infection generally does not produce symptoms in healthy adults, high-risk groups, including immunocompromised organ transplant recipients and HIV-infected individuals, are at risk of developing CMV-associated disease. CMV infection is also a risk for immune immature individuals, such as neonates. Transmission to neonates can be from the mother, congenital CMV, or from the environment.

### SUMMARY

Provided herein are compounds, compositions and methods for inhibition of a herpesvirus. In some cases, the subject compounds inhibit a herpesvirus in a cell. Also provided are compounds, compositions and methods for treating a herpesvirus in an individual. In some cases, the methods include administering to an individual a therapeutically effective amount of a subject compound to treat the individual for the herpesvirus. In certain embodiments, the compounds disclosed herein are cytomegalovirus (CMV) inhibitors. In certain embodiments, the compounds disclosed herein are human cytomegalovirus (CMV) inhibitors. In certain embodiments, the subject compounds possess antiviral activity. In some cases, the subject compounds possess CMV DNA polymerase activity. In some cases, the subject compounds possess both antiviral and CMV DNA polymerase activity.

These and other advantages and features of the disclosure will become apparent to those persons skilled in the art upon reading the details of the compositions and methods of use, which are more fully described below.

### BRIEF DESCRIPTION OF THE FIGURES

FIG. 1 provides DNA sequencing analysis of pFstBac-UL54-6H (c2), UL54 encoding region is in bold text, and cloning sites (NedI and HidIII) are highlighted grey.

FIG. 2, panels A-B, show analysis of purified HCMV DNA Polymerase. After separation by sizing exclusion column (Superdex 200), peak fractions of HCMV DNA polymerase (UL) were pooled, concentrated, dialyzed and analyzed by either SDS gel visualized by coomassie blue staining (panel A), or Western blot using the monoclonal antibody against 6His tag (panel B). M=protein standards in kDa.

### DEFINITIONS

To facilitate understanding of the disclosure set forth herein, a number of terms are defined below.

Generally, the nomenclature used herein and the laboratory procedures in organic chemistry, medicinal chemistry, and pharmacology described herein are those known and commonly employed in the art. Unless defined otherwise, all technical and scientific terms used herein generally have the same meaning as commonly understood by one of ordinary skill in the art to which this disclosure belongs.

As used herein, the term “herpesvirus” is well understood in the art, and refers to any member of the family Herpesviridae. Herpesviruses include, e.g., cytomegalovirus (CMV), herpes simplex virus-1 (HSV-1), herpes simplex virus-2 (HSV-2), varicella-zoster virus (VZV), Epstein-Barr virus (EBV), and Kaposi's sarcoma-associated herpesvirus (KSHV; also known as human herpesvirus-8 or HHV-8).

The term “cytomegalovirus,” also known as CMV, refers to a member of the herpesvirus family in any species, including human. CMV is also referred to as a betaherpesviridae. CMV is a herpesvirus that infects mononuclear cells and lymphocytes.

The term “human cytomegalovirus,” also known as HCMV, indicates a member of the CMV family that infects humans. HCMV is a beta human herpesvirus with a genome size of 230 Kbp, coding more than 70 viral proteins. HCMV is also designated as human herpesvirus 5 (HHV-5). Mouse CMV (mCMV) indicates a member of the CMV family that infects mice. Rhesus monkey CMV (rhCMV) indicates a member of the CMV family that infects rhesus monkeys.

The terms “individual,” “subject,” “host,” and “patient,” used interchangeably herein, refer to a mammal, including, but not limited to, murines (rats, mice), non-human primates, humans, canines, felines, ungulates (e.g., equines, bovines, ovines, porcines, caprines), etc. In some cases, the individual is a human. In some cases, the individual is a non-human primate.

As used herein, the terms “treatment,” “treating,” and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment,” as used herein, covers any treatment of a disease in a mammal, particularly in a human, and includes: (a) preventing the disease from occurring in a subject which may be predisposed to the disease but has not yet been diagnosed as having it; (b) inhibiting the disease, i.e., arresting its development; and (c) relieving the disease, i.e., causing regression of the disease.

A “therapeutically effective amount” or “efficacious amount” refers to the amount of a compound that, when administered to a mammal or other subject for treating a disease, is sufficient to effect such treatment for the disease. The “therapeutically effective amount” will vary depending on the compound or the cell, the disease and its severity and the age, weight, etc., of the subject to be treated. A therapeutically effective amount” can be a “prophylactically effective amount”.

The term “pharmaceutically acceptable salt” means a salt which is acceptable for administration to a patient, such as a mammal (salts with counterions having acceptable mammalian safety for a given dosage regime). Such salts can be derived from pharmaceutically acceptable inorganic or organic bases and from pharmaceutically acceptable inorganic or organic acids. “Pharmaceutically acceptable salt” refers to pharmaceutically acceptable salts of a compound, which salts are derived from a variety of organic and inorganic counter ions well known in the art and include, by way of example only, sodium, potassium, calcium, magnesium, ammonium, tetraalkylammonium, and the like; and when the molecule contains a basic functionality, salts of organic or inorganic acids, such as hydrochloride, hydrobromide, formate, tartrate, besylate, mesylate, acetate, maleate, oxalate, and the like.

As used herein, the terms “determining,” “measuring,” “assessing,” and “assaying” are used interchangeably and include both quantitative and qualitative determinations.

The term “pharmaceutically acceptable excipient” refers to a pharmaceutically-acceptable material, composition, or vehicle, such as a liquid or solid filler, diluent, solvent, or encapsulating material. In one embodiment, each component is “pharmaceutically acceptable” in the sense of being compatible with the other ingredients of a pharmaceutical formulation, and suitable for use in contact with the tissue or organ of humans and animals without excessive toxicity, irritation, allergic response, immunogenicity, or other problems or complications, commensurate with a reasonable benefit/risk ratio. See, Remington: The Science and Practice of Pharmacy, 21st ed.; Lippincott Williams & Wilkins: Philadelphia, Pa., 2005; Handbook of Pharmaceutical Excipients, 6th ed.; Rowe et al., Eds.; The Pharmaceutical Press and the American Pharmaceutical Association: 2009; Handbook of Pharmaceutical Additives, 3rd ed.; Ash and Ash Eds.; Gower Publishing Company: 2007; Pharmaceutical Preformulation and Formulation, 2nd ed.; Gibson Ed.; CRC Press LLC: Boca Raton, Fla., 2009.

As used herein, a “pharmaceutical composition” is meant to encompass a composition suitable for administration to a subject, such as a mammal, especially a human. In general, a “pharmaceutical composition” is sterile, and preferably free of contaminants that are capable of eliciting an undesirable response within the subject (e.g., the compound(s) in the pharmaceutical composition is pharmaceutical grade). Pharmaceutical compositions can be designed for administration to subjects or patients in need thereof via a number of different routes of administration including oral, buccal, rectal, parenteral, intraperitoneal, intradermal, intracheal, intramuscular, subcutaneous, and the like.

As used herein, the phrase “having the formula” or “having the structure” is not intended to be limiting and is used in the same way that the term “comprising” is commonly used. The term “independently selected from” is used herein to indicate that the recited elements, e.g., R groups or the like, can be identical or different.

As used herein, the terms “may,” “optional,” “optionally,” or “may optionally” mean that the subsequently described circumstance may or may not occur, so that the description includes instances where the circumstance occurs and instances where it does not. For example, the phrase “optionally substituted” means that a non-hydrogen substituent may or may not be present on a given atom, and, thus, the description includes structures wherein a non-hydrogen substituent is present and structures wherein a non-hydrogen substituent is not present.

The terms “about” or “approximately” mean an acceptable error for a particular value as determined by one of ordinary skill in the art, which depends in part on how the value is measured or determined. In certain embodiments, the terms “about” or “approximately” mean within 1, 2, 3, or 4 standard deviations. In certain embodiments, the term “about” or “approximately” means within 50%, 20%, 15%, 10%, 9%, 8%, 7%, 6%, 5%, 4%, 3%, 2%, 1%, 0.5%, or 0.05% of a given value or range.

The term “modulator,” as used herein, refers to a compound that alters an activity of a molecule. For example, a modulator includes a compound that causes an increase or a decrease in the magnitude of a certain activity of a molecule compared to the magnitude of the activity in the absence of the modulator. In certain embodiments, a modulator decreases the magnitude of one or more activities of a molecule. In certain embodiments, a modulator completely prevents one or more activities of a molecule. In certain embodiments, a modulator increases the magnitude of at least one activity of a molecule. In certain embodiments the presence of a modulator results in an activity that does not normally occur in the absence of the modulator.

The terms “enhance” or “enhancing,” as used herein, means to increase or prolong either in potency or duration a desired effect.

“Acyl” refers to the groups H—C(O)—, alkyl-C(O)—, substituted alkyl-C(O)—, alkenyl-C(O)—, substituted alkenyl-C(O)—, alkynyl-C(O)—, substituted alkynyl-C(O)—, cycloalkyl-C(O)—, substituted cycloalkyl-C(O)—, cycloalkenyl-C(O)—, substituted cycloalkenyl-C(O)—, aryl-C(O)—, substituted aryl-C(O)—, heteroaryl-C(O)—, substituted heteroaryl-C(O)—, heterocyclyl-C(O)—, and substituted heterocyclyl-C(O)—, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein. For example, acyl includes the “acetyl” group CH₃C(O)—

The term “alkyl” as used herein refers to a branched or unbranched saturated hydrocarbon group (i.e., a mono-radical) typically although not necessarily containing 1 to about 24 carbon atoms, such as methyl, ethyl, n-propyl, isopropyl, n-butyl, isobutyl, t-butyl, octyl, decyl, and the like, as well as cycloalkyl groups such as cyclopentyl, cyclohexyl and the like. Generally, although not necessarily, alkyl groups herein may contain 1 to about 18 carbon atoms, and such groups may contain 1 to about 12 carbon atoms. Illustrative examples of alkyl include, but are not limited to, methyl, ethyl, n-propyl, iso-propyl, cyclopropyl, n-butyl, sec-butyl, tert-butyl, cyclobutyl, n-pentyl, isopentyl, neopentyl, cyclopentyl, n-hexyl, 3-methylhexyl, 2,2-dimethylpentyl, 2,3-dimethylhexyl, cyclohexyl, n-heptyl, n-octyl, n-nonyl, and n-decyl. The term “lower alkyl” intends an alkyl group of 1 to 6 carbon atoms.

The term “alkenyl” refers to a linear, branched or cyclic hydrocarbon group of 2 to about 24 carbon atoms containing at least one double bond, such as ethenyl, n-propenyl, isopropenyl, n-butenyl, isobutenyl, octenyl, decenyl, tetradecenyl, hexadecenyl, eicosenyl, tetracosenyl, and the like. Generally, although again not necessarily, alkenyl groups herein may contain 2 to about 18 carbon atoms, and for example may contain 2 to 12 carbon atoms. The term “lower alkenyl” intends an alkenyl group of 2 to 6 carbon atoms. The term “substituted alkenyl” refers to alkenyl substituted with one or more substituent groups, and the terms “heteroatom-containing alkenyl” and “heteroalkenyl” refer to alkenyl in which at least one carbon atom is replaced with a heteroatom. If not otherwise indicated, the terms “alkenyl” and “lower alkenyl” include linear, branched, cyclic, unsubstituted, substituted, and/or heteroatom-containing alkenyl and lower alkenyl, respectively.

The term “alkynyl” refers to a linear or branched hydrocarbon group of 2 to 24 carbon atoms containing at least one triple bond, such as ethynyl, n-propynyl, and the like. Generally, although again not necessarily, alkynyl groups herein may contain 2 to about 18 carbon atoms, and such groups may further contain 2 to 12 carbon atoms. The term “lower alkynyl” intends an alkynyl group of 2 to 6 carbon atoms. The term “substituted alkynyl” refers to alkynyl substituted with one or more substituent groups, and the terms “heteroatom-containing alkynyl” and “heteroalkynyl” refer to alkynyl in which at least one carbon atom is replaced with a heteroatom. If not otherwise indicated, the terms “alkynyl” and “lower alkynyl” include linear, branched, unsubstituted, substituted, and/or heteroatom-containing alkynyl and lower alkynyl, respectively.

The term “C₁-C₆-alkyl” as used herein, means a straight, branched chain, or cyclic hydrocarbon containing from 1-6 carbon atoms. Representative examples of C₁-C₆-alkyl include, but are not limited to, methyl, ethyl, n-propyl, iso-propyl, cyclopropyl, n-butyl, sec-butyl, tert-butyl, cyclobutyl, n-pentyl, isopentyl, neopentyl, cyclopentyl, n-hexyl and cyclohexyl.

The term “C₁-C₃-alkyl” as used herein, means a straight, branched chain, or cyclic hydrocarbon containing from 1-3 carbon atoms. Representative examples of C₁-C₃-alkyl include, but are not limited to, methyl, ethyl, n-propyl, iso-propyl, and cyclopropyl.

The term “C₃-C₆-alkyl” as used herein, means a straight, branched chain, or cyclic hydrocarbon containing from 3-6 carbon atoms. Representative examples of C₃-C₆-alkyl include, but are not limited to, n-propyl, iso-propyl, cyclopropyl, n-butyl, sec-butyl, tert-butyl, cyclobutyl, n-pentyl, isopentyl, neopentyl, cyclopentyl, n-hexyl and cyclohexyl. “Substituted alkyl” refers to alkyl substituted with one or more substituent groups, and this includes instances wherein two hydrogen atoms from the same carbon atom in an alkyl substituent are replaced, such as in a carbonyl group (i.e., a substituted alkyl group may include a —C(═O)— moiety). The terms “heteroatom-containing alkyl” and “heteroalkyl” refer to an alkyl substituent in which at least one carbon atom is replaced with a heteroatom, as described in further detail infra. If not otherwise indicated, the terms “alkyl” and “lower alkyl” include linear, branched, cyclic, unsubstituted, substituted, and/or heteroatom-containing alkyl or lower alkyl, respectively.

The term “substituted alkyl” is meant to include an alkyl group as defined herein wherein one or more carbon atoms in the alkyl chain have been optionally replaced with a heteroatom such as —O—, —N—, —S—, —S(O)ₙ— (where n is 0 to 2), —NR— (where R is hydrogen or alkyl) and having from 1 to 5 substituents selected from the group consisting of alkoxy, substituted alkoxy, cycloalkyl, substituted cycloalkyl, cycloalkenyl, substituted cycloalkenyl, acyl, acylamino, acyloxy, amino, aminoacyl, aminoacyloxy, oxyaminoacyl, azido, cyano, halogen, hydroxyl, oxo, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, —SO-alkyl, —SO-aryl, —SO-heteroaryl, —SO₂-alkyl, —SO₂-aryl, —SO₂-heteroaryl, and —NR′R″, wherein R′ and R″ may be the same or different and are chosen from hydrogen, optionally substituted alkyl, cycloalkyl, alkenyl, cycloalkenyl, alkynyl, aryl, heteroaryl and heterocyclic.

The term “alkoxy” as used herein intends an alkyl group bound through a single, terminal ether linkage; that is, an “alkoxy” group may be represented as —O-alkyl where alkyl is as defined above. A “lower alkoxy” group intends an alkoxy group containing 1 to 6 carbon atoms, and includes, for example, methoxy, ethoxy, n-propoxy, isopropoxy, t-butyloxy, pentyloxy, hexyloxy etc. Substituents identified as “C₁-C₆ alkoxy” or “lower alkoxy” herein may, for example, may contain 1 to 3 carbon atoms, and as a further example, such substituents may contain 1 or 2 carbon atoms (i.e., methoxy and ethoxy).

The term “substituted alkoxy” refers to the groups substituted alkyl-O—, substituted alkenyl-O—, substituted cycloalkyl-O—, substituted cycloalkenyl-O—, and substituted alkynyl-O— where substituted alkyl, substituted alkenyl, substituted cycloalkyl, substituted cycloalkenyl and substituted alkynyl are as defined herein.

The term “haloalkyl,” as used herein, means an alkyl group, as defined herein, in which at least one hydrogen is replaced with a halogen atom. In certain embodiments in which two or more hydrogen atoms are replaced with halogen atoms, the halogen atoms are all the same as one another. In other embodiments in which two or more hydrogen atoms are replaced with halogen atoms, the halogen atoms are not all the same as one another.

The term “aromatic” as used herein, refers to a planar ring having a delocalized π-electron system containing 4n+2 π electrons, where n is an integer. In certain embodiments, aromatic rings are formed by five, six, seven, eight, nine, or more than nine atoms. In certain embodiments, aromatics are unsubstituted or substituted one or more substituents as defined herein). The term includes monocyclic or fused-ring polycyclic (i.e., rings which share adjacent pairs of carbon atoms) groups.

The term “aryl” as used herein, and unless otherwise specified, refers to an aromatic substituent generally, although not necessarily, containing 5 to 30 carbon atoms and containing a single aromatic ring or multiple aromatic rings that are fused together, directly linked, or indirectly linked (such that the different aromatic rings are bound to a common group such as a methylene or ethylene moiety). Aryl groups may, for example, contain 5 to 20 carbon atoms, and as a further example, aryl groups may contain 5 to 12 carbon atoms. For example, aryl groups may contain one aromatic ring or two or more fused or linked aromatic rings (i.e., biaryl, aryl-substituted aryl, etc.). Examples include phenyl, naphthyl, biphenyl, diphenylether, diphenylamine, benzophenone, and the like. “Substituted aryl” refers to an aryl moiety substituted with one or more substituent groups, and the terms “heteroatom-containing aryl” and “heteroaryl” refer to aryl substituent, in which at least one carbon atom is replaced with a heteroatom, as will be described in further detail infra. Aryl is intended to include stable cyclic, heterocyclic, polycyclic, and polyheterocyclic unsaturated C₃-C₁₄ moieties, exemplified but not limited to phenyl, biphenyl, naphthyl, pyridyl, furyl, thiophenyl, imidazoyl, pyrimidinyl, and oxazoyl; which may further be substituted with one to five members selected from the group consisting of hydroxy, C₁-C₈ alkoxy, C₁-C₈ branched or straight-chain alkyl, acyloxy, carbamoyl, amino, N-acylamino, nitro, halogen, trifluoromethyl, cyano, and carboxyl (see e.g. Katritzky, Handbook of Heterocyclic Chemistry). If not otherwise indicated, the term “aryl” includes unsubstituted, substituted, and/or heteroatom-containing aromatic substituents.

The term “amino” is used herein to refer to the group —NRR′ wherein R and R′ are independently hydrogen or nonhydrogen substituents, with nonhydrogen substituents including, for example, alkyl, aryl, alkenyl, aralkyl, and substituted and/or heteroatom-containing variants thereof.

The terms “halo” and “halogen” are used in the conventional sense to refer to a chloro, bromo, fluoro or iodo substituent.

“Carboxyl,” “carboxy” or “carboxylate” refers to —CO₂H or salts thereof.

“Cycloalkyl” refers to cyclic alkyl groups of from 3 to 10 carbon atoms having single or multiple cyclic rings including fused, bridged, and spiro ring systems. Examples of suitable cycloalkyl groups include, for instance, adamantyl, cyclopropyl, cyclobutyl, cyclopentyl, cyclooctyl and the like. Such cycloalkyl groups include, by way of example, single ring structures such as cyclopropyl, cyclobutyl, cyclopentyl, cyclooctyl, and the like, or multiple ring structures such as adamantanyl, and the like.

The term “substituted cycloalkyl” refers to cycloalkyl groups having from 1 to 5 substituents, or from 1 to 3 substituents, selected from alkyl, substituted alkyl, alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, oxo, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, —SO-alkyl, —SO-substituted alkyl, —SO-aryl, —SO-heteroaryl, —SO₂-alkyl, —SO₂-substituted alkyl, —SO₂-aryl and —SO₂-heteroaryl.

The term “heteroatom” as used herein refers to an atom other than carbon or hydrogen. Heteroatoms are typically independently selected from among oxygen, sulfur, nitrogen, silicon and phosphorus, but are not limited to these atoms. In embodiments in which two or more heteroatoms are present, the two or more heteroatoms are all the same as one another, or some or all of the two or more heteroatoms are each different from the others.

The term “heteroatom-containing” as in a “heteroatom-containing alkyl group” (also termed a “heteroalkyl” group) or a “heteroatom-containing aryl group” (also termed a “heteroaryl” group) refers to a molecule, linkage or substituent in which one or more carbon atoms are replaced with an atom other than carbon, e.g., nitrogen, oxygen, sulfur, phosphorus or silicon, typically nitrogen, oxygen or sulfur. Similarly, the term “heteroalkyl” refers to an alkyl substituent that is heteroatom-containing, the terms “heterocyclic” or “heterocycle” refer to a cyclic substituent that is heteroatom-containing, the terms “heteroaryl” and “heteroaromatic” respectively refer to “aryl” and “aromatic” substituents that are heteroatom-containing, and the like. Examples of heteroalkyl groups include alkoxyaryl, alkylsulfanyl-substituted alkyl, N-alkylated amino alkyl, and the like. Examples of heteroaryl substituents include pyrrolyl, pyrrolidinyl, pyridinyl, quinolinyl, indolyl, furyl, pyrimidinyl, imidazolyl, 1,2,4-triazolyl, tetrazolyl, etc., and examples of heteroatom-containing alicyclic groups are pyrrolidino, morpholino, piperazino, piperidino, tetrahydrofuranyl, etc.

“Heteroaryl” or “heteroaromatic” refers to an aromatic group of from 1 to 15 carbon atoms, such as from 1 to 10 carbon atoms and 1 to 10 heteroatoms selected from the group consisting of oxygen, nitrogen, and sulfur within the ring. Such heteroaryl groups can have a single ring (such as, pyridinyl, imidazolyl or furyl) or multiple condensed rings in a ring system (for example as in groups such as, indolizinyl, quinolinyl, benzofuran, benzimidazolyl or benzothienyl), wherein at least one ring within the ring system is aromatic and at least one ring within the ring system is aromatic, provided that the point of attachment is through an atom of an aromatic ring. In certain embodiments, the nitrogen and/or sulfur ring atom(s) of the heteroaryl group are optionally oxidized to provide for the N-oxide (N→O), sulfinyl, or sulfonyl moieties. This term includes, by way of example, pyridinyl, pyrrolyl, indolyl, thiophenyl, and furanyl. Unless otherwise constrained by the definition for the heteroaryl substituent, such heteroaryl groups can be optionally substituted with 1 to 5 substituents, or from 1 to 3 substituents, selected from acyloxy, hydroxy, thiol, acyl, alkyl, alkoxy, alkenyl, alkynyl, cycloalkyl, cycloalkenyl, substituted alkyl, substituted alkoxy, substituted alkenyl, substituted alkynyl, substituted cycloalkyl, substituted cycloalkenyl, amino, substituted amino, aminoacyl, acylamino, alkaryl, aryl, aryloxy, azido, carboxyl, carboxylalkyl, cyano, halogen, nitro, heteroaryl, heteroaryloxy, heterocyclyl, heterocyclooxy, aminoacyloxy, oxyacylamino, thioalkoxy, substituted thioalkoxy, thioaryloxy, thioheteroaryloxy, —SO-alkyl, —SO-substituted alkyl, —SO-aryl, —SO-heteroaryl, —SO₂-alkyl, —SO₂-substituted alkyl, —SO₂-aryl and —SO₂-heteroaryl, and trihalomethyl.

Examples of monocyclic heteroaryl groups include, but are not limited to, furanyl, imidazolyl, isothiazolyl, isoxazolyl, oxadiazolyl, oxadiazolyl, oxazolyl, pyrazinyl, pyrazolyl, pyridazinyl, pyridyl, pyrimidinyl, pyrrolyl, thiadiazolyl, thiazolyl, thienyl, tetrazolyl, triazinyl, and triazolyl. Examples of bicyclic heteroaryl groups include, but are not limited to, benzofuranyl, benzimidazolyl, benzoisoxazolyl, benzopyranyl, benzothiadiazolyl, benzothiazolyl, benzothienyl, benzotriazolyl, benzoxazolyl, furopyridyl, imidazopyridinyl, imidazothiazolyl, indolizinyl, indolyl, indazolyl, isobenzofuranyl, isobenzothienyl, isoindolyl, isoquinolinyl, isothiazolyl, naphthyridinyl, oxazolopyridinyl, phthalazinyl, pteridinyl, purinyl, pyridopyridyl, pyrrolopyridyl, quinolinyl, quinoxalinyl, quinazolinyl, thiadiazolopyrimidyl, and thienopyridyl. Examples of tricyclic heteroaryl groups include, but are not limited to, acridinyl, benzindolyl, carbazolyl, dibenzofuranyl, perimidinyl, phenanthrolinyl, phenanthridinyl, phenarsazinyl, phenazinyl, phenothiazinyl, phenoxazinyl, and xanthenyl. Illustrative of heteroaryl groups include, but are not limited to, the following moieties:

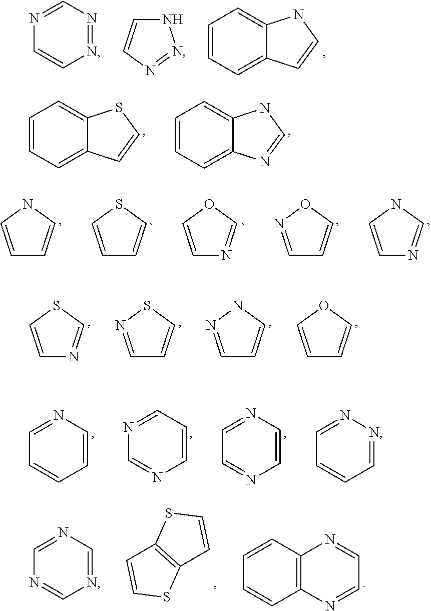

In certain embodiments, depending on the structure, a heteroaryl group is a monoradical or a diradical (i.e., a heteroarylene group).

As used herein, the terms “Heterocycle,” “heterocyclic,” “heterocycloalkyl,” and “heterocyclyl” refer to a saturated or unsaturated group having a single ring or multiple condensed rings, including fused bridged and spiro ring systems, and having from 3 to 15 ring atoms, including 1 to 4 hetero atoms. These ring atoms are selected from the group consisting of nitrogen, sulfur, or oxygen, wherein, in fused ring systems, one or more of the rings can be cycloalkyl, aryl, or heteroaryl, provided that the point of attachment is through the non-aromatic ring. In certain embodiments, the nitrogen and/or sulfur atom(s) of the heterocyclic group are optionally oxidized to provide for the N-oxide, —S(O)—, or —SO₂— moieties.

Examples of monocyclic heterocyclic groups include, but are not limited to, aziridinyl, azetidinyl, 2,3,4,5-tetrahydrofuranyl, pyrrolidinyl, pyrrolidinone, imidazolidinone, pyrazolinyl, tetrahydrothiophenyl, sulfolanyl, 2-oxazolidinone, piperidinyl, piperazinyl, tetrahydropyranyl, morpholinyl, thiomorpholinyl, and azepanyl.

Examples of bicyclic heterocyle groups include, but are not limited to, decahydroquinolinyl, decahydroisoquinolinyl, 1,2,3,4-tetrahydroquinolinyl, 1,2,3,4-tetrahydroisoquinolinyl, phenoxazinyl, phenothiazinyl, and 2-azaadamantanyl. Illustrative of heterocycle groups include, but are not limited to, the following moieties:

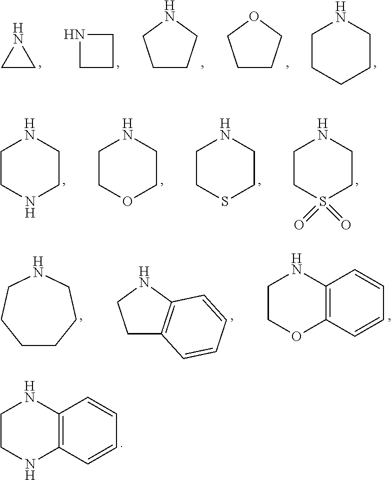

In certain embodiments, depending on the structure, a heterocyclic group is connected at the heteroatom, such as a nitrogen atom. In certain embodiments, a heterocyclic group is connected at a carbon atom.

Unless otherwise constrained by the definition for the heterocyclic substituent, such heterocyclic groups can be optionally substituted with 1 to 5, or from 1 to 3 substituents, selected from alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, oxo, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, —SO-alkyl, —SO-substituted alkyl, —SO-aryl, —SO-heteroaryl, —SO₂-alkyl, —SO₂-substituted alkyl, —SO₂-aryl, —SO₂-heteroaryl, and fused heterocycle.

By “substituted” as in “substituted hydrocarbyl,” “substituted alkyl,” “substituted aryl,” and the like, as alluded to in some of the aforementioned definitions, is meant that in the hydrocarbyl, alkyl, aryl, or other moiety, at least one hydrogen atom bound to a carbon (or other) atom is replaced with one or more non-hydrogen substituents.

Examples of such substituents include, without limitation, functional groups, and the hydrocarbyl moieties C1-C24 alkyl (including C1-C18 alkyl, further including C1-C12 alkyl, and further including C1-C6 alkyl), C2-C24 alkenyl (including C2-C18 alkenyl, further including C2-C12 alkenyl, and further including C2-C6 alkenyl), C2-C24 alkynyl (including C2-C18 alkynyl, further including C2-C12 alkynyl, and further including C2-C6 alkynyl), C5-C30 aryl (including C5-C20 aryl, and further including C5-C12 aryl), and C6-C30 aralkyl (including C6-C20 aralkyl, and further including C6-C12 aralkyl). The above-mentioned hydrocarbyl moieties may be further substituted with one or more functional groups or additional hydrocarbyl moieties such as those specifically enumerated. Unless otherwise indicated, any of the groups described herein are to be interpreted as including substituted and/or heteroatom-containing moieties, in addition to unsubstituted groups.

“Sulfonyl” refers to the group SO₂-alkyl, SO₂-substituted alkyl, SO₂-alkenyl, SO₂-substituted alkenyl, SO₂-cycloalkyl, SO₂-substituted cycloalkyl, SO₂-cycloalkenyl, SO₂-substituted cylcoalkenyl, SO₂-aryl, SO₂-substituted aryl, SO₂-heteroaryl, SO₂-substituted heteroaryl, SO₂-heterocyclic, and SO₂-substituted heterocyclic, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein. Sulfonyl includes, by way of example, methyl-SO₂—, phenyl-SO₂—, and 4-methylphenyl-SO₂—.

By the term “functional groups” is meant chemical groups such as halo, hydroxyl, sulfhydryl, C1-C24 alkoxy, C2-C24 alkenyloxy, C2-C24 alkynyloxy, C5-C20 aryloxy, acyl (including C2-C24 alkylcarbonyl (—CO-alkyl) and C6-C20 arylcarbonyl (—CO-aryl)), acyloxy (—O-acyl), C2-C24 alkoxycarbonyl (—(CO)—O-alkyl), C6-C20 aryloxycarbonyl (—(CO)—O-aryl), halocarbonyl (—CO)—X where X is halo), C2-C24 alkylcarbonato (—O—(CO)—O-alkyl), C6-C20 arylcarbonato (—O—(CO)—O-aryl), carboxy (—COOH), carboxylato (—COO—), carbamoyl (—(CO)—NH2), mono-substituted C1-C24 alkylcarbamoyl (—(CO)—NH(C1-C24 alkyl)), di-substituted alkylcarbamoyl (—(CO)—N(C1-C24 alkyl)2), mono-substituted arylcarbamoyl (—(CO)—NH-aryl), thiocarbamoyl (—(CS)—NH2), carbamido (—NH—(CO)—NH2), cyano (—C≡N), isocyano (—N+=C—), cyanato (—O—C≡N), isocyanato (—O—N+≡C—), isothiocyanato (—S—C≡N), azido (—N═N+=N—), formyl (—(CO)—H), thioformyl (—(CS)—H), amino (—NH2), mono- and di-(C1-C24 alkyl)-substituted amino, mono- and di-(C5-C20 aryl)-substituted amino, C2-C24 alkylamido (—NH—(CO)-alkyl), C5-C20 arylamido (—NH—(CO)-aryl), imino (—CR═NH where R=hydrogen, C1-C24 alkyl, C5-C20 aryl, C6-C20 alkaryl, C6-C20 aralkyl, etc.), alkylimino (—CR═N(alkyl), where R=hydrogen, alkyl, aryl, alkaryl, etc.), arylimino (—CR═N(aryl), where R=hydrogen, alkyl, aryl, alkaryl, etc.), nitro (—NO2), nitroso (—NO), sulfo (—SO₂—OH), sulfonato (—SO₂—O—), C1-C24 alkylsulfanyl (—S-alkyl; also termed “alkylthio”), arylsulfanyl (—S-aryl; also termed “arylthio”), C1-C24 alkylsulfinyl (—(SO)-alkyl), C5-C20 arylsulfinyl (—(SO)-aryl), C1-C24 alkylsulfonyl (—SO2-alkyl), C5-C20 arylsulfonyl (—SO2-aryl), phosphono (—P(O)(OH)2), phosphonato (—P(O)(O—)₂), phosphinato (—P(O)(O—)), phospho (—PO₂), and phosphino (—PH₂), mono- and di-(C1-C24 alkyl)-substituted phosphino, mono- and di-(C5-C20 aryl)-substituted phosphine. In addition, the aforementioned functional groups may, if a particular group permits, be further substituted with one or more additional functional groups or with one or more hydrocarbyl moieties such as those specifically enumerated above.

By “linking” or “linker” as in “linking group,” “linker moiety,” etc., is meant a bivalent radical moiety that connects two groups via covalent bonds. Examples of such linking groups include alkylene, alkenylene, alkynylene, arylene, alkarylene, aralkylene, and linking moieties containing functional groups including, without limitation: amido (—NH—CO—), ureylene (—NH—CO—NH—), imide (—CO—NH—CO—), epoxy (—O—), epithio (—S—), epidioxy (—O—O—), carbonyldioxy (—O—CO—O—), alkyldioxy (—O—(CH2)n-O—), epoxyimino (—O—NH—), epimino (—NH—), carbonyl (—CO—), etc. Any convenient orientation and/or connections of the linkers to the linked groups may be used.

When the term “substituted” appears prior to a list of possible substituted groups, it is intended that the term apply to every member of that group. For example, the phrase “substituted alkyl and aryl” is to be interpreted as “substituted alkyl and substituted aryl.”

In addition to the disclosure herein, the term “substituted,” when used to modify a specified group or radical, can also mean that one or more hydrogen atoms of the specified group or radical are each, independently of one another, replaced with the same or different substituent groups as defined below.

In addition to the groups disclosed with respect to the individual terms herein, substituent groups for substituting for one or more hydrogens (any two hydrogens on a single carbon can be replaced with ═O, ═NR⁷⁰, ═N—OR⁷⁰, ═N₂ or ═S) on saturated carbon atoms in the specified group or radical are, unless otherwise specified, —R⁶⁰, halo, ═O, —OR⁷⁰, —SR⁷⁰, —NR⁸⁰R⁸⁰, trihalomethyl, —CN, —OCN, —SCN, —NO, —NO₂, ═N₂, —N₃, —SO₂R⁷⁰, —SO₂O⁻M⁺, —SO₂OR⁷⁰, —OSO₂R⁷⁰, —OSO₂O⁻M⁺, —OSO₂OR⁷⁰, —P(O)(O⁻)₂(M⁺)₂, —P(O)(OR⁷⁰)O⁻M⁺, —P(O)(OR⁷⁰)₂, —C(O)R⁷⁰, —C(S)R⁷⁰, —C(NR⁷⁰)R⁷⁰, —C(O)O⁻M⁺, —C(O)OR⁷⁰, —C(S)OR⁷⁰, —C(O)NR⁸⁰R⁸⁰, —C(NR⁷⁰)NR⁸⁰R⁸⁰, —OC(O)R⁷⁰, —OC(S)R⁷⁰, —OC(O)O⁻M⁺, —OC(O)OR⁷⁰, —OC(S)OR⁷⁰, —NR⁷⁰C(O)R⁷⁰, —NR⁷⁰C(S)R⁷⁰, —NR⁷⁰CO₂—M⁺, —NR⁷⁰CO₂R⁷⁰, —NR⁷⁰C(S)OR⁷⁰, —NR⁷⁰C(O)NR⁸OR⁸⁰, —NR⁷⁰C(NR⁷⁰)R⁷⁰ and —NR⁷⁰C(NR⁷⁰)NR⁸⁰R⁸⁰, where R⁶⁰ is selected from the group consisting of optionally substituted alkyl, cycloalkyl, heteroalkyl, heterocycloalkylalkyl, cycloalkylalkyl, aryl, arylalkyl, heteroaryl and heteroarylalkyl, each R⁷⁰ is independently hydrogen or R⁶⁰; each R⁸⁰ is independently R⁷⁰ or alternatively, two R⁸⁰'s, taken together with the nitrogen atom to which they are bonded, form a 5-, 6- or 7-membered heterocycloalkyl which may optionally include from 1 to 4 of the same or different additional heteroatoms selected from the group consisting of O, N and S, of which N may have —H or C₁-C₃ alkyl substitution; and each M⁺ is a counter ion with a net single positive charge. Each M⁺ may independently be, for example, an alkali ion, such as K⁺, Na⁺, Li⁺; an ammonium ion, such as ⁺N(R⁶⁰)₄; or an alkaline earth ion, such as [Ca²⁺]_{0.5}, [Mg²⁺]_{0.5}, or [Ba²⁺]_{0.5} (“subscript 0.5 means that one of the counter ions for such divalent alkali earth ions can be an ionized form of a compound of the invention and the other a typical counter ion such as chloride, or two ionized compounds disclosed herein can serve as counter ions for such divalent alkali earth ions, or a doubly ionized compound of the invention can serve as the counter ion for such divalent alkali earth ions). As specific examples, —NR⁸⁰R⁸⁰ is meant to include —NH₂, —NH-alkyl, N-pyrrolidinyl, N-piperazinyl, 4N-methyl-piperazin-1-yl and N-morpholinyl.

In addition to the disclosure herein, substituent groups for hydrogens on unsaturated carbon atoms in “substituted” alkene, alkyne, aryl and heteroaryl groups are, unless otherwise specified, —R⁶⁰, halo, —O⁻M⁺, —OR⁷⁰, —SR⁷⁰, —S⁻M⁺, —NR⁸⁰R⁸⁰, trihalomethyl, —CF₃, —CN, —OCN, —SCN, —NO, —NO₂, —N₃, —SO₂R⁷⁰, —SO₃⁻M⁺, —SO₃R⁷⁰, —OSO₂R⁷⁰, —OSO₃⁻M⁺, —OSO₃R⁷⁰, —PO₃⁻²(M⁺)₂, —P(O)(OR⁷⁰)O⁻M⁺, —P(O)(OR⁷⁰)₂, —C(O)R⁷⁰, —C(S)R⁷⁰, —C(NR⁷⁰)R⁷⁰, —CO₂⁻M⁺, —CO₂R⁷⁰, —C(S)OR⁷⁰, —C(O)NR⁸⁰R⁸⁰, —C(NR⁷⁰)NR⁸OR⁸⁰, —OC(O)R⁷⁰, —OC(S)R⁷⁰, —OCO₂⁻M⁺, —OCO₂R⁷⁰, —OC(S)OR⁷⁰, —NR⁷⁰C(O)R⁷⁰, —NR⁷⁰C(S)R⁷⁰, —NR⁷⁰CO₂—M⁺, —NR⁷⁰CO₂R⁷⁰, —NR⁷⁰C(S)OR⁷⁰, —NR⁷⁰C(O)NR⁸OR⁸⁰, —NR⁷⁰C(NR⁷⁰)R⁷⁰ and —NR⁷⁰C(NR⁷⁰)NR⁸⁰R⁸⁰, where R⁶⁰, R⁷⁰, R⁸⁰ and M⁺ are as previously defined, provided that in case of substituted alkene or alkyne, the substituents are not —O⁻M⁺, —OR⁷⁰, —SR⁷⁰, or —S⁻M⁺.

In addition to the groups disclosed with respect to the individual terms herein, substituent groups for hydrogens on nitrogen atoms in “substituted” heteroalkyl and cycloheteroalkyl groups are, unless otherwise specified, —R⁶⁰, —O⁻M⁺, —OR⁷⁰, —SR⁷⁰, —S⁻M⁺, —NR⁸⁰R⁸⁰, trihalomethyl, —CF₃, —CN, —NO, —NO₂, —S(O)₂R⁷⁰, —S(O)₂O⁻M⁺, —S(O)₂OR⁷⁰, —OS(O)₂R⁷⁰, —OS(O)₂O⁻M⁺, —OS(O)₂OR⁷⁰, —P(O)(O⁻)₂(M⁺)₂, —P(O)(OR⁷⁰)O⁻M⁺, —P(O)(OR⁷⁰)(OR⁷⁰), —C(O)R⁷⁰, —C(S)R⁷⁰, —C(NR⁷⁰)R⁷⁰, —C(O)OR⁷⁰, —C(S)OR⁷⁰, —C(O)NR⁸OR⁸⁰, —C(NR⁷⁰)NR⁸OR⁸⁰, —OC(O)R⁷⁰, —OC(S)R⁷⁰, —OC(O)OR⁷⁰, —OC(S)OR⁷⁰, —NR⁷⁰C(O)R⁷⁰, —NR⁷⁰C(S)R⁷⁰, —NR⁷⁰C(O)OR⁷⁰, —NR⁷⁰C(S)OR⁷⁰, —NR⁷⁰C(O)NR⁸OR⁸⁰, —NR⁷⁰C(NR⁷⁰)R⁷⁰ and —NR⁷⁰C(NR⁷⁰)NR⁸⁰R⁸⁰, where R⁶⁰, R⁷⁰, R⁸⁰ and M⁺ are as previously defined.

In addition to the disclosure herein, in a certain embodiment, a group that is substituted has 1, 2, 3, or 4 substituents, 1, 2, or 3 substituents, 1 or 2 substituents, or 1 substituent.

Unless indicated otherwise, the nomenclature of substituents that are not explicitly defined herein are arrived at by naming the terminal portion of the functionality followed by the adjacent functionality toward the point of attachment. For example, the substituent “arylalkyloxycarbonyl” refers to the group (aryl)-(alkyl)-O—C(O)—.

As to any of the groups disclosed herein which contain one or more substituents, it is understood, of course, that such groups do not contain any substitution or substitution patterns which are sterically impractical and/or synthetically non-feasible. In addition, the subject compounds include all stereochemical isomers arising from the substitution of these compounds.

In certain embodiments, a substituent may contribute to optical isomerism and/or stereo isomerism of a compound. Salts, solvates, hydrates, and prodrug forms of a compound are also of interest. All such forms are embraced by the present disclosure.

Thus the compounds described herein include salts, solvates, hydrates, prodrug and isomer forms thereof, including the pharmaceutically acceptable salts, solvates, hydrates, prodrugs and isomers thereof. In certain embodiments, a compound may be a metabolized into a pharmaceutically active derivative.

As used herein and unless otherwise specified, the term “prodrug” means a derivative of a compound that can hydrolyze, oxidize, or otherwise react under biological conditions (in vitro or in vivo) to provide an active form of a compound. Examples of prodrugs include, but are not limited to, derivatives and metabolites that include biohydrolyzable moieties such as biohydrolyzable amides, biohydrolyzable esters, biohydrolyzable carbamates, biohydrolyzable carbonates, biohydrolyzable ureides, and biohydrolyzable phosphate analogues. Prodrugs can typically be prepared using well known methods, such as those described by 1 Burger's Medicinal Chemistry and Drug Discovery, 172-178, 949-982 (Manfred E. Wolff ed., 5th ed. 1995). Various forms of prodrugs are well known in the art and are described in:
a) The Practice of Medicinal Chemistry, Camille G. Wermuth et al., Ch. 31, (Academic Press, 1996);
b) Design of Prodrugs, edited by H. Bundgaard, (Elsevier, 1985);
c) A Textbook of Drug Design and Development, P. Krogsgaard-Larson and H. Bundgaard, eds. Ch. 5, 113-191 (Harwood Academic Publishers, 1991); and
d) Hydrolysis in Drug and Prodrug Metabolism, Bernard Testa and Joachim M. Mayer, (Wiley-VCH, 2003).

In some embodiments, compounds of the described herein exist as stereoisomers, wherein asymmetric or chiral centers are present. Stereoisomers are designated (R) or (S) depending on the configuration of substituents around the chiral carbon atom. The term (R) and (S) used herein are configurations as defined in IUPAC 1974 Recommendations for Section E, Fundamental Stereochemistry, Pure Appl. Chem., (1976), 45:13-30, hereby incorporated by reference. The embodiments described herein specifically includes the various stereoisomers and mixtures thereof. Stereoisomers include enantiomers, diastereomers, and mixtures of enantiomers or diastereomers. In some embodiments, individual stereoisomers of compounds are prepared synthetically from commercially available starting materials which contain asymmetric or chiral centers or by preparation of racemic mixtures followed by resolution. These methods of resolution are exemplified by (1) attachment of a mixture of enantiomers to a chiral axillary, separation of the resulting mixture of diastereomers by recrystallization or chromatography and liberation of the optically pure product from the auxiliary or (2) direct separation of the mixture of optical enantiomers on chiral chromatographic column.

The term “isotopic variant” of a compound refers to a compound that contains an unnatural proportion of an isotope at one or more of the atoms that constitute such a compound. In certain embodiments, an isotopic variant of a compound contains unnatural proportions of one or more isotopes, including, but not limited to, hydrogen (¹H), deuterium (²H), tritium (³H), carbon-11 (¹¹C), carbon-12 (¹²C), carbon-13 (¹³C), carbon-14 (¹⁴C), nitrogen-13 (¹³N), nitrogen-14 (¹⁴N), nitrogen-15 (¹⁵N), oxygen-14 (¹⁴O), oxygen-15 (¹⁵O), oxygen-16 (¹⁶O), oxygen-17 (¹⁷O), oxygen-18 (¹⁸O), fluorine-17 (¹⁷F), fluorine-18 (¹⁸F), fluorine-19 (¹⁹F), phosphorus-31 (³¹P), phosphorus-32 (³²P), phosphorus-33 (³³P), sulfur-32 (³²S), sulfur-33 (³³S), sulfur-34 (³⁴S), sulfur-35 (³⁵S), sulfur-36 (³⁶S), chlorine-35 (³⁵Cl), chlorine-36 (³⁶Cl), chlorine-37 (³⁷Cl), bromine-79 (⁷⁹Br), bromine-81 (⁸¹Br), iodine-123 (¹²³I), iodine-125 (¹²⁵I), iodine-127 (¹²⁷I), iodine-129 (¹²⁹I), and iodine-131 (¹³¹I). In certain embodiments, an isotopic variant of a compound is in a stable form, that is, non-radioactive. In certain embodiments, an isotopic variant of a compound contains unnatural proportions of one or more isotopes, including, but not limited to, hydrogen (¹H), deuterium (²H), carbon-12 (¹²C), carbon-13 (¹³C), nitrogen-14 (¹⁴N), nitrogen-15 (¹⁵N), oxygen-16 (¹⁶O), oxygen-17 (¹⁷O), oxygen-18 (¹⁸O), fluorine-17 (¹⁷F), phosphorus-31 (³¹P), sulfur-32 (³²S), sulfur-33 (³³S), sulfur-34 (³⁴S), sulfur-36 (³⁶S), chlorine-35 (³⁵Cl), chlorine-37 (³⁷Cl), bromine-79 (⁷⁹Br), bromine-81 (⁸¹Br), and iodine-127 (¹²⁷I). In certain embodiments, an isotopic variant of a compound is in an unstable form, that is, radioactive. In certain embodiments, an isotopic variant of a compound contains unnatural proportions of one or more isotopes, including, but not limited to, tritium (³H), carbon-11 (¹¹C), carbon-14 (¹⁴C), nitrogen-13 (¹³N), oxygen-14 (¹⁴O), oxygen-15 (¹⁵O), fluorine-18 (¹⁸F), phosphorus-32 (³²P), phosphorus-33 (³³P), sulfur-35 (³⁵S), chlorine-36 (³⁶Cl), iodine-123 (¹²³I), iodine-125 (¹²⁵I), iodine-129 (¹²⁹I), and iodine-131 (¹³¹I). It will be understood that, in a compound as provided herein, any hydrogen can be ²H, as example, or any carbon can be ¹³C, as example, or any nitrogen can be ¹⁵N, as example, or any oxygen can be ¹⁸O, as example, or any fluorine can be ¹⁸F, as example, where feasible according to the judgment of one of ordinary skill in the art.

The term “solvate” refers to a complex or aggregate formed by one or more molecules of a solute, e.g., a compound provided herein, and one or more molecules of a solvent, which present in stoichiometric or non-stoichiometric amount. Suitable solvents include, but are not limited to, water, methanol, ethanol, n-propanol, isopropanol, and acetic acid. In certain embodiments, the solvent is pharmaceutically acceptable. In one embodiment, the complex or aggregate is in a crystalline form. In another embodiment, the complex or aggregate is in a noncrystalline form. Where the solvent is water, the solvate is a hydrate. Examples of hydrates include, but are not limited to, a hemihydrate, monohydrate, dihydrate, trihydrate, tetrahydrate, and pentahydrate.

The phrase “an enantiomer, a mixture of enantiomers, a mixture of two or more diastereomers, a tautomer, a mixture of two or more tautomers, or an isotopic variant thereof; or a pharmaceutically acceptable salt, solvate, hydrate, or prodrug thereof” has the same meaning as the phrase “(i) an enantiomer, a mixture of enantiomers, a mixture of two or more diastereomers, a tautomer, a mixture of two or more tautomers, or an isotopic variant of the compound referenced therein; or (ii) a pharmaceutically acceptable salt, solvate, hydrate, or prodrug of the compound referenced therein, or (iii) a pharmaceutically acceptable salt, solvate, hydrate, or prodrug of an enantiomer, a mixture of enantiomers, a mixture of two or more diastereomers, a tautomer, a mixture of two or more tautomers, or an isotopic variant of the compound referenced therein.”

Definitions of other terms and concepts appear throughout the detailed description.

### DETAILED DESCRIPTION

Provided herein are compounds, compositions and methods for inhibition of a herpesvirus. In some cases, the subject compounds inhibit a herpesvirus in a cell. Also provided are compounds, compositions and methods for treating a herpesvirus in an individual. In some cases, the methods include administering to an individual a therapeutically effective amount of a subject compound to treat the individual for the herpesvirus. In certain embodiments, the compounds disclosed herein are cytomegalovirus (CMV) inhibitors. In certain embodiments, the compounds disclosed herein are human cytomegalovirus (CMV) inhibitors. In certain embodiments, the subject compounds possess antiviral activity. In some cases, the subject compounds possess CMV DNA polymerase activity. In some cases, the subject compounds possess both antiviral and CMV DNA polymerase activity.

These compounds and methods find use in a variety of applications in which inhibition of a herpesvirus is desired.

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

While the apparatus and method has or will be described for the sake of grammatical fluidity with functional explanations, it is to be expressly understood that the claims, unless expressly formulated under 35 U.S.C. § 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 U.S.C. § 112 are to be accorded full statutory equivalents under 35 U.S.C. § 112.

Compounds

As summarized above, provided herein are compounds useful as inhibitors of a herpesvirus. In certain embodiments, the compounds disclosed herein are cytomegalovirus inhibitors. In certain embodiments, the compounds disclosed herein are human cytomegalovirus inhibitors. In certain embodiments, the subject compounds possess antiviral activity. In some cases, the subject compounds possess CMV DNA polymerase activity. In some cases, the subject compounds possess both antiviral and CMV DNA polymerase activity.

The subject compounds can include a core structure based on a heteroaryl ring system, e.g., a pyridopyrazine, a pyrido[2,3-b]pyrazin-6(5H)-one, a naphthyridine, a 1,5-naphthyridin-2(1H)-one, which is substituted with a heterocyclic group. The core heteroaryl ring system can be further substituted. Exemplary herpesvirus inhibitor compounds of interest are set forth in any of formulae (I)-(V) and the any of compounds 1-46, as illustrated in Table 1.

In some cases, the subject compound is of formula (I)

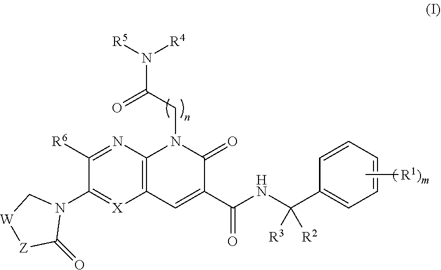

wherein:

each R¹ is independently selected from H, alkyl, substituted alkyl, halogen, nitrile, nitro, trifluoromethyl, hydroxyl, amino, and substituted amino;

R² and R³ are each independently selected from H, alkyl and substituted alkyl;

R⁴ and R⁵ are each independently selected from H, alkyl, substituted alkyl, cycloalkyl, substituted cycloalkyl, cycloalkenyl, substituted cycloalkenyl, aryl, substituted aryl, heterocycle, substituted heterocycle, heteroaryl, and substituted heteroaryl, or

R⁴ and R⁵ together with the nitrogen to which they are attached form a cycle selected from heteroaryl, substituted heteroaryl, heterocycle, and substituted heterocycle, (e.g., azetidine, spiroazetidine);

X is CR⁷ or N;

R⁶ and R⁷ are each independently selected from H, alkyl, substituted alkyl, alkoxy, substituted alkoxy, halogen, nitrile, nitro, trifluoromethyl, hydroxyl, amino, substituted amino, cycloalkyl, substituted cycloalkyl, cycloalkenyl, substituted cycloalkenyl, aryl, substituted aryl, heterocycle, substituted heterocycle, heteroaryl, and substituted heteroaryl;

Z is selected from NR⁸, O, and C(R⁹)₂;

W is selected from C(R^{9a})₂, and C(O);

R⁸ is selected from H, alkyl, substituted alkyl, cycloalkyl, substituted cycloalkyl, cycloalkenyl, substituted cycloalkenyl, aryl, substituted aryl, heterocycle, substituted heterocycle, heteroaryl, and substituted heteroaryl;

each R⁹ and R^{9a} is independently selected from H, alkyl, substituted alkyl, alkoxy, substituted alkoxy, halogen, nitrile, nitro, trifluoromethyl, hydroxyl, amino, substituted amino, cycloalkyl, substituted cycloalkyl, cycloalkenyl, substituted cycloalkenyl, aryl, substituted aryl, heterocycle, substituted heterocycle, heteroaryl, and substituted heteroaryl;

n is an integer from 1 to 6; and

m is an integer from 1 to 5;

or a pharmaceutically acceptable salt, solvate, or hydrate thereof.

In certain embodiments of formula (I), R² and R³ are each H. In certain cases, at least one of R² or R³ is selected from alkyl and substituted alkyl. In certain cases, both R² and R³ are alkyl or substituted alkyl. In certain cases, both R² and R³ are C₁₋₃ alkyl.

In certain cases, n is an integer from 1-5, such as 1-4, 1-3 or 1-2. In certain cases, n is 1.

In certain embodiments of formula (I), at least one R¹ group is selected from, alkyl, substituted alkyl, halogen, nitrile, nitro, trifluoromethyl, hydroxyl, amino, and substituted amino. In some cases, at least one R¹ group is C₁₋₆alkyl. In some cases, at least one R¹ group is methyl. In some cases, at least one R¹ group is halogen. In certain cases, at least one R¹ group is selected from F and Cl. In certain cases, at least one R¹ group is nitrile. In certain cases, at least one R¹ group is trifluoromethyl. In certain cases m is 1-4, such as 1-3, or 1-2. In some cases, m is 2, such that the compound of formula (I) includes two R¹ groups. In some cases m is 1, such that the compound of formula (I) includes one R¹ group.

In certain embodiments of formula (I), R⁴ and R⁵ are each independently H. In certain cases, at least one of R⁴ or R⁵ is selected from alkyl and substituted alkyl. In certain cases, at least one of R⁴ or R⁵ is selected from, cycloalkyl, and substituted cycloalkyl. In certain cases, at least one of R⁴ or R⁵ is selected from, cycloalkenyl, and substituted cycloalkenyl. In certain cases, at least one of R⁴ or R⁵ is selected from aryl, and substituted aryl. In certain cases, at least one of R⁴ or R⁵ is selected from, heterocycle, and substituted heterocycle. In certain other cases, at least one of R⁴ or R⁵ is selected from, heteroaryl, and substituted heteroaryl.

In certain cases of formula (I), R⁴ and R⁵ together with the nitrogen to which they are attached form a cycle selected from heteroaryl, substituted heteroaryl, heterocycle, and substituted heterocycle. In certain cases, R⁴ and R⁵ together with the nitrogen to which they are attached form a heterocycle, wherein the heterocycle is optionally substituted. In certain cases, R⁴ and R⁵ together with the nitrogen to which they are attached form a cycle selected from azetidine, and spiroazetidine, wherein the azetidine and the spiroazetidine are optionally substituted. In certain cases, R⁴ and R⁵ together with the nitrogen to which they are attached form a substituted azetidine. In certain cases, R⁴ and R⁵ together with the nitrogen to which they are attached form a spiroazetidine.

In certain cases of formula (I), R⁴ and R⁵ together with the nitrogen to which they are attached form a cycle selected from one of the following structures:

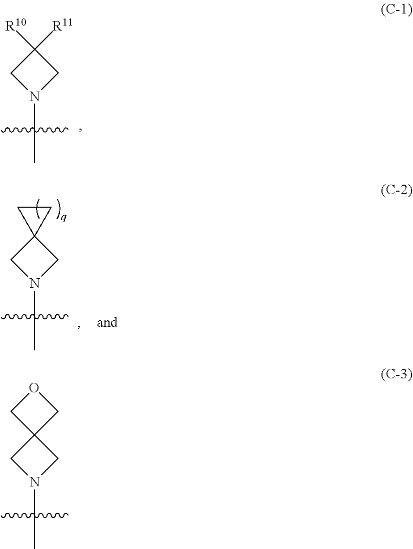

wherein:

R¹⁰ and R¹¹ are each independently selected from C₁₋₆ alkyl, hydroxyl, alkoxy, —(CH₂)ₚOH, and halogen;

p is an integer from 1 to 6; and

q is an integer from 1 to 4.

In certain cases of formula (I), R⁴ and R⁵ together with the nitrogen to which they are attached form a cycle of formula (C-1). In certain cases of the formula (C-1), R¹⁰ and R¹¹ are both C₁₋₆ alkyl. In certain cases, both R¹⁰ and R¹¹ are methyl. In certain cases of the formula (C-1), R¹⁰ is a halogen and R¹¹ is C₁₋₆ alkyl. In certain cases, R¹⁰ is F, and R¹¹ is methyl. In certain cases of the formula (C-1), both R¹⁰ and R¹¹ are halogen. In certain cases, both R¹⁰ and R¹¹ are F.

In certain cases of formula (I), R⁴ and R⁵ together with the nitrogen to which they are attached form a cycle of formula (C-2). In certain cases of the formula (C-2), q is 1.

In certain cases of formula (I), R⁴ and R⁵ together with the nitrogen to which they are attached form a cycle of formula (C-3).

In certain embodiments of formula (I), X is N. In certain cases, X is CR⁷. In certain cases, X is CR⁷, and R⁷ is H. In certain cases, R⁷ is a substituent selected from alkyl, substituted alkyl, alkoxy, substituted alkoxy, halogen, nitrile, nitro, trifluoromethyl, hydroxyl, amino, substituted amino, cycloalkyl, substituted cycloalkyl, cycloalkenyl, substituted cycloalkenyl, aryl, substituted aryl, heterocycle, substituted heterocycle, heteroaryl, and substituted heteroaryl. In certain cases, R⁷ is C₁₋₆ alkyl. In certain cases, R⁷ is alkoxy. In certain cases, R⁷ is halogen. In certain cases, R⁷ is nitrile. In certain cases, R⁷ is trifluoromethyl. In some cases, R⁷ is hydroxyl. In some cases, R⁷ is amino or substituted amino. In some cases R⁷ is a cyclic group selected from cycloalkyl, substituted cycloalkyl, cycloalkenyl, substituted cycloalkenyl, aryl, substituted aryl, heterocycle, substituted heterocycle, heteroaryl, and substituted heteroaryl.

In certain embodiments of formula (I), R⁶ is H. In certain cases R⁶ is selected from, alkyl, substituted alkyl, alkoxy, substituted alkoxy, halogen, nitrile, nitro, trifluoromethyl, hydroxyl, amino, substituted amino, cycloalkyl, substituted cycloalkyl, cycloalkenyl, substituted cycloalkenyl, aryl, substituted aryl, heterocycle, substituted heterocycle, heteroaryl, and substituted heteroaryl. In certain cases, R⁶ is C₁₋₆ alkyl. In certain cases, R⁶ is alkoxy. In certain cases, R⁶ is halogen. In certain cases, R⁶ is nitrile. In certain cases, R⁶ is trifluoromethyl. In some cases, R⁶ is hydroxyl. In some cases, R⁶ is amino or substituted amino. In some cases R⁶ is a cyclic group selected from cycloalkyl, substituted cycloalkyl, cycloalkenyl, substituted cycloalkenyl, aryl, substituted aryl, heterocycle, substituted heterocycle, heteroaryl, and substituted heteroaryl.

In some embodiments, the compound of formula (I) is of the formula (II):

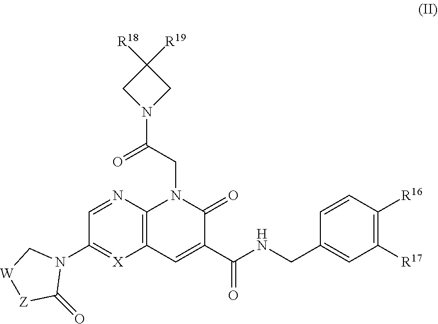

wherein:

X is CR⁷ or N;

R⁷ is selected from H, alkyl, substituted alkyl, alkoxy, substituted alkoxy, halogen, nitrile, nitro, trifluoromethyl, hydroxyl, amino, substituted amino, cycloalkyl, substituted cycloalkyl, cycloalkenyl, substituted cycloalkenyl, aryl, substituted aryl, heterocycle, substituted heterocycle, heteroaryl, and substituted heteroaryl;

Z is selected from NR⁸, O, and C(R⁹)₂;

W is selected from C(R^{9a})₂, and C(O);

R⁸ is selected from H, alkyl, substituted alkyl, cycloalkyl, substituted cycloalkyl, cycloalkenyl, substituted cycloalkenyl, aryl, substituted aryl, heterocycle, substituted heterocycle, heteroaryl, and substituted heteroaryl;

each R⁹ and R^{9a} is independently selected from H, alkyl, substituted alkyl, alkoxy, substituted alkoxy, halogen, nitrile, nitro, trifluoromethyl, hydroxyl, amino, substituted amino, cycloalkyl, substituted cycloalkyl, cycloalkenyl, substituted cycloalkenyl, aryl, substituted aryl, heterocycle, substituted heterocycle, heteroaryl, and substituted heteroaryl;

R¹⁶ is selected from C₁₋₆ alkyl, halogen, nitrile, and trifluoromethyl;

R¹⁷ is selected from hydrogen, and halogen;

R¹⁸ and R¹⁹ are each independently selected from C₁₋₆ alkyl, hydroxyl, alkoxy, —(CH₂)ₚOH, and halogen, or

R¹⁸ and R¹⁹ together with the carbon to which they are attached form a 3-6 membered cycle selected from cycloalkyl, substituted cycloalkyl, cycloalkenyl, substituted cycloalkenyl, aryl, substituted aryl, heteroaryl, substituted heteroaryl, heterocycle, and substituted heterocycle; and

p is an integer from 1 to 6.

In certain embodiments of formula (II), X is N. In certain cases, X is CR⁷. In certain cases, X is CR⁷, and R⁷ is H. In certain cases, R⁷ is a substituent selected from alkyl, substituted alkyl, alkoxy, substituted alkoxy, halogen, nitrile, nitro, trifluoromethyl, hydroxyl, amino, substituted amino, cycloalkyl, substituted cycloalkyl, cycloalkenyl, substituted cycloalkenyl, aryl, substituted aryl, heterocycle, substituted heterocycle, heteroaryl, and substituted heteroaryl. In certain cases, R⁷ is C₁₋₆ alkyl. In certain cases, R⁷ is alkoxy. In certain cases, R⁷ is halogen. In certain cases, R⁷ is nitrile. In certain cases, R⁷ is trifluoromethyl. In some cases, R⁷ is hydroxyl. In some cases, R⁷ is amino or substituted amino. In some cases R⁷ is a cyclic group selected from cycloalkyl, substituted cycloalkyl, cycloalkenyl, substituted cycloalkenyl, aryl, substituted aryl, heterocycle, substituted heterocycle, heteroaryl, and substituted heteroaryl.

In certain embodiments of formula (II) R¹⁸ and R¹⁹ are independently selected from C₁₋₆ alkyl, and halogen. In certain cases of the formula (II), R¹⁸ and R¹⁹ are both C₁₋₆ alkyl. In certain cases, both R¹⁸ and R¹⁹ are methyl. In certain cases of the formula (II), R¹⁸ is a halogen and R¹⁹ is C₁₋₆ alkyl. In certain cases, R¹⁸ is F, and R¹⁹ is methyl. In certain cases of the formula (II), both R¹⁸ and R¹⁹ are halogen. In certain cases, both R¹⁸ and R¹⁹ are F.

In certain other embodiments of formula (II), R¹⁸ and R¹⁹ together with the carbon to which they are attached form a cycle selected from C₃₋₆cycloalkyl, substituted C₃₋₆cycloalkyl, C₃₋₆heterocycle, and substituted C₃₋₆heterocycle. In certain cases of the formula (II), form a cycle selected from C₃₋₆cycloalkyl and substituted C₃₋₆cycloalky. In certain cases, the cycle is a cyclopropyl, or a substituted cyclopropyl. In certain cases of the formula (II), form a cycle selected from C₃₋₆heterocycle and substituted C₃₋₆heterocycle. In certain cases, the cycle is an oxetane or a substituted oxetane.

In certain embodiments of formula (II), R¹⁶ is C₁₋₆ alkyl. In some cases, R¹⁶ is methyl. In some cases, R¹⁶ is selected from F, Cl, methyl and nitrile. In some cases, R¹⁶ is halogen. In certain cases, the halogen is selected from F and Cl. In some cases R¹⁶ is Cl. In certain cases, R¹⁶ is nitrile. In certain cases, R¹⁶ is trifluoromethyl.

In certain embodiments of formula (II), R¹⁷ is hydrogen. In certain other embodiments, R¹⁷ is halogen. In some cases, the halogen is F.

In certain embodiments of formula (II), R¹⁶ is C₁₋₆ alkyl and R¹⁷ is H. In some cases, R¹⁶ is methyl and R¹⁷ is H. In some cases, R¹⁶ is halogen and R¹⁷ is H. In certain cases, the halogen is selected from F and Cl. In certain cases, R¹⁶ is nitrile and R¹⁷ is H. In certain cases, R¹⁶ is trifluoromethyl and R¹⁷ is H. In certain other cases, R¹⁶ is C₁₋₆ alkyl and R¹⁷ is halogen. In some cases, R¹⁶ is methyl and R¹⁷ is F. In some cases, both R¹⁶ and R¹⁷ are halogen. In certain cases, the halogen is selected from F and Cl. In certain cases, R¹⁶ is nitrile and R¹⁷ is halogen. In certain cases, R¹⁶ is trifluoromethyl and R¹⁷ is halogen.

In certain embodiments of formula (I) or (II), Z is C(R⁹)₂, where each R⁹ is independently selected from H, alkyl, substituted alkyl, alkoxy, substituted alkoxy, halogen, nitrile, nitro, trifluoromethyl, hydroxyl, amino, substituted amino, cycloalkyl, substituted cycloalkyl, cycloalkenyl, substituted cycloalkenyl, aryl, substituted aryl, heterocycle, substituted heterocycle, heteroaryl, and substituted heteroaryl. In certain cases, at least one R⁹ is selected from alkyl, and substituted alkyl. In certain cases, the alkyl is C₁₋₆ alkyl, or substituted C₁₋₆ alkyl. In certain cases, at least one R⁹ is selected from cycloalkyl, and substituted cycloalkyl. In certain cases, at least one R⁹ is selected from C₃₋₆cycloalkyl, and substituted C₃₋₆cycloalkyl. In certain cases, at least one R⁹ is selected from hydroxyl and alkoxy. In certain cases, at least one R⁹ is selected from amino, and substituted amino. In certain cases, at least one R⁹ is selected from aryl and substituted aryl. In certain cases, at least one R⁹ is selected from heterocycle and substituted heterocycle. In certain cases, at least one R⁹ is selected from heteroaryl and substituted heteroaryl.

In certain embodiments of formula (I) or (II), Z is C(R⁹)₂, and both R⁹ groups are H, such than Z is CH₂.

In certain embodiments of formula (I) or (II), Z is C(R⁹)₂, where at least one R⁹ is selected from C₁₋₆ alkyl, C₃₋₆cycloalkyl, substituted C₃₋₆cycloalkyl, hydroxyl, alkoxy, —(CH₂)ₚOR¹², —(CH₂)ₚCO₂R¹², and —(CH₂)ₚN(R¹³)₂, where R¹² is selected from H, alkyl and substituted alkyl; each R¹³ is independently selected from H, C₁₋₆ alkyl, substituted C₁₋₆ alkyl, S(O)₂R¹⁴, and C(O)R¹⁴, or two R¹³ together with the nitrogen to which they are attached form a cycle selected from heteroaryl, substituted heteroaryl, heterocycle, and substituted heterocycle; R¹⁴ is selected from C₁₋₆ alkyl, and substituted C₁₋₆ alkyl; and p is an integer from 1 to 6. In certain cases, at least one R⁹ is C₁₋₆ alkyl, e.g., methyl, ethyl, propyl, butyl, pentyl or hexyl. In certain cases, at least one R⁹ is, C₃₋₆cycloalkyl, e.g., cyclopropyl, cyclobutyl, cyclopentyl or cyclohexyl. In certain cases, at least one R⁹ is, substituted C₃₋₆cycloalkyl. In certain cases, at least one R⁹ is, hydroxy. In certain cases, at least one R⁹ is, alkoxy. In certain cases, at least one R⁹ is —(CH₂)ₚOR¹², and R¹² is selected from H, alkyl and substituted alkyl. In certain cases, at least one R⁹ is —(CH₂)ₚCO₂R¹², R¹² is selected from H, alkyl and substituted alkyl and p is an integer from 1 to 6. In certain cases, at least one R⁹ is —(CH₂)ₚN(R¹³)₂, where each R¹³ are independently or together as defined above. In certain cases, R⁹ is —(CH₂)ₚN(R¹³)₂, and each R¹³ is independently selected from H, C₁₋₆ alkyl, substituted C₁₋₆ alkyl, S(O)₂R¹⁴, and C(O)R¹⁴, and R¹⁴ is C₁₋₆ alkyl, or substituted C₁₋₆ alkyl. In other cases, R⁹ is —(CH₂)ₚN(R¹³)₂, and the two R¹³ groups together with the nitrogen to which they are attached form a cycle selected from heteroaryl, substituted heteroaryl, heterocycle, and substituted heterocycle.

In certain embodiments of formula (I) or (II), Z is C(R⁹)H, wherein R⁹ is selected from H, C₁₋₆alkyl, C₃₋₆cycloalkyl, substituted C₃₋₆cycloalkyl, hydroxyl, alkoxy, —(CH₂)ₚOR¹², —(CH₂)ₚCO₂R¹², and —(CH₂)ₚN(R¹³)₂; R¹² is selected from H, alkyl and substituted alkyl; each R¹³ is independently selected from H, C₁₋₆alkyl, substituted C₁₋₆alkyl, S(O)₂R¹⁴, and C(O)R¹⁴, or two R¹³ together with the nitrogen to which they are attached form a cycle selected from heteroaryl, substituted heteroaryl, heterocycle, and substituted heterocycle; R¹⁴ is selected from C₁₋₆ alkyl, and substituted C₁₋₆ alkyl; and p is an integer from 1 to 6. In certain cases, Z is C(R⁹)H, and R⁹ is H. In certain cases, Z is C(R⁹)H and R⁹ is C₁₋₆ alkyl, e.g., methyl, ethyl, propyl, butyl, pentyl or hexyl. In certain cases, Z is C(R⁹)H and R⁹ is, C₃₋₆cycloalkyl, e.g., cyclopropyl, cyclobutyl, cyclopentyl or cyclohexyl. In certain cases, Z is C(R⁹)H and R⁹ is, substituted C₃₋₆cycloalkyl. In certain cases, Z is C(R⁹)H and R⁹ is, hydroxy. In certain cases, Z is C(R⁹)H and R⁹ is, alkoxy. In certain cases, Z is C(R⁹)H, R⁹ is —(CH₂)ₚOR¹², and R¹² is selected from H, alkyl and substituted alkyl. In certain cases, Z is C(R⁹)H, R⁹ is —(CH₂)ₚCO₂R¹², R¹² is selected from H, alkyl and substituted alkyl and p is an integer from 1 to 6. In certain cases, Z is C(R⁹)H, R⁹ is —(CH₂)ₚN(R¹³)₂, where each R¹³ are independently or together as defined above. In certain cases, Z is C(R⁹)H, R⁹ is —(CH₂)ₚN(R¹³)₂, and each R¹³ is independently selected from H, C₁₋₆ alkyl, substituted C₁₋₆ alkyl, S(O)₂R¹⁴, and C(O)R¹⁴, and R¹⁴ is C₁₋₆ alkyl, or substituted C₁₋₆ alkyl. In other cases, Z is C(R⁹)H, R⁹ is —(CH₂)ₚN(R¹³)₂, and the two R¹³ groups together with the nitrogen to which they are attached form a cycle selected from heteroaryl, substituted heteroaryl, heterocycle, and substituted heterocycle.

In certain embodiments of formula (I) or (II), Z is CH₂. In certain embodiments of formula (I) or (II), Z is C(R⁹)H, and R⁹ is methyl.

In certain embodiments of formula (I) or (II) Z is NR⁸, wherein R⁸ is selected from H, alkyl, substituted alkyl, cycloalkyl, substituted cycloalkyl, cycloalkenyl, substituted cycloalkenyl, aryl, substituted aryl, heterocycle, substituted heterocycle, heteroaryl, and substituted heteroaryl. In certain cases, R⁸ is H. In certain cases, R⁸ is selected from alkyl, and substituted alkyl. In certain cases, R⁸ is selected from cycloalkyl, and substituted cycloalkyl. In certain cases, R⁸ is selected from cycloalkenyl, and substituted cycloalkenyl. In certain cases, R⁸ is selected from aryl and substituted aryl. In certain cases R⁸ is selected from heterocycle and substituted heterocycle. In certain cases, R⁸ is selected from heteroaryl and substituted heteroaryl.

In certain embodiments of formula (I) or (II) Z is NR⁸, wherein R⁸ is selected from H, C₁₋₆ alkyl, substituted C₁₋₆ alkyl, —C(O)(CH₂)ₚN(R¹³)₂, C₃₋₆cycloalkyl, substituted C₃₋₆cycloalkyl, C₃₋₆heterocycle, and substituted C₃₋₆heterocycle; each R¹³ is independently selected from H, C₁₋₆ alkyl, substituted C₁₋₆ alkyl, S(O)₂R¹⁴, and C(O)R¹⁴, or two R¹³ together with the nitrogen to which they are attached form a cycle selected from heteroaryl, substituted heteroaryl, heterocycle, and substituted heterocycle; R¹⁴ is selected from C₁₋₆ alkyl, and substituted C₁₋₆ alkyl; and p is an integer from 1 to 6. In certain cases, R⁸ is H. In certain cases, R⁸ is C₁₋₆ alkyl, e.g., methyl, ethyl, propyl, butyl, pentyl or hexyl. In certain cases, R⁸ is, substituted C₁₋₆ alkyl (e.g., as described herein). In certain cases, R⁸ is, —C(O)(CH₂)ₚN(R¹³)₂, where each R¹³ are independently or together as defined above. In certain cases, R⁸ is, —C(O)(CH₂)ₚN(R¹³)₂, and each R¹³ is independently selected from H, C₁₋₆ alkyl, substituted C₁₋₆ alkyl, S(O)₂R¹⁴, and C(O)R¹⁴, and R¹⁴ is C₁₋₆ alkyl, or substituted C₁₋₆ alkyl. In other cases, R⁸ is, —C(O)(CH₂)ₚN(R¹³)₂, and the two R¹³ groups together with the nitrogen to which they are attached form a cycle selected from heteroaryl, substituted heteroaryl, heterocycle, and substituted heterocycle. In certain cases, R⁸ is C₃₋₆cycloalkyl. In certain cases, R⁸ is substituted C₃₋₆cycloalkyl. In certain cases, R⁸ is C₃₋₆heterocycle. In certain other cases, R⁸ is substituted C₃₋₆heterocycle.

In certain embodiments of formula (I) or (II) Z is NR⁸, and R⁸ is C₁₋₆ alkyl substituted with one or two R¹⁵ groups, wherein each R¹⁵ is independently selected from heterocycle, substituted heterocycle, CH₃, CH₂F, CHF₂, CF₃, CN, C(NH)NH₂, OH, CH₂OH, CO₂H, N(R¹³)₂, CH₂N(R¹³)₂, C(O)N(R¹³)₂, C(O)NHCN, and S(O)₂N(R¹³)₂. In certain cases, R⁸ is C₁₋₃ alkyl (e.g., methyl, ethyl or propyl) substituted with one or two R¹⁵ groups. In certain cases R¹⁵ is selected from heterocycle and substituted heterocycle. In some cases, R¹⁵ is CHF₂. In some cases, R¹⁵ is CN. In some cases, R¹⁵ is C(NH)NH₂. In some cases, R¹⁵ is OH. In some cases, R¹⁵ is CH₂OH. In some cases, R¹⁵ is CO₂H. In some cases, R¹⁵ is N(R¹³)₂. In some cases, R¹⁵ is CH₂N(R¹³)₂. In some cases, R¹⁵ is C(O)N(R¹³)₂. In some cases, R¹⁵ is C(O)NHCN. In some other cases, R¹⁵ is S(O)₂N(R¹³)₂.

In certain embodiments of formula (I) or (II) Z is NR⁸, wherein R⁸ is selected from H, C₁₋₃ alkyl, —CH₂(R¹⁵), —CH(R¹⁵)₂, —C(O)(CH₂)ₚN(R¹³)₂, and cyclopropyl; each R¹⁵ is independently selected from heterocycle, substituted heterocycle, CH₃, CH₂F, CHF₂, CF₃, CN, C(NH)NH₂, OH, CH₂OH, CO₂H, N(R¹³)₂, CH₂N(R¹³)₂, C(O)N(R¹³)₂, C(O)NHCN, S(O)₂R¹⁴ and S(O)₂N(R¹³)₂; and each R¹³ is independently selected from H, C₁₋₆ alkyl, substituted C₁₋₆ alkyl, S(O)₂R¹⁴, and C(O)R¹⁴, or two R¹³ together with the nitrogen to which they are attached form a cycle selected from heteroaryl, substituted heteroaryl, heterocycle, and substituted heterocycle. In certain cases, R⁸ is C₁₋₃ alkyl. In certain cases, R⁸ is —CH₂(R¹⁵), where R¹⁵ is as defined above. In certain cases, R⁸ is —CH₂(R¹⁵), where R¹⁵ is CHF₂. In certain cases, R⁸ is —CH₂(R¹⁵), where R¹⁵ is CN. In certain cases, R⁸ is —CH₂(R¹⁵), where R¹⁵ is C(NH)NH₂. In certain cases, R⁸ is —CH₂(R¹⁵), where R¹⁵ is OH. In certain cases, R⁸ is —CH₂(R¹⁵), where R¹⁵ is CH₂OH. In certain cases, R⁸ is —CH₂(R¹⁵), where R¹⁵ is CO₂H. In certain cases, R⁸ is —CH₂(R¹⁵), where R¹⁵ is N(R¹³)₂, and each R¹³ independently or together are as defined above. In certain cases, R⁸ is —CH₂(R¹⁵), where R¹⁵ is CH₂N(R¹³)₂, and each R¹³ independently or together are as defined above. In certain cases, R⁸ is —CH₂(R¹⁵), where R¹⁵ is C(O)N(R¹³)₂, and each R¹³ independently or together are as defined above. In certain cases, R⁸ is —CH₂(R¹⁵), where R¹⁵ is C(O)NHCN. In certain cases, R⁸ is —CH₂(R¹⁵), where R¹⁵ is S(O)₂R¹⁴, and R¹⁴ is as defined above. In certain cases, R⁸ is —CH₂(R¹⁵), where R¹⁵ is S(O)₂N(R¹³)₂ and each R¹³ independently or together are as defined above. In certain cases, R⁸ is —CH(R¹⁵)₂, where each R¹⁵ is independently as described above. In certain cases, R⁸ is —C(O)(CH₂)ₚN(R¹³)₂, where R¹³ independently or together are as defined above. In certain cases, R⁸ is cyclopropyl.

In certain embodiments of formula (I) or (II), Z is NR⁸, wherein R⁸ is selected from H, methyl, and ethyl.

In certain embodiments of formula (I) or (II), Z is NR⁸, wherein R⁸ wherein R⁸ is selected from —CH₂CO₂H, —C(CH₂NH₂)HCO₂H, —CH₂C(═O)N(CH₃)₂, —CH₂C(═O)N(CH₃)H, —CH₂C(═O)NH₂, —CH₂CH₂NH₂, —CH₂CH₂N(CH₃)₂, —CH₂CH₂NHC(═O)CH₂NH₂, —CH₂CH₂NHC(═O)CH(CH(CH₃)₂)(NH2), —CH(CH₃)(CO₂H), —C(CH₃)₂(CO₂H),

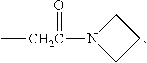

—CH₂CH₂N(CH₃)H,

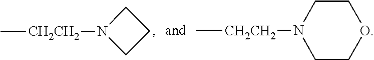

In certain embodiments of formula (I) or (II), Z is O.

In certain embodiments of formula (I) or (II), W is C(R^{9a})₂, where each R^{9a} is independently selected from H, alkyl, substituted alkyl, alkoxy, substituted alkoxy, halogen, nitrile, nitro, trifluoromethyl, hydroxyl, amino, substituted amino, cycloalkyl, substituted cycloalkyl, cycloalkenyl, substituted cycloalkenyl, aryl, substituted aryl, heterocycle, substituted heterocycle, heteroaryl, and substituted heteroaryl. In certain cases, at least one R^{9a} is selected from alkyl, and substituted alkyl. In certain cases, the alkyl is C₁₋₆ alkyl, or substituted C₁₋₆ alkyl. In certain cases, at least one R^{9a} is selected from cycloalkyl, and substituted cycloalkyl. In certain cases, at least one R^{9a} is selected from C₃₋₆cycloalkyl, and substituted C₃₋₆cycloalkyl. In certain cases, at least one R^{9a} is selected from hydroxyl and alkoxy. In certain cases, at least one R^{9a} is selected from amino, and substituted amino. In certain cases, at least one R^{9a} is selected from aryl and substituted aryl. In certain cases, at least one R^{9a} is selected from heterocycle and substituted heterocycle. In certain cases, at least one R^{9a} is selected from heteroaryl and substituted heteroaryl.

In certain embodiments of formula (I) or (II), W is C(R^{9a})₂, and both R^{9a} groups are H, such that W is CH₂.

In certain embodiments of formula (I) or (II), W is C(R^{9a})₂, where at least one R^{9a} is selected from C₁₋₆ alkyl, C₃₋₆cycloalkyl, substituted C₃₋₆cycloalkyl, hydroxyl, alkoxy, —(CH₂)ₚCO₂R¹², —(CH₂)ₚN(R¹³)₂, and —(CH₂)ₚOR^{9b}, where R^{9b} is selected from H, —C(O)CH₃, —(CH₂)ₚN(R¹³)₂, and —(CH₂)ₚCO₂R¹², R¹² is selected from H, alkyl and substituted alkyl; each R¹³ is independently selected from H, C₁₋₆ alkyl, substituted C₁₋₆ alkyl, S(O)₂R¹⁴, and C(O)R¹⁴, or two R¹³ together with the nitrogen to which they are attached form a cycle selected from heteroaryl, substituted heteroaryl, heterocycle, and substituted heterocycle; R¹⁴ is selected from C₁₋₆ alkyl, and substituted C₁₋₆ alkyl; and p is an integer from 1 to 6. In certain cases, at least one R^{9a} is C₁₋₆ alkyl, e.g., methyl, ethyl, propyl, butyl, pentyl or hexyl. In certain cases, at least one R^{9a} is, C₃₋₆cycloalkyl, e.g., cyclopropyl, cyclobutyl, cyclopentyl or cyclohexyl. In certain cases, at least one R^{9a} is, substituted C₃₋₆cycloalkyl. In certain cases, at least one R^{9a} is, hydroxy. In certain cases, at least one R^{9a} is, alkoxy. In certain cases, at least one R^{9a} is (CH₂)ₚCO₂R¹², R¹² is selected from H, alkyl and substituted alkyl, and p is an integer from 1 to 6. In certain cases, at least one R^{9a} is —(CH₂)ₚN(R¹³)₂, where each R¹³ are independently or together as defined above. In certain cases, R^{9a} is —(CH₂)ₚN(R¹³)₂, and each R¹³ is independently selected from H, C₁₋₆ alkyl, substituted C₁₋₆ alkyl, S(O)₂R¹⁴, and C(O)R¹⁴, and R¹⁴ is C₁₋₆ alkyl, or substituted C₁₋₆ alkyl. In other cases, R^{9a} is —(CH₂)ₚN(R¹³)₂, and the two R¹³ groups together with the nitrogen to which they are attached form a cycle selected from heteroaryl, substituted heteroaryl, heterocycle, and substituted heterocycle. In certain cases, at least one R^{9a} is —(CH₂)ₚOR^{9b}, where R^{9b} is as defined above.

In certain embodiments of formula (I) or (II), W is C(R^{9a})H, wherein R^{9a} is selected from H, C₁₋₆ alkyl, C₃₋₆cycloalkyl, substituted C₃₋₆cycloalkyl, hydroxyl, alkoxy, —(CH₂)ₚCO₂R¹², —(CH₂)ₚN(R¹³)₂, and —(CH₂)ₚOR^{9b}; R^{9b} is selected from H, —C(O)CH₃, —(CH₂)ₚN(R¹³)₂, and —(CH₂)ₚCO₂R¹²; R¹² is selected from H, alkyl and substituted alkyl; each R¹³ is independently selected from H, C₁₋₆ alkyl, substituted C₁₋₆alkyl, S(O)₂R¹⁴, and C(O)R¹⁴, or two R¹³ together with the nitrogen to which they are attached form a cycle selected from heteroaryl, substituted heteroaryl, heterocycle, and substituted heterocycle; R¹⁴ is selected from C₁₋₆ alkyl, and substituted C₁₋₆ alkyl; and p is an integer from 1 to 6. In certain cases, W is C(R^{9a})H, and R^{9a} is H. In certain cases, W is C(R^{9a})H and R^{9a} is C₁₋₆ alkyl, e.g., methyl, ethyl, propyl, butyl, pentyl or hexyl. In certain cases, W is C(R^{9a})H and R^{9a} is, C₃₋₆cycloalkyl, e.g., cyclopropyl, cyclobutyl, cyclopentyl or cyclohexyl. In certain cases, W is C(R^{9a})H and R^{9a} is, substituted C₃₋₆cycloalkyl. In certain cases, W is C(R^{9a})H and R⁹ is, hydroxy. In certain cases, W is C(R^{9a})H and R^{9a} is, alkoxy. In certain cases, W is C(R^{9a})H, R^{9a} is —(CH₂)ₚCO₂R¹², R¹² is selected from H, alkyl and substituted alkyl and p is an integer from 1 to 6. In certain cases, W is C(R^{9a})H, R^{9a} is —(CH₂)ₚN(R¹³)₂, where each R¹³ are independently or together as defined above. In certain cases, W is C(R^{9a})H, R^{9a} is —(CH₂)ₚN(R¹³)₂, and each R¹³ is independently selected from H, C₁₋₆ alkyl, substituted C₁₋₆ alkyl, S(O)₂R¹⁴, and C(O)R¹⁴, and R¹⁴ is C₁₋₆ alkyl, or substituted C₁₋₆ alkyl. In other cases, W is C(R^{9a})H, R^{9a} is —(CH₂)ₚN(R¹³)₂, and the two R¹³ groups together with the nitrogen to which they are attached form a cycle selected from heteroaryl, substituted heteroaryl, heterocycle, and substituted heterocycle. In certain cases, at least one R^{9a} is —(CH₂)ₚOR^{9b}, where R^{9b} is as defined above. In certain cases, at least one R^{9a} is —(CH₂)ₚOR^{9b}, and R^{9b} is H. In certain cases, at least one R^{9a} is —(CH₂)ₚOR^{9b}, and R^{9b} is —C(O)CH₃. In certain cases, at least one R^{9a} is —(CH₂)ₚOR^{9b}, and R^{9b} is —(CH₂)ₚN(R¹³)₂, where R¹³ independently or together are as defined above. In certain cases, at least one R^{9a} is —(CH₂)ₚOR^{9b}, and R^{9b} is —(CH₂)ₚCO₂R¹², where R¹² is as defined above.

In certain embodiments of formula (I) or (II), W is CH₂. In certain other cases, W is C(O).

In certain embodiments of formula (I) or (II), R¹³ is independently selected from H, methyl and ethyl. In certain cases, R¹³ is H. In certain cases, R¹³ is methyl. In certain other cases, R¹³ is ethyl.

In certain cases of formula (I) or (II), two R¹³ together with the nitrogen to which they are attached forms a cycle selected from, azetidine, pyrrolidine, pyrazolidine, imidazolidine, pyrrole, morpholine, piperidine, piperazine, thiomorpholine, tetrazole, triazole, imidazole, pyrazole, pyridine, pyrimidine, pyrazine, and trizine, wherein the cycle is optionally substituted (e.g., with one or more substituents).

In certain embodiments of formula (I) or (II), R¹⁵ is selected from tetrazole, —CO₂H, N(R¹³)₂, —CH₂N(R¹³)₂, —C(O)N(R¹³)₂, C(O)NHCN, and S(O)₂N(R¹³)₂. In certain cases, R¹⁵ is selected from CO₂H, N(R¹³)₂, —CH₂N(R¹³)₂, and —C(O)N(R¹³)₂. In certain cases, R¹⁵ is CO₂H. In certain cases, R¹⁵ is N(R¹³)₂. In certain cases, R¹⁵ is —CH₂N(R¹³)₂. In certain cases, R¹⁵ is —C(O)N(R¹³)₂.

In certain cases of formula (I) or (II), R¹⁴ is substituted C₁₋₆ alkyl, wherein one or more substituents is selected from halogen, C₃₋₆cycloalkyl, substituted C₃₋₆cycloalkyl, C₃₋₆heterocycle, and substituted C₃₋₆heterocycle. In certain cases, one or more substituent is selected from F, cyclopropyl, oxetane. In some cases, one or more substituent is F. In some cases, one or more substituent is cyclopropyl. In some cases, one or more substituent is oxetane.

In certain cases of any of formula (I) or (II), R¹⁴ is selected from methyl, ethyl, and CHF₂. In certain cases, R¹⁴ is methyl. In certain cases, R¹⁴ is ethyl. In certain cases, R¹⁴ is CHF₂.

In certain embodiments, the compound of formula (II) is of any one of formulae (IIIA) to (IIIC):

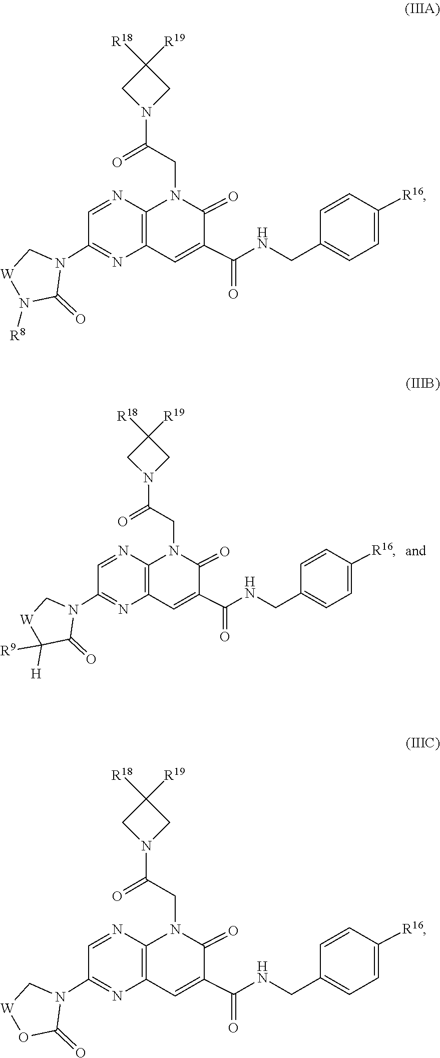

wherein:

each W is independently selected from C(R^{9a})₂, and C(O);

R⁸ is selected from H, C₁₋₆ alkyl, substituted C₁₋₆ alkyl, —C(O)(CH₂)ₚN(R¹³)₂, C₃₋₆cycloalkyl, substituted C₃₋₆cycloalkyl, C₃₋₆heterocycle, and substituted C₃₋₆heterocycle; each R⁹ and R^{9a} is independently selected from H, C₁₋₆ alkyl, C₃₋₆ cycloalkyl, substituted C₃₋₆heterocycle, hydroxyl, alkoxy, —(CH₂)ₚOR¹², —(CH₂)ₚCO₂R¹², and —(CH₂)ₚN(R¹³)₂;

R¹² is selected from H, alkyl and substituted alkyl;

each R¹³ is independently selected from H, C₁₋₆ alkyl, substituted C₁₋₆ alkyl, S(O)₂R¹⁴, and C(O)R¹⁴, or

two R¹³ together with the nitrogen to which they are attached form a cycle selected from heteroaryl, substituted heteroaryl, heterocycle, and substituted heterocycle;

R¹⁴ is selected from C₁₋₆ alkyl, and substituted C₁₋₆ alkyl;

R¹⁶ is selected from C₁₋₆ alkyl, halogen, nitrile, and trifluoromethyl;

R¹⁸ and R¹⁹ are each independently selected from C₁₋₆ alkyl, hydroxyl, alkoxy, —(CH₂)ₚOH, and halogen, or

R¹⁸ and R¹⁹ together with the carbon to which they are attached form a 3-6 membered cycle selected from, cycloalkyl, substituted cycloalkyl, cycloalkenyl, substituted cycloalkenyl, aryl, substituted aryl, heteroaryl, substituted heteroaryl, heterocycle, and substituted heterocycle; and

each p is independently an integer from 1 to 6.

In certain other embodiments, the compound of formula (II) is of any one of (IIID) to (IIIF):

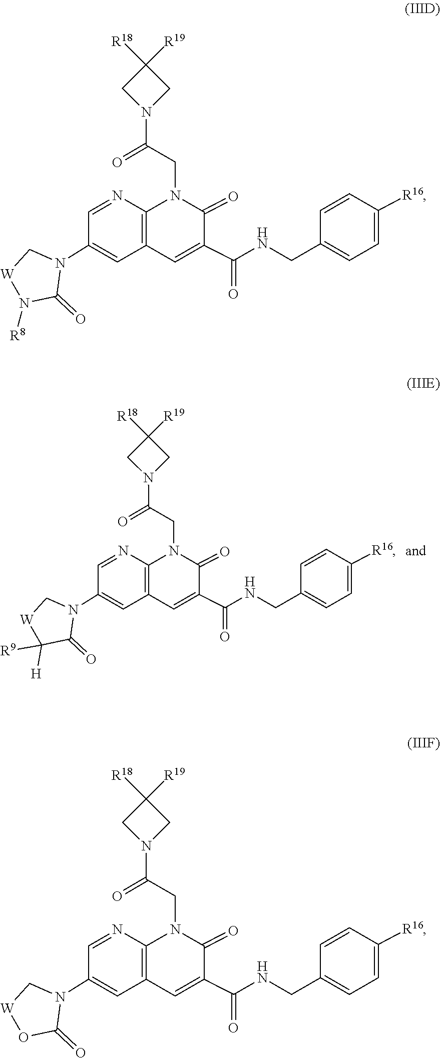

wherein:

each W is independently selected from C(R^{9a})₂, and C(O);

R⁸ is selected from H, C₁₋₆ alkyl, substituted C₁₋₆ alkyl, —C(O)(CH₂)ₚN(R¹³)₂, C₃₋₆cycloalkyl, substituted C₃₋₆cycloalkyl, C₃₋₆heterocycle, and substituted C₃₋₆heterocycle;

each R⁹ and R^{9a} is independently selected from H, C₁₋₆ alkyl, C₃₋₆ cycloalkyl, substituted C₃₋₆heterocycle, hydroxyl, alkoxy, —(CH₂)ₚOR¹², —(CH₂)ₚCO₂R¹², and —(CH₂)ₚN(R¹³)₂;

R¹² is selected from H, alkyl and substituted alkyl;

each R¹³ is independently selected from H, C₁₋₆ alkyl, substituted C₁₋₆ alkyl, S(O)₂R¹⁴, and C(O)R¹⁴, or

two R¹³ together with the nitrogen to which they are attached form a cycle selected from heteroaryl, substituted heteroaryl, heterocycle, and substituted heterocycle;

R¹⁴ is selected from C₁₋₆ alkyl, and substituted C₁₋₆ alkyl;

R¹⁶ is selected from C₁₋₆ alkyl, halogen, nitrile, and trifluoromethyl;

R¹⁸ and R¹⁹ are each independently selected from C₁₋₆ alkyl, hydroxyl, alkoxy, —(CH₂)ₚOH, and halogen, or

R¹⁸ and R¹⁹ together with the carbon to which they are attached form a 3-6 membered cycle selected from, cycloalkyl, substituted cycloalkyl, cycloalkenyl, substituted cycloalkenyl, aryl, substituted aryl, heteroaryl, substituted heteroaryl, heterocycle, and substituted heterocycle; and

each p is independently an integer from 1 to 6.

In certain embodiments of any of formulae (IIIA)-(IIIF), W is C(R^{9a})₂,

In certain embodiments of any of formulae (IIA)-(IIIF) W is C(R^{9a})₂, where each R^{9a} is independently selected from C₁₋₆ alkyl, C₃₋₆cycloalkyl, substituted C₃₋₆cycloalkyl, hydroxyl, alkoxy, —(CH₂)ₚOR¹², —(CH₂)ₚCO₂R¹², —(CH₂)ₚN(R¹³)₂, and —(CH₂)ₚOR^{9b}, where R^{9b} is selected from H, —C(O)CH₃, —(CH₂)ₚN(R¹³)₂, and —(CH₂)ₚCO₂R¹², R¹² is selected from H, alkyl and substituted alkyl; each R¹³ is independently selected from H, C₁₋₆ alkyl, substituted C₁₋₆ alkyl, S(O)₂R¹⁴, and C(O)R¹⁴, or two R¹³ together with the nitrogen to which they are attached form a cycle selected from heteroaryl, substituted heteroaryl, heterocycle, and substituted heterocycle; R¹⁴ is selected from C₁₋₆ alkyl, and substituted C₁₋₆ alkyl; and p is an integer from 1 to 6. In certain cases, at least one R^{9a} is H. In certain cases, at least one R^{9a} is C₁₋₆ alkyl, e.g., methyl, ethyl, propyl, butyl, pentyl or hexyl. In certain cases, at least one R^{9a} is, C₃₋₆cycloalkyl, e.g., cyclopropyl, cyclobutyl, cyclopentyl or cyclohexyl. In certain cases, at least one R^{9a} is, substituted C₃₋₆cycloalkyl. In certain cases, at least one R^{9a} is, hydroxy. In certain cases, at least one R^{9a} is, alkoxy. In certain cases, at least one R^{9a} is (CH₂)ₚOR¹², R¹² is selected from H, alkyl and substituted alkyl, and p is an integer from 1 to 6. In certain cases, at least one R^{9a} is (CH₂)ₚCO₂R¹², R¹² is selected from H, alkyl and substituted alkyl, and p is an integer from 1 to 6. In certain cases, at least one R^{9a} is —(CH₂)ₚN(R¹³)₂, where each R¹³ are independently or together as defined above. In certain cases, R^{9a} is —(CH₂)ₚN(R¹³)₂, and each R¹³ is independently selected from H, C₁₋₆ alkyl, substituted C₁₋₆ alkyl, S(O)₂R¹⁴, and C(O)R¹⁴, and R¹⁴ is C₁₋₆ alkyl, or substituted C₁₋₆ alkyl. In other cases, R^{9a} is —(CH₂)ₚN(R¹³)₂, and the two R¹³ groups together with the nitrogen to which they are attached form a cycle selected from heteroaryl, substituted heteroaryl, heterocycle, and substituted heterocycle. In certain cases, at least one R^{9a} is —(CH₂)ₚOR^{9b}, where R^{9b} is as defined above.

In certain embodiments of any of formulae (IIIA)-(IIIF), W is C(R^{9a})H, wherein R^{9a} is selected from H, C₁₋₆ alkyl, C₃₋₆cycloalkyl, substituted C₃₋₆cycloalkyl, hydroxyl, alkoxy, —(CH₂)ₚOR¹², —(CH₂)ₚCO₂R¹², —(CH₂)ₚN(R¹³)₂, and —(CH₂)ₚOR^{9b}; R^{9b} is selected from H, —C(O)CH₃, —(CH₂)ₚN(R¹³)₂, and —(CH₂)ₚCO₂R¹²; R¹² is selected from H, alkyl and substituted alkyl; each R¹³ is independently selected from H, C₁₋₆ alkyl, substituted C₁₋₆ alkyl, S(O)₂R¹⁴, and C(O)R¹⁴, or two R¹³ together with the nitrogen to which they are attached form a cycle selected from heteroaryl, substituted heteroaryl, heterocycle, and substituted heterocycle; R¹⁴ is selected from C₁₋₆ alkyl, and substituted C₁₋₆ alkyl; and p is an integer from 1 to 6. In certain cases, W is C(R^{9a})H, and R^{9a} is H. In certain cases, W is C(R^{9a})H and R^{9a} is C₁₋₆ alkyl, e.g., methyl, ethyl, propyl, butyl, pentyl or hexyl. In certain cases, W is C(R^{9a})H and R^{9a} is, C₃₋₆cycloalkyl, e.g., cyclopropyl, cyclobutyl, cyclopentyl or cyclohexyl. In certain cases, W is C(R^{9a})H and R^{9a} is, substituted C₃₋₆cycloalkyl. In certain cases, W is C(R^{9a})H and R⁹ is, hydroxy. In certain cases, W is C(R^{9a})H and R^{9a} is, alkoxy. In certain cases, W is C(R^{9a})H, R^{9a} is —(CH₂)ₚOR¹², R¹² is selected from H, alkyl and substituted alkyl and p is an integer from 1 to 6. In certain cases, W is C(R^{9a})H, R^{9a} is —(CH₂)ₚCO₂R¹², R¹² is selected from H, alkyl and substituted alkyl and p is an integer from 1 to 6. In certain cases, W is C(R^{9a})H, R^{9a} is —(CH₂)ₚN(R¹³)₂, where each R¹³ are independently or together as defined above. In certain cases, W is C(R^{9a})H, R^{9a} is —(CH₂)ₚN(R¹³)₂, and each R¹³ is independently selected from H, C₁₋₆ alkyl, substituted C₁₋₆ alkyl, S(O)₂R¹⁴, and C(O)R¹⁴, and R¹⁴ is C₁₋₆alkyl, or substituted C₁₋₆alkyl. In other cases, W is C(R^{9a})H, R^{9a} is —(CH₂)ₚN(R¹³)₂, and the two R¹³ groups together with the nitrogen to which they are attached form a cycle selected from heteroaryl, substituted heteroaryl, heterocycle, and substituted heterocycle. In certain cases, at least one R^{9a} is —(CH₂)ₚOR^{9b}, where R^{9b} is as defined above. In certain cases, at least one R^{9a} is —(CH₂)ₚOR^{9b}, and R^{9b} is H. In certain cases, at least one R^{9a} is —(CH₂)ₚOR^{9b}, and R^{9b} is —C(O)CH₃. In certain cases, at least one R^{9a} is —(CH₂)ₚOR^{9b}, and R^{9b} is —(CH₂)ₚN(R¹³)₂, where R¹³ independently or together are as defined above. In certain cases, at least one R^{9a} is —(CH₂)ₚOR^{9b}, and R^{9b} is —(CH₂)ₚCO₂R¹², where R¹² is as defined above.

In certain embodiments of any one of formulae (IIIA)-(IIIF), W is CH₂. In certain other cases of any one of formulae (IIIA)-(IIIF), W is C(O).

In certain embodiments, the compound is of any one of formulae (IVA) to (IVC):

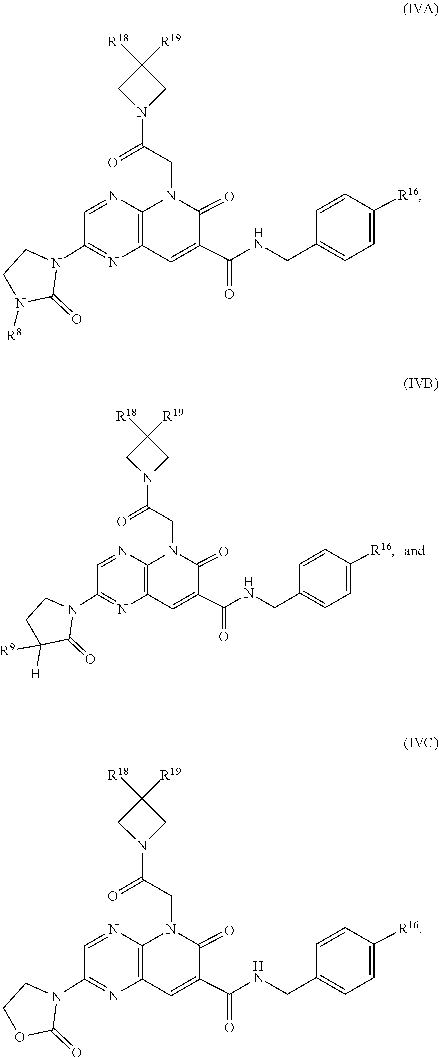

In certain embodiments, the compound is of any one of formulae (IVD) to (IVF):

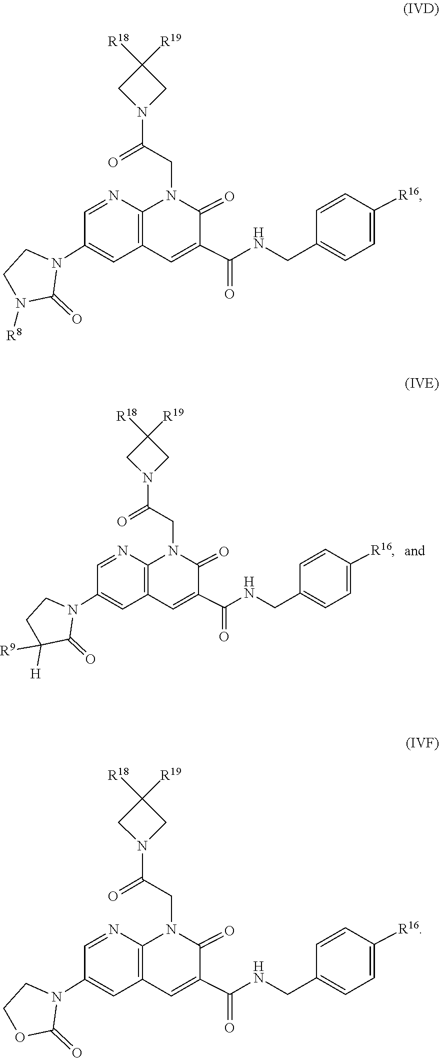

In certain embodiments of any of formulae (IIIA)-(IIIF) or (IVA)-(IVF), R¹⁸ and R¹⁹ are independently selected from C₁₋₆ alkyl, and halogen. In certain cases, R¹⁸ and R¹⁹ are both C₁₋₆ alkyl. In certain cases, both R¹⁸ and R¹⁹ are methyl. In certain cases of the formula (II), R¹⁸ is a halogen and R¹⁹ is C₁₋₆ alkyl. In certain cases, R¹⁸ is F, and R¹⁹ is methyl. In certain cases of the formula (II), both R¹⁸ and R¹⁹ are halogen. In certain cases, both R¹⁸ and R¹⁹ are F.

In certain other embodiments of any of formulae (IIIA)-(IIIF) or (IVA)-(IVF), R¹⁸ and R¹⁹ together with the carbon to which they are attached form a cycle selected from C₃₋₆cycloalkyl, substituted C₃₋₆cycloalkyl, C₃₋₆heterocycle, and substituted C₃₋₆heterocycle.

In certain cases, R¹⁸ and R¹⁹ together with the carbon to which they are attached form a cycle selected from C₃₋₆cycloalkyl and substituted C₃₋₆cycloalky. In certain cases, the cycle is a cyclopropyl, or a substituted cyclopropyl. In certain cases, R¹⁸ and R¹⁹ together with the carbon to which they are attached form a cycle selected from C₃₋₆heterocycle and substituted C₃₋₆heterocycle. In certain cases, the cycle is an oxetane or a substituted oxetane.

In certain embodiments of any of formulae (IIIA)-(IIIF) or (IVA)-(IVF), R¹⁶ is C₁₋₆ alkyl. In some cases, R¹⁶ is methyl. In some cases, R¹⁶ is selected from F, Cl, methyl and nitrile. In some cases, R¹⁶ is halogen. In certain cases, the halogen is selected from F and Cl. In some cases R¹⁶ is Cl. In certain cases, R¹⁶ is nitrile. In certain cases, R¹⁶ is trifluoromethyl.

In certain embodiments of formula (IIIB), (IIIE), (IVB), or (IVE), R⁹ is H. In certain cases, R⁹ is C₁₋₆ alkyl, e.g., methyl, ethyl, propyl, butyl, pentyl or hexyl. In certain cases, R⁹ is, C₃₋₆cycloalkyl, e.g., cyclopropyl, cyclobutyl, cyclopentyl or cyclohexyl. In certain cases, R⁹ is, substituted C₃₋₆cycloalkyl. In certain cases, R⁹ is, hydroxy. In certain cases, R⁹ is, alkoxy. In certain cases, R⁹ is —(CH₂)ₚOR¹², and R¹² is selected from H, alkyl and substituted alkyl. In certain cases, R⁹ is —(CH₂)ₚCO₂R¹², R¹² is selected from H, alkyl and substituted alkyl and p is an integer from 1 to 6. In certain cases, R⁹ is —(CH₂)ₚN(R¹³)₂, where each R¹³ are independently or together as defined herein. In certain cases, R⁹ is —(CH₂)ₚN(R¹³)₂, and each R¹³ is independently selected from H, C₁₋₆ alkyl, substituted C₁₋₆ alkyl, S(O)₂R¹⁴, and C(O)R¹⁴, and R¹⁴ is C₁₋₆ alkyl, or substituted C₁₋₆ alkyl. In other cases, R⁹ is —(CH₂)ₚN(R¹³)₂, and the two R¹³ groups together with the nitrogen to which they are attached form a cycle selected from heteroaryl, substituted heteroaryl, heterocycle, and substituted heterocycle.

In certain embodiments of formula (IIIB), (IIIE), (IVB), or (IVE), R⁹ is H, or C₁₋₃alkyl. In certain embodiments of formula (IIIB), (IIIB), (IVB) or (IVE), R⁹ is methyl.

In certain embodiments of formula (IIIA), (IIID), (IVA), or (IVD), R⁸ is H. In certain cases, R⁸ is C₁₋₆ alkyl, e.g., methyl, ethyl, propyl, butyl, pentyl or hexyl. In certain cases, R⁸ is, substituted C₁₋₆ alkyl (e.g., as described herein). In certain cases, R⁸ is, —C(O)(CH₂)ₚN(R¹³)₂, where each R¹³ are independently or together as defined above. In certain cases, R⁸ is, —C(O)(CH₂)ₚN(R¹³)₂, and each R¹³ is independently selected from H, C₁₋₆ alkyl, substituted C₁₋₆ alkyl, S(O)₂R¹⁴, and C(O)R¹⁴, and R¹⁴ is C₁₋₆ alkyl, or substituted C₁₋₆ alkyl. In other cases, R⁸ is, —C(O)(CH₂)ₚN(R¹³)₂, and the two R¹³ groups together with the nitrogen to which they are attached form a cycle selected from heteroaryl, substituted heteroaryl, heterocycle, and substituted heterocycle. In certain cases, R⁸ is C₃₋₆cycloalkyl. In certain cases, R⁸ is substituted C₃₋₆cycloalkyl. In certain cases, R⁸ is C₃₋₆heterocycle. In certain other cases, R⁸ is substituted C₃₋₆heterocycle.

In certain embodiments of formula (IIIA), (IIID), (IVA), or (IVD), R⁸ is C₁₋₆ alkyl substituted with one or two R¹⁵ groups, wherein each R¹⁵ is independently selected from heterocycle, substituted heterocycle, CH₃, CH₂F, CHF₂, CF₃, CN, C(NH)NH₂, OH, CH₂OH, CO₂H, N(R¹³)₂, CH₂N(R¹³)₂, C(O)N(R¹³)₂, C(O)NHCN, and S(O)₂N(R¹³)₂. In certain cases, R⁸ is C₁₋₃ alkyl (e.g., methyl, ethyl or propyl) substituted with one or two R¹⁵ groups. In certain cases R¹⁵ is selected from heterocycle and substituted heterocycle. In some cases, R¹⁵ is CHF₂. In some cases, R¹⁵ is CN. In some cases, R¹⁵ is C(NH)NH₂.

In some cases, R¹⁵ is OH. In some cases, R¹⁵ is CH₂OH. In some cases, R¹⁵ is CO₂H. In some cases, R¹⁵ is N(R¹³)₂. In some cases, R¹⁵ is CH₂N(R¹³)₂. In some cases, R¹⁵ is C(O)N(R¹³)₂. In some cases, R¹⁵ is C(O)NHCN. In some other cases, R¹⁵ is S(O)₂N(R¹³)₂.

In certain embodiments of formula (IIIA), (IIID), (IVA), or (IVD), R⁸ is selected from H, C₁₋₃ alkyl, —CH₂(R¹⁵), —CH(R¹⁵)₂, —C(O)(CH₂)ₚN(R¹³)₂, and cyclopropyl; each R¹⁵ is independently selected from heterocycle, substituted heterocycle, CHF₂, CN, C(NH)NH₂, OH, CH₂OH, CO₂H, N(R¹³)₂, CH₂N(R¹³)₂, C(O)N(R¹³)₂, C(O)NHCN, S(O)₂R¹⁴ and S(O)₂N(R¹³)₂; and each R¹³ is independently selected from H, C₁₋₆ alkyl, substituted C₁₋₆ alkyl, S(O)₂R¹⁴, and C(O)R¹⁴, or two R¹³ together with the nitrogen to which they are attached form a cycle selected from heteroaryl, substituted heteroaryl, heterocycle, and substituted heterocycle. In certain cases, R⁸ is C₁₋₃ alkyl. In certain cases, R⁸ is —CH₂(R¹⁵), where R¹⁵ is as defined above. In certain cases, R⁸ is —CH₂(R¹⁵), where R¹⁵ is CHF₂. In certain cases, R⁸ is —CH₂(R¹⁵), where R¹⁵ is CN. In certain cases, R⁸ is —CH₂(R¹⁵), where R¹⁵ is C(NH)NH₂. In certain cases, R⁸ is —CH₂(R¹⁵), where R¹⁵ is OH. In certain cases, R⁸ is —CH₂(R¹⁵), where R¹⁵ is CH₂OH. In certain cases, R⁸ is —CH₂(R¹⁵), where R¹⁵ is CO₂H. In certain cases, R⁸ is —CH₂(R¹⁵), where R¹⁵ is, N(R¹³)₂, and each R¹³ independently or together are as defined above. In certain cases, R⁸ is —CH₂(R¹⁵), where R¹⁵ is CH₂N(R¹³)₂, and each R¹³ independently or together are as defined above. In certain cases, R⁸ is —CH₂(R¹⁵), where R¹⁵ is C(O)N(R¹³)₂, and each R¹³ independently or together are as defined above. In certain cases, R⁸ is —CH₂(R¹⁵), where R¹⁵ is C(O)NHCN. In certain cases, R⁸ is —CH₂(R¹⁵), where R¹⁵ is S(O)₂R¹⁴, and R¹⁴ is as defined above. In certain cases, R⁸ is —CH₂(R¹⁵), where R¹⁵ is S(O)₂N(R¹³)₂ and each R¹³ independently or together are as defined above. In certain cases, R⁸ is —CH(R¹⁵)₂, where each R¹⁵ is independently as described above. In certain cases, R⁸ is —C(O)(CH₂)ₚN(R¹³)₂, where R¹³ independently or together are as defined above. In certain cases, R⁸ is cyclopropyl.

In certain embodiments of formula (IIIA), (IIID), (IVA), or (IVD), R⁸ is selected from H, methyl, and ethyl.

In certain embodiments of formula (IIIA), (IIID), (IVA), or (IVD), R⁸ wherein R⁸ is selected from —CH₂CO₂H, —C(CH₂NH₂)HCO₂H, —CH₂C(═O)N(CH₃)₂, —CH₂C(═O)N(CH₃)H, —CH₂C(═O)NH₂, —CH₂CH₂NH₂, —CH₂CH₂N(CH₃)₂, —CH₂CH₂NHC(═O)CH₂NH₂, —CH₂CH₂NHC(═O)CH(CH(CH₃)₂)(NH₂), —CH(CH₃)(CO₂H), —C(CH₃)₂(CO₂H),

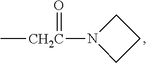

—CH₂CH₂N(CH₃)H,

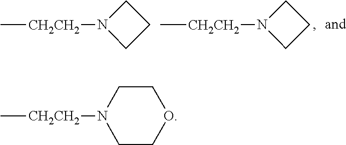

In certain embodiments of formula of any of formulae (IIIA)-(IIIB), (IIID)-(IIIE), (IVA)-(IVB) or (IVD)-(IVE), R¹³ is independently selected from H, methyl and ethyl. In certain cases, R¹³ is H. In certain cases, R¹³ is methyl. In certain other cases, R¹³ is ethyl.

In certain cases of formula of any of formulae (IIIA)-(IIIB), (IIID)-(IIIE), (IVA)-(IVB) or (IVD)-(IVE), two R¹³ together with the nitrogen to which they are attached forms a cycle selected from, azetidine, pyrrolidine, pyrazolidine, imidazolidine, pyrrole, morpholine, piperidine, piperazine, thiomorpholine, tetrazole, triazole, imidazole, pyrazole, pyridine, pyrimidine, pyrazine, and trizine, wherein the cycle is optionally substituted (e.g., with one or more substituents).

In certain embodiments of formula of any of formulae (IIIA)-(IIIB), (IIID)-(IIIE), (IVA)-(IVB) or (IVD)-(IVE), R¹⁵ is selected from tetrazole, —CO₂H, N(R¹³)₂, —CH₂N(R¹³)₂, —C(O)N(R¹³)₂, C(O)NHCN, and S(O)₂N(R¹³)₂. In certain cases, R¹⁵ is selected from CO₂H, N(R¹³)₂, —CH₂N(R¹³)₂, and —C(O)N(R¹³)₂. In certain cases, R¹⁵ is CO₂H. In certain cases, R¹⁵ is N(R¹³)₂. In certain cases, R¹⁵ is —CH₂N(R¹³)₂. In certain cases, R¹⁵ is —C(O)N(R¹³)₂.

In certain cases of formula of any of formulae (IIIA)-(IIIB), (IIID)-(IIIE), (IVA)-(IVB) or (IVD)-(IVE), R¹⁴ is substituted C₁₋₆ alkyl, wherein one or more substituents is selected from halogen, C₃₋₆cycloalkyl, substituted C₃₋₆cycloalkyl, C₃₋₆heterocycle, and substituted C₃₋₆heterocycle. In certain cases, one or more substituent is selected from F, cyclopropyl, oxetane. In some cases, one or more substituent is F. In some cases, one or more substituent is cyclopropyl. In some cases, one or more substituent is oxetane.

In certain cases of any of formula of any of formulae (IIIA)-(IIIB), (IIID)-(IIIE), (IVA)-(IVB) or (IVD)-(IVE), R¹⁴ is selected from methyl, ethyl, and CHF₂. In certain cases, R¹⁴ is methyl. In certain cases, R¹⁴ is ethyl. In certain cases, R¹⁴ is CHF₂.

In certain embodiments, the compound is of formula (V):

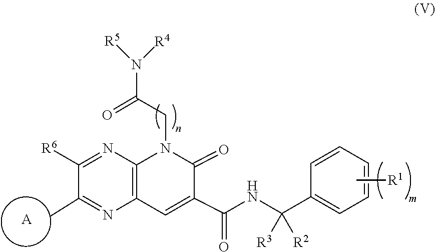

wherein:

each R¹ is independently selected from H, alkyl, substituted alkyl, halogen, nitrile, nitro, trifluoromethyl, hydroxyl, amino, and substituted amino;

R² and R³ are each independently selected from H, alkyl and substituted alkyl;

R⁴ and R⁵ are each independently selected from H, alkyl, substituted alkyl, cycloalkyl, substituted cycloalkyl, cycloalkenyl, substituted cycloalkenyl, aryl, substituted aryl, heterocycle, substituted heterocycle, heteroaryl, and substituted heteroaryl, or

R⁴ and R⁵ together with the nitrogen to which they are attached form a cycle selected from, heteroaryl, substituted heteroaryl, heterocycle, and substituted heterocycle, (e.g., azetidine, spiroazetidine);

R⁶ is selected from hydrogen, alkyl, substituted alkyl, alkoxy, substituted alkoxy, halogen, nitrile, nitro, trifluoromethyl, hydroxyl, amino, substituted amino, cycloalkyl, substituted cycloalkyl, cycloalkenyl, substituted cycloalkenyl, aryl, substituted aryl, heterocycle, substituted heterocycle, heteroaryl, and substituted heteroaryl;

Ring A is selected from cycloalkyl, substituted cycloalkyl, cycloalkenyl, substituted cycloalkenyl, aryl, substituted aryl, heterocycle, substituted heterocycle, heteroaryl, and substituted heteroaryl;

n is an integer from 1 to 6; and

m is an integer from 1 to 5;

or a pharmaceutically acceptable salt, solvate, or hydrate thereof.

In certain embodiments of the compound of formula (V), ring A is cycloalkyl or substituted cycloalkyl (e.g., as described herein). In certain cases, ring A is cycloalkenyl, or substituted cycloalkenyl. In certain cases, ring A is aryl or substituted aryl. In certain cases, ring A is heterocycle or substituted heterocycle. In certain cases ring A is heteroaryl or substituted heteroaryl.

In certain embodiments of formula (V), ring A is a monocyclic heterocycle selected from imidazolidinone, pyrrolidinone, aziridinyl, azetidinyl, furanyl, pyrrolidinyl, pyrazolinyl, tetrahydrothiophenyl, sulfolanyl, 2-oxazolidinone, piperidinyl, piperazinyl, tetrahydorpyranyl, morpholinyl, thiomorpholinyl, and azepanyl.

In certain cases, ring A is a bicyclic heterocycle selected from decahydroquinolinyl, decahydroisoquinolinyl, 1,2,3,4-tetrahydroquinolinyl, 1,2,3,4-tetrahydroisoquinolinyl, phenoxazinyl, phenothiazinyl, and 2-azaadamantanyl.

In certain embodiments, the compound is described by the structure of one of the compounds of Table 1.

TABLE 1
Compounds

| Compound Number | Structure and Name |
|---|---|
| 1 | 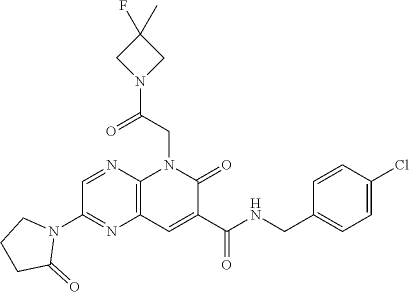 [SMILES: CC1(F)CN(C(=O)Cn2c(=O)c(C(=O)NCc3ccc(Cl)cc3)cc3nc(N4CCCC4=O)cnc32)C1] |
| | N-[(4-chlorophenyl)methyl]-5-[2-(3-fluoro-3- |
| | methylazetidin-1-yl)-2-oxoethyl]-6-oxo-2-(2- |
| | oxopyrrolidin-1-yl)-5,6-dihydropyrido[2,3-b]pyrazine- |
| | 7-carboxamide |
| 2 | 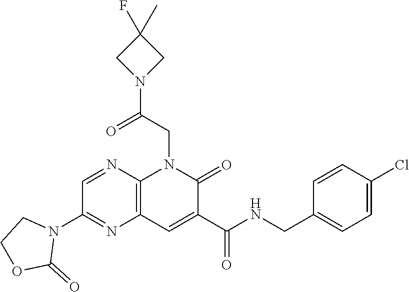 [SMILES: CC1(F)CN(C(=O)Cn2c(=O)c(C(=O)NCc3ccc(Cl)cc3)cc3nc(N4CCOC4=O)cnc32)C1] |
| | N-[(4-chlorophenyl)methyl]-5-[2-(3-fluoro-3- |
| | methylazetidin-1-yl)-2-oxoethyl]-6-oxo-2-(2-oxo-1,3- |
| | oxazolidin-3-yl)-5,6-dihydropyrido[2,3-b]pyrazine-7- |
| | carboxamide |
| 3 | 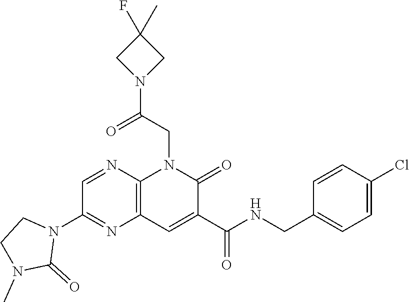 [SMILES: CN1CCN(c2cnc3c(cc(C(=O)NCc4ccc(Cl)cc4)c(=O)n3CC(=O)N3CC(C)(F)C3)n2)C1=O] |
| | N-[(4-chlorophenyl)methyl]-5-[2-(3-fluoro-3- |
| | methylazetidin-1-yl)-2-oxoethyl]-2-(3-methyl-2- |
| | oxoimidazolidin-1-yl)-6-oxo-5,6-dihydropyrido[2,3- |
| | b]pyrazine-7-carboxamide |
| 4 | 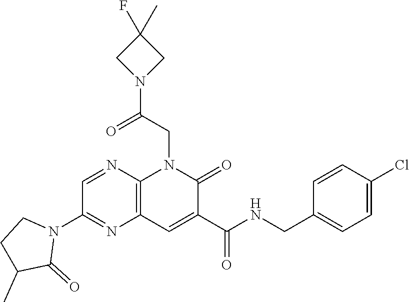 [SMILES: CC1CCN(c2cnc3c(cc(C(=O)NCc4ccc(Cl)cc4)c(=O)n3CC(=O)N3CC(C)(F)C3)n2)C1=O] |
| | N-[(4-chlorophenyl)methyl]-5-[2-(3-fluoro-3- |
| | methylazetidin-1-yl)-2-oxoethyl]-2-(3-methyl-2- |
| | oxopyrrolidin-1-yl)-6-oxo-5,6-dihydropyrido[2,3- |
| | b]pyrazine-7-carboxamide |
| 5 | 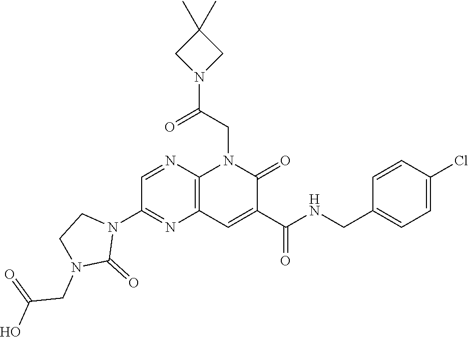 [SMILES: CC1(C)CN(C(=O)Cn2c(=O)c(C(=O)NCc3ccc(Cl)cc3)cc3nc(N4CCN(CC(=O)O)C4=O)cnc32)C1] |
| | [3-(7-{[(4-chlorophenyl)methyl]carbamoyl}-5-[2-(3,3- |
| | dimethylazetidin-1-yl)-2-oxoethyl]-6-oxo-5,6- |
| | dihydropyrido[2,3-b]pyrazin-2-yl)-2-oxoimidazolidin- |
| | 1-yl]acetic acid |
| 6 | 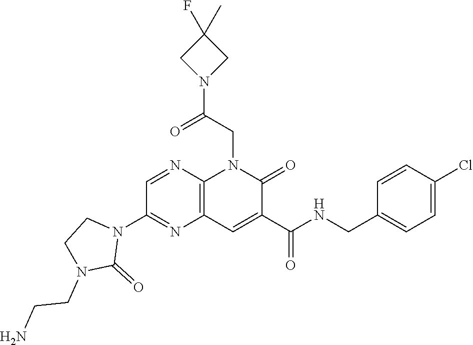 [SMILES: CC1(F)CN(C(=O)Cn2c(=O)c(C(=O)NCc3ccc(Cl)cc3)cc3nc(N4CCN(CCN)C4=O)cnc32)C1] |
| | 2-[3-(2-aminoethyl)-2-oxoimidazolidin-1-yl]-N-[(4- |
| | chlorophenyl)methyl]-5-[2-(3-fluoro-3- |
| | methylazetidin-1-yl)-2-oxoethyl]-6-oxo-5,6- |
| | dihydropyrido[2,3-blpyrazine-7-carboxamide |
| 7 | 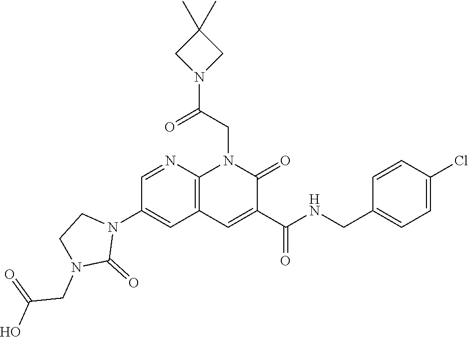 [SMILES: CC1(C)CN(C(=O)Cn2c(=O)c(C(=O)NCc3ccc(Cl)cc3)cc3cc(N4CCN(CC(=O)O)C4=O)cnc32)C1] |
| | [3-(6-{[(4-chlorophenyl)methyl]carbamoyl}-8-[2-(3,3- |
| | dimethylazetidin-1-yl)-2-oxoethyl]-7-oxo-7,8- |
| | dihydro-1,8-naphthyridin-3-yl)-2-oxoimidazolidin-1- |
| | yl]acetic acid |
| 8 | 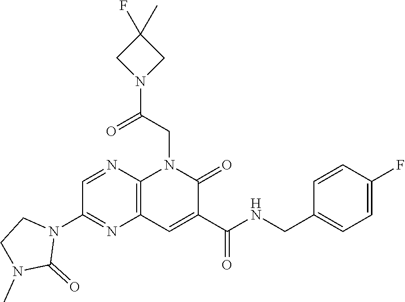 [SMILES: CN1CCN(c2cnc3c(cc(C(=O)NCc4ccc(F)cc4)c(=O)n3CC(=O)N3CC(C)(F)C3)n2)C1=O] |
| | 5-[2-(3-fluoro-3-methylazetidin-1-yl)-2-oxoethyl]-n- |
| | [(4-fluorophenyl)methyl]-2-(3-methyl-2- |
| | oxoimidazolidin-1-yl)-6-oxo-5,6-dihydropyrido[2,3- |
| | b]pyrazine-7-carboxamide |
| 9 | 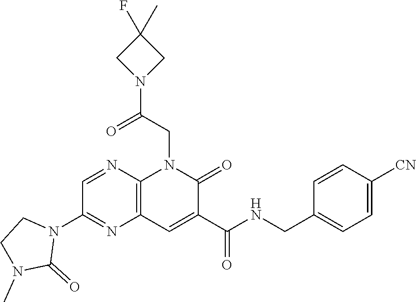 [SMILES: CN1CCN(c2cnc3c(cc(C(=O)NCc4ccc(C#N)cc4)c(=O)n3CC(=O)N3CC(C)(F)C3)n2)C1=O] |
| | N-[(4-cyanophenyl)methyl]-5-[2-(3-fluoro-3- |
| | methylazetidin-1-yl)-2-oxoethyl]-2-(3-methyl-2- |
| | oxoimidazolidin-1-yl)-6-oxo-5,6-dihydropyrido[2,3- |
| | b]pyrazine-7-carboxamide |
| 10 | 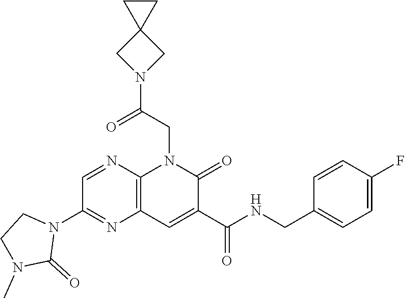 [SMILES: CN1CCN(c2cnc3c(cc(C(=O)NCc4ccc(F)cc4)c(=O)n3CC(=O)N3CC4(CC4)C3)n2)C1=O] |
| | 5-[2-(5-azaspiro[2.3]hexan-5-yl)-2-oxoethyl]-N-[(4- |
| | fluorophenyl)methyl]-2-(3-methyl-2-oxoimidazolidin- |
| | 1-yl)-6-oxo-5,6-dihydropyrido[2,3-b]pyrazine-7- |
| | carboxamide |
| 11 | 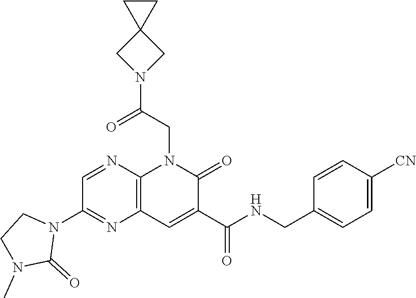 [SMILES: CN1CCN(c2cnc3c(cc(C(=O)NCc4ccc(C#N)cc4)c(=O)n3CC(=O)N3CC4(CC4)C3)n2)C1=O] |
| | 5-[2-(5-azaspiro[2.3]hexan-5-yl)-2-oxoethyl]-N-[(4- |
| | cyanophenyl)methyl]-2-(3-methyl-2-oxoimidazolidin- |
| | 1-yl)-6-oxo-5,6-dihydropyrido[2,3-b]pyrazine-7- |
| | carboxamide |
| 12 | 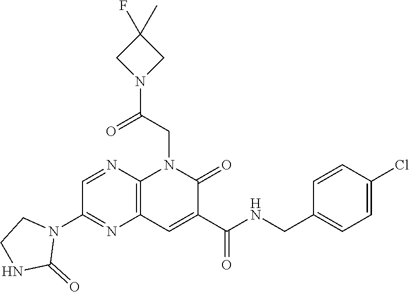 [SMILES: CC1(F)CN(C(=O)Cn2c(=O)c(C(=O)NCc3ccc(Cl)cc3)cc3nc(N4CCNC4=O)cnc32)C1] |
| | N-[(4-chlorophenyl)methyl]-5-[2-(3-fluoro-3- |
| | methylazetidin-1-yl)-2-oxoethyl]-6-oxo-2-(2- |
| | oxoimidazolidin-1-yl)-5,6-dihydropyrido[2,3- |
| | b]pyrazine-7-carboxamide |
| 13 | 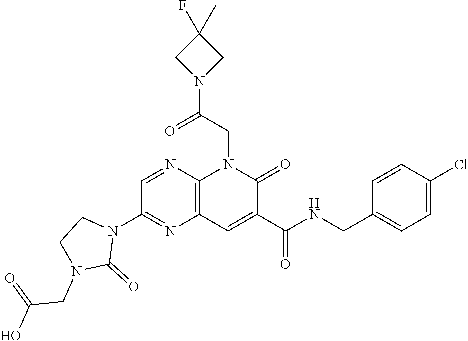 [SMILES: CC1(F)CN(C(=O)Cn2c(=O)c(C(=O)NCc3ccc(Cl)cc3)cc3nc(N4CCN(CC(=O)O)C4=O)cnc32)C1] |
| | [3-(7-{[(4-chlorophenyl)methyl]carbamoyl}-5-[2-(3- |
| | fluoro-3-methylazetidin-1-yl)-2-oxoethyl]-6-oxo-5,6- |
| | dihydropyrido[2,3-b]pyrazin-2-yl)-2-oxoimidazolidin- |
| | 1-yl]acetic acid |
| 14 | 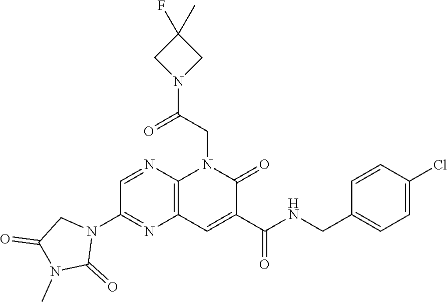 [SMILES: CN1C(=O)CN(c2cnc3c(cc(C(=O)NCc4ccc(Cl)cc4)c(=O)n3CC(=O)N3CC(C)(F)C3)n2)C1=O] |
| | N-[(4-chlorophenyl)methyl]-5-[2-(3-fluoro-3- |
| | methylazetidin-1-yl)-2-oxoethyl]-2-(3-methyl-2,4- |
| | dioxoimidazolidin-1-yl)-6-oxo-5,6-dihydropyrido[2,3- |
| | b]pyrazine-7-carboxamide |
| 15 | 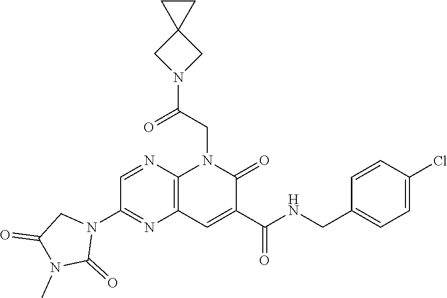 [SMILES: CN1C(=O)CN(c2cnc3c(cc(C(=O)NCc4ccc(Cl)cc4)c(=O)n3CC(=O)N3CC4(CC4)C3)n2)C1=O] |
| | 5-[2-(5-azaspiro[2.3]hexan-5-yl)-2-oxoethyl]-N-[(4- |
| | chlorophenyl)methyl]-2-(3-methyl-2,4- |
| | dioxoimidazolidin-1-yl)-6-oxo-5,6-dihydropyrido[2,3- |
| | b]pyrazine-7-carboxamide |
| 16 | 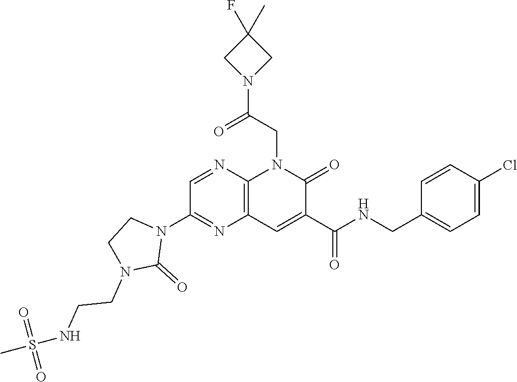 [SMILES: CC1(F)CN(C(=O)Cn2c(=O)c(C(=O)NCc3ccc(Cl)cc3)cc3nc(N4CCN(CCNS(C)(=O)=O)C4=O)cnc32)C1] |
| | N-[(4-chlorophenyl)methyl]-5-[2-(3-fluoro-3- |
| | methylazetidin-1-yl)-2-oxoethyl]-2-(3-{2- |
| | [(methanesulfonyl)amino]ethyl}-2-oxoimidazolidin-1- |
| | yl)-6-oxo-5,6-dihydropyrido[2,3-b]pyrazine-7- |
| | carboxamide |
| 17 | 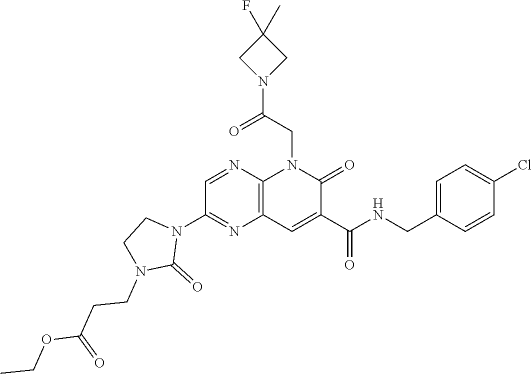 [SMILES: CCOC(=O)CCN1CCN(c2cnc3c(cc(C(=O)NCc4ccc(Cl)cc4)c(=O)n3CC(=O)N3CC(C)(F)C3)n2)C1=O] |
| | ethyl 3-[3-(7-{[(4-chlorophenyl)methyl]carbamoyl}-5- |
| | [2-(3-fluoro-3-methylazetidin-1-yl)-2-oxoethyl]-6- |
| | oxo-5,6-dihydropyrido[2,3-b]pyrazin-2-yl)-2- |
| | oxoimidazolidin-1-yl]propanoate |
| 18 | 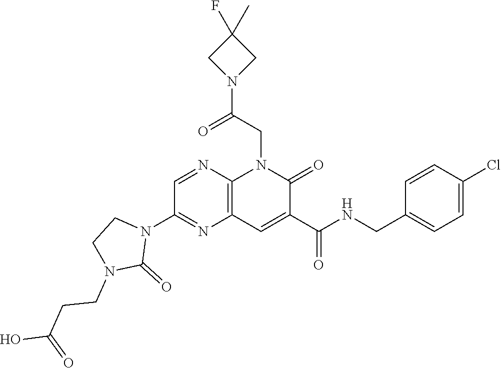 [SMILES: CC1(F)CN(C(=O)Cn2c(=O)c(C(=O)NCc3ccc(Cl)cc3)cc3nc(N4CCN(CCC(=O)O)C4=O)cnc32)C1] |
| | 3-[3-(7-{[(4-chlorophenyl)methyl]carbamoyl}-5-[2-(3- |
| | fluoro-3-methylazetidin-1-yl)-2-oxoethyl]-6-oxo-5,6- |
| | dihydropyrido[2,3-b]pyrazin-2-yl)-2-oxoimidazolidin- |
| | 1-yl]propanoic acid |
| 19 | 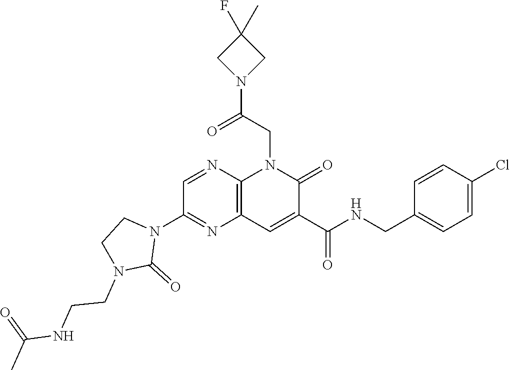 [SMILES: CC(=O)NCCN1CCN(c2cnc3c(cc(C(=O)NCc4ccc(Cl)cc4)c(=O)n3CC(=O)N3CC(C)(F)C3)n2)C1=O] |
| | 2-[3-(2-acetamidoethyl)-2-oxoimidazolidin-1-yl]-N- |
| | [(4-chlorophenyl)methyl]-5-[2-(3-fluoro-3- |
| | methylazetidin-1-yl)-2-oxoethyl]-6-oxo-5,6- |
| | dihydropyrido[2,3-blpyrazine-7-carboxamide |
| 20 | 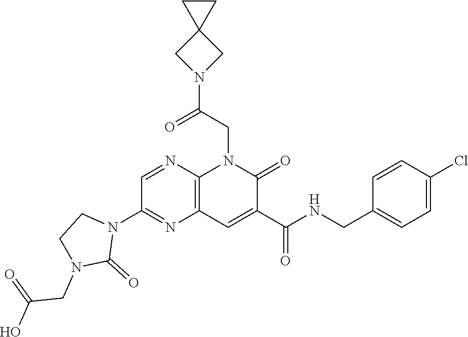 [SMILES: O=C(O)CN1CCN(c2cnc3c(cc(C(=O)NCc4ccc(Cl)cc4)c(=O)n3CC(=O)N3CC4(CC4)C3)n2)C1=O] |
| | [3-(5-[2-(5-azaspiro[2.3]hexan-5-yl)-2-oxoethyl]-7- |
| | {[(4-chlorophenyl)methyl]carbamoyl}-6-oxo-5,6- |
| | dihydropyrido[2,3-b]pyrazin-2-yl)-2-oxoimidazolidin- |
| | 1-yl]acetic acid |
| 21 | 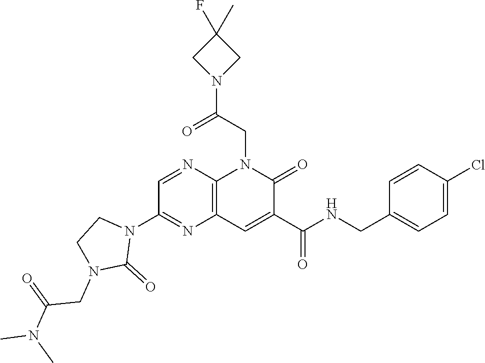 [SMILES: CN(C)C(=O)CN1CCN(c2cnc3c(cc(C(=O)NCc4ccc(Cl)cc4)c(=O)n3CC(=O)N3CC(C)(F)C3)n2)C1=O] |
| | N-[(4-chlorophenyl)methyl]-2-{3-[2-(dimethylamino)- |
| | 2-oxoethyl]-2-oxoimidazolidin-1-yl}-5-[2-(3-fluoro-3- |
| | methylazetidin-1-yl)-2-oxoethyl]-6-oxo-5,6- |
| | dihydropyrido[2,3-blpyrazine-7-carboxamide |
| 22 | 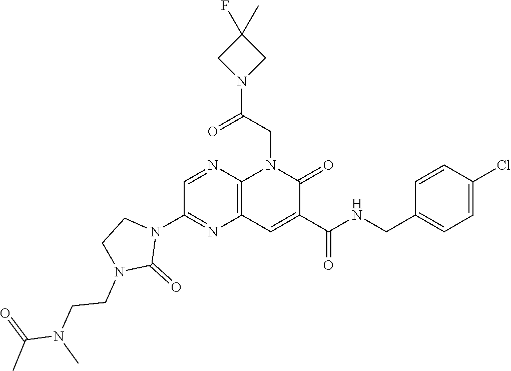 [SMILES: CC(=O)N(C)CCN1CCN(c2cnc3c(cc(C(=O)NCc4ccc(Cl)cc4)c(=O)n3CC(=O)N3CC(C)(F)C3)n2)C1=O] |
| | 2-(3-{2-[acetyl(methyl)amino]ethyl}-2- |
| | oxoimidazolidin-1-yl)-N-[(4-chlorophenyl)methyl]-5- |
| | [2-(3-fluoro-3-methylazetidin-1-yl)-2-oxoethyl]-6- |
| | oxo-5,6-dihydropyrido[2,3-b]pyrazine-7- |
| | carboxamide |
| 23 | 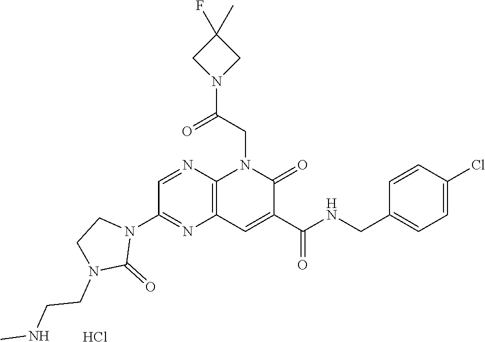 [SMILES: CNCCN1CCN(c2cnc3c(cc(C(=O)NCc4ccc(Cl)cc4)c(=O)n3CC(=O)N3CC(C)(F)C3)n2)C1=O ; Cl] |
| | N-[(4-chlorophenyl)methyl]-5-[2-(3-fluoro-3- |
| | methylazetidin-1-yl)-2-oxoethyl]-2-{3-[2- |
| | (methylamino)ethyl]-2-oxoimidazolidin-1-yl}-6-oxo- |
| | 5,6-dihydropyrido[2,3-b]pyrazine-7-carboxamide- |
| | hydrogen chloride |
| 24 | 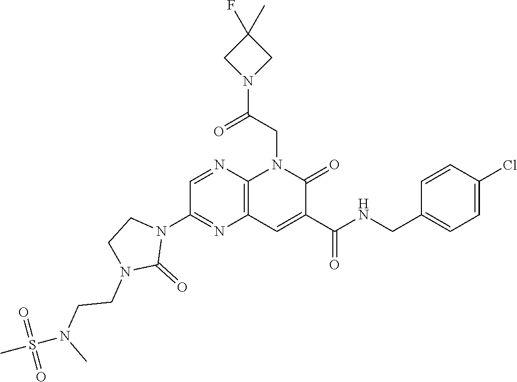 [SMILES: CN(CCN1CCN(c2cnc3c(cc(C(=O)NCc4ccc(Cl)cc4)c(=O)n3CC(=O)N3CC(C)(F)C3)n2)C1=O)S(C)(=O)=O] |
| | N-[(4-chlorophenyl)methyl]-5-[2-(3-fluoro-3- |
| | methylazetidin-1-yl)-2-oxoethyl]-2-(3-{2- |
| | [(methanesulfonyl)(methyl)amino]ethyl}-2- |
| | oxoimidazolidin-1-yl)-6-oxo-5,6-dihydropyrido[2,3- |
| | b]pyrazine-7-carboxamide |
| 25 | 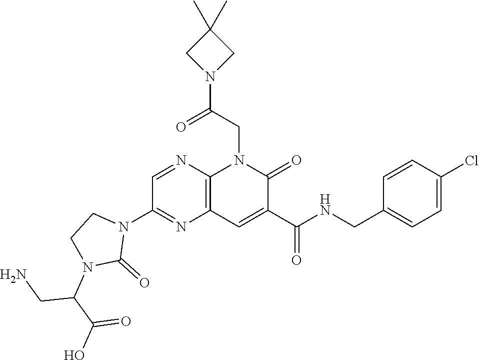 [SMILES: CC1(C)CN(C(=O)Cn2c(=O)c(C(=O)NCc3ccc(Cl)cc3)cc3nc(N4CCN(C(CN)C(=O)O)C4=O)cnc32)C1] |
| | 3-amino-2-[3-(7-{[(4- |
| | chlorophenyl)methyl]carbamoyl}-5-[2-(3,3- |
| | dimethylazetidin-1-yl)-2-oxoethyl]-6-oxo-5,6- |
| | dihydropyrido[2,3-b]pyrazin-2-yl)-2-oxoimidazolidin- |
| | 1-yl]propanoic acid |
| 26 | 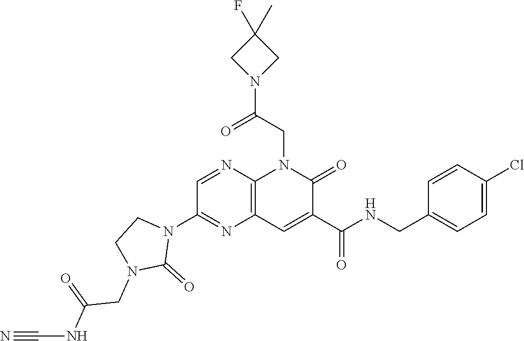 [SMILES: CC1(F)CN(C(=O)Cn2c(=O)c(C(=O)NCc3ccc(Cl)cc3)cc3nc(N4CCN(CC(=O)NC#N)C4=O)cnc32)C1] |
| | N-[(4-chlorophenyl)methyl]-2-{3-[2-(cyanoamino)-2- |
| | oxoethyl]-2-oxoimidazolidin-1-yl}-5-[2-(3-fluoro-3- |
| | methylazetidin-1-yl)-2-oxoethyl]-6-oxo-5,6- |
| | dihydropyrido[2,3-b]pyrazine-7-carboxamide |
| 27 | 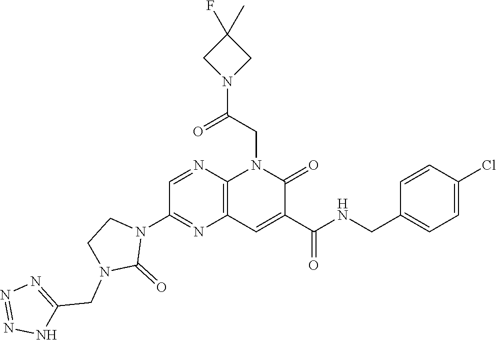 [SMILES: CC1(F)CN(C(=O)Cn2c(=O)c(C(=O)NCc3ccc(Cl)cc3)cc3nc(N4CCN(Cc5nnn[nH]5)C4=O)cnc32)C1] |
| | N-[(4-chlorophenyl)methyl]-5-[2-(3-fluoro-3- |
| | methylazetidin-1-yl)-2-oxoethyl]-6-oxo-2-{2-oxo-3- |
| | [(1H-tetrazol-5-yl)methyl]imidazolidin-1-yl}-5,6- |
| | dihydropyrido[2,3-b]pyrazine-7-carboxamide |
| 28 | 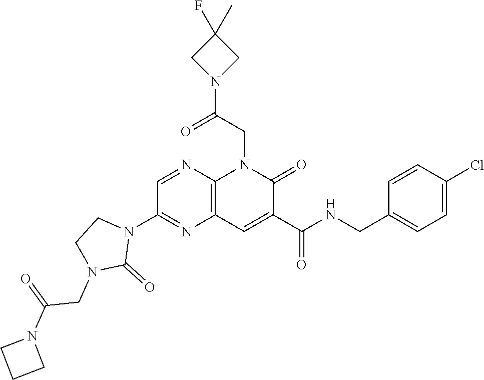 [SMILES: CC1(F)CN(C(=O)Cn2c(=O)c(C(=O)NCc3ccc(Cl)cc3)cc3nc(N4CCN(CC(=O)N5CCC5)C4=O)cnc32)C1] |
| | 2-{3-[2-(azetidin-1-yl)-2-oxoethyl]-2-oxoimidazolidin- |
| | 1-yl}-N-[(4-chlorophenyl)methyl]-5-[2-(3-fluoro-3- |
| | methylazetidin-1-yl)-2-oxoethyl]-6-oxo-5,6- |
| | dihydropyrido[2,3-b]pyrazine-7-carboxamide |
| 29 | 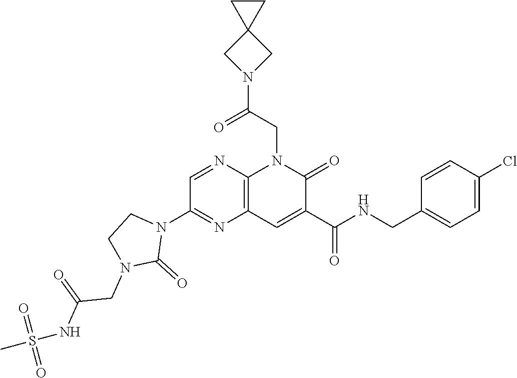 [SMILES: CS(=O)(=O)NC(=O)CN1CCN(c2cnc3c(cc(C(=O)NCc4ccc(Cl)cc4)c(=O)n3CC(=O)N3CC4(CC4)C3)n2)C1=O] |
| | 5-[2-(5-azaspiro[2.3]hexan-5-yl)-2-oxoethyl]-N-[(4- |
| | chlorophenyl)methyl]-2-(3-{2- |
| | [(methanesulfonyl)amino]-2-oxoethyl}-2- |
| | oxoimidazolidin-1-yl)-6-oxo-5,6-dihydropyrido[2,3- |
| | b]pyrazine-7-carboxamide |
| 30 | 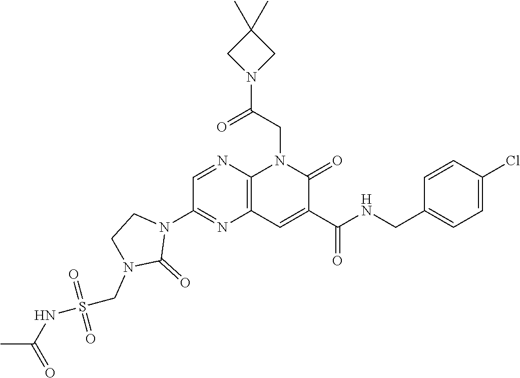 [SMILES: CC(=O)NS(=O)(=O)CN1CCN(c2cnc3c(cc(C(=O)NCc4ccc(Cl)cc4)c(=O)n3CC(=O)N3CC(C)(C)C3)n2)C1=O] |
| | 2-{3-[(acetylsulfamoyl)methyl]-2-oxoimidazolidin-1- |
| | yl}-N-[(4-chlorophenyl)methyl]-5-[2-(3,3- |
| | dimethylazetidin-1-yl)-2-oxoethyl]-6-oxo-5,6- |
| | dihydropyrido[2,3-b\|pyrazine-7-carboxamide |
| 31 | 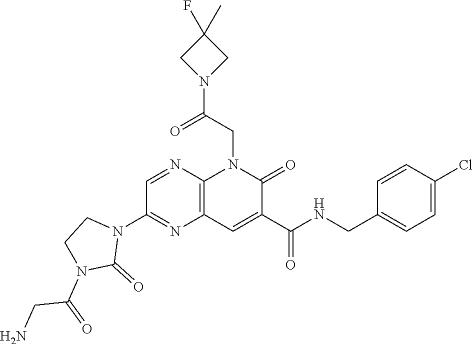 [SMILES: CC1(F)CN(C(=O)Cn2c(=O)c(C(=O)NCc3ccc(Cl)cc3)cc3nc(N4CCN(C(=O)CN)C4=O)cnc32)C1] |
| | N-[(4-chlorophenyl)methyl]-5-[2-(3-fluoro-3- |
| | methylazetidin-1-yl)-2-oxoethyl]-2-(3-glycyl-2- |
| | oxoimidazolidin-1-yl)-6-oxo-5,6-dihydropyrido[2,3- |
| | b]pyrazine-7-carboxamid |
| 32 | 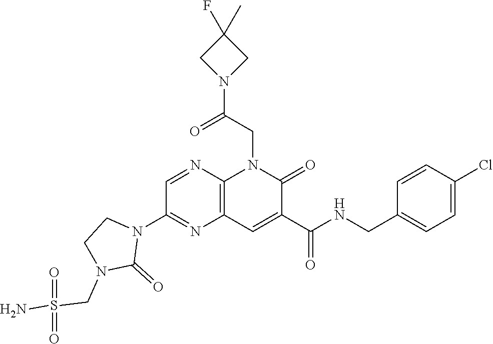 [SMILES: CC1(F)CN(C(=O)Cn2c(=O)c(C(=O)NCc3ccc(Cl)cc3)cc3nc(N4CCN(CS(N)(=O)=O)C4=O)cnc32)C1] |
| | N-[(4-chlorophenyl)methyl]-5-[2-(3-fluoro-3- |
| | methylazetidin-1-yl)-2-oxoethyl]-6-oxo-2-[2-oxo-3- |
| | (sulfamoylmethyl)imidazolidin-1-yl]-5,6- |
| | dihydropyrido[2,3-blpyrazine-7-carboxamide |
| 33 | 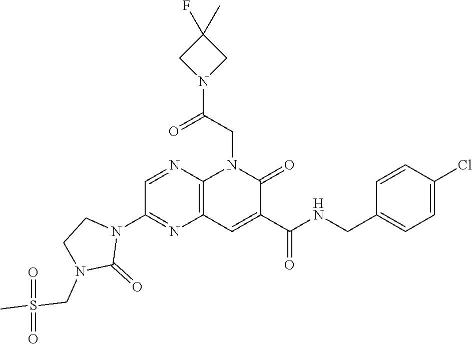 [SMILES: CC1(F)CN(C(=O)Cn2c(=O)c(C(=O)NCc3ccc(Cl)cc3)cc3nc(N4CCN(CS(C)(=O)=O)C4=O)cnc32)C1] |
| | N-[(4-chlorophenyl)methyl]-5-[2-(3-fluoro-3- |
| | methylazetidin-1-yl)-2-oxoethyl]-2-{3- |
| | [(methanesulfonyl)methyl]-2-oxoimidazolidin-1-yl}-6- |
| | oxo-5,6-dihydropyrido[2,3-b]pyrazine-7- |
| | carboxamide |
| 34 | 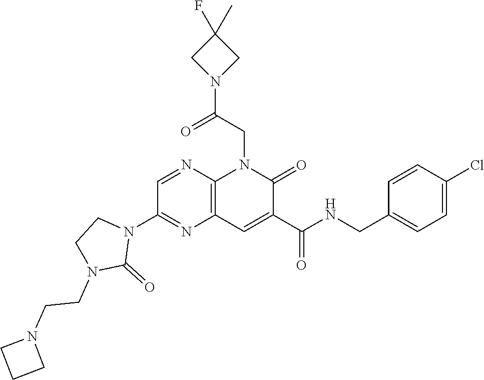 [SMILES: CC1(F)CN(C(=O)Cn2c(=O)c(C(=O)NCc3ccc(Cl)cc3)cc3nc(N4CCN(CCN5CCC5)C4=O)cnc32)C1] |
| | 2-{3-[2-(azetidin-1-yl)ethyl]-2-oxoimidazolidin-1-yl}- |
| | N-[(4-chlorophenyl)methyl]-5-[2-(3-fluoro-3- |
| | methylazetidin-1-yl)-2-oxoethyl]-6-oxo-5,6- |
| | dihydropyrido[2,3-b]pyrazine-7-carboxamid |
| 35 | 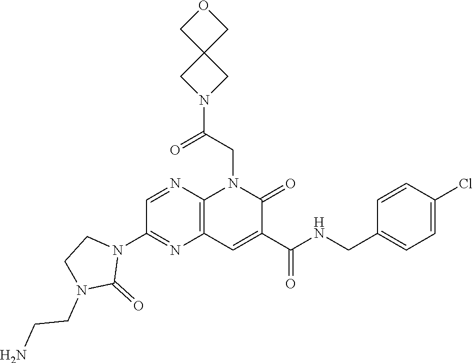 [SMILES: NCCN1CCN(c2cnc3c(cc(C(=O)NCc4ccc(Cl)cc4)c(=O)n3CC(=O)N3CC4(COC4)C3)n2)C1=O] |
| | 2-[3-(2-aminoethyl)-2-oxoimidazolidin-1-yl]-N-[(4- |
| | chlorophenyl)methyl]-5-[2-(2-oxa-6- |
| | azaspiro[3.3]heptan-6-yl)-2-oxoethyl]-6-oxo-5,6- |
| | dihydropyrido[2,3-blpyrazine-7-carboxamide |
| 36 | 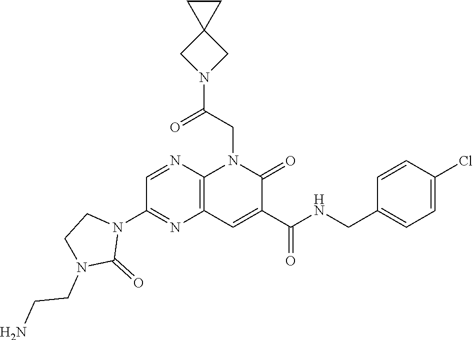 [SMILES: NCCN1CCN(c2cnc3c(cc(C(=O)NCc4ccc(Cl)cc4)c(=O)n3CC(=O)N3CC4(CC4)C3)n2)C1=O] |
| | 2-[3-(2-aminoethyl)-2-oxoimidazolidin-1-yl]-5-[2-(5- |
| | azaspiro[2.3]hexan-5-yl)-2-oxoethyl]-N-[(4- |
| | chlorophenyl)methyl]-6-oxo-5,6-dihydropyrido[2,3- |
| | b]pyrazine-7-carboxamide |
| 37 | 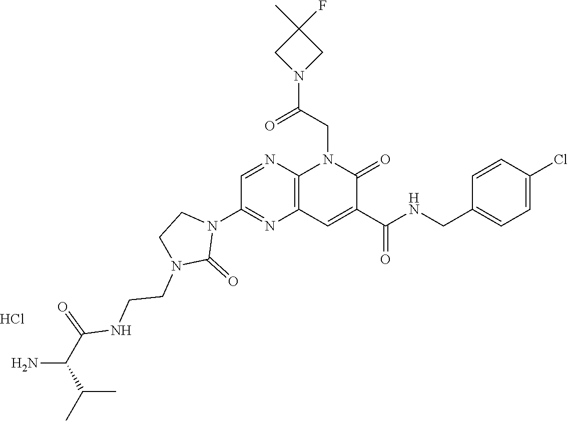 [SMILES: Cl ; CC(C)[C@H](N)C(=O)NCCN1CCN(c2cnc3c(cc(C(=O)NCc4ccc(Cl)cc4)c(=O)n3CC(=O)N3CC(C)(F)C3)n2)C1=O] |
| | N-[(4-chlorophenyl)methyl]-5-[2-(3-fluoro-3- |
| | methylazetidin-1-yl)-2-oxoethyl]-6-oxo-2-{2-oxo-3- |
| | [2-(L-valylamino)ethyl]imidazolidin-1-yl}-5,6- |
| | dihydropyrido[2,3-b]pyrazine-7-carboxamide- |
| | hydrochloride salt |
| 38 | 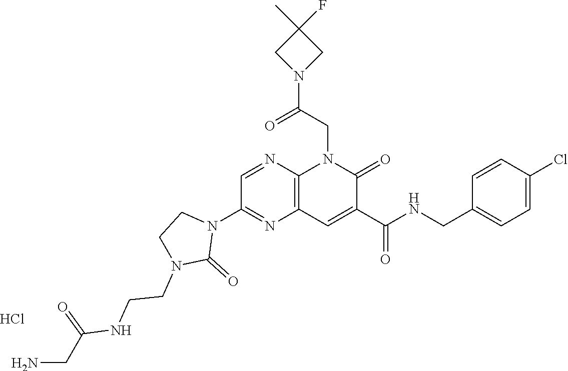 [SMILES: Cl ; CC1(F)CN(C(=O)Cn2c(=O)c(C(=O)NCc3ccc(Cl)cc3)cc3nc(N4CCN(CCNC(=O)CN)C4=O)cnc32)C1] |
| | N-[(4-chlorophenyl)methyl]-5-[2-(3-fluoro-3- |
| | methylazetidin-1-yl)-2-oxoethyl]-2-{3-[2- |
| | (glycylamino)ethyl]-2-oxoimidazolidin-1-yl}-6-oxo- |
| | 5,6-dihydropyrido[2,3-b]pyrazine-7-carboxamide- |
| | hydrochloride salt |
| 39 | 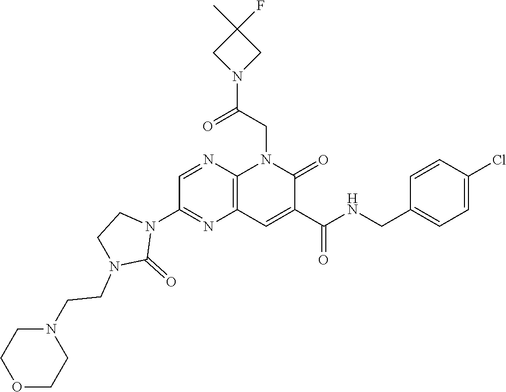 [SMILES: CC1(F)CN(C(=O)Cn2c(=O)c(C(=O)NCc3ccc(Cl)cc3)cc3nc(N4CCN(CCN5CCOCC5)C4=O)cnc32)C1] |
| | N-[(4-chlorophenyl)methyl]-5-[2-(3-fluoro-3- |
| | methylazetidin-1-yl)-2-oxoethyl]-2-{3-[2-(morpholin- |
| | 4-yl)ethyl]-2-oxoimidazolidin-1-yl}-6-oxo-5,6- |
| | dihydropyrido[2,3-b]pyrazine-7-carboxamide |
| 40 | 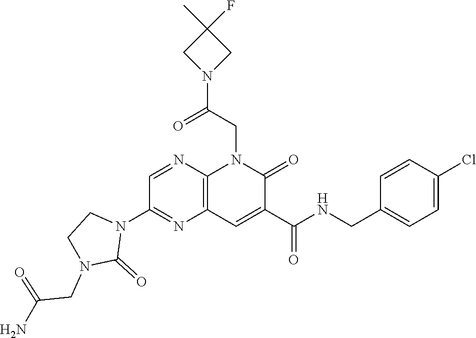 [SMILES: CC1(F)CN(C(=O)Cn2c(=O)c(C(=O)NCc3ccc(Cl)cc3)cc3nc(N4CCN(CC(N)=O)C4=O)cnc32)C1] |
| | 2-[3-(2-amino-2-oxoethyl)-2-oxoimidazolidin-1-yl]-N- |
| | [(4-chlorophenyl)methyl]-5-[2-(3-fluoro-3- |
| | methylazetidin-1-yl)-2-oxoethyl]-6-oxo-5,6- |
| | dihydropyrido[2,3-b]pyrazine-7-carboxamide |
| 41 | 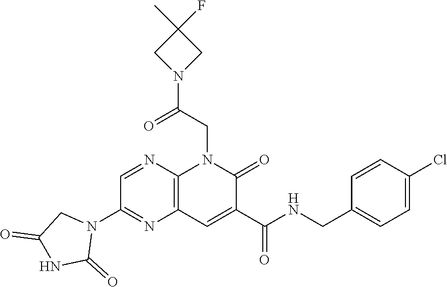 [SMILES: CC1(F)CN(C(=O)Cn2c(=O)c(C(=O)NCc3ccc(Cl)cc3)cc3nc(N4CC(=O)NC4=O)cnc32)C1] |
| | N-[(4-chlorophenyl)methyl]-2-(2,4-dioxoimidazolidin- |
| | 1-yl)-5-[2-(3-fluoro-3-methylazetidin-1-yl)-2- |
| | oxoethyl]-6-oxo-5,6-dihydropyrido[2,3-b]pyrazine-7- |
| | carboxamide |
| 42 | 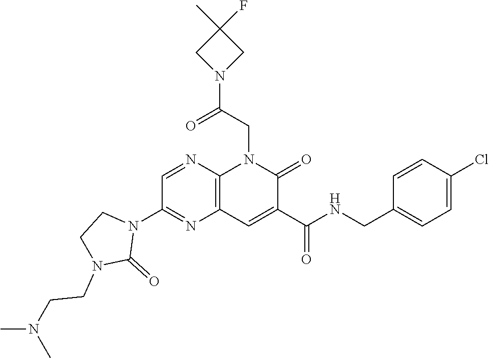 [SMILES: CN(C)CCN1CCN(c2cnc3c(cc(C(=O)NCc4ccc(Cl)cc4)c(=O)n3CC(=O)N3CC(C)(F)C3)n2)C1=O] |
| | N-[(4-chlorophenyl)methyl]-2-{3-[2- |
| | (dimethylamino)ethyl]-2-oxoimidazolidin-1-yl}-5-[2- |
| | (3-fluoro-3-methylazetidin-1-yl)-2-oxoethyl]-6-oxo- |
| | 5,6-dihydropyrido[2,3-b]pyrazine-7-carboxamide |
| 43 | 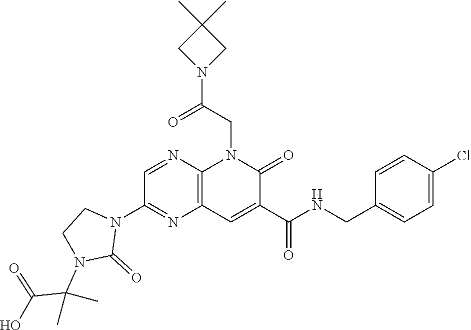 [SMILES: CC1(C)CN(C(=O)Cn2c(=O)c(C(=O)NCc3ccc(Cl)cc3)cc3nc(N4CCN(C(C)(C)C(=O)O)C4=O)cnc32)C1] |
| | 2-[3-(7-{[(4-chlorophenyl)methyl]carbamoyl}-5-[2- |
| | (3,3-dimethylazetidin-1-yl)-2-oxoethyl]-6-oxo-5,6- |
| | dihydropyrido[2,3-b]pyrazin-2-yl)-2-oxoimidazolidin- |
| | 1-yl]-2-methylpropanoic acid |
| 44 | 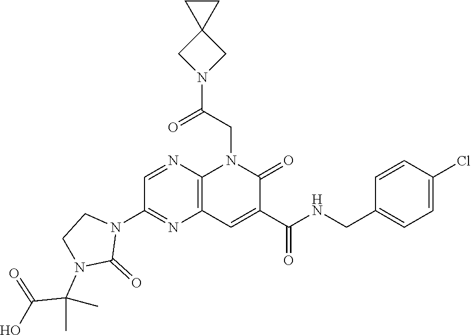 [SMILES: CC(C)(C(=O)O)N1CCN(c2cnc3c(cc(C(=O)NCc4ccc(Cl)cc4)c(=O)n3CC(=O)N3CC4(CC4)C3)n2)C1=O] |
| | 2-[3-(5-[2-(5-azaspiro[2.3]hexan-5-yl)-2-oxoethyl]-7- |
| | {[(4-chlorophenyl)methyl]carbamoyl}-6-oxo-5,6- |
| | dihydropyrido[2,3-b]pyrazin-2-yl)-2-oxoimidazolidin- |
| | 1-yl]-2-methylpropanoic acid |
| 45 | 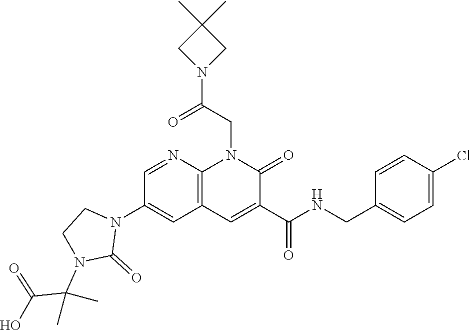 [SMILES: CC1(C)CN(C(=O)Cn2c(=O)c(C(=O)NCc3ccc(Cl)cc3)cc3cc(N4CCN(C(C)(C)C(=O)O)C4=O)cnc32)C1] |
| | 2-(3-(6-{[(4-chlorophenyl)methyl]carbamoyl}-8-(2- |
| | (3,3-dimethylazetidin-1-yl)-2-oxoethyl)-7-oxo-7,8- |
| | dihydro-1,8-naphthyridin-3-yl)-2-oxoimidazolidin-1- |
| | yl)-2-methylpropanoic acid |
| 46 | 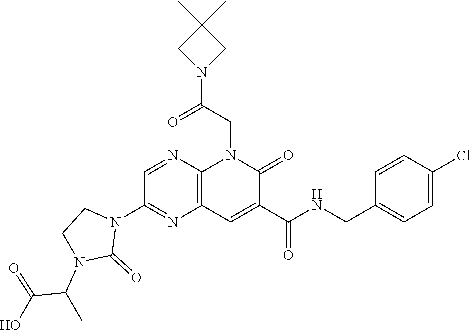 [SMILES: CC(C(=O)O)N1CCN(c2cnc3c(cc(C(=O)NCc4ccc(Cl)cc4)c(=O)n3CC(=O)N3CC(C)(C)C3)n2)C1=O] |
| | 2-[3-(7-{[(4-chlorophenyl)methyl]carbamoyl}-5-[2- |
| | (3,3-dimethylazetidin-1-yl)-2-oxoethyl]-6-oxo-5,6- |
| | dihydropyrido[2,3-b]pyrazin-2-yl)-2-oxoimidazolidin- |
| | 1-yl]propanoic acid |

In certain embodiments, the compound is described by the structure selected from any one of compounds 1-6, 8-24, 26-28 and 36-46. In some cases, the compound is described by the structure of compound 7. In some cases, the compound is described by the structure of compound 25. In some cases, the compound is described by the structure of any one of compounds 29-35.

In certain embodiments, the compound is described by the structure of one of the compounds of Table 1. It is understood that any of the compounds shown in Table 1 may be present in a salt form. In some cases, the salt form of the compound is a pharmaceutically acceptable salt. It is understood that any of the compounds shown in Table 1 may be present in a prodrug form.

Aspects of the present disclosure include herpesvirus inhibitor compounds (e.g., as described herein), salts thereof (e.g., pharmaceutically acceptable salts), and/or solvate, hydrate and/or prodrug forms thereof. In addition, it is understood that, in any compound described herein having one or more chiral centers, if an absolute stereochemistry is not expressly indicated, then each center may independently be of R-configuration or S-configuration or a mixture thereof. It will be appreciated that all permutations of salts, solvates, hydrates, prodrugs and stereoisomers are meant to be encompassed by the present disclosure.

The compounds provided herein are intended to encompass all tautomers and possible stereo, optical and geometrical isomers, unless a particular stereochemistry is specified. Where structural isomers are interconvertible, the compound may exist as a single tautomer or a mixture of tautomers. This can take the form of proton tautomerism in the compound that contains, for example, an imino, keto, or oxime group; or so-called valence tautomerism in the compound that contain an aromatic moiety. It follows that a single compound may exhibit more than one type of isomerism.

The compounds provided herein may be enantiomerically pure, such as a single enantiomer or a single diastereomer, or be stereoisomeric mixtures, such as a mixture of enantiomers, e.g., a racemic mixture of two enantiomers; or a mixture of two or more diastereomers. As such, one of skill in the art will recognize that administration of a compound in its (R) form is equivalent, for compounds that undergo epimerization in vivo, to administration of the compound in its (S) form. Conventional techniques for the preparation/isolation of individual enantiomers include synthesis from a suitable optically pure precursor, asymmetric synthesis from achiral starting materials, or resolution of an enantiomeric mixture, for example, chiral chromatography, recrystallization, resolution, diastereomeric salt formation, or derivatization into diastereomeric adducts followed by separation.

In some embodiments, the subject herpesvirus inhibitor compounds, or a prodrug form thereof, are provided in the form of pharmaceutically acceptable salts. Compounds containing an amine or nitrogen containing heteroaryl group may be basic in nature and accordingly may react with any number of inorganic and organic acids to form pharmaceutically acceptable acid addition salts. When the compound provided herein contains an acidic or basic moiety, it may also be provided as a pharmaceutically acceptable salt. See, Berge et al. (1977) J. Pharm. Sci. 66:1-19; and Handbook of Pharmaceutical Salts, Properties, and Use; Stahl and Wermuth, Ed.; Wiley-VCH and VHCA: Zurich, Switzerland, 2002.

Suitable acids for use in the preparation of pharmaceutically acceptable salts include, but are not limited to, acetic acid, 2,2-dichloroacetic acid, acylated amino acids, adipic acid, alginic acid, ascorbic acid, L-aspartic acid, benzenesulfonic acid, benzoic acid, 4-acetamidobenzoic acid, boric acid, (+)-camphoric acid, camphorsulfonic acid, (+)-(1S)-camphor-10-sulfonic acid, capric acid, caproic acid, caprylic acid, cinnamic acid, citric acid, cyclamic acid, cyclohexanesulfamic acid, dodecylsulfuric acid, ethane-1,2-disulfonic acid, ethanesulfonic acid, 2-hydroxy-ethanesulfonic acid, formic acid, fumaric acid, galactaric acid, gentisic acid, glucoheptonic acid, D-gluconic acid, D-glucuronic acid, L-glutamic acid, α-oxoglutaric acid, glycolic acid, hippuric acid, hydrobromic acid, hydrochloric acid, hydroiodic acid, (+)-L-lactic acid, (±)-DL-lactic acid, lactobionic acid, lauric acid, maleic acid, (−)-L-malic acid, malonic acid, (±)-DL-mandelic acid, methanesulfonic acid, naphthalene-2-sulfonic acid, naphthalene-1,5-disulfonic acid, 1-hydroxy-2-naphthoic acid, nicotinic acid, nitric acid, oleic acid, orotic acid, oxalic acid, palmitic acid, pamoic acid, perchloric acid, phosphoric acid, L-pyroglutamic acid, saccharic acid, salicylic acid, 4-amino-salicylic acid, sebacic acid, stearic acid, succinic acid, sulfuric acid, tannic acid, (+)-L-tartaric acid, thiocyanic acid, p-toluenesulfonic acid, undecylenic acid, and valeric acid.

Suitable bases for use in the preparation of pharmaceutically acceptable salts, including, but not limited to, inorganic bases, such as magnesium hydroxide, calcium hydroxide, potassium hydroxide, zinc hydroxide, or sodium hydroxide; and organic bases, such as primary, secondary, tertiary, and quaternary, aliphatic and aromatic amines, including L-arginine, benethamine, benzathine, choline, deanol, diethanolamine, diethylamine, dimethylamine, dipropylamine, diisopropylamine, 2-(diethylamino)-ethanol, ethanolamine, ethylamine, ethylenediamine, isopropylamine, N-methyl-glucamine, hydrabamine, 1H-imidazole, L-lysine, morpholine, 4-(2-hydroxyethyl)-morpholine, methylamine, piperidine, piperazine, propylamine, pyrrolidine, 1-(2-hydroxyethyl)-pyrrolidine, pyridine, quinuclidine, quinoline, isoquinoline, secondary amines, triethanolamine, trimethylamine, triethylamine, N-methyl-D-glucamine, 2-amino-2-(hydroxymethyl)-1,3-propanediol, and tromethamine.

In some embodiments, the subject compounds are provided in a prodrug form. “Prodrug” refers to a derivative of an active agent that requires a transformation within the body to release the active agent. In certain embodiments, the transformation is an enzymatic transformation. Prodrugs are frequently, although not necessarily, pharmacologically inactive until converted to the active agent. “Promoiety” refers to a form of protecting group that, when used to mask a functional group within an active agent, converts the active agent into a prodrug. In some cases, the promoiety will be attached to the drug via bond(s) that are cleaved by enzymatic or non-enzymatic means in vivo. Any convenient prodrug forms of the subject compounds can be prepared, e.g., according to the strategies and methods described by Rautio et al. (“Prodrugs: design and clinical applications”, Nature Reviews Drug Discovery 7, 255-270 (February 2008)). The prodrug may also have enhanced solubility in pharmaceutical compositions over the parent compound. A prodrug may be converted into the parent drug by various mechanisms, including enzymatic processes and metabolic hydrolysis. Serafin et al. (2009) Mini Rev. Med. Chem. 9:81-497; Jornada et al. (2016) Molecules 21(1):42.

In some embodiments, the subject compounds, prodrugs, stereoisomers or salts thereof are provided in the form of a solvate (e.g., a hydrate). The term “solvate” as used herein refers to a complex or aggregate formed by one or more molecules of a solute, e.g. a prodrug or a pharmaceutically-acceptable salt thereof, and one or more molecules of a solvent. Such solvates are typically crystalline solids having a substantially fixed molar ratio of solute and solvent. Representative solvents include by way of example, water, methanol, ethanol, isopropanol, acetic acid, and the like. When the solvent is water, the solvate formed is a hydrate.

In some embodiments, the subject compounds are provided by oral dosing and absorbed into the bloodstream. In some embodiments, the oral bioavailability of the subject compounds is 30% or more. Modifications may be made to the subject compounds or their formulations using any convenient methods to increase absorption across the gut lumen or their bioavailability. In certain other embodiments, the compounds are provided by topical administration. In yet other embodiments, the compounds are provided by intravenous therapy.

In some embodiments, the subject compounds are metabolically stable (e.g., remain substantially intact in vivo during the half-life of the compound). In certain embodiments, the compounds have a half-life (e.g., an in vivo half-life) of 5 minutes or more, such as 10 minutes or more, 12 minutes or more, 15 minutes or more, 20 minutes or more, 30 minutes or more, 60 minutes or more, 2 hours or more, 6 hours or more, 12 hours or more, 24 hours or more, or even more.

Synthesis

In certain embodiments, subject compounds (e.g., of formula (I), as described herein) are synthesized using methods and conditions that are known to one of ordinary skill in the art. In certain cases, a subject compound may be synthesized according to the reaction sequence depicted in Scheme 1:

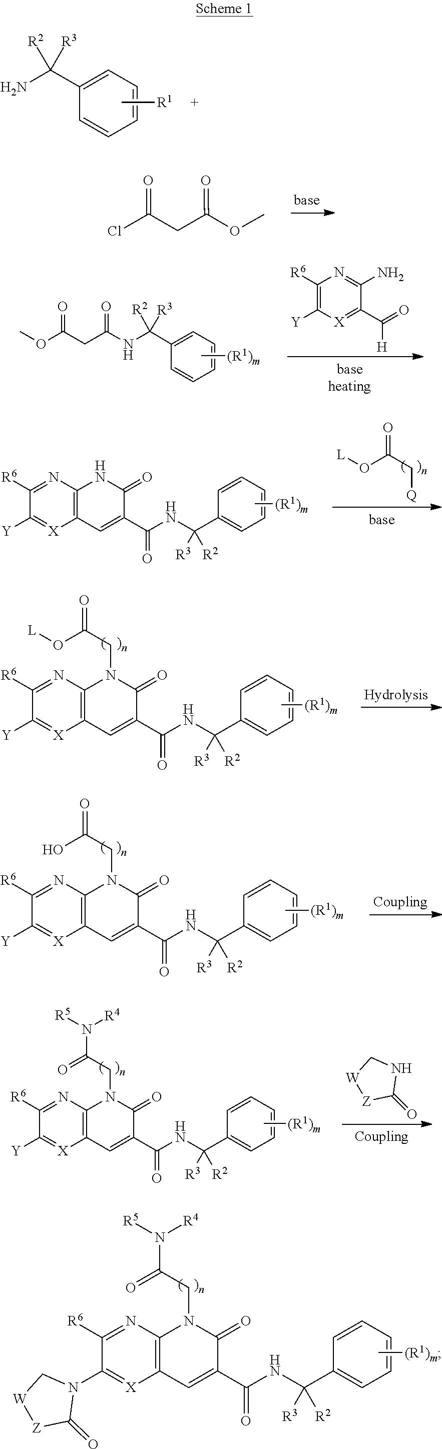

wherein R¹, R², R³, R⁴, R⁵, R⁶, n, m, W, X, Y and Z are as defined herein. L is alkyl or branched alkyl, such as methyl, ethyl, or tert-butyl. Q is a leaving group, such as bromo, iodo, mesylate or triflate. Y is chloro, bromo, or iodo.

The starting materials and reagents employed in Scheme 1 may be obtained commercially or through techniques known to one of ordinary skill in the art.

In certain embodiments, compounds of Formula (II) are synthesized using methods and conditions that are known to one of ordinary skill in the art, e.g., as depicted in Scheme 2:

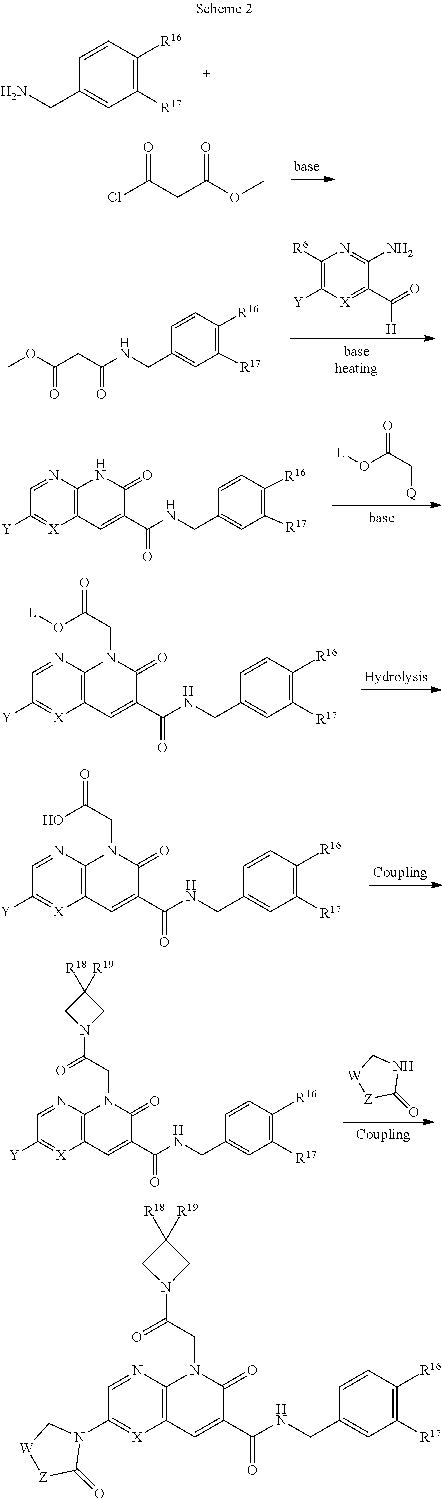

wherein R¹⁶, R¹⁷, R¹⁸, R¹⁹, W, X, Y, and Z are as defined herein. L is alkyl or branched alkyl, such as methyl, ethyl, or tert-butyl. Q is a leaving group, such as bromo, iodo, mesylate or triflate. Y is chloro, bromo, or iodo.

The starting materials and reagents employed in Scheme 2 may be obtained commercially or through techniques known to one of ordinary skill in the art.

In certain embodiments, compounds of Formula (V) are synthesized using methods and conditions that are known to one of ordinary skill in the art, e.g., as depicted in Scheme 3:

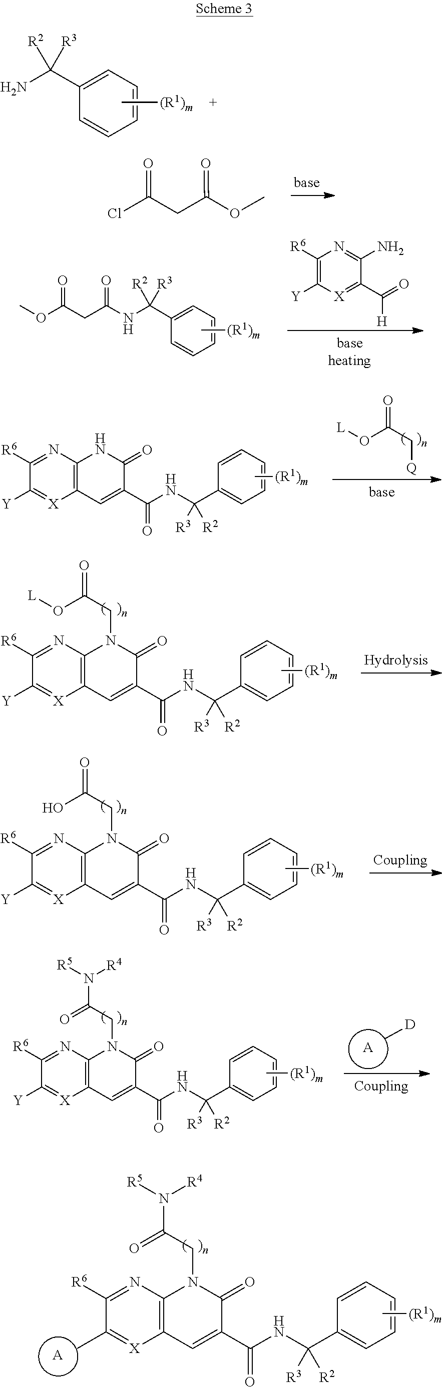

wherein A, R¹, R², R³, R⁴, R⁵, R⁶, n, m, W, X, Y, and Z are as defined herein. L is alkyl or branched alkyl, such as methyl, ethyl, or tert-butyl. D is H, I, OsO₂CF₃, B(OH)₂ or Sn(Bu)₃. Q is a leaving group, such as bromo, iodo, mesylate or triflate. Y is chloro, bromo, or iodo.

The starting materials and reagents employed in Scheme 3 may be obtained commercially or through techniques known to one of ordinary skill in the art.

Schemes 1, 2, 3 are meant to be by way of non-limiting examples only, and one of ordinary skill in the art will understand that alternate reagents, solvents or starting materials can be used to make compounds of any one of the Formula (I)-(V) or compounds 1-25 as described herein.

Methods

As summarized above, provided herein are methods for inhibiting a herpesvirus in a cell, and treating a herpesvirus in an individual. In some embodiments the herpesvirus is cytomegalovirus (CMV). In certain embodiments, the herpesvirus is human cytomegalovirus (CMV) inhibitors.

Methods of Inhibiting a Herpesvirus

As summarized above, aspects of the present disclosure include herpesvirus inhibitors, and methods of inhibition using the same. In some embodiments, the subject compounds provide inhibition of a herpesvirus, with no significant human ether-a-go-go related gene product (hERG) inhibition. Without being bound to any particular theory, inhibition of the hERG (Kv11.1) potassium channel (also known as inhibition of IKr) has been associated with cardiac toxicity, and has resulted in the withdrawal of certain drugs from the market (Danker, et al., Frontiers in Pharmacology 5 (Article 203), 1-11 (2014).

The Herpesvirus family contains eight known viruses that infect humans (Brown, et. al., Current Opinion in Virology 1(2), 142-149 (2011)). These members of Herpesviridae are the herpes simplex viruses (HSV-1 and HSV-2), varicella-zoster virus (VZV), Epstein-Barr Virus (EBV), human cytomegalovirus (HCMV), and human herpes viruses (HHV-6A/B, HHV-7 and HHV-8). Infections by members of this family are responsible for a wide range of diseases. For patients with suppressed immune systems as a result of chemotherapy or old-age, serious morbidity and mortality can occur (Villarreal E. C., Current and potential therapies for the treatment of herpesvirus infections. In: Jucker E. (eds) Progress in Drug Research. vol 60. (2003) Birkhsuser, Basel, 263-307). For transplant patients who are immunocompromised, HCMV infection, whether from donor or primary infection, is a serious event and even with current therapies can result in morbidity and mortality. The incidence of CMV infection for transplant patients reported depends on the type of transplant and incidence can be as high as 75% for heart-lung transplantation and around 50% for kidney-pancreas transplantation (Azevedo, et al., Clinics 70 (7), 515-523 (2015)).

As disclosed herein, in some cases the herpesvirus inhibiting compounds are compounds that inhibit the herpesvirus in a cell. In some cases, Experiments conducted by the inventors indicate that the herpesvirus inhibiting compounds inhibit the herpesvirus, without significant hERG inhibition. In certain cases, the subject compounds exhibit a hERG inhibition profile with an IC₅₀>10 microM, such as >20, >30, or greater than 40 microM, while maintaining herpesvirus inhibition.

Accordingly, provided herein are methods of inhibiting a herpesvirus in a cell infected with a herpesvirus, the method comprising contacting the cell with a compound as described herein. In certain cases, the compound is of any one of the formulae (I)-(V). In certain cases, the compound is of any one of the structures 1-46.

In certain embodiments of the methods, the herpesvirus is selected from cytomegalovirus (CMV), herpes simplex virus-1 (HSV-1), herpes simplex virus-2 (HSV-2), varicella-zoster virus, Epstein-Barr virus (EBV), and Kaposi's sarcoma-associated herpesvirus (KSHV). In certain cases, the herpesvirus is cytomegalovirus (CMV). In certain cases the herpesvirus is human cytomegalovirus (HCMV).

By inhibiting a herpesvirus it is meant that the activity of the virus is decreased by 10% or more, such as 20% or more, 30% or more, 40% or more, 50% or more, 60% or more, 70% or more, 80% or more, 90% or more, 95% or more (e.g., relative to a control in any convenient in vitro inhibition assay). In some cases, inhibiting a herpesvirus means decreasing the activity of the virus by a factor of 2 or more, such as 3 or more, 5 or more, 10 or more, 100 or more, or 1000 or more, relative to its normal activity (e.g., relative to a control as measured by any convenient assay).

In some cases, the method is a method of inhibiting herpesvirus in a sample. The term “sample” as used herein relates to a material or mixture of materials, typically, although not necessarily, in fluid form, containing one or more components of interest.

In some embodiments, the subject compounds have a herpesvirus inhibition profile that reflects activity against additional enzymes. In some embodiments, the subject compounds specifically inhibit a herpesvirus without undesired inhibition of one or more other enzymes. In some embodiments, the subject compounds specifically inhibit a herpesvirus without undesired inhibition of hERG.

In some embodiments, the subject compounds inhibit a herpesvirus, as determined by an inhibition assay, e.g., by an assay that determines the level of activity of the herpesvirus either in a cell-free system or in a cell after treatment with a subject compound, relative to a control, by measuring the IC₅₀ or EC₅₀ value, respectively. In certain embodiments, the subject compounds have an IC₅₀ value (or EC₅₀ value) of 10 μM or less, such as 3 μM or less, 1 μM or less, 500 nM or less, 300 nM or less, 200 nM or less, 100 nM or less, 50 nM or less, 30 nM or less, 10 nM or less, 5 nM or less, 3 nM or less, 1 nM or less, or even lower.

As summarized above, aspects of the disclosure include methods of inhibiting a herpesvirus. A subject compound (e.g., as described herein) may inhibit at activity of a herpesvirus in the range of 10% to 100%, e.g., by 10% or more, 20% or more, 30% or more, 40% or more, 50% or more, 60% or more, 70% or more, 80% or more, or 90% or more. In certain assays, a subject compound may inhibit its target with an IC₅₀ of 1×10⁻⁶ M or less (e.g., 1×10⁻⁶ M or less, 1×10⁻⁷ M or less, 1×10⁻⁸ M or less, 1×10⁻⁹ M or less, 1×10⁻¹⁰ M or less, or 1×10⁻¹¹ M or less).

The protocols that may be employed in determining herpesvirus activity are numerous, and include but are not limited to cell-free assays, e.g., binding assays; assays using purified enzymes, cellular assays in which a cellular phenotype is measured, e.g., gene expression assays; and in vivo assays that involve a particular animal (which, in certain embodiments may be an animal model for a condition related to the target pathogen).

In some embodiments, the subject method is an in vitro method that includes contacting a sample with a subject compound that specifically inhibits a herpesvirus. In certain embodiments, the sample is suspected of containing a herpesvirus and the subject method further comprises evaluating whether the compound inhibits the herpesvirus.

In certain embodiments, the subject compound is a modified compound that includes a label, e.g., a fluorescent label, and the subject method further includes detecting the label, if present, in the sample, e.g., using optical detection.

In certain embodiments, the compound is modified with a support or with affinity groups that bind to a support (e.g. biotin), such that any sample that does not bind to the compound may be removed (e.g., by washing).

In another embodiment of the subject method, the sample is known to contain a herpesvirus.

In some embodiments, the method is a method of reducing herpesvirus cell proliferation, where the method includes contacting the cell with an effective amount of a subject herpesvirus inhibitor compound (e.g., as described herein) to reduce herpesvirus cell proliferation. In certain cases, the subject methods can be performed in combination with one or more additional active agents, e.g., a chemotherapeutic agent. The herpesvirus cells can be in vitro or in vivo. In certain instances, the method includes contacting the cell with a herpesvirus inhibitor compound (e.g., as described herein) and contacting the cell with one or more additional active agents (e.g., a chemotherapeutic agent). Any convenient herpesvirus can be targeted.

Methods of Treatment

Aspects of the present disclosure include methods of treating or preventing a herpesvirus infection in an individual, the method comprising administering to the individual an effective amount of a subject compound (e.g., as described herein). In certain embodiments, the method is a method of treating a herpesvirus infection in an individual. In certain embodiments, the method is a method of preventing a herpesvirus infection in an individual.

In certain embodiments of the methods, the herpesvirus is selected from cytomegalovirus (CMV), herpes simplex virus-1 (HSV-1), herpes simplex virus-2 (HSV-2), varicella-zoster virus, Epstein-Barr virus (EBV), and Kaposi's sarcoma-associated herpesvirus (KSHV). In certain cases, the herpesvirus is cytomegalovirus (CMV). In certain cases, the herpesvirus is human cytomegalovirus (HCMV).

In certain embodiments of the methods, individual is one who is diagnosed with or suspected of having a herpesvirus. In certain cases, the individual has been diagnosed with a herpesvirus selected from cytomegalovirus (CMV), herpes simplex virus-1 (HSV-1), herpes simplex virus-2 (HSV-2), varicella-zoster virus (VZV), Epstein-Barr virus (EBV), and Kaposi's sarcoma-associated herpesvirus (KSHV). In certain cases, the individual has been diagnosed with cytomegalovirus (CMV).

Any convenient herpesvirus inhibitors can be used in the subject methods of treating or preventing a herpesvirus. In certain cases, the herpesvirus inhibitor compound is a compound as described herein. In certain cases, the herpesvirus inhibitor is a compound of any one of formulae (I)-(V) (e.g., as described herein). In certain cases, the herpesvirus inhibitor is selected from any one of compounds 1-46.

As such, aspects of the method include administering a therapeutically effective amount of a subject compound (e.g., as described above) to an individual under conditions by which the compound inhibits a herpesvirus. Any convenient protocol for administering the compound may be employed. Depending upon the potency of the compound, the cells of interest, the manner of administration, the number of cells present, various protocols may be employed.

The subject compound may be administered as part of a pharmaceutical composition (e.g., as described herein). In certain instances of the method, the compound that is administered as part of a pharmaceutical composition is a compound of one of formulae (I)-(V). In certain instances of the method, the compound that is administered is described by one of the compounds of Table 1 (e.g., compounds 1-46).

In some embodiments, an “effective amount” is an amount of a subject compound that, when administered to an individual in one or more doses, in monotherapy or in combination therapy, is effective to inhibit the herpesvirus by about 20% (20% inhibition), at least about 30% (30% inhibition), at least about 40% (40% inhibition), at least about 50% (50% inhibition), at least about 60% (60% inhibition), at least about 70% (70% inhibition), at least about 80% (80% inhibition), or at least about 90% (90% inhibition), compared to the herpesvirus activity in the individual in the absence of treatment with the compound, or alternatively, compared to the herpesvirus activity in the individual before or after treatment with the compound.

In some embodiments, a “therapeutically effective amount” of a compound is an amount that, when administered in one or more doses to an individual having a herpesvirus, is effective to achieve a 1.5-log, a 2-log, a 2.5-log, a 3-log, a 3.5-log, a 4-log, a 4.5-log, or a 5-log reduction in herpesvirus activity. A therapeutically effective amount can be a “prophylactically effective amount” as prophylaxis can be considered therapy.

In some embodiments, an effective amount of a compound is an amount that ranges from about 50 ng/ml to about 50 μg/ml (e.g., from about 50 ng/ml to about 40 μg/ml, from about 30 ng/ml to about 20 μg/ml, from about 50 ng/ml to about 10 μg/ml, from about 50 ng/ml to about 1 μg/ml, from about 50 ng/ml to about 800 ng/ml, from about 50 ng/ml to about 700 ng/ml, from about 50 ng/ml to about 600 ng/ml, from about 50 ng/ml to about 500 ng/ml, from about 50 ng/ml to about 400 ng/ml, from about 60 ng/ml to about 400 ng/ml, from about 70 ng/ml to about 300 ng/ml, from about 60 ng/ml to about 100 ng/ml, from about 65 ng/ml to about 85 ng/ml, from about 70 ng/ml to about 90 ng/ml, from about 200 ng/ml to about 900 ng/ml, from about 200 ng/ml to about 800 ng/ml, from about 200 ng/ml to about 700 ng/ml, from about 200 ng/ml to about 600 ng/ml, from about 200 ng/ml to about 500 ng/ml, from about 200 ng/ml to about 400 ng/ml, or from about 200 ng/ml to about 300 ng/ml).

In some embodiments, an effective amount of a compound is an amount that ranges from about 10 pg to about 100 mg, e.g., from about 10 pg to about 50 pg, from about 50 pg to about 150 pg, from about 150 pg to about 250 pg, from about 250 pg to about 500 pg, from about 500 pg to about 750 pg, from about 750 pg to about 1 ng, from about 1 ng to about 10 ng, from about 10 ng to about 50 ng, from about 50 ng to about 150 ng, from about 150 ng to about 250 ng, from about 250 ng to about 500 ng, from about 500 ng to about 750 ng, from about 750 ng to about 1 pg, from about 1 pg to about 10 pg, from about 10 pg to about 50 pg, from about 50 pg to about 150 pg, from about 150 pg to about 250 pg, from about 250 pg to about 500 pg, from about 500 pg to about 750 pg, from about 750 pg to about 1 mg, from about 1 mg to about 50 mg, from about 1 mg to about 100 mg, or from about 50 mg to about 100 mg. The amount can be a single dose amount or can be a total daily amount. The total daily amount can range from 10 pg to 100 mg, or can range from 100 mg to about 500 mg, or can range from 500 mg to about 1000 mg.

In some embodiments, a single dose of a compound is administered. In other embodiments, multiple doses are administered. Where multiple doses are administered over a period of time, the compound can be administered twice daily (qid), daily (qd), every other day (qod), every third day, three times per week (tiw), or twice per week (biw) over a period of time. For example, a compound is administered qid, qd, qod, tiw, or biw over a period of from one day to about 2 years or more. For example, a compound is administered at any of the aforementioned frequencies for one week, two weeks, one month, two months, six months, one year, or two years, or more, depending on various factors.

In some embodiments, the subject is mammalian. In certain instances, the subject is human. Other subjects can include domestic pets (e.g., dogs and cats), livestock (e.g., cows, pigs, goats, horses, and the like), rodents (e.g., mice, guinea pigs, and rats, e.g., as in animal models of disease), as well as non-human primates (e.g., chimpanzees, and monkeys). The subject may be in need of treatment for a herpesvirus. In some instances, the subject methods include diagnosing a herpesvirus infection, including any one of the herpesviruses described herein. In some embodiments, the compound is administered as a pharmaceutical preparation.

Individuals suitable for treatment with a method of the present disclosure for treating a herpesvirus infection include individuals who have been diagnosed as having a herpesvirus infection. Subjects suitable for treatment with a method of the present disclosure for treating a herpesvirus infection include individuals who have not been diagnosed as having a herpesvirus infection. In some cases, the individual does not have a herpesvirus infection, but is at greater risk than the general population of contracting a herpesvirus infection or is at increased likelihood of complications associated with herpesvirus infection. Subjects suitable for treatment with a method of the present disclosure for treating a herpesvirus infection include individuals who have been diagnosed with a herpesvirus infection that is resistant to treatments with other approved treatments such as nucleosides, for example acyclovir and ganciclovir or their prodrugs (valacylovir and valganciclovir), or others, for example foscarnet and letermovir.

In some cases, the individual has a herpesvirus infection, and also has an immunodeficiency virus (e.g., human immunodeficiency virus; HIV) infection. In some cases, the individual is an organ transplant recipient. In some cases, the individual is a liver transplant recipient. In some cases, the individual is a kidney transplant recipient. In some cases, the individual is a liver transplant recipient. In some cases, the individual is a bone marrow transplant recipient. In some cases, the individual is a lung transplant recipient. In some cases, the individual is a cancer patient. In some cases, the individual is elderly. In some cases, the individual is a neonate.

In some cases, the individual does not have a herpesvirus infection; and is a prospective organ transplant recipient. In some cases, the individual does not have a herpesvirus infection; and is a prospective liver transplant recipient. In some cases, the individual does not have a herpesvirus infection; and is a prospective kidney transplant recipient. In some cases, the individual does not have a herpesvirus infection; and is a prospective bone marrow transplant recipient. In some cases, the individual does not have a herpesvirus infection; and is a prospective lung transplant recipient. In some cases, the individual is a neonate at risk of developing a herpesvirus infection from exposure to another individual, for example their mother.

In certain embodiments, the herpesvirus inhibitor compound is a modified compound that includes a label, and the method further includes detecting the label in the subject. The selection of the label depends on the means of detection. Any convenient labeling and detection systems may be used in the subject methods, see e.g., Baker, “The whole picture,” Nature, 463, 2010, p 977-980. In certain embodiments, the compound includes a fluorescent label suitable for optical detection. In certain embodiments, the compound includes a radiolabel for detection using positron emission tomography (PET) or single photon emission computed tomography (SPECT). In some cases, the compound includes a paramagnetic label suitable for tomographic detection. The subject compound may be labeled, as described above, although in some methods, the compound is unlabeled and a secondary labeling agent is used for imaging.

### Combination Therapies

The subject compounds can be administered to a subject alone or in combination with an additional, i.e., second, active agent. Combination therapeutic methods where the subject herpesvirus inhibitor compounds may be used in combination with a second active agent or an additional therapy. For example, herpesvirus inhibitor compounds can be administered alone or in conjunction with one or more other drugs, such as drugs employed in the treatment of diseases including but not limited to, immunomodulatory diseases and conditions, and cancer. In some embodiments, the subject method further includes coadministering concomitantly or in sequence a second agent, e.g., a small molecule, a chemotherapeutic, an antibody, an antibody fragment, an antibody-drug conjugate, an aptamer, a protein, or a checkpoint inhibitor. In certain cases, the subject herpesvirus inhibitors (e.g., as described herein) are administered in combination with an antiviral agent. In some embodiments, the subject compound and an antiviral agent, e.g. interferon, ribavirin, Enfuvirtide; RFI-641 (4,4″-bis-{4,6-bis-[3-(bis-carbamoylmethyl-sulfamoyl)-phenylamino]-(1,3,5)triazin-2-ylamino}-biphenyl-2,2″-disulfonic acid); BMS-433771 (2H-Imidazo(4,5-c)pyridin-2-one, 1-cyclopropyl-1,3-dihydro-3-((1-(3-hydroxypropyl)-1H-benzimidazol-2-yl)methyl)); arildone; Pleconaril (3-(3,5-Dimethyl-4-(3-(3-methyl-5-isoxazolyl)propoxy)phenyl)-5-(trifluoromethyl)-1,2,4-oxadiazole); Amantadine (tricyclo[3.3.1.1.3,7]decane-1-amine hydrochloride); Rimantadine (alpha-methyltricyclo[3.3.1.1.3,7]decane-1-methanamine hydrochloride); Acyclovir (acycloguanosine); Valaciclovir; Penciclovir (9-(4-hydroxy-3-hydroxymethyl-but-1-yl)guanine); Famciclovir (diacetyl ester of 9-(4-hydroxy-3-hydroxymethyl-but-1-yl)-6-deoxyguanine); Gancyclovir (9-(1,3-dihydroxy-2-propoxymethyl)guanine); Ara-A (adenosine arabinoside); Zidovudine (3′-azido-2′,3′-dideoxythymidine); Cidofovir (1-[(S)-3-hydroxy-2-(phosphonomethoxy)propyl]cytosine dihydrate); Dideoxyinosine (2′,3′-dideoxyinosine); Zalcitabine (2′,3′-dideoxycytidine); Stavudine (2′,3′-didehydro-2′,3′-dideoxythymidine); Lamivudine ((−)-β-L-3′-thia-2′,3′-dideoxycytidine); Abacavir (1S,4R)-4-[2-amino-6-(cyclopropylamino)-9H-purin-9-yl]-2-cyclopentene-1-methanol succinate); Emtricitabine (−)-β-L-3′-thia-2′,3′-dideoxy-5-fluorocytidine); Tenofovir disoproxil (Fumarate salt of bis(isopropoxycarbonyloxymethyl) ester of (R)-9-(2-phosphonylmethoxypropyl)adenine); Bromovinyl deoxyuridine (Brivudin); Iodo-deoxyuridine (Idoxuridine); Trifluorothymidine (Trifluridine); Nevirapine (11-cyclopropyl-5,11-dihydro-4-methyl-6H-dipyrido[3,2-b:2′,3′-f][1,4]diazepin-6-one); Delavirdine (1-(5-methanesulfonamido-1H-indol-2-yl-carbonyl)-4-[3-(1-methylethyl-amino)pyridinyl) piperazine monomethane sulfonated); Efavirenz ((−)6-chloro-4-cyclopropylethynyl-4-trifluoromethyl-1,4-dihydro-2H-3,1-benzoxazin-2-one); Foscarnet (trisodium phosphonoformate); Letermovir {(4S)-8-Fluoro-2-[4-(3-methoxyphenyl)-1-piperazinyl]-3-[2-methoxy-5-(trifluoromethyl)phenyl]-3,4-dihydro-4-quinazolinyl}acetic acid; Ribavirin (1-β-D-ribofuranosyl-1H-1,2,4-triazole-3-carboxamide); Raltegravir (N-[(4-Fluorophenyl)methyl]-1,6-dihydro-5-hydroxy-1-methyl-2-[1-methyl-1-[[(5-methyl-1,3,4-oxadiazol-2-yl)carbonyl]amino]ethyl]-6-oxo-4-pyrimidinecarboxamide monopotassium salt); Neplanocin A; Fomivirsen; Saquinavir (SQ); Ritonavir ([5S-(5R,8R,10R,11R)]-10-hydroxy-2-methyl-5-(1-methylethyl)-1-[2-(methylethyl)-4-thiazolyl]-3,6-dioxo-8,11-bis(phenylmethyl)-2,4,7,12-tetraazatridecan-13-oic acid 5-thiazolylmethyl ester); Indinavir ([(1 S,2R,5(S)-2,3,5-trideoxy-N-(2,3-dihydro-2-hydroxy-1H-inden-1-yl)-5-[2-[[(1,1-dimethylethyl)amino]carbonyl]-4-pyridinylmethyl)-1-piperazinyl]-2-(phenylmethyl-erythro)pentonamide); Amprenavir; Nelfinavir; Lopinavir; Atazanavir; Bevirimat; Indinavir; Relenza; Zanamivir; Oseltamivir; Tarvacin; etc. are administered to individuals in a formulation (e.g., in the same or in separate formulations) with a pharmaceutically acceptable excipient(s).

In some embodiments, the herpesvirus inhibitor disclosed herein can be administered in combination with another herpesvirus inhibitor, such as nucleosides, for example acyclovir and ganciclovir or their prodrugs (valacylovir and valganciclovir), or others, for example foscarnet and letermovir. In certain cases, the subject herpesvirus inhibitor is administered in combination with letermovir. In certain cases the subject herpesvirus inhibitors are administered as part of a highly active antiretroviral therapy (HAART) regimen.

The terms “co-administration” and “in combination with” include the administration of two or more therapeutic agents either simultaneously, concurrently or sequentially within no specific time limits. In one embodiment, the agents are present in the cell or in the subject's body at the same time or exert their biological or therapeutic effect at the same time. In one embodiment, the therapeutic agents are in the same composition or unit dosage form. In other embodiments, the therapeutic agents are in separate compositions or unit dosage forms. In certain embodiments, a first agent can be administered prior to (e.g., minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapeutic agent.

“Concomitant administration” of a known therapeutic drug or additional therapy with a pharmaceutical composition of the present disclosure means administration of the compound and second agent or additional therapy at such time that both the known drug and the composition of the present invention will have a therapeutic effect. Such concomitant administration may involve concurrent (i.e. at the same time), prior, or subsequent administration of the drug with respect to the administration of a subject compound. Routes of administration of the two agents may vary, where representative routes of administration are described in greater detail below. A person of ordinary skill in the art would have no difficulty determining the appropriate timing, sequence and dosages of administration for particular drugs or therapies and compounds of the present disclosure.

In some embodiments, the compounds (e.g., a subject compound and the at least one additional compound or therapy) are administered to the subject within twenty-four hours of each other, such as within 12 hours of each other, within 6 hours of each other, within 3 hours of each other, or within 1 hour of each other. In certain embodiments, the compounds are administered within 1 hour of each other. In certain embodiments, the compounds are administered substantially simultaneously. By administered substantially simultaneously is meant that the compounds are administered to the subject within about minutes or less of each other, such as 5 minutes or less, or 1 minute or less of each other.

Also provided are pharmaceutical preparations of the subject compounds and the second active agent. In pharmaceutical dosage forms, the compounds may be administered in the form of their pharmaceutically acceptable salts, or they may also be used alone or in appropriate association, as well as in combination, with other pharmaceutically active compounds.

In certain instances, the combination provides an enhanced effect relative to either component alone; in some cases, the combination provides a supra-additive or synergistic effect relative to the combined or additive effects of the components. A variety of combinations of the subject compounds and the chemotherapeutic agent may be employed, used either sequentially or simultaneously. For multiple dosages, the two agents may directly alternate, or two or more doses of one agent may be alternated with a single dose of the other agent, for example. Simultaneous administration of both agents may also be alternated or otherwise interspersed with dosages of the individual agents. In some cases, the time between dosages may be for a period from about 1-6 hours, to about 6-12 hours, to about 12-24 hours, to about 1-2 days, to about 1-2 week or longer following the initiation of treatment.

Compositions

Aspects of the invention also include pharmaceutical compositions comprising the compounds disclosed herein. For example, in certain embodiments the pharmaceutical compositions comprise a compound provided herein, for example, a compound of any one of Formulae (I)-(V), any one of compounds 1-46, as an active ingredient, including a single enantiomer, a racemic mixture, a mixture of diastereomers, or an isotopic variant thereof; or a pharmaceutically acceptable salt, solvate, or prodrug thereof; or a pharmaceutically acceptable salt, solvate, hydrate, or prodrug; in combination with a pharmaceutically acceptable excipient, or a mixture thereof.

In certain embodiments the pharmaceutical compositions comprise the compound of Formula (I)-(V), compounds 1-46, or the pharmaceutically acceptable salt or solvate thereof, and a pharmaceutically acceptable excipient.

Suitable excipients are well known to those skilled in the art, and non-limiting examples of suitable excipients are provided herein. Whether a particular excipient is suitable for incorporation into a pharmaceutical composition or dosage form depends on a variety of factors well known in the art, including, but not limited to, the method of administration. The suitability of a particular excipient may also depend on the specific active ingredients in the dosage form.

In certain embodiments, the pharmaceutical compositions provided herein are formulated for oral administration. In certain aspects, the oral formulations provided herein comprise compounds described herein that are formulated with pharmaceutically acceptable carriers or excipients. Such carriers enable the compounds described herein to be formulated as tablets, powders, pills, capsules, liquids, gels, syrups, elixirs, slurries, suspensions and the like, for oral ingestion by a patient to be treated.

In certain embodiments, pharmaceutical compositions provided herein for oral administration are prepared by mixing one or more solid excipient with one or more of the compounds described herein, optionally grinding the resulting mixture, and processing the mixture of granules, after adding suitable auxiliaries, if desired, to obtain tablets. Suitable excipients include, in particular, fillers such as sugars, including lactose, sucrose, mannitol, or sorbitol; cellulose preparations such as: for example, maize starch, wheat starch, rice starch, potato starch, gelatin, gum tragacanth, methylcellulose, microcrystalline cellulose, hydroxypropylmethylcellulose, sodium carboxymethylcellulose; or others such as: polyvinylpyrrolidone (PVP or povidone) or calcium phosphate. If desired, disintegrating agents are optionally added, such as the cross-linked croscarmellose sodium, polyvinylpyrrolidone, agar, or alginic acid or a salt thereof such as sodium alginate.

Methods for the preparation of the pharmaceutical compositions provided herein include formulating the compounds with one or more inert, pharmaceutically acceptable excipients or carriers to form a solid, semi-solid or liquid. Solid compositions include, but are not limited to, powders, tablets, dispersible granules, capsules, cachets, and suppositories. Liquid compositions include solutions in which a compound is dissolved, emulsions comprising a compound, or a solution containing liposomes, micelles, or nanoparticles comprising a compound as disclosed herein. Semi-solid compositions include, but are not limited to, gels, suspensions and creams. In various embodiments, the compositions are in liquid solutions or suspensions, solid forms suitable for solution or suspension in a liquid prior to use, or as emulsions. These compositions optionally contain minor amounts of nontoxic, auxiliary substances, such as wetting or emulsifying agents, pH buffering agents, and so forth.

The compounds provided herein, for example, the compound of any one of formulae (I)-(V) or any one of compounds 1-46, or the pharmaceutically acceptable salt or solvate thereof, may be administered alone, or in combination with one or more other compounds provided herein. The pharmaceutical compositions that comprise a subject compound (e.g., as described herein), including an enantiomer, a mixture of enantiomers, a mixture of two or more diastereomers, a tautomer, a mixture of two or more tautomers, or an isotopic variant thereof, or a pharmaceutically acceptable salt, solvate, hydrate, or prodrug thereof, can be formulated in various dosage forms for oral, parenteral, and topical administration. The pharmaceutical compositions can also be formulated as modified release dosage forms. These dosage forms can be prepared according to conventional methods and techniques known to those skilled in the art (see, Remington: The Science and Practice of Pharmacy, supra; Modified-Release Drug Delivery Technology, 2nd ed.; Rathbone et al., Eds.; Marcel Dekker, Inc.: New York, N.Y., 2008).

In one embodiment, the pharmaceutical compositions are provided in a dosage form for oral administration, which comprise a compound provided herein, for example, a subject compound (e.g., as described herein), including an enantiomer, a mixture of enantiomers, a mixture of two or more diastereomers, a tautomer, a mixture of two or more tautomers, or an isotopic variant thereof; or a pharmaceutically acceptable salt, solvate, hydrate, or prodrug thereof; and one or more pharmaceutically acceptable excipients or carriers.

The pharmaceutical compositions provided herein can be provided in a unit-dosage form or multiple-dosage form. A unit-dosage form, as used herein, refers to physically discrete a unit suitable for administration to a human and animal subject, and packaged individually as is known in the art. Each unit-dose contains a predetermined quantity of an active ingredient(s) sufficient to produce the desired therapeutic effect, in association with the required pharmaceutical carriers or excipients. Examples of a unit-dosage form include an ampoule, syringe, and individually packaged tablet and capsule. For example, a 100 mg unit dose contains about 100 mg of an active ingredient in a packaged tablet or capsule. A unit-dosage form may be administered in fractions or multiples thereof. A multiple-dosage form is a plurality of identical unit-dosage forms packaged in a single container to be administered in segregated unit-dosage form. Examples of a multiple-dosage form include a vial, bottle of tablets or capsules, or bottle of pints or gallons.

The pharmaceutical compositions provided herein can be administered at once, or multiple times at intervals of time. It is understood that the precise dosage and duration of treatment may vary with the age, weight, and condition of the patient being treated, and may be determined empirically using known testing protocols or by extrapolation from in vivo or in vitro test or diagnostic data. It is further understood that for any particular individual, specific dosage regimens should be adjusted over time according to the individual need and the professional judgment of the person administering or supervising the administration of the formulations.

Kits

Aspects of the invention further include kits for use in practicing the subject methods and compositions. The compounds of the invention can be included as reagents in kits for use in, for example, the methodologies described above.

A kit can include a compound (e.g., as described herein); and one or more components selected from the group consisting of an additional active agent, a buffer, a solvent, a standard and instructions for use.

The one or more components of the kit may be provided in separate containers (e.g., separate tubes, bottles, or wells in a multi-well strip or plate).

The compounds of the kits may be provided in a liquid composition, such as any suitable buffer. Alternatively, the compounds of the kits may be provided in a dry composition (e.g., may be lyophilized), and the kit may optionally include one or more buffers for reconstituting the dry compound. In certain aspects, the kit may include aliquots of the compound provided in separate containers (e.g., separate tubes, bottles, or wells in a multi-well strip or plate).

In addition, one or more components may be combined into a single container, e.g., a glass or plastic vial, tube or bottle. In certain instances, the kit may further include a container (e.g., such as a box, a bag, an insulated container, a bottle, tube, etc.) in which all of the components (and their separate containers) are present. The kit may further include packaging that is separate from or attached to the kit container and upon which is printed information about the kit, the components of the and/or instructions for use of the kit.

In addition to the above components, the subject kits may further include instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, etc. Yet another means would be a computer readable medium, e.g., diskette, CD, DVD, portable flash drive, etc., on which the information has been recorded. Yet another means that may be present is a website address which may be used via the Internet to access the information at a removed site. Any convenient means may be present in the kits.

Utility

The compounds and methods of the invention, e.g., as described herein, find use in applications where the inhibition and treatment of a herpesvirus, (e.g., HCMV) is desired.

The subject compounds and methods find use in therapeutic applications. Therapeutic applications of interest include the treatment of a human herpesviruses. For example, the subject compounds may find use in the treatment of cytomegalovirus (CMV), herpes simplex virus-1 (HSV-1), herpes simplex virus-2 (HSV-2), varicella-zoster virus (VZV), Epstein-Barr virus (EBV), and Kaposi's sarcoma-associated herpesvirus (KSHV).

The following example(s) is/are offered by way of illustration and not by way of limitation.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

General methods in molecular and cellular biochemistry can be found in such standard textbooks as Molecular Cloning: A Laboratory Manual, 3rd Ed. (Sambrook et al., HaRBor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998), the disclosures of which are incorporated herein by reference. Reagents, cloning vectors, cells, and kits for methods referred to in, or related to, this disclosure are available from commercial vendors such as BioRad, Agilent Technologies, Thermo Fisher Scientific, Sigma-Aldrich, New England Biolabs (NEB), Takara Bio USA, Inc., and the like, as well as repositories such as e.g., Addgene, Inc., American Type Culture Collection (ATCC), and the like.

All reagents and solvents were used as purchased from commercial sources. Moisture sensitive reactions were carried out under a nitrogen atmosphere. Reactions were monitored by thin layer chromatography (TLC) using pre-coated silica gel aluminum plates containing a fluorescent indicator (F-254). Detection was done with ultraviolet (UV) light (254 nm). Alternatively, the progress of a reaction was monitored by liquid chromatography coupled to a mass spectrometer (LC/MS). Specifically, but without limitation, the following abbreviations were used, in addition to the other ones described herein, in the examples: DCM or CH₂Cl₂ (dichloromethane); dioxane (1,4-dioxane); DIPEA (N,N-diisopropylethylamine); DMF (N,N-dimethylformamide); EtOH (ethanol); ether or Et₂O (diethyl ether); Et₃N (triethylamine); NaHCO₃ (sodium bicarbonate); HATU (1-[bis(dimethylamino)methylene]-1H-1,2,3-triazolo[4,5-b]pyridinium 3-oxide hexafluorophosphate or N-[(dimethylamino)-1H-1,2,3-triazolo-[4,5-b]pyridin-1-ylmethylene]-N-methylmethanaminium hexafluorophosphate N-oxide); Pd₂(dba)₃ (tris(dibenzylideneacetone)dipalladium(0)); Xantphos (4,5-bis(diphenylphosphino)-9,9-dimethylxanthene); Cs₂CO₃ (cesium carbonate); K₂CO₃ (potassium carbonate); CuI (copper (I) iodide; CDI (carbonyldiimidazole); LiOH (lithium hydroxide); TFA (trifluoroacetic acid); CHCl₃ (chloroform); MeOH (methanol); MTBE (methyl tert-butyl ether); EtOAc (ethyl acetate); ρW or MW (microwave); O/N (overnight); RT or rt (room or ambient temperature); THF (tetrahydrofuran); HCl (hydrochloric acid); Na₂SO₄ (sodium sulfate). ¹H NMR spectra were recorded at RT with a Bruker Avanche III 600 MHz NMR spectrometer equipped with a Bruker's 5 mm PABBO probe. Chemical shifts in proton NMR are reported in ppm downfield from tetramethylsilane using residual solvent signals as internal reference. Chemical shifts in fluorine NMR are reported in ppm downfield from trichlorofluoromethane using residual solvent signals as internal reference. Fluorine NMR was recorded at 565 MHz. NMR data were processed utilizing ACD/Spectrus processor (v2016.1.1, ACD/Labs Inc.). Nomenclature for the naming of compounds, such as for Compound Examples and intermediate compounds, were performed using ACD/Name (Chemists' Version from ACD/Labs Inc.) to generate the IUPAC-style names. Naming of commercial or literature compounds utilized SciFinder, ACD/Names, and common or trivial names known to those skilled in the art.

Microwave assisted reactions were performed using an Anton Paar “Monowave 200” Microwave Synthesis Reactor with magnetron power 850W. Unless stated otherwise the temperature was reached as fast as possible and controlled by built-in IR sensor (temperature uncertainty ±5° C.). Reaction was carried out either in 10 mL or 30 mL vials, with the default stirrer speed 600 rpm.

The LC/MS system used for monitoring the progress of reactions, assessing the purity (absorbance at 254 nm) and identity of the product consisted of Dionex ULTIMATE 3000 uHPLC module and Thermo Scientific LTQ XL mass-spectrometer with electrospray ionization and Ion-Trap type of detector (alternating positive-negative mode). Separation was performed with Thermo Scientific™ Accucore™ aQ C18 Polar Endcapped LC column (100 mm×2.1 mm; particle size 2.6 μm, 80 Å). The column was maintained at 40° C. Commercial HPLC-grade methanol, acetonitrile and domestic ‘millipore (Milli-Q)’ water used for chromatography were modified by adding 0.1% (v/v) of formic acid. The eluent was delivered with constant flow rate of 0.4 mL/min, column was equilibrated for 5 min with the corresponding eluent prior to injection of the sample (1 μL) and one of the following separation conditions were used:

Eluent Systems:
A—Gradient of MeOH-Water, 45 to 95% in 5.25 min, followed by 5 min of isocratic MeOH—water 95%;
B—Gradient of Methanol-Water, 30 to 65% in 4.75 min, then to 95% in 2.5 min, followed by 4 min of isocratic MeOH—water 95%; or
C—Gradient of MeOH-Water, 15% to 65% in 5 min, 65% to 95% in 2.5 min, followed by 4 min of isocratic MeOH—water 95%.

### Example 1: Synthesis of Exemplary Compounds

### N-(4-chlorobenzyl)-5-(2-(3-fluoro-3-methylazetidin-1-yl)-2-oxoethyl)-6-oxo-2-(2-oxopyrrolidin-1-yl)-5,6-dihydropyrido[2,3-b]pyrazine-7-carboxamide, (Compound 1)

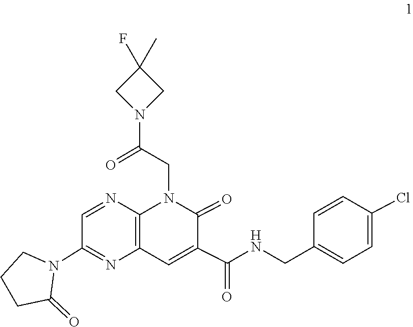 [SMILES: CC1(F)CN(C(=O)Cn2c(=O)c(C(=O)NCc3ccc(Cl)cc3)cc3nc(N4CCCC4=O)cnc32)C1]

1 was synthesized as in Scheme 4.

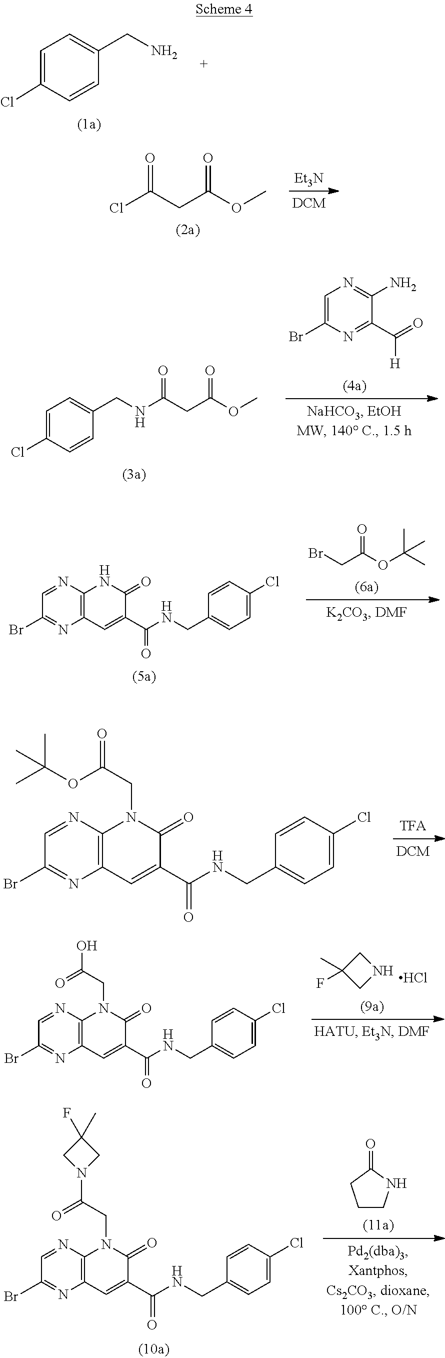

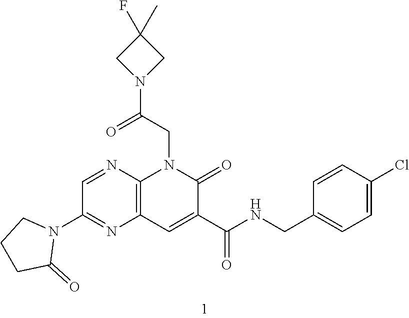 [SMILES: CC1(F)CN(C(=O)Cn2c(=O)c(C(=O)NCc3ccc(Cl)cc3)cc3nc(N4CCCC4=O)cnc32)C1]

Preparation of methyl 3-{[(4-chlorophenyl)methyl]amino}-3-oxopropanoate, (3a). To a solution of 4-chlorobenzylamine (1a) (7.8 mL, 57.5 mmol) in DCM was added Et₃N (24 mL, 172.6 mmol) and the mixture was cooled in a −78° C. bath. A solution of methyl 3-chloro-3-oxopropionate (2a) (7.0 mL, 57.5 mmol) in DCM (20 mL) was added via a dropping funnel over 10 min. The mixture was stirred at −78° C. for 30 min and then it was slowly warmed to room temperature. After overnight, the reaction was quenched with saturated NaHCO₃ solution. The layers were separated and the organic layer was washed with brine, dried over Na₂SO₄, filtered and concentrated under reduced pressure. The solid was suspended in ˜10 mL EtOAc and further diluted with 200 mL hexanes. The resulting precipitate was filtered, washed with hexanes providing (3a) as a light yellow solid (11.3 g, 81.3% yield).

Preparation of 2-bromo-N-[(4-chlorophenyl)methyl]-6-oxo-5,6-dihydropyrido[2,3-b]pyrazine-7-carboxamide, (5a). To a 30 mL microwave reaction vial, was added 3-amino-6-bromopyrazine-2-carbaldehyde (4a) (0.75 g, 3.7 mmol), (3a) (1.34 g, 5.6 mmol), NaHCO₃ (0.93 g, 11.1 mmol), and absolute ethanol (10 mL). The microwave reactor was set at 140° C. for 1.5 h. Similarly, one more batch was carried out with 3-amino-6-bromopyrazine-2-carbaldehyde (4a) (0.75 g mg). After completion of reaction(s), the two batch were combined. The mixture was transferred to a round bottom flask and the solvent was removed under reduced pressure. To the mixture was added diethyl ether (50 mL) and filtered under vacuum. The solid was collected using vacuum filtration, washed with ether, and dried, which provided (5a) along with inorganics. This material was used as such in the next step without further purification.

Preparation of tert-butyl [2-bromo-7-{[(4-chlorophenyl)methyl]carbamoyl}-6-oxopyrido[2,3-b]pyrazin-5(6H)-yl]acetate, (7a). To a solution of (5a) in DMF (30 mL) as added tert-butyl bromoacetate (6a) (1.1 mL, 7.4 mmol), followed by K₂CO₃ (3.0 g, 22.2 mmol) at room temperature. After 4 h, the solvent was removed under reduced pressure. The residue was diluted with water and extracted with DCM. The organic layer was washed with brine, dried over anhydrous Na₂SO₄, filtered and concentrated under reduced pressure. The crude material was absorbed on silica gel and product purified by column chromatography on silica gel with a gradient of 0% to 50% EtOAc in hexanes, which generated (7a) as a light yellow solid (1.36 g, 36% yield over two steps).

Preparation of [2-bromo-7-{[(4-chlorophenyl)methyl]carbamoyl}-6-oxopyrido[2,3-b]pyrazin-5(6H)-yl]acetic acid (8a). To a solution of (7a) (1.3 g, 2.6 mmol) in DCM (20 mL) was added trifluoroacetic acid (10 mL) at room temperature. After 4 h, the solvent was removed under reduced pressure, and then the residue was co-evaporated with DCM (2×20 mL). The crude was dissolved in DCM and solidified with ether. The solid was filtered under vacuum, washed with ether and dried, which provided (8a) as a light yellow solid (1.12 g, 97% yield).

Preparation of 2-bromo-N-[(4-chlorophenyl)methyl]-5-[2-(3-fluoro-3-methylazetidin-1-yl)-2-oxoethyl]-6-oxo-5,6-dihydropyrido[2,3-b]pyrazine-7-carboxamide, (10a). A mixture of (8a) (2.0 g, 4.43 mmol), HATU (2.02 g, 5.31 mmol) in DMF (10 mL) was cooled to in an ice-water bath. To this, a solution of 3-fluoro-3-methylazetidine hydrochloride (9a) (1.12 g, 1.9 mmol) and DIPEA (3.9 mL, 22.14 mmol) in DMF (2 mL) was added slowly. After 1 h, the reaction was quenched with water (50 mL) and extracted with chloroform (3×100 mL), washed the organic layer with brine (1×100 mL), dried over anhydrous Na₂SO₄, filtered and concentrated under reduced pressure. Purification of the product by column chromatography on silica gel with a gradient of 0% to 100% EtOAc in hexanes produced (10a) (1.9 g, 82% yield).

Preparation of N-(4-chlorobenzyl)-5-(2-(3-fluoro-3-methylazetidin-1-yl)-2-oxoethyl)-6-oxo-2-(2-oxopyrrolidin-1-yl)-5,6-dihydropyrido[2,3-b]pyrazine-7-carboxamide, 1. To a solution of (10a) (80 mg, 0.15 mmol), pyrrolidin-2-one (11a) (130 mg, 1.53 mmol), in dioxane (5 mL) was added Pd₂(dba)₃ (6.8 mg, 0.008 mmol), Xantphos (13.2 mg, 0.02 mmol), followed by Cs₂CO₃ (69 mg, 0.2 mmol). The reaction mixture was heated with an oil bath at 100° C. overnight. The mixture was cooled to room temperature, adsorbed on silica gel. Purification of the product was accomplished by column chromatography on silica gel with a gradient of 0% to 10% MeOH in EtOAc, which generated 1 as a light yellow solid (64 mg, 79% yield). ¹H NMR (600 MHz, DMSO-d₆) δ 9.86 (t, J=6.0 Hz, 1H), 9.63 (s, 1H), 8.68 (s, 1H), 7.44-7.34 (m, 4H), 5.19-5.09 (m, 2H), 4.56 (d, J=6.8 Hz, 2H), 4.55-4.42 (m, 2H), 4.08-4.01 (m, 3H), 4.01-3.98 (m, 1H), 2.65 (t, J=8.1 Hz, 2H), 2.19-2.11 (m, 2H), 1.64 (d, J=22.2 Hz, 3H). ¹⁹F NMR (565 MHz, DMSO-d₆) δ−138.88-−139.15 (m, 1F). LC/MS: Eluent system A (retention time: 5.81 min); ESI-MS 527 [M+H]⁺.

### Synthesis of N-(4-chlorobenzyl)-5-(2-(3-fluoro-3-methylazetidin-1-yl)-2-oxoethyl)-6-oxo-2-(2-oxooxazolidin-3-yl)-5,6-dihydropyrido[2,3-b]pyrazine-7-carboxamide, (Compound 2)

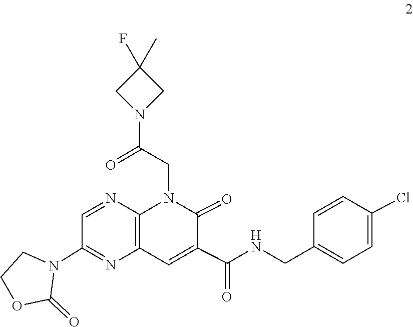 [SMILES: CC1(F)CN(C(=O)Cn2c(=O)c(C(=O)NCc3ccc(Cl)cc3)cc3nc(N4CCOC4=O)cnc32)C1]

Preparation of N-(4-chlorobenzyl)-5-(2-(3-fluoro-3-methylazetidin-1-yl)-2-oxoethyl)-6-oxo-2-(2-oxooxazolidin-3-yl)-5,6-dihydropyrido[2,3-b]pyrazine-7-carboxamide, 2. A similar procedure as described for compound 1 was followed: with 2-bromo-N-[(4-chlorophenyl)methyl]-5-[2-(3-fluoro-3-methylazetidin-1-yl)-2-oxoethyl]-6-oxo-5,6-dihydropyrido[2,3-b]pyrazine-7-carboxamide (10a) (80 mg, 0.15 mmol), 2-oxazolidinone (133 mg, 1.53 mmol), Pd₂(dba)₃ (6.8 mg, 0.008 mmol), Xantphos (13.2 mg, 0.02 mmol), and Cs₂CO₃ (69 mg, 0.2 mmol) in dioxane (5 mL), followed by silica gel column purification (eluted with a gradient of 0% to 10% MeOH in EtOAc) afforded 2 (39 mg, 48% yield) as a light yellow solid. ¹H NMR (600 MHz, DMSO-d₆) δ 9.86 (t, J=6.0 Hz, 1H), 9.46 (s, 1H), 8.68 (s, 1H), 7.43-7.36 (m, 4H), 5.20-5.09 (m, 2H), 4.62-4.56 (m, 4H), 4.56-4.42 (m, 2H), 4.28-4.20 (m, 2H), 4.07-3.97 (m, 2H), 1.64 (d, J=22.2 Hz, 3H). ¹⁹F NMR (565 MHz, DMSO-d₆) δ−138.86-−139.19 (m, 1F). LC/MS: Eluent system A (retention time: 5.43 min); ESI-MS 529 [M+H]⁺.

### Synthesis of N-(4-chlorobenzyl)-5-(2-(3-fluoro-3-methylazetidin-1-yl)-2-oxoethyl)-2-(3-methyl-2-oxoimidazolidin-1-yl)-6-oxo-5,6-dihydropyrido[2,3-b]pyrazine-7-carboxamide, (Compound 3)

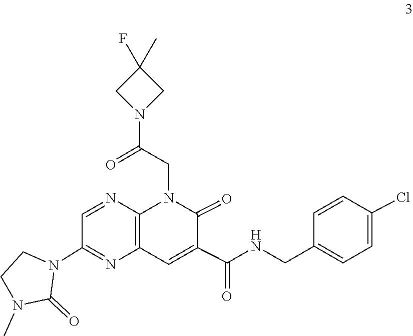 [SMILES: CN1CCN(c2cnc3c(cc(C(=O)NCc4ccc(Cl)cc4)c(=O)n3CC(=O)N3CC(C)(F)C3)n2)C1=O]

Preparation of N-(4-chlorobenzyl)-5-(2-(3-fluoro-3-methylazetidin-1-yl)-2-oxoethyl)-2-(3-methyl-2-oxoimidazolidin-1-yl)-6-oxo-5,6-dihydropyrido[2,3-b]pyrazine-7-carboxamide, 3. A similar procedure as described for compound 1 was followed: with 2-bromo-N-[(4-chlorophenyl)methyl]-5-[2-(3-fluoro-3-methylazetidin-1-yl)-2-oxoethyl]-6-oxo-5,6-dihydropyrido[2,3-b]pyrazine-7-carboxamide (10a) (75 mg, 0.14 mmol), 1-methyl-2-imidazolidone (72 mg, 0.72 mmol), Pd₂(dba)₃ (6.6 mg, 0.007 mmol), Xantphos (12.4 mg, 0.02 mmol), and Cs₂CO₃ (65 mg, 0.2 mmol) in dioxane (5 mL), followed by silica gel column purification (0% to 10% MeOH in EtOAc) produced 3 (36.3 mg, 47% yield) as a light yellow solid. ¹H NMR (600 MHz, DMSO-d₆) δ 9.91 (t, J=6.0 Hz, 1H), 9.61 (s, 1H), 8.65 (s, 1H), 7.43-7.34 (m, 4H), 5.18-5.08 (m, 2H), 4.56 (d, J=6.4 Hz, 2H), 4.54-4.41 (m, 2H), 4.06-3.96 (m, 4H), 3.59-3.52 (m, 2H), 2.84 (s, 3H), 1.63 (d, J=22.2 Hz, 3H). ¹⁹F NMR (565 MHz, DMSO-d₆) δ −138.85-−139.17 (m, 1 F). LC/MS: Eluent system A (retention time: 5.87 min); ESI-MS 542 [M+H]⁺.

### Synthesis of N-(4-chlorobenzyl)-5-(2-(3-fluoro-3-methylazetidin-1-yl)-2-oxoethyl)-2-(3-methyl-2-oxopyrrolidin-1-yl)-6-oxo-5,6-dihydropyrido[2,3-b]pyrazine-7-carboxamide, (Compound 4)

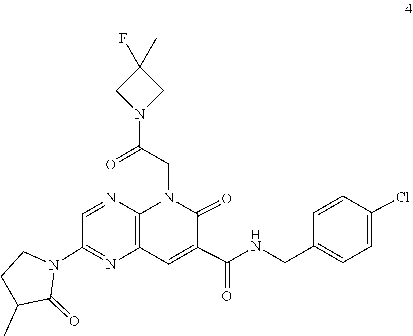 [SMILES: CC1CCN(c2cnc3c(cc(C(=O)NCc4ccc(Cl)cc4)c(=O)n3CC(=O)N3CC(C)(F)C3)n2)C1=O]

Preparation of N-(4-chlorobenzyl)-5-(2-(3-fluoro-3-methylazetidin-1-yl)-2-oxoethyl)-2-(3-methyl-2-oxopyrrolidin-1-yl)-6-oxo-5,6-dihydropyrido[2,3-b]pyrazine-7-carboxamide, 4. A similar procedure as described for compound 1 was followed: with 2-bromo-N-[(4-chlorophenyl)methyl]-5-[2-(3-fluoro-3-methylazetidin-1-yl)-2-oxoethyl]-6-oxo-5,6-dihydropyrido[2,3-b]pyrazine-7-carboxamide (10a) (65 mg, 0.12 mmol), 3-methyl-2-pyrrolidinone (61 mg, 0.62 mmol), Pd₂(dba)₃ (5.6 mg, 0.006 mmol), Xantphos (10.8 mg, 0.019 mmol), and Cs₂CO₃ (56.6 mg, 0.17 mmol) in dioxane (5 mL), followed by silica gel column purification (eluent: a gradient of 0% to 10% MeOH in EtOAc) provided 4 (58 mg, 86% yield) as a light yellow solid. ¹H NMR (600 MHz, DMSO-d₆) δ−9.86 (t, J=6.0 Hz, 1H), 9.65 (s, 1H), 8.68 (s, 1H), 7.43-7.35 (m, 4H), 5.22-5.08 (m, 2H), 4.56 (d, J=6.8 Hz, 2H), 4.54-4.43 (m, 2H), 4.13-4.07 (m, 1H), 4.06-3.98 (m, 2H), 3.91-3.85 (m, 1H), 2.86-2.78 (m, 1H), 2.42-2.35 (m, 1H), 1.84-1.74 (m, 1H), 1.63 (d, J=22.2 Hz, 3H), 1.21 (d, J=6.8 Hz, 3H). ¹⁹F NMR (565 MHz, DMSO-d₆) δ−138.89-−139.16 (m, 1F). LC/MS: Eluent system A (retention time: 6.13 min); ESI-MS 541 [M+H]⁺.

### Synthesis of [3-(7-{[(4-chlorophenyl)methyl]carbamoyl}-5-[2-(3,3-dimethylazetidin-1-yl)-2-oxoethyl]-6-oxo-5,6-dihydropyrido[2,3-b]pyrazin-2-yl)-2-oxoimidazolidin-1-yl]acetic Acid, (Compound 5)

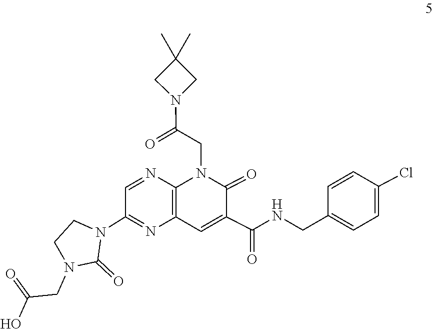 [SMILES: CC1(C)CN(C(=O)Cn2c(=O)c(C(=O)NCc3ccc(Cl)cc3)cc3nc(N4CCN(CC(=O)O)C4=O)cnc32)C1]

5 was synthesized as in Scheme 5.

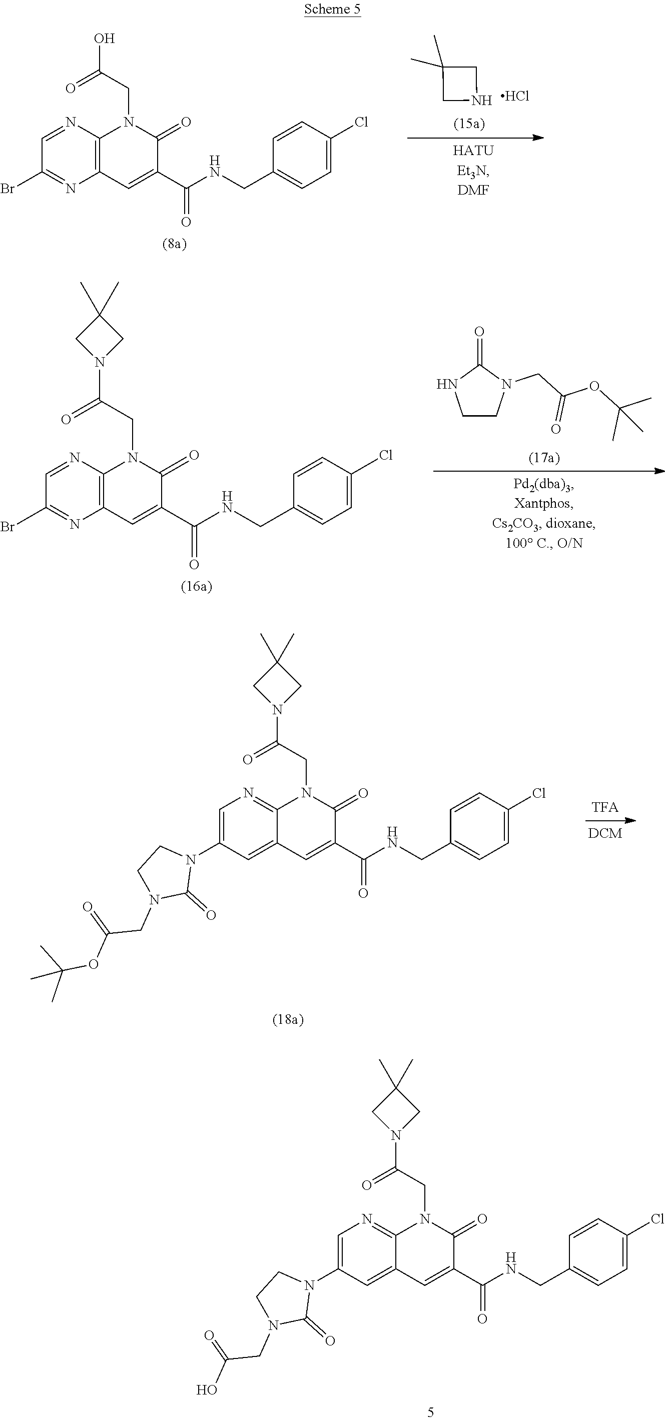

Preparation of 2-bromo-N-[(4-chlorophenyl)methyl]-5-[2-(3,3-dimethylazetidin-1-yl)-2-oxoethyl]-6-oxo-5,6-dihydropyrido[2,3-b]pyrazine-7-carboxamide, (16a). To a solution of 2-bromo-7-{[(4-chlorophenyl)methyl]carbamoyl}-6-oxopyrido[2,3-b]pyrazin-5(6H)-yl]acetic acid, (8a) (380 mg, 0.84 mmol) and 3,3-dimethylazetidine hydrochloride (15a) (153 mg, 1.26 mmol) in DMF was added HATU (510 mg, 1.34 mmol) and triethylamine (0.6 mL, 4.2 mmol). After 1 h at room temperature, the solvent was removed under reduced pressure. The residue was diluted with water and extracted with DCM. The organic layer was washed with brine, dried over anhydrous Na₂SO₄, filtered and concentrated under reduced pressure. The product was purified by column chromatography on silica gel with a gradient of 25% to 75% EtOAc in hexanes providing (16a) (280 mg, 64% yield) as a yellow solid.

Preparation of tert-butyl [3-(7-{[(4-chlorophenyl)methyl]carbamoyl}-5-[2-(3,3-dimethylazetidin-1-yl)-2-oxoethyl]-6-oxo-5,6-dihydropyrido[2,3-b]pyrazin-2-yl)-2-oxoimidazolidin-1-yl]acetate, (18a). To a solution of (16a) (70 mg, 0.14 mmol) and tert-butyl 2-(2-oxoimidazolidin-1-yl)acetate (17a) (40 mg, 0.2 mmol) in dioxane (5 mL) was added Pd₂(dba)₃ (6.2 mg, 0.007 mmol), Xantphos (11.7 mg, 0.020 mmol), followed by Cs₂CO₃ (62 mg, 0.19 mmol). The reaction mixture was heated with an oil bath at 100° C. for overnight. It was then cooled to room temperature and adsorbed on silica gel. The product was purified by column chromatography on silica gel with gradient of 0% to 10% MeOH in EtOAc, which provided (18a) (73 mg, 85% yield) as a light yellow solid.

Preparation of [3-(7-{[(4-chlorophenyl)methyl]carbamoyl}-5-[2-(3,3-dimethylazetidin-1-yl)-2-oxoethyl]-6-oxo-5,6-dihydropyrido[2,3-b]pyrazin-2-yl)-2-oxoimidazolidin-1-yl]acetic acid, 5. To a solution of (18a) (73 mg, 0.11 mmol) in DCM (3 mL) was added trifluoroacetic acid (2 mL) at room temperature and stirred for 2 h. After completion of the reaction, the solvent was removed under reduced pressure, and then the residue was co-evaporated with DCM/ether (1:1, 3×10 mL). The crude was dissolved in DCM and solidified with ether. The solid was collected by filtration, washed with ether, and dried, which afforded 5 (48 mg, 72% yield) as a light yellow solid. ¹H NMR (600 MHz, DMSO-d₆) δ 12.96 (br s, 1H), 9.92 (t, J=6.0 Hz, 1H), 9.58 (s, 1H), 8.66 (s, 1H), 7.43-7.36 (m, 4H), 5.08 (s, 2H), 4.57 (d, J=6.0 Hz, 2H), 4.10-4.00 (m, 6H), 3.69-3.63 (m, 2H), 3.59 (s, 2H), 1.28 (s, 6H). LC/MS: Eluent system A (retention time: 5.95 min); ESI-MS 582.26 [M+H]⁺. 580 [M−H]⁻

### Synthesis of 2-[3-(2-aminoethyl)-2-oxoimidazolidin-1-yl]-N-[(4-chlorophenyl)methyl]-5-[2-(3-fluoro-3-methylazetidin-1-yl)-2-oxoethyl]-6-oxo-5,6-dihydropyrido[2,3-b]pyrazine-7-carboxamide, (Compound 6)

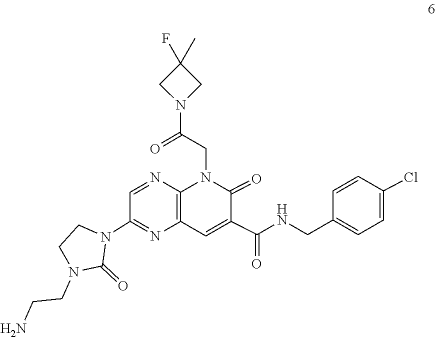 [SMILES: CC1(F)CN(C(=O)Cn2c(=O)c(C(=O)NCc3ccc(Cl)cc3)cc3nc(N4CCN(CCN)C4=O)cnc32)C1]

6 was synthesized as in Scheme 6.

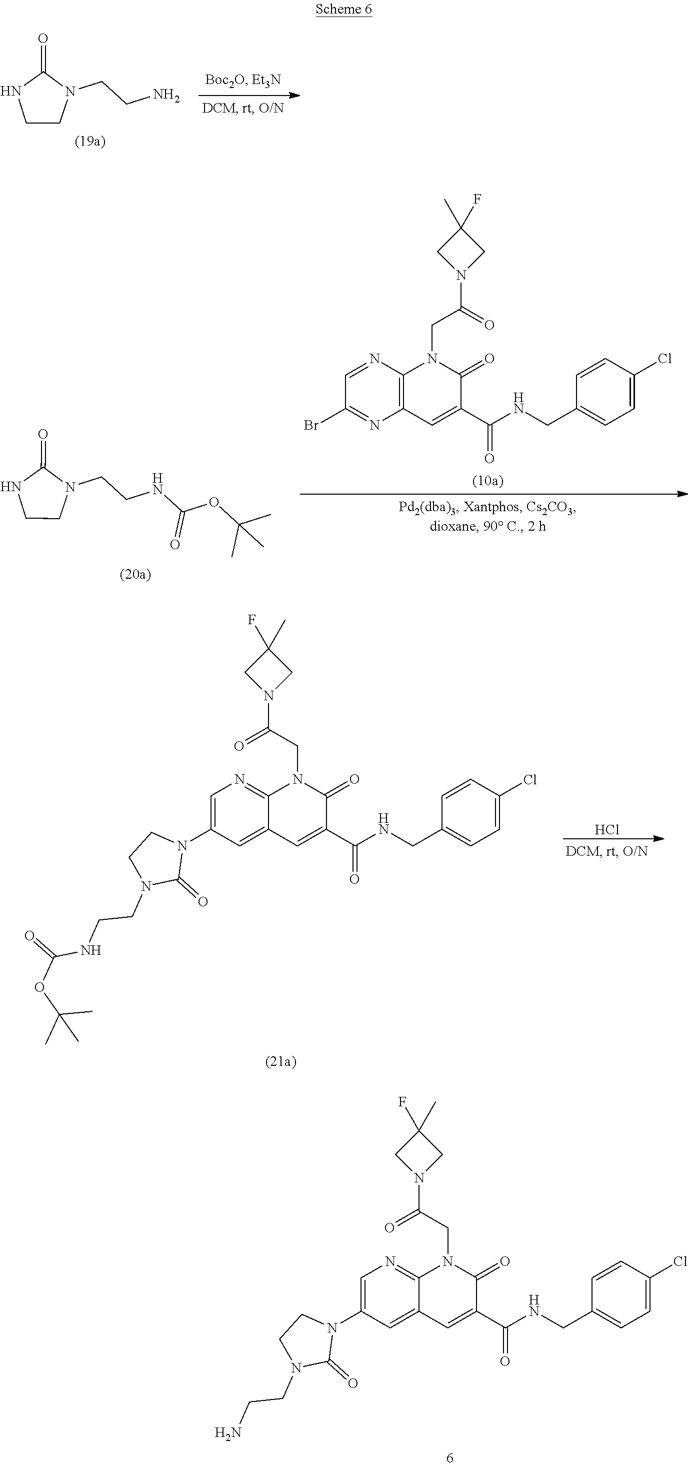 [SMILES: NCCN1CCNC1=O ; CC(C)(C)OC(=O)NCCN1CCNC1=O ; CC1(F)CN(C(=O)Cn2c(=O)c(C(=O)NCc3ccc(Cl)cc3)cc3nc(Br)cnc32)C1 ; CC1(F)CN(C(=O)Cn2c(=O)c(C(=O)NCc3ccc(Cl)cc3)cc3cc(N4CCN(CCNC(=O)OC(C)(C)C)C4=O)cnc32)C1 ; Cl ; CC1(F)CN(C(=O)Cn2c(=O)c(C(=O)NCc3ccc(Cl)cc3)cc3cc(N4CCN(CCN)C4=O)cnc32)C1]

Preparation of tert-butyl [2-(2-oxoimidazolidin-1-yl)ethyl]carbamate, (20a). To a solution of 1-(2-aminoethyl)imidazolidin-2-one (19a) (200 mg, 1.6 mmol) in DCM (25 mL) containing triethylamine (0.81 g, 8.0 mmol) was added boc anhydride (0.51 g, 2.3 mmol). After overnight at room temperature, the mixture was concentrated under reduced pressure. The residue was dissolved in a small amount of DCM and product purified by silica gel column chromatography (eluted with a gradient of 0% to 100% ethyl acetate-hexane), which provided (20a) (299.0 mg, 81% yield) as a white solid.

Preparation of tert-butyl {2-[3-(7-{[(4-chlorophenyl)methyl]carbamoyl}-5-[2-(3-fluoro-3-methylazetidin-1-yl)-2-oxoethyl]-6-oxo-5,6-dihydropyrido[2,3-b]pyrazin-2-yl)-2-oxoimidazolidin-1-yl]ethyl}carbamate, (21a). A mixture of 2-bromo-N-[(4-chlorophenyl)methyl]-5-[2-(3-fluoro-3-methylazetidin-1-yl)-2-oxoethyl]-6-oxo-5,6-dihydropyrido[2,3-b]pyrazine-7-carboxamide (10a) (65.0 mg, 0.125 mmol), tert-butyl [2-(2-oxoimidazolidin-1-yl)ethyl]carbamate (20a) (57.3 mg, 0.25 mmol), Pd₂(dba)₃ (5.7 mg, 0.0063 mmol), Xantphos (10.2 mg, 0.019 mmol), cesium carbonate (48.9 mg, 0.15 mmol) in dioxane (5 mL) was heated at 90° C. for 2 h. After cooling to ambient temperature, the mixture was concentrated under reduced pressure. The product was purified by column chromatography on silica gel [eluted with a gradient of 0% to 2.5% MeOH in EtOAc], which afforded (21a) (80.0 mg, 95% yield) as a light yellow solid.

Preparation of 2-[3-(2-aminoethyl)-2-oxoimidazolidin-1-yl]-N-[(4-chlorophenyl)methyl]-5-[2-(3-fluoro-3-methylazetidin-1-yl)-2-oxoethyl]-6-oxo-5,6-dihydropyrido[2,3-b]pyrazine-7-carboxamide, 6. To a solution of (21a) (80.0 mg, 0.12 mmol) in DCM (10 mL) was added HCl (0.75 mL, 3.0 mmol; 4 M solution in dioxane). After overnight at room temperature, the mixture was concentrated under reduced pressure, then the residue was dissolved in DCM (50 mL), washed with aq. NaHCO₃ (1 mL) and product purified by column chromatography (eluted with a gradient of 0% to 20% MeOH in chloroform), which generated δ (30.0 mg, 43% yield) as a light yellow solid. ¹H NMR (600 MHz, DMSO-d₆) δ 9.93 (t, J=6.0 Hz, 1H), 9.63 (s, 1H), 8.66 (s, 1H), 7.43-7.37 (m, 4H), 5.09-5.19 (m, 2H), 4.43-4.60 (m, 4H), 3.97-4.06 (m, 4H), 3.59-3.66 (m, 2H), 3.22-3.28 (m, 2H), 2.69-2.77 (m, 2H), 2.50-2.54 (m, 2H), 1.59-1.70 (m, 3H). ¹⁹F NMR (565 MHz, DMSO-d₆) δ −139.14-−138.91 (m, 1F). LC/MS: Eluent system A (retention time: 4.37 min); ESI-MS: 571 [M+H]⁺.

### Synthesis of [3-(6-{[(4-chlorophenyl)methyl]carbamoyl}-8-[2-(3,3-dimethylazetidin-1-yl)-2-oxoethyl]-7-oxo-7,8-dihydro-1,8-naphthyridin-3-yl)-2-oxoimidazolidin-1-yl]acetic Acid, (Compound 7)

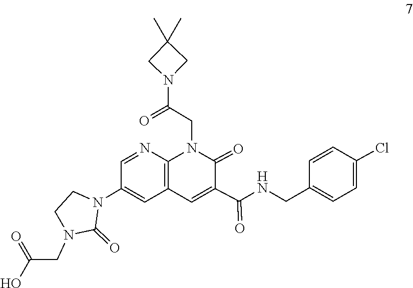 [SMILES: CC1(C)CN(C(=O)Cn2c(=O)c(C(=O)NCc3ccc(Cl)cc3)cc3cc(N4CCN(CC(=O)O)C4=O)cnc32)C1]

7 was synthesized as in Scheme 7.

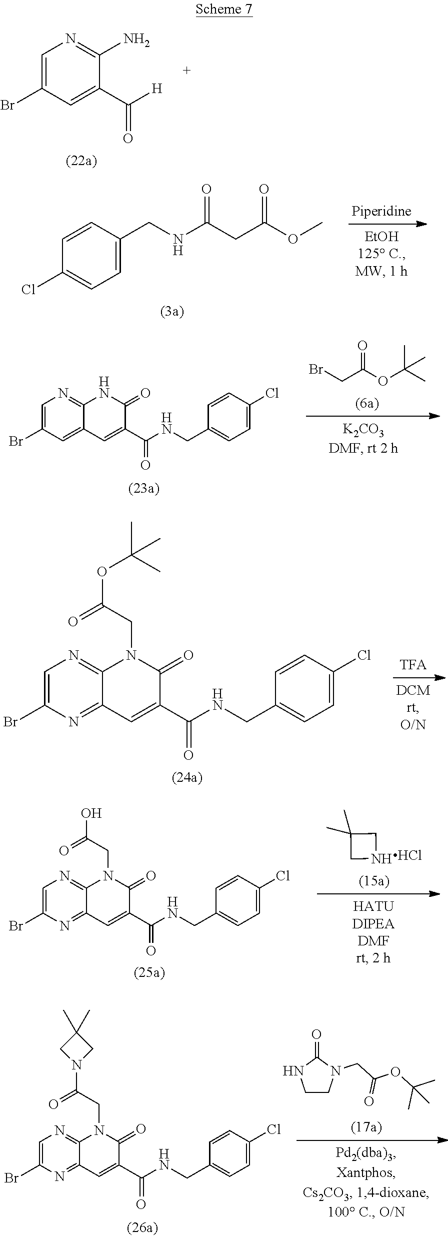

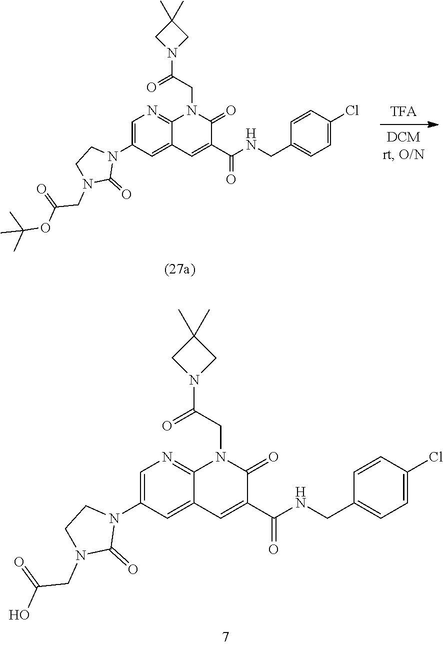 [SMILES: CC1(C)CN(C(=O)Cn2c(=O)c(C(=O)NCc3ccc(Cl)cc3)cc3cc(N4CCN(CC(=O)OC(C)(C)C)C4=O)cnc32)C1 ; CC1(C)CN(C(=O)Cn2c(=O)c(C(=O)NCc3ccc(Cl)cc3)cc3cc(N4CCN(CC(=O)O)C4=O)cnc32)C1]

Preparation of 6-bromo-N-(4-chlorobenzyl)-2-oxo-1,2-dihydro-1,8-naphthyridine-3-carboxamide, (23a). A microwave vial (30 mL) with the mixture of 2-amino-5-bromonicotinaldehyde (22a) (150 mg, 0.75 mmol), methyl 3-((4-chlorobenzyl)amino)-3-oxopropanoate (3a) (450 mg, 1.76 mmol), and piperidine (160 mg, 1.87 mmol) in absolute ethanol (12 mL) was irradiated in a microwave reactor for 1 hour at 125° C. After cooling to room temperature, the resulting suspension was filtered and the solid was washed with ethyl acetate (20 mL) and then dried under the vacuum for 4 h to provide 6-bromo-N-(4-chlorobenzyl)-2-oxo-1,2-dihydro-1,8-naphthyridine-3-carboxamide (23a) (256 mg, 87% yield) as an off-white powder. LC/MS: Eluent system A (retention time: 5.85 min); ESI-MS 392 [M+H]⁺.

Preparation of 6-bromo-N-(4-chlorobenzyl)-1-(2-(3,3-dimethylazetidin-1-yl)-2-oxoethyl)-2-oxo-1,2-dihydro-1,8-naphthyridine-3-carboxamide, (26a). To a solution of 6-bromo-N-(4-chlorobenzyl)-2-oxo-1,2-dihydro-1,8-naphthyridine-3-carboxamide (23a) (200 mg, 0.51 mmol) in DMF (8 mL) was added tert-butyl 2-bromoacetate (6a) (300 mg, 1.5 mmol) and potassium carbonate (300 mg, 2.2 mmol). After 2 h at room temperature, the mixture was filtered through a plug of Celite and the filtrate was concentrated under reduced pressure producing tert-butyl 2-(6-bromo-3-((4-chlorobenzyl)carbamoyl)-2-oxo-1,8-naphthyridin-1(2H)-yl)acetate (24a), which was dissolved in DCM (5 mL) and TFA (2 mL) was added. After overnight, the volatile components were removed in vacuo to produce 2-(6-bromo-3-((4-chlorobenzyl)carbamoyl)-2-oxo-1,8-naphthyridin-1(2H)-yl)acetic acid (25a). The residue was dissolved in DMF (8 mL), and 3,3-dimethylazetidine hydrochloride (15a) (121 mg, 1 mmol) was added, followed by HATU (380 mg, 1 mmol) and DIPEA (250 mg, 1.9 mmol). After 2 h at room temperature, the mixture was concentrated under reduced pressure, dissolved in chloroform and loaded on silica gel column. The column was eluted with a gradient of 0% to 7% MeOH in CHCl₃, providing 6-bromo-N-(4-chlorobenzyl)-1-(2-(3,3-dimethylazetidin-1-yl)-2-oxoethyl)-2-oxo-1,2-dihydro-1,8-naphthyridine-3-carboxamide (26a) (196 mg, 74% yield over three steps) as a yellow amorphous powder. LC/MS: Eluent system A (retention time: 6.66 min); ESI-MS 517 [M+H]⁺.

Preparation of tert-butyl 2-(3-(6-((4-chlorobenzyl)carbamoyl)-8-(2-(3,3-dimethylazetidin-1-yl)-2-oxoethyl)-7-oxo-7,8-dihydro-1,8-naphthyridin-3-yl)-2-oxoimidazolidin-1-yl)acetate, (27a). A mixture of (26a) (100 mg, 0.19 mmol), tert-butyl 2-(2-oxoimidazolidin-1-yl)acetate (17a) (100 mg, 0.5 mmol), Pd₂(dba)₃ (23 mg, 0.025 mmol), Xantphos (43 mg, 0.075 mmol), and caesium carbonate (245 mg, 0.75 mmol) in anhydrous 1,4-dioxane (15 mL) was overnight at 100° C., then filtered through a plug of Celite, washed with chloroform (2×10 mL) and the filtrate was concentrated under reduced pressure. The residue was purified by column chromatography on silica gel (eluted with a gradient of ethyl acetate-MeOH 0% to 5%), which provided (27a) (84 mg, 70% yield) as a yellow amorphous powder. LC/MS: Eluent system A (retention time: 6.81 min); ESI-MS 637.4 [M+H]⁺.

Preparation of [3-(6-{[(4-chlorophenyl)methyl]carbamoyl}-8-[2-(3,3-dimethylazetidin-1-yl)-2-oxoethyl]-7-oxo-7,8-dihydro-1,8-naphthyridin-3-yl)-2-oxoimidazolidin-1-yl]acetic acid, 7. A solution of (27a) (50 mg, 0.08 mmol) in DCM (8 mL) was treated with TFA (3 mL). After overnight at room temperature, the mixture was concentrated under reduced pressure. The residue was co-evaporated with toluene (10 mL), which provided 7 (30 mg, 67% yield) as a yellow amorphous powder. ¹H NMR (600 MHz, DMSO-d₆) δ 12.94 (br s, 1H), 9.97 (t, J=6.1 Hz, 1H), 9.16 (d, J=2.7 Hz, 1H), 8.93 (s, 1H), 8.47 (d, J=2.7 Hz, 1H), 7.43-7.36 (m, 4H), 5.10 (s, 2H), 4.57 (d, J=6.0 Hz, 2H), 4.02 (s, 2H), 3.99 (s, 2H), 3.97-3.93 (m, 2H), 3.66-3.62 (m, 2H), 3.59 (s, 2H), 1.29 (s, 6H). LC/MS: Eluent system A (retention time: 5.83 min); ESI-MS 581 [M+H]⁺. 579 [M−H]⁻.

### Synthesis of 5-(2-(3-fluoro-3-methylazetidin-1-yl)-2-oxoethyl)-N-(4-fluorobenzyl)-2-(3-methyl-2-oxoimidazolidin-1-yl)-6-oxo-5,6-dihydropyrido[2,3-b]pyrazine-7-carboxamide, (Compound 8)

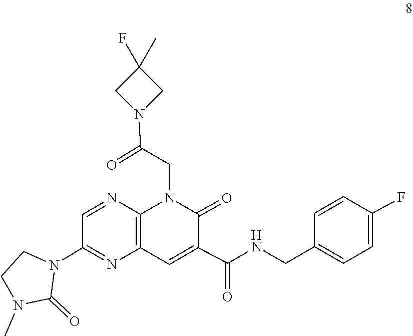 [SMILES: CN1CCN(c2cnc3c(cc(C(=O)NCc4ccc(F)cc4)c(=O)n3CC(=O)N3CC(C)(F)C3)n2)C1=O]

8 was synthesized as in Scheme 8.

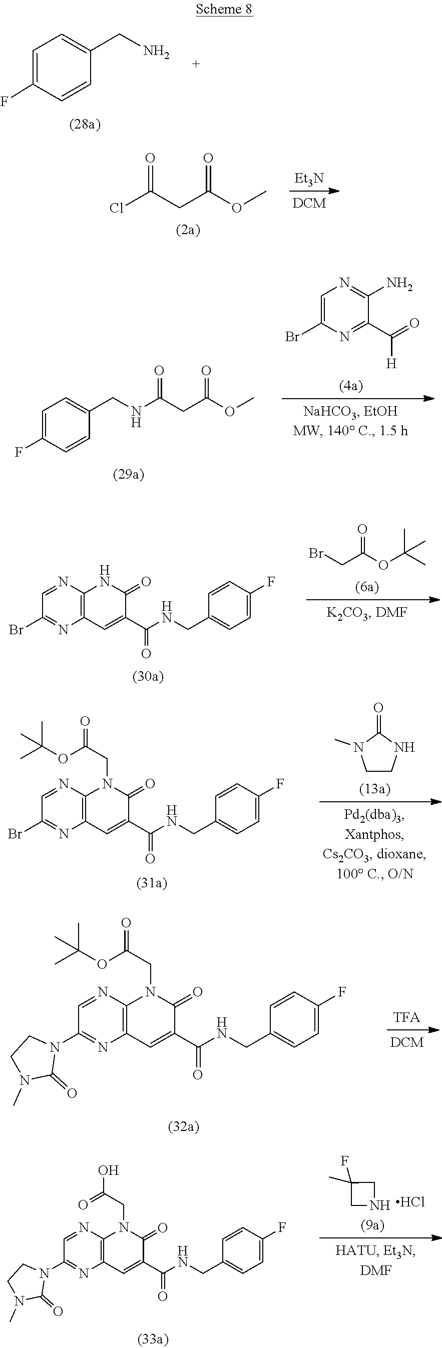

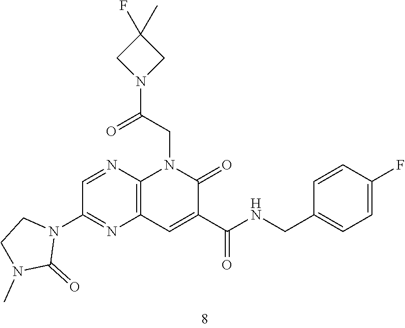 [SMILES: CN1CCN(c2cnc3c(cc(C(=O)NCc4ccc(F)cc4)c(=O)n3CC(=O)N3CC(C)(F)C3)n2)C1=O]

Preparation methyl 3-{[(4-fluorophenyl)methyl]amino}-3-oxopropanoate, (29a). To a solution of 4-fluorobenzylamine (28a) (3.2 g, 25.8 mmol) in DCM (50 mL) was added Et₃N (10.8 mL, 77.4 mmol) and the solution was cooled in a −78° C. bath, upon which a solution of methyl 3-chloro-3-oxopropionate (2a) (2.8 mL, 25.8 mmol) in DCM (20 mL) was added via a dropping funnel over 10 min. After 30 min in the −78° C. bath, the bath was removed. After overnight at room temperature to the mixture was added a saturated NaHCO₃ solution. The separated organic phase was washed with brine, dried over Na₂SO₄, filtered and concentrated under reduced pressure. The crude material was suspended in ˜10 mL EtOAc and further diluted with by 200 mL hexanes. The resulting solid was filtered, washed with hexanes, and dried, which provided (29a) as a light yellow solid (3.9 g, 67% yield).

Preparation of 2-bromo-N-[(4-fluorophenyl)methyl]-6-oxo-5,6-dihydropyrido[2,3-b]pyrazine-7-carboxamide, (30a). To a microwave reaction vial was added 3-amino-6-bromopyrazine-2-carbaldehyde (4a) (0.75 g, 3.7 mmol), methyl 3-{[(4-fluorophenyl)methyl]amino}-3-oxopropanoate (29a) (1.2 g, 5.2 mmol), and NaHCO₃ (0.93 g, 11.1 mmol) in absolute ethanol (10 mL). The microwave reactor was set at 140° C. for 1 h. The solvent was removed under reduced pressure. Added diethyl ether and the solid was collected by filtration under vacuum, and dried, which afforded (30a) as a brown solid along with inorganics. This material was used in the next step without further purification.

Preparation of tert-butyl [2-bromo-7-{[(4-fluorophenyl)methyl]carbamoyl}-6-oxopyrido[2,3-b]pyrazin-5(6H)-yl]acetate, (31a). To a suspension of (30a) in DMF was added tert-butyl bromoacetate (6a) (0.55 mL, 3.7 mmol), followed by K₂CO₃ (1.5 g, 11.1 mmol) at room temperature and the reaction mixture was stirred for 4 h. After completion of the reaction, the solvent was removed under reduced pressure. The residue was diluted with water and extracted with DCM, washed with brine. The organic layer was dried over anhydrous Na₂SO₄, filtered and concentrated under reduced pressure. The crude was absorbed on silica gel and product purified by column chromatography on silica (eluted with 0% to 75% EtOAc in hexanes), which generated (31a) as a light yellow solid (1.0 g, 55% yield over two steps).

Preparation of tert-butyl [7-{[(4-fluorophenyl)methyl]carbamoyl}-2-(3-methyl-2-oxoimidazolidin-1-yl)-6-oxopyrido[2,3-b]pyrazin-5(6H)-yl]acetate, (32a). To a solution of (31a) (500 mg, 1.0 mmol) and 1-methyl-2-imidazolidone (13a) (306 mg, 3.06 mmol) in dioxane (15 mL) was added Pd₂(dba)3 (47 mg, 0.005 mmol), Xantphos (89 mg, 0.15 mmol), followed by Cs₂CO₃ (465 mg, 1.4 mmol). The reaction mixture was heated with an oil bath at 100° C. for overnight. After cooling to room temperature, it was adsorbed on silica gel. The product was purification by column chromatography on silica gel with a gradient of 0% to 10% MeOH in EtOAc, which afforded (32a) (510 mg, 98% yield) as a light yellow solid.

Preparation of [7-{[(4-fluorophenyl)methyl]carbamoyl}-2-(3-methyl-2-oxoimidazolidin-1-yl)-6-oxopyrido[2,3-b]pyrazin-5(6H)-yl]acetic acid, (33a). To a solution of (32a) (510 mg, 1.0 mmol) in DCM (10 mL) was added trifluoroacetic acid (5 mL) at room temperature. After stirring 3 h, the solvent was removed under reduced pressure, and the residue was co-evaporated with DCM/ether (1:1, 3×20 mL). The residue was dissolved in DCM and solidified with ether. The solid was collected by filtration and dried, which provided (33a) (380 mg, 84% yield) as a light yellow solid.

Preparation of 5-[2-(3-fluoro-3-methylazetidin-1-yl)-2-oxoethyl]-N-[(4-fluorophenyl)methyl]-2-(3-methyl-2-oxoimidazolidin-1-yl)-6-oxo-5,6-dihydropyrido[2,3-b]pyrazine-7-carboxamide, 8. To a solution of (33a) (70 mg, 0.15 mmol) and 3-fluoro-3-methylazetidine hydrochloride (9a) (29 mg, 0.23 mmol) in DMF was added HATU (87 mg, 0.23 mmol) and triethylamine (0.11 mL, 0.77 mmol). After 2 h at room temperature, the solvent was removed under vacuum. The residue was dissolved in DCM and adsorbed on silica gel. The product was purified by column chromatography on silica gel (eluted with a gradient of 0% to 10% MeOH in EtOAc), which generated 8 (27 mg, 29% yield) as a light yellow solid. 1H NMR (600 MHz, DMSO-d₆) δ 9.90 (t, J=6.0 Hz, 1H), 9.62-9.61 (m, 1H), 8.67-8.66 (m, 1H), 7.45-7.37 (m, 2H), 7.20-7.16 (m, 2H), 5.18-5.08 (m, 2H), 4.59-4.41 (m, 4H), 4.06-3.96 (m, 4H), 3.60-3.54 (m, 2H), 2.85 (s, 3H), 1.64 (d, J=22.2 Hz, 3H). ¹⁹F NMR (565 MHz, DMSO-d₆) δ −115.71-−115.79 (m, 1F), −138.86-−139.17 (m, 1F). LC/MS: Eluent system A (retention time: 5.36 min); ESI-MS 526 [M+H]⁺.

### Synthesis of N-(4-cyanobenzyl)-5-(2-(3-fluoro-3-methylazetidin-1-yl)-2-oxoethyl)-2-(3-methyl-2-oxoimidazolidin-1-yl)-6-oxo-5,6-dihydropyrido[2,3-b]pyrazine-7-carboxamide, (Compound 9)

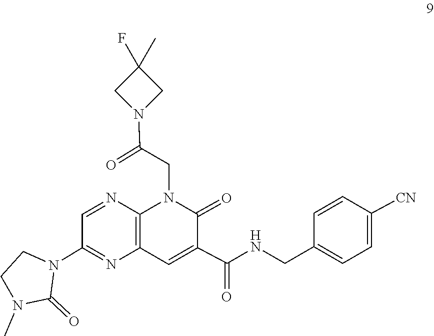 [SMILES: CN1CCN(c2cnc3c(cc(C(=O)NCc4ccc(C#N)cc4)c(=O)n3CC(=O)N3CC(C)(F)C3)n2)C1=O]

9 was synthesized as in Scheme 9.

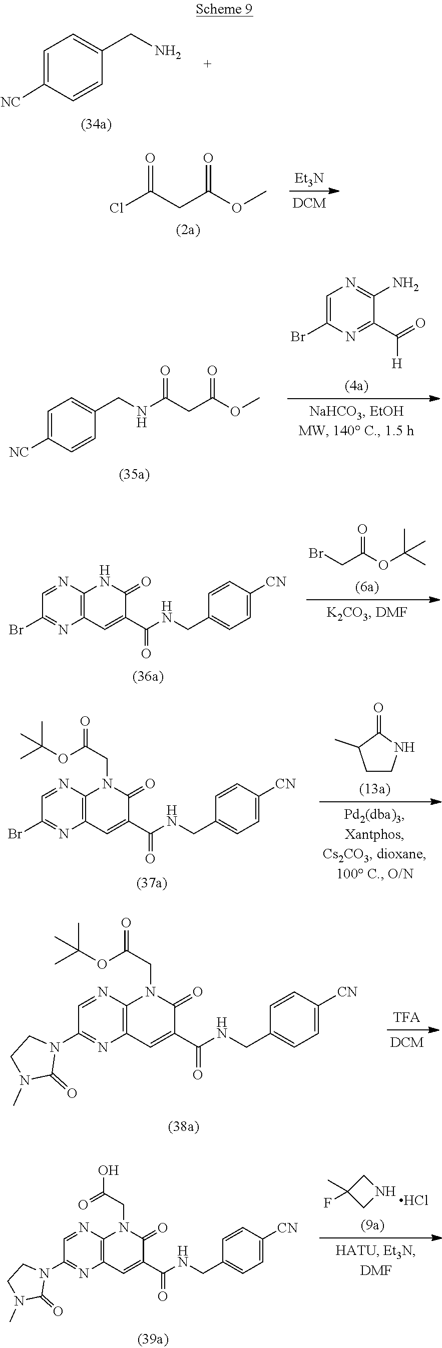

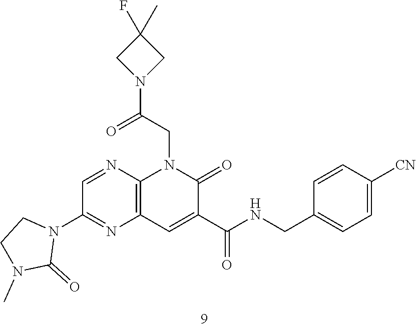 [SMILES: CN1CCN(c2cnc3c(cc(C(=O)NCc4ccc(C#N)cc4)c(=O)n3CC(=O)N3CC(C)(F)C3)n2)C1=O]

Preparation methyl 3-{[(4-cyanophenyl)methyl]amino}-3-oxopropanoate, (35a). To a solution of 4-cyanobenzylamine (34a) (3.1 g, 23.5 mmol) in DCM (50 mL) was added Et₃N (9.8 mL, 70.5 mmol) and after cooling in a −78° C. bath, a solution of methyl 3-chloro-3-oxopropionate (2a) (2.5 mL, 23.5 mmol) in DCM (20 mL) was added via a dropping funnel over 10 min. After 30 min, the −78° C. bath was removed. After overnight at room temperature, to the mixture was added a saturated NaHCO₃ solution. The layers were separated and the organic layer was washed with brine, dried over anhydrous Na₂SO₄, filtered and concentrated under vacuum. To the crude material was added about 10 mL EtOAc followed by 200 mL hexanes. The precipitate was filtered, washed with hexanes and dried, which afforded (35a) as a light yellow solid (4.5 g, 82% yield).

Preparation of 2-bromo-N-[(4-cyanophenyl)methyl]-6-oxo-5,6-dihydropyrido[2,3-b]pyrazine-7-carboxamide, (36a). To a microwave reaction vial was added 3-amino-6-bromopyrazine-2-carbaldehyde (4a) (0.75 g, 3.7 mmol), methyl 3-{[(4-cyanophenyl)methyl]amino}-3-oxopropanoate (35a) (1.2 g, 5.2 mmol), and NaHCO₃ (0.93 g, 11.1 mmol) in absolute ethanol (10 mL). The microwave reactor was set to 140° C. for 1.5 h. After completion of the reaction, concentrated under vacuum. The residue was added diethyl ether and filtered, washed with ether and dried, which provided (36a) as a brown solid along with inorganics. This material was used in the next step without further purification.

Preparation of tert-butyl [2-bromo-7-{[(4-cyanophenyl)methyl]carbamoyl}-6-oxopyrido[2,3-b]pyrazin-5(6H)-yl]acetate, (37a). To a suspension of crude (36a) in DMF (10 mL) was added tert-butyl bromoacetate (6a) (0.55 mL, 3.7 mmol) followed by K₂CO₃ (1.5 g, 11.1 mmol). After 4 h, the solvent was removed under reduced pressure. The residue was diluted with water and extracted with DCM and washed with brine. The organic layer was dried over anhydrous Na₂SO₄, filtered and concentrated under reduced pressure. The crude was absorbed with silica gel and product purified by column chromatography (eluted with 0% to 75% EtOAc in hexanes), which generated (37a) as a light yellow solid (710 mg, 38% yield over two steps).

Preparation of tert-butyl [7-{[(4-cyanophenyl)methyl]carbamoyl}-2-(3-methyl-2-oxoimidazolidin-1-yl)-6-oxopyrido[2,3-b]pyrazin-5(6H)-yl]acetate, (38a). To a solution of (37a) (490 mg, 0.98 mmol) and 1-methyl-2-imidazolidone (13a) (295 mg, 2.95 mmol) in dioxane (15 mL) was added Pd₂(dba)₃ (45 mg, 0.049 mmol), Xantphos (85 mg, 0.15 mmol), followed by Cs₂CO₃ (447 mg, 1.37 mmol). The reaction mixture was heated overnight with an oil bath at 100° C. After which it was cooled to room temperature, adsorbed on silica gel, and the product purified by column chromatography on silica with a gradient of 0% to 10% MeOH in EtOAc, which provided (38a) (356 mg, 70% yield) as a light yellow solid.

Preparation of [7-{[(4-cyanophenyl)methyl]carbamoyl}-2-(3-methyl-2-oxoimidazolidin-1-yl)-6-oxopyrido[2,3-b]pyrazin-5(6H)-yl]acetic acid, (39a). To a solution of (38a) (340 mg, 0.66 mmol) in DCM (10 mL) was added trifluoroacetic acid (5 mL). After stirring 3 h at room temperature, the solvent was removed under reduced pressure, and then the residue was co-evaporated with DCM/ether (1:1, 3×20 mL). The crude was dissolved in DCM and solidified with ether. The solid was filtered and dried under vacuum, which generated (39a) (240 mg, 79% yield) as a light yellow solid.

Preparation of N-(4-cyanobenzyl)-5-(2-(3-fluoro-3-methylazetidin-1-yl)-2-oxoethyl)-2-(3-methyl-2-oxoimidazolidin-1-yl)-6-oxo-5,6-dihydropyrido[2,3-b]pyrazine-7-carboxamide, 9. To a solution of (39a) (80 mg, 0.17 mmol) and 3-fluoro-3-methylazetidine hydrochloride (9a) (33 mg, 0.26 mmol) in DMF was added HATU (99 mg, 0.26 mmol) and triethylamine (0.12 mL, 0.87 mmol). After 2 h, the solvent was removed under reduced pressure. The residue was dissolved in DCM and adsorbed on silica gel. The product was purified by column chromatography on silica with a gradient of 0% to 10% MeOH in EtOAc, which provided 9 (27 mg, 29% yield) as a light yellow solid. ¹H NMR (600 MHz, DMSO-d₆) δ 9.99 (t, J=6.2 Hz, 1H), 9.63 (s, 1H), 8.65 (s, 1H), 7.85-7.79 (m, 2H), 7.56-7.53 (m, 2H), 5.19-5.09 (m, 2H), 4.67 (d, J=6.0 Hz, 2H), 4.55-4.43 (m, 2H), 4.07-3.97 (m, 4H), 3.60-3.54 (m, 2H), 2.85 (s, 3H), 1.63 (d, J=22.2 Hz, 3H). ¹⁹F NMR (565 MHz, DMSO-d₆) δ −138.93-−139.12 (m, 1F). LC/MS: Eluent system A (retention time: 4.55 min); ESI-MS 533 [M+H]⁺.

### Synthesis of 5-[2-(5-azaspiro[2.3]hexan-5-yl)-2-oxoethyl]-N-[(4-fluorophenyl)methyl]-2-(3-methyl-2-oxoimidazolidin-1-yl)-6-oxo-5,6-dihydropyrido[2,3-b]pyrazine-7-carboxamide, (Compound 10)

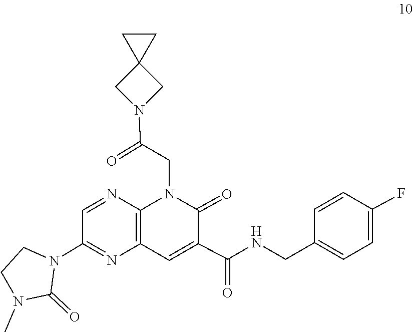 [SMILES: CN1CCN(c2cnc3c(cc(C(=O)NCc4ccc(F)cc4)c(=O)n3CC(=O)N3CC4(CC4)C3)n2)C1=O]

Preparation of 5-[2-(5-azaspiro[2.3]hexan-5-yl)-2-oxoethyl]-N-[(4-fluorophenyl)methyl]-2-(3-methyl-2-oxoimidazolidin-1-yl)-6-oxo-5,6-dihydropyrido[2,3-b]pyrazine-7-carboxamide, 10. To a solution of (33a) (80 mg, 0.18 mmol) 5-azaspiro-[2.3]-hexane hydrochloride (40a) (31 mg, 0.26 mmol) in DMF (3 mL) was added HATU (100 mg, 0.26 mmol) and triethylamine (120 μL, 0.87 mmol). After 2 h at room temperature, the solvent was removed under reduced pressure. The residue was dissolved in DCM and adsorbed on silica gel. The product was purified by column chromatography (eluted with 0% to 10% MeOH in CHCl₃, provided 10 (28 mg, 31% yield) as a light yellow solid. ¹H NMR (600 MHz, DMSO-d6) δ 9.93 (t, J=6.0 Hz, 1H), 9.63 (s, 1H), 8.67 (s, 1H), 7.44-7.38 (m, 2H), 7.22-7.14 (m, 2H), 5.12 (s, 2H), 4.57 (d, J=6.0 Hz, 2H), 4.40 (s, 2H), 4.03-3.98 (m, 2H), 3.97 (s, 2H), 3.61-3.55 (m, 2H), 2.86 (s, 3H), 0.75-0.63 (m, 4H). ¹⁹F NMR (565 MHz, DMSO-d6) δ−115.71-−115.81 (m, 1F). LC/MS: Eluent system A (retention time: 5.71 min); ESI-MS 520 [M+H]⁺.

### Synthesis of 5-[2-(5-azaspiro[2.3]hexan-5-yl)-2-oxoethyl]-N-[(4-cyanophenyl)methyl]-2-(3-methyl-2-oxoimidazolidin-1-yl)-6-oxo-5,6-dihydropyrido[2,3-b]pyrazine-7-carboxamide, (Compound 11)

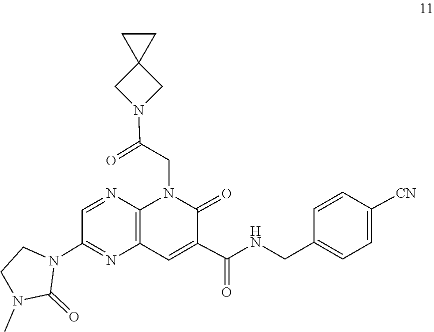 [SMILES: CN1CCN(c2cnc3c(cc(C(=O)NCc4ccc(C#N)cc4)c(=O)n3CC(=O)N3CC4(CC4)C3)n2)C1=O]

Preparation of 5-[2-(5-azaspiro[2.3]hexan-5-yl)-2-oxoethyl]-N-[(4-cyanophenyl)methyl]-2-(3-methyl-2-oxoimidazolidin-1-yl)-6-oxo-5,6-dihydropyrido[2,3-b]pyrazine-7-carboxamide, 11. To a solution of (39a) (80 mg, 0.17 mmol) 5-azaspiro-[2.3]-hexane hydrochloride (40a) (31 mg, 0.26 mmol) in DMF (5 mL) was added HATU (99 mg, 0.26 mmol) and triethylamine (0.12 mL, 0.87 mmol) at room temperature. The mixture was stirred for 2 h. After completion of the reaction, the solvent was removed under reduced pressure. The residue was dissolved in DCM and adsorbed silica gel and the product was purified by column chromatography on silica, with a gradient of 0% to 10% MeOH in CHCl₃, provided 11 (33 mg, 36% yield) as a light yellow solid. ¹H NMR (600 MHz, DMSO-d₆) δ 10.01 (t, J=6.0 Hz, 1H), 9.63 (s, 1H), 8.64 (s, 1H), 7.84-7.79 (m, 2H), 7.55-7.53 (m, 2H), 5.13 (s, 2H), 4.66 (d, J=6.4 Hz, 2H), 4.40 (s, 2H), 4.03-3.94 (m, 4H), 3.59-3.53 (m, 2H), 2.85 (s, 3H), 0.75-0.63 (m, 4H). LC/MS: Eluent system A (retention time: 5.56 min); ESI-MS 527 [M+H]⁺.

### Synthesis of N-(4-chlorobenzyl)-5-(2-(3-fluoro-3-methylazetidin-1-yl)-2-oxoethyl)-6-oxo-2-(2-oxoimidazolidin-1-yl)-5,6-dihydropyrido[2,3-b]pyrazine-7-carboxamide, (Compound 12)

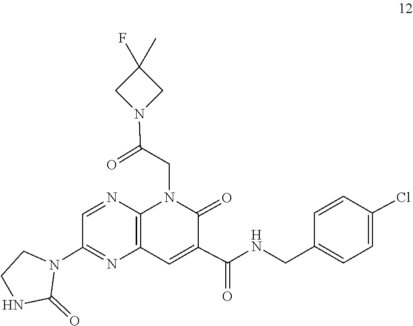 [SMILES: CC1(F)CN(C(=O)Cn2c(=O)c(C(=O)NCc3ccc(Cl)cc3)cc3nc(N4CCNC4=O)cnc32)C1]

Preparation of N-(4-chlorobenzyl)-5-(2-(3-fluoro-3-methylazetidin-1-yl)-2-oxoethyl)-6-oxo-2-(2-oxoimidazolidin-1-yl)-5,6-dihydropyrido[2,3-b]pyrazine-7-carboxamide, 12. A similar procedure as described for compound 1 was followed: with 2-bromo-N-[(4-chlorophenyl)methyl]-5-[2-(3-fluoro-3-methylazetidin-1-yl)-2-oxoethyl]-6-oxo-5,6-dihydropyrido[2,3-b]pyrazine-7-carboxamide (10a) (80 mg, 0.15 mmol), 2-imidazolidinone (41a) (132 mg, 1.53 mmol), Pd₂(dba)₃ (6.5 mg, 0.008 mmol), Xantphos (13.3 mg, 0.023 mmol), and Cs₂CO₃ (69 mg, 0.2 mmol) in dioxane (5 mL), followed by silica gel column purification (eluted with a gradient of 0% to 10% MeOH in EtOAc), afforded 12 (13 mg, 16% yield) as a light yellow solid. ¹H NMR (600 MHz, DMSO-d₆) δ 9.92 (t, J=6.0 Hz, 1H), 9.60 (s, 1H), 8.65 (s, 1H), 7.59 (s, 1H), 7.44-7.35 (m, 4H), 5.21-5.06 (m, 2H), 4.63-4.40 (m, 4H), 4.13-3.95 (m, 4H), 3.58-3.48 (m, 2H), 1.63 (d, J=22.2 Hz, 3H). ¹⁹F NMR (565 MHz, DMSO-d₆) δ −138.85-−139.18 (m, 1F). LC/MS: Eluent system A (retention time: 5.56 min); ESI-MS 528 [M+H]⁺.

### Synthesis of [3-(7-{[(4-chlorophenyl)methyl]carbamoyl}-5-[2-(3-fluoro-3-methylazetidin-1-yl)-2-oxoethyl]-6-oxo-5,6-dihydropyrido[2,3-b]pyrazin-2-yl)-2-oxoimidazolidin-1-yl]acetic Acid, (Compound 13)

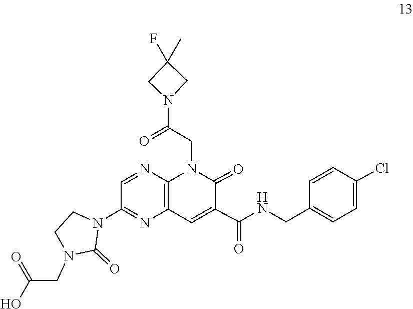 [SMILES: CC1(F)CN(C(=O)Cn2c(=O)c(C(=O)NCc3ccc(Cl)cc3)cc3nc(N4CCN(CC(=O)O)C4=O)cnc32)C1]

13 was synthesized as in Scheme 10.

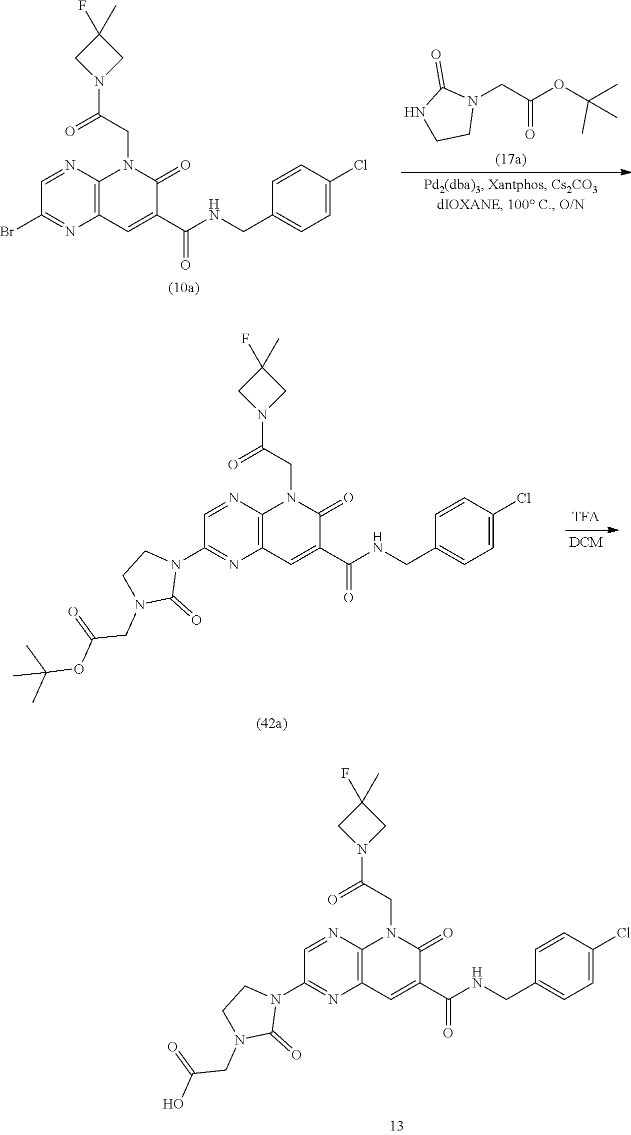 [SMILES: CC1(F)CN(C(=O)Cn2c(=O)c(C(=O)NCc3ccc(Cl)cc3)cc3nc(Br)cnc32)C1 ; CC(C)(C)OC(=O)CN1CCNC1=O ; CC1(F)CN(C(=O)Cn2c(=O)c(C(=O)NCc3ccc(Cl)cc3)cc3nc(N4CCN(CC(=O)OC(C)(C)C)C4=O)cnc32)C1 ; CC1(F)CN(C(=O)Cn2c(=O)c(C(=O)NCc3ccc(Cl)cc3)cc3nc(N4CCN(CC(=O)O)C4=O)cnc32)C1]

Preparation of tert-butyl [3-(7-{[(4-chlorophenyl)methyl]carbamoyl}-5-[2-(3-fluoroazetidin-1-yl)-2-oxoethyl]-6-oxo-5,6-dihydropyrido[2,3-b]pyrazin-2-yl)-2-oxoimidazolidin-1-yl]acetate, (42a). A similar procedure as described for compound 1 was followed: with 2-bromo-N-[(4-chlorophenyl)methyl]-5-[2-(3-fluoro-3-methylazetidin-1-yl)-2-oxoethyl]-6-oxo-5,6-dihydropyrido[2,3-b]pyrazine-7-carboxamide (10a) (80 mg, 0.15 mmol), tert-butyl (2-oxoimidazolidin-1-yl)acetate (17a) (132 mg, 1.53 mmol), Pd₂(dba)₃ (6.5 mg, 0.008 mmol), Xantphos (13.3 mg, 0.023 mmol), and Cs₂CO₃ (69 mg, 0.2 mmol) in dioxane (5 mL), followed by silica gel column purification (eluent: 0% to 5% MeOH in EtOAc) provided (42a) (75 mg, 76% yield) as a light yellow solid.

Preparation of [3-(7-{[(4-chlorophenyl)methyl]carbamoyl}-5-[2-(3-fluoro-3-methylazetidin-1-yl)-2-oxoethyl]-6-oxo-5,6-dihydropyrido[2,3-b]pyrazin-2-yl)-2-oxoimidazolidin-1-yl]acetic acid, 13. To a solution of (42a) (72 mg, 0.12 mmol) in DCM (4 mL) was added trifluoroacetic acid (2 mL). After 2 h at room temperature, the solvent was removed under reduced pressure, and then the residue was co-evaporated with DCM (2×20 mL). The residue was dissolved in DCM and solidified with ether and the solid collected by vacuum filtration, washed with ether and dried, which afforded 13 (58 mg, 85% yield) as a light yellow solid. ¹H NMR (600 MHz, DMSO-d₆) δ 9.92 (t, J=6.0 Hz, 1H), 9.62 (s, 1H), 8.66 (s, 1H), 7.44-7.36 (m, 4H), 5.24-5.06 (m, 2H), 4.62-4.38 (m, 2H), 4.06-3.94 (m, 4H), 3.73-3.58 (m, 4H), 1.63 (d, J=22.2 Hz, 3H). ¹⁹F NMR (565 MHz, DMSO-d₆) δ− 139.87-−140.14 (m, 1F). LC/MS: Eluent system A (retention time: 5.39 min); ESI-MS 586 [M+H]⁺.

### Synthesis of N-[(4-chlorophenyl)methyl]-5-[2-(3-fluoro-3-methylazetidin-1-yl)-2-oxoethyl]-2-(3-methyl-2,4-dioxoimidazolidin-1-yl)-6-oxo-5,6-dihydropyrido[2,3-b]pyrazine-7-carboxamide, (Compound 14)

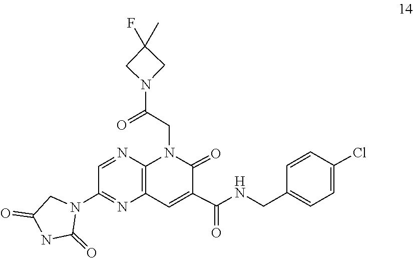 [SMILES: CC1(F)CN(C(=O)Cn2c(=O)c(C(=O)NCc3ccc(Cl)cc3)cc3nc(N4CC(=O)NC4=O)cnc32)C1]

Preparation of N-[(4-chlorophenyl)methyl]-5-[2-(3-fluoro-3-methylazetidin-1-yl)-2-oxoethyl]-2-(3-methyl-2,4-dioxoimidazolidin-1-yl)-6-oxo-5,6-dihydropyrido[2,3-b]pyrazine-7-carboxamide, 14. A mixture of 2-bromo-N-[(4-chlorophenyl)methyl]-5-[2-(3-fluoro-3-methylazetidin-1-yl)-2-oxoethyl]-6-oxo-5,6-dihydropyrido[2,3-b]pyrazine-7-carboxamide (10a) (65.0 mg, 0.125 mmol), 3-methylimidazolidine-2,4-dione (43a) (28.5 mg, 0.25 mmol), N,N′-dimethylethylenediamine (44a) (1.1 mg, 0.013 mmol), copper(I) iodide (2.5 mg, 0.013 mmol), potassium carbonate (69.1 mg, 0.50 mmol) in dioxane (8 mL) was placed in a microwave reactor vial that was set to 160° C. for 1 h. After cooling to ambient temperature, the mixture was concentrated under reduced pressure, and the resulting residue was dissolved in chloroform (50 mL) and filtered. The product was purified by column chromatography on silica gel [eluted with a gradient of 0% to 2.5% MeOH in EtOAc/MTBE (1:1)] produced 14 (11.0 mg, 16% yield) as a light yellow solid. 1H NMR (600 MHz, DMSO-d₆) δ 9.86 (t, J=6.02 Hz, 1H), 9.54 (s, 1H), 8.69 (s, 1H), 7.44-7.37 (m, 4H), 5.21-5.10 (m, 2H), 4.61-4.56 (m, 4H), 4.55-4.43 (m, 2H), 4.06-3.99 (m, 2H), 3.00 (s, 3H), 1.64 (d, J=22.2 Hz, 3H). ¹⁹F NMR (565 MHz, DMSO-d₆) δ−139.78-−138.46 (m, 1 F). LC/MS: Eluent system A (retention time: 5.50 min); ESI-MS: 556 [M+H]⁺.

### Synthesis of 5-[2-(5-azaspiro[2.3]hexan-5-yl)-2-oxoethyl]-N-[(4-chlorophenyl)methyl]-2-(3-methyl-2,4-dioxoimidazolidin-1-yl)-6-oxo-5,6-dihydropyrido[2,3-b]pyrazine-7-carboxamide, (Compound 15)

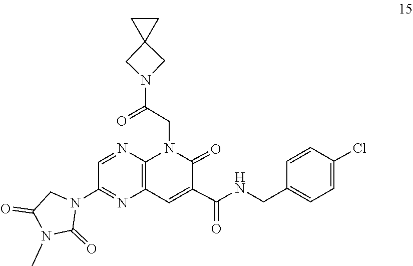 [SMILES: CN1C(=O)CN(c2cnc3c(cc(C(=O)NCc4ccc(Cl)cc4)c(=O)n3CC(=O)N3CC4(CC4)C3)n2)C1=O]

15 was synthesized as in Scheme 11.

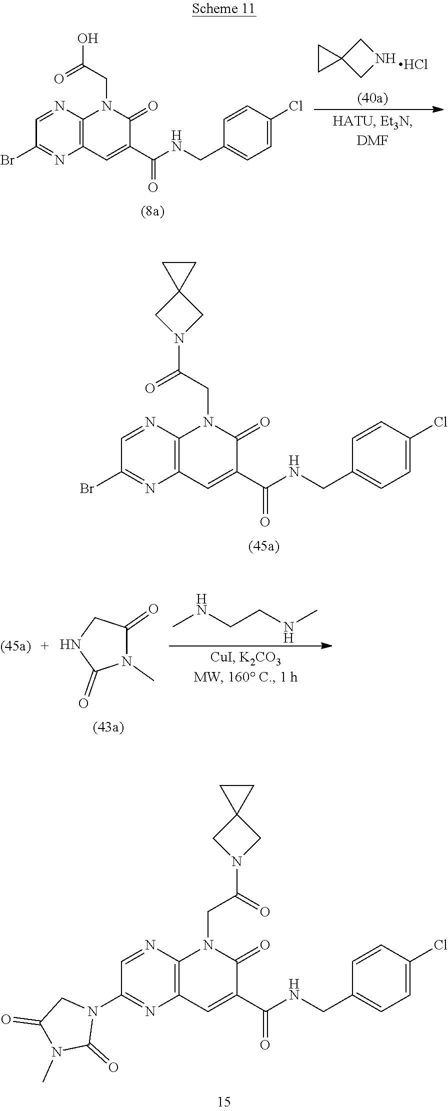

Preparation of 5-[2-(5-azaspiro[2.3]hexan-5-yl)-2-oxoethyl]-2-bromo-N-[(4-chlorophenyl)methyl]-6-oxo-5,6-dihydropyrido[2,3-b]pyrazine-7-carboxamide, (45a). To a mixture of [2-bromo-7-{[(4-chlorophenyl)methyl]carbamoyl}-6-oxopyrido[2,3-b]pyrazin-5(6H)-yl]acetic acid (8a) (750 mg, 1.66 mmol), HATU (884 mg, 2.33 mmol), 5-azaspiro-[2.3]-hexane hydrochloride (40a) (238 mg, 1.99 mmol) in DMF (10 mL), was added DIPEA (1.45 mL, 8.3 mmol). After 1 h at room temperature, the reaction was quenched with water (50 mL) and extracted with chloroform (3×50 mL). The organic layer was washed with brine (1×50 mL), dried over anhydrous Na₂SO₄, and concentrated under vacuum. Purification of the product by column chromatography on silica gel with a gradient of 0% to 20%, MeOH in EtOAc produced (45a) (680 mg, 79% yield).

Preparation of 5-[2-(5-azaspiro[2.3]hexan-5-yl)-2-oxoethyl]-N-[(4-chlorophenyl)methyl]-2-(3-methyl-2,4-dioxoimidazolidin-1-yl)-6-oxo-5,6-dihydropyrido[2,3-b]pyrazine-7-carboxamide, 15. A mixture of 5-[2-(5-azaspiro[2.3]hexan-5-yl)-2-oxoethyl]-2-bromo-N-[(4-chlorophenyl)methyl]-6-oxo-5,6-dihydropyrido[2,3-b]pyrazine-7-carboxamide (45a) (100.0 mg, 0.20 mmol), 3-methylimidazolidine-2,4-dione (43a) (45.7 mg, 0.40 mmol), N,N′-dimethylethylenediamine (1.8 mg, 0.02 mmol), copper(I) iodide (3.8 mg, 0.02 mmol), potassium carbonate (110.6 mg, 0.80 mmol) in dioxane (8 mL) was placed in a microwave reactor vial that was set to 160° C. for 1 h and then after cooling to ambient temperature was concentrated under reduced pressure. The resulting residue was dissolved in chloroform (50 mL) and filtered. The product was purified by column chromatography on silica gel [eluted with a gradient of 0% to 2.5% MeOH in EtOAc/MTBE (1:1)] produced 15 (40.9 mg, 37% yield) as a light yellow solid. ¹H NMR (600 MHz, DMSO-d₆) δ 9.88 (t, J=6.02 Hz, 1H), 9.56 (s, 1H), 8.69 (s, 1H), 7.44-7.38 (m, 4H), 5.15 (br s, 2H), 4.60-4.56 (m, 4H), 4.42 (br s, 2H), 3.98 (br s, 2H), 3.01 (s, 3H), 0.75-0.66 (m, 4H). LC/MS: Eluent system A (retention time: 5.89 min); ESI-MS: 550 [M+H]⁺.

### Synthesis of N-[(4-chlorophenyl)methyl]-5-[2-(3-fluoro-3-methylazetidin-1-yl)-2-oxoethyl]-2-(3-{2-[(methanesulfonyl)amino]ethyl}-2-oxoimidazolidin-1-yl)-6-oxo-5,6-dihydropyrido[2,3-b]pyrazine-7-carboxamide, (Compound 16)

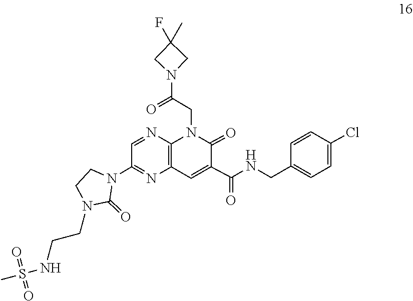 [SMILES: CC1(F)CN(C(=O)Cn2c(=O)c(C(=O)NCc3ccc(Cl)cc3)cc3nc(N4CCN(CCNS(C)(=O)=O)C4=O)cnc32)C1]

16 was synthesized as in Scheme 12.

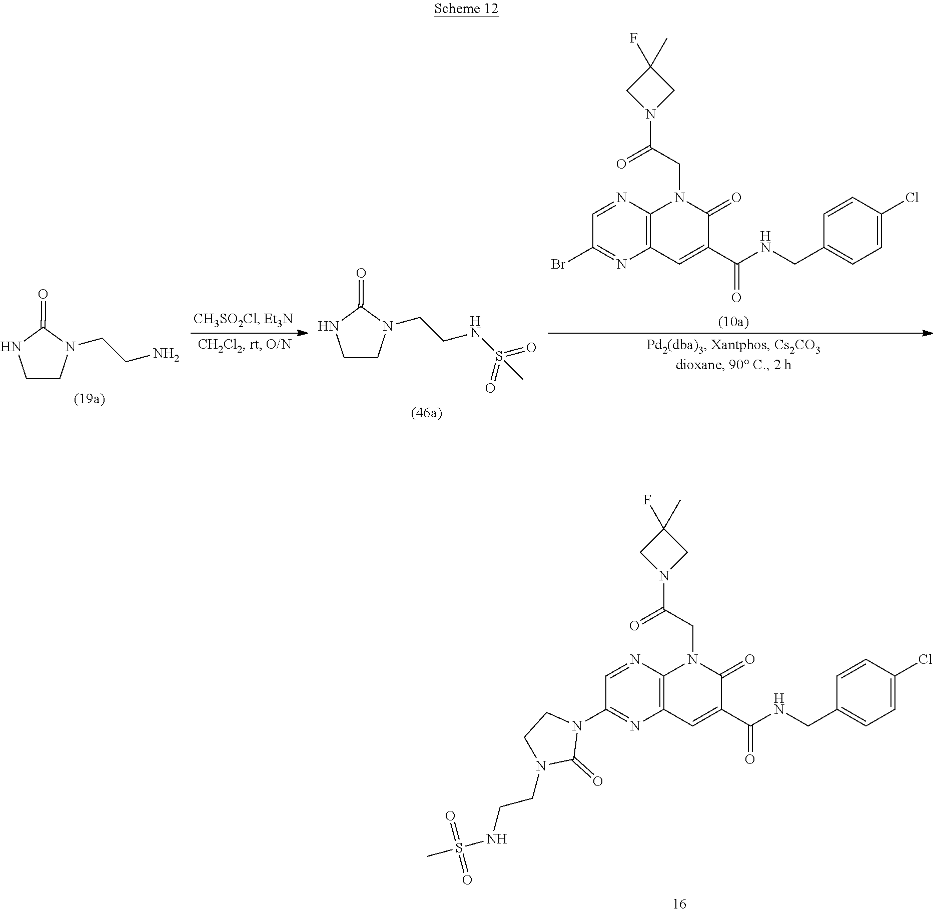 [SMILES: NCCN1CCNC1=O ; CS(=O)(=O)NCCN1CCNC1=O ; CC1(F)CN(C(=O)Cn2c(=O)c(C(=O)NCc3ccc(Cl)cc3)cc3nc(Br)cnc32)C1 ; CC1(F)CN(C(=O)Cn2c(=O)c(C(=O)NCc3ccc(Cl)cc3)cc3nc(N4CCN(CCNS(C)(=O)=O)C4=O)cnc32)C1]

Preparation of N-[2-(2-oxoimidazolidin-1-yl)ethyl]methanesulfonamide, (46a). To the solution of 1-(2-aminoethyl)imidazolidin-2-one (19a) (100 mg, 0.78 mmol) in DCM (10 mL) containing triethylamine (121.5 mg, 1.2 mmol) was added slowly methanesulfonyl chloride (107.2 mg, 0.94 mmol). After overnight at room temperature, the mixture was concentrated under reduced pressure, and the resulting residue was dissolved in a small amount of DCM and the product purified by column chromatography on silica gel (eluted with a gradient of 0% to 100% EtOAc in hexane), which provided (46a) (124.6 mg, 77% yield) as a white solid.

Preparation of N-[(4-chlorophenyl)methyl]-5-[2-(3-fluoro-3-methylazetidin-1-yl)-2-oxoethyl]-2-(3-{2-[(methanesulfonyl)amino]ethyl}-2-oxoimidazolidin-1-yl)-6-oxo-5,6-dihydropyrido[2,3-b]pyrazine-7-carboxamide, 16. A mixture of 2-bromo-N-[(4-chlorophenyl)methyl]-5-[2-(3-fluoro-3-methylazetidin-1-yl)-2-oxoethyl]-6-oxo-5,6-dihydropyrido[2,3-b]pyrazine-7-carboxamide (10a) (65.0 mg, 0.125 mmol), N-[2-(2-oxoimidazolidin-1-yl)ethyl]methanesulfonamide (46a) (51.8 mg, 0.25 mmol), Pd₂(dba)₃ (5.7 mg, 0.0063 mmol), Xantphos (10.2 mg, 0.019 mmol), cesium carbonate (48.9 mg, 0.15 mmol) in dioxane (5 mL) was heated at 90° C. for 2 h. After cooling to ambient temperature, the mixture was concentrated under reduced pressure. The product was purified by column chromatography on silica gel [eluted with a gradient of 0% to 2.5% MeOH in EtOAc] produced 16 (36.0 mg, 44% yield) as a light yellow solid. ¹H NMR (600 MHz, DMSO-d₆) δ 9.92 (t, J=6.0 Hz, 1H), 9.62 (s, 1H), 8.67 (s, 1H), 7.45-7.35 (m, 4H), 7.21 (t, J=6.2 Hz, 1H), 5.19-5.14 (m, 2H), 4.61-4.43 (m, 4H), 4.08-3.97 (m, 4H), 3.69-3.60 (m, 2H), 3.41-3.38 (m, 2H), 3.23-3.18 (m, 2H), 2.94 (s, 3H), 1.64 (d, J=22.2 Hz, 3H). ¹⁹F NMR (565 MHz, DMSO-d₆) δ−139.43-−138.57 (m, 1 F). LC/MS: Eluent system A (retention time: 5.27 min); ESI-MS: 649 [M+H]⁺.

### Synthesis of ethyl 3-[3-(7-{[(4-chlorophenyl)methyl]carbamoyl}-5-[2-(3-fluoro-3-methylazetidin-1-yl)-2-oxoethyl]-6-oxo-5,6-dihydropyrido[2,3-b]pyrazin-2-yl)-2-oxoimidazolidin-1-yl]propanoate, (Compound 17)

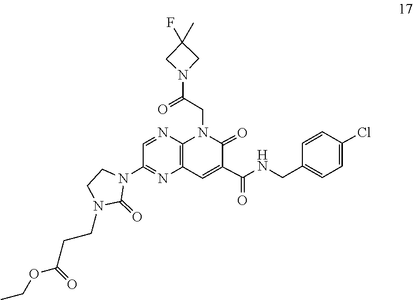 [SMILES: CCOC(=O)CCN1CCN(c2cnc3c(cc(C(=O)NCc4ccc(Cl)cc4)c(=O)n3CC(=O)N3CC(C)(F)C3)n2)C1=O]

17 was synthesized as in Scheme 13.

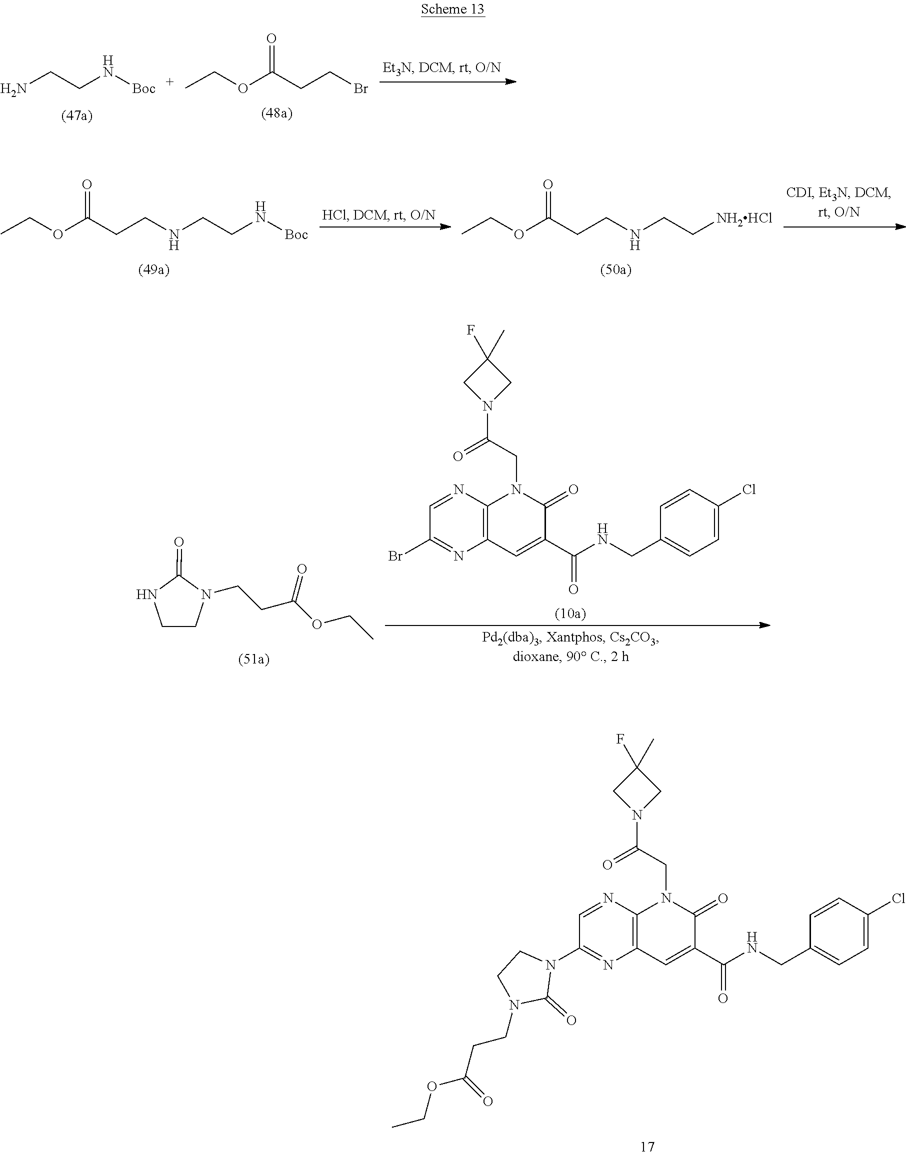

Preparation of ethyl N-{2-[(tert-butoxycarbonyl)amino]ethyl}-3-alaninate, (49a). To a solution of tert-butyl (2-aminoethyl)carbamate (47a) (3.0 g, 18.7 mmol) in DCM (50 mL) containing triethylamine (2.3 g, 22.5 mmol) was added slowly a solution of ethyl bromopropionate (48a) (3.8 g, 20.6 mmol) in DCM (25 mL) over 30 min. After overnight at room temperature, the mixture was concentrated under reduced pressure, and the residue was dissolved in ethyl acetate (100 mL) and washed with water, dried over Na₂SO₄, filtered and concentrated, which provided (49a) (2.59 g, 53% yield) as a gum.

Preparation of ethyl N-(2-aminoethyl)-β-alaninate hydrogen chloride salt, (50a). To a solution of ethyl N-{2-[(tert-butoxycarbonyl)amino]ethyl}-β-alaninate (49a) (2.5 g, 9.6 mmol) in DCM, was passed a slow stream of HCl gas for 5 min. After overnight at room temperature, the mixture was concentrated under reduced pressure. The resulting residue was suspended in ether (25 mL) and the solid collected by filtration, which provided (50a) (1.4 g, 62% yield) as a white solid.

Preparation of ethyl 3-(2-oxoimidazolidin-1-yl)propanoate, (51a). To a solution of ethyl N-(2-aminoethyl)-β-alaninate hydrogen chloride salt (50a) (0.50 g, 2.14 mmol) in DCM (25 mL) containing triethylamine (0.54 g, 5.35 mmol) was added CDI (0.35 g, 2.14 mmol) in three portions over 30 min. After overnight at room temperature, the mixture was washed with dil. HCl and water, dried over Na₂SO₄, filtered and concentrated, which provided (51a) (0.21 g, 52% yield) as a white solid.

Preparation of ethyl 3-[3-(7-{[(4-chlorophenyl)methyl]carbamoyl}-5-[2-(3-fluoro-3-methylazetidin-1-yl)-2-oxoethyl]-6-oxo-5,6-dihydropyrido[2,3-b]pyrazin-2-yl)-2-oxoimidazolidin-1-yl]propanoate, 17. A mixture of 2-bromo-N-[(4-chlorophenyl)methyl]-5-[2-(3-fluoro-3-methylazetidin-1-yl)-2-oxoethyl]-6-oxo-5,6-dihydropyrido[2,3-b]pyrazine-7-carboxamide (10) (65.0 mg, 0.13 mmol), ethyl 3-(2-oxoimidazolidin-1-yl)propanoate (51a) (46.6 mg, 0.25 mmol), Pd₂(dba)₃ (5.7 mg, 0.0063 mmol), Xantphos (10.2 mg, 0.019 mmol), cesium carbonate (48.9 mg, 0.15 mmol) in dioxane (5 mL) was heated at 90° C. for 2 h. After cooling to ambient temperature, the mixture was concentrated under reduced pressure. The product was purified by column chromatography on silica gel [eluted with a gradient of 0% to 2.5% MeOH in EtOAc] which produced 17 (75.0 mg, 95% yield) as a light yellow solid. ¹H NMR (600 MHz, DMSO-d₆) δ 9.92 (t, J=6.0 Hz, 1H), 9.60 (s, 1H), 8.66 (s, 1H), 7.43-7.37 (m, 4H), 5.19-5.09 (m, 2H), 4.60-4.43 (m, 4H), 4.09 (q, J=7.2 Hz, 2H), 4.06-3.97 (m, 4H), 3.63-3.56 (m, 2H), 3.53 (t, J=7.0 Hz, 2H), 2.65-2.60 (m, 2H), 1.64 (d, J=22.2 Hz, 3H), 1.19 (t, J=7.2 Hz, 3H). ¹⁹F NMR (565 MHz, DMSO-d₆) δ−142.00-−136.92 (m, 1F). LC/MS: Eluent system A (retention time: 6.18 min); ESI-MS: 628 [M+H]⁺.

### Synthesis of 3-[3-(7-{[(4-chlorophenyl)methyl]carbamoyl}-5-[2-(3-fluoro-3-methylazetidin-1-yl)-2-oxoethyl]-6-oxo-5,6-dihydropyrido[2,3-b]pyrazin-2-yl)-2-oxoimidazolidin-1-yl]propanoic Acid, (Compound 18)

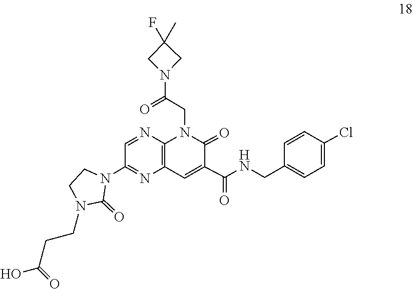 [SMILES: CC1(F)CN(C(=O)Cn2c(=O)c(C(=O)NCc3ccc(Cl)cc3)cc3nc(N4CCN(CCC(=O)O)C4=O)cnc32)C1]

Preparation of 3-[3-(7-{[(4-chlorophenyl)methyl]carbamoyl}-5-[2-(3-fluoro-3-methylazetidin-1-yl)-2-oxoethyl]-6-oxo-5,6-dihydropyrido[2,3-b]pyrazin-2-yl)-2-oxoimidazolidin-1-yl]propanoic acid, 18. To a solution of ethyl 3-[3-(7-{[(4-chlorophenyl)methyl]carbamoyl}-5-[2-(3-fluoro-3-methylazetidin-1-yl)-2-oxoethyl]-6-oxo-5,6-dihydropyrido[2,3-b]pyrazin-2-yl)-2-oxoimidazolidin-1-yl]propanoate 17 (50.0 mg, 0.08 mmol) in THF (5 mL) was added a solution of LiOH (9.6 mg, 0.40 mmol) in water (1 mL). After 4 h at room temperature, the mixture was concentrated under reduced pressure. The resulting residue was suspended in water (1 mL) and pH was adjusted to 6 with dil. HCl, which provided 18 (28.0 mg, 58% yield) as a yellow solid after filtration and drying. ¹H NMR (600 MHz, DMSO-d₆) δ 12.40 (br s, 1H), 9.92 (t, J=6.0 Hz, 1H), 9.61 (s, 1H), 8.66 (s, 1H), 7.46-7.34 (m, 4H), 5.21-5.07 (m, 2H), 4.60-4.43 (m, 4H), 4.07-3.95 (m, 4H), 3.66-3.55 (m, 2H), 3.50 (t, J=7.1 Hz, 2H), 2.59-2.53 (m, 2H), 1.63 (d, J=22.2 Hz, 3H). ¹⁹F NMR (565 MHz, DMSO-d₆) δ −140.73-−136.92 (m, 1F). LC/MS: Eluent system A (retention time: 5.53 min); ESI-MS: 600 [M+H]⁺.

### Synthesis of 2-[3-(2-acetamidoethyl)-2-oxoimidazolidin-1-yl]-N-[(4-chlorophenyl)methyl]-5-[2-(3-fluoro-3-methylazetidin-1-yl)-2-oxoethyl]-6-oxo-5,6-dihydropyrido[2,3-b]pyrazine-7-carboxamide, (Compound 19)

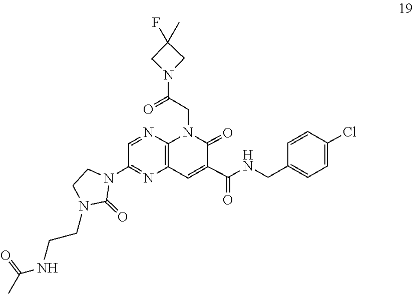 [SMILES: CC(=O)NCCN1CCN(c2cnc3c(cc(C(=O)NCc4ccc(Cl)cc4)c(=O)n3CC(=O)N3CC(C)(F)C3)n2)C1=O]

19 was synthesized as in Scheme 14.

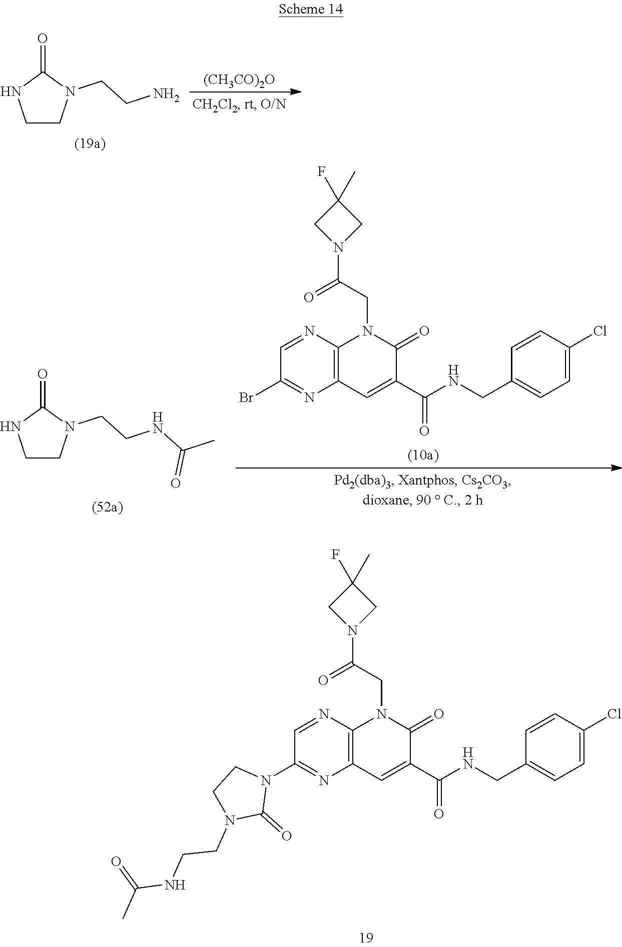 [SMILES: NCCN1CCNC1=O ; "(CH3CO)2O" ; CC(=O)NCCN1CCNC1=O ; CC1(F)CN(C(=O)Cn2c(=O)c(C(=O)NCc3ccc(Cl)cc3)cc3nc(Br)cnc32)C1 ; CC(=O)NCCN1CCN(c2cnc3c(cc(C(=O)NCc4ccc(Cl)cc4)c(=O)n3CC(=O)N3CC(C)(F)C3)n2)C1=O]

Preparation of N-[2-(2-oxoimidazolidin-1-yl)ethyl]acetamide, (52a). To a solution of 1-(2-aminoethyl)imidazolidin-2-one (19a) (100 mg, 0.78 mmol) in DCM (25 mL) was added slowly acetic anhydride (178.0 mg, 1.55 mmol). After overnight at room temperature, the mixture was concentrated under reduced pressure, which provided (52a) (132.5 mg, 99% yield) as a gum that was used in the next step without further purification.

Preparation of 2-[3-(2-acetamidoethyl)-2-oxoimidazolidin-1-yl]-N-[(4-chlorophenyl)methyl]-5-[2-(3-fluoro-3-methylazetidin-1-yl)-2-oxoethyl]-6-oxo-5,6-dihydropyrido[2,3-b]pyrazine-7-carboxamide, 19. A mixture of 2-bromo-N-[(4-chlorophenyl)methyl]-5-[2-(3-fluoro-3-methylazetidin-1-yl)-2-oxoethyl]-6-oxo-5,6-dihydropyrido[2,3-b]pyrazine-7-carboxamide (10a) (65.0 mg, 0.125 mmol), N-[2-(2-oxoimidazolidin-1-yl)ethyl]acetamide (52a) (57.3 mg, 0.25 mmol), Pd₂(dba)₃ (5.7 mg, 0.0063 mmol), Xantphos (10.2 mg, 0.019 mmol), cesium carbonate (48.9 mg, 0.15 mmol) in dioxane (10 mL) was heated at 90° C. for overnight. After cooling to ambient temperature, the mixture was concentrated under reduced pressure. The product was purified by column chromatography on silica gel [eluted with a gradient of 0% to 2.5% MeOH in EtOAc], which produced 19 (58.0 mg, 76% yield) as a light yellow solid. 1H NMR (600 MHz, DMSO-d₆) δ 9.92 (t, J=6.0 Hz, 1H), 9.61 (s, 1H), 8.66 (s, 1H), 7.97 (t, J=5.7 Hz, 1H), 7.45-7.35 (m, 4H), 5.14 (q, J=15.8 Hz, 2H), 4.60-4.43 (m, 4H), 4.07-3.96 (m, 4H), 3.68-3.58 (m, 2H), 3.36-3.24 (m, 4H), 1.79 (s, 3H), 1.64 (d, J=22.2 Hz, 3H). ¹⁹F NMR (565 MHz, DMSO-d₆) δ −139.17-−138.89 (m, 1F). LC/MS: Eluent system A (retention time: 5.89 min); ESI-MS: 613 [M+H]⁺.

### Synthesis of [3-(5-[2-(5-azaspiro[2.3]hexan-5-yl)-2-oxoethyl]-7-{[(4-chlorophenyl)methyl]carbamoyl}-6-oxo-5,6-dihydropyrido[2,3-b]pyrazin-2-yl)-2-oxoimidazolidin-1-yl]acetic Acid, (Compound 20)

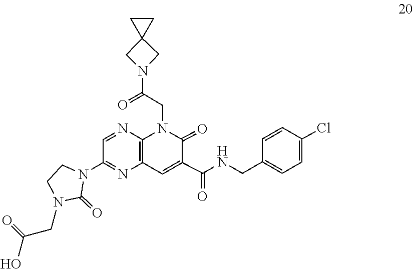 [SMILES: O=C(O)CN1CCN(c2cnc3c(cc(C(=O)NCc4ccc(Cl)cc4)c(=O)n3CC(=O)N3CC4(CC4)C3)n2)C1=O]

20 was synthesized as in Scheme 15.

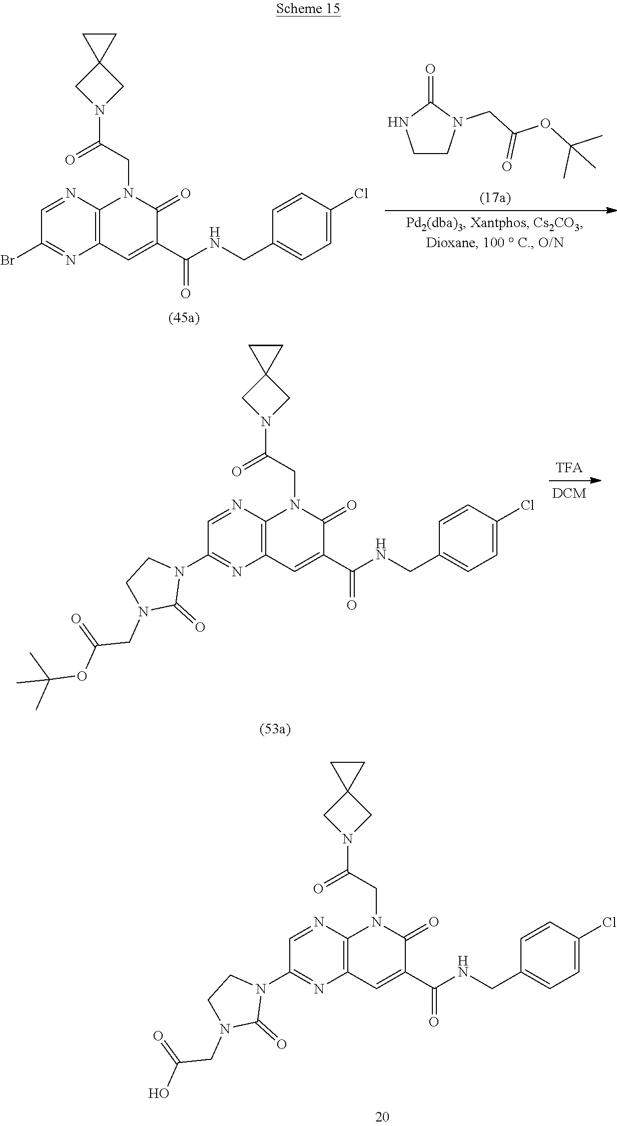 [SMILES: O=C(NCc1ccc(Cl)cc1)c1cc2nc(Br)cnc2n(CC(=O)N2CC3(CC3)C2)c1=O ; CC(C)(C)OC(=O)CN1CCNC1=O ; CC(C)(C)OC(=O)CN1CCN(c2cnc3c(cc(C(=O)NCc4ccc(Cl)cc4)c(=O)n3CC(=O)N3CC4(CC4)C3)n2)C1=O ; O=C(O)CN1CCN(c2cnc3c(cc(C(=O)NCc4ccc(Cl)cc4)c(=O)n3CC(=O)N3CC4(CC4)C3)n2)C1=O]

Preparation of tert-butyl [3-(5-[2-(5-azaspiro[2.3]hexan-5-yl)-2-oxoethyl]-7-{[(4-chlorophenyl)methyl]carbamoyl}-6-oxo-5,6-dihydropyrido[2,3-b]pyrazin-2-yl)-2-oxoimidazolidin-1-yl]acetate, (53a). To a solution of 5-[2-(5-azaspiro[2.3]hexan-5-yl)-2-oxoethyl]-2-bromo-N-[(4-chlorophenyl)methyl]-6-oxo-5,6-dihydropyrido[2,3-b]pyrazine-7-carboxamide (45a) (70 mg, 0.14 mmol) and tert-butyl 2-(2-oxoimidazolidin-1-yl)acetate (17a) (81 mg, 0.41 mmol) in dioxane (15 mL) was added Pd₂(dba)₃ (6.2 mg, 0.007 mmol), Xantphos (11.6 mg, 0.020 mmol), followed by Cs₂CO₃ (62 mg, 0.19 mmol). The reaction mixture was heated overnight with an oil bath at 100° C. After cooling to room temperature, silica gel was added and the suspension was concentrated under reduced pressure. The product was purified by column chromatography on silica gel with a gradient of 0% to 10% MeOH in EtOAc, which provided (53a) (61 mg, 71% yield) as a light yellow solid.

Preparation of [3-(5-[2-(5-azaspiro[2.3]hexan-5-yl)-2-oxoethyl]-7-{[(4-chlorophenyl)methyl]carbamoyl}-6-oxo-5,6-dihydropyrido[2,3-b]pyrazin-2-yl)-2-oxoimidazolidin-1-yl]acetic acid, 20. To a solution of (53a) (61 mg, 0.096 mmol) in DCM (3 mL) was added trifluoroacetic acid (2 mL). After 2 h at room temperature. the volatiles were removed under reduced pressure, and the residue was co-evaporated with DCM/ether (1:1, 3×20 mL). The crude was dissolved in DCM and solidified with ether. The solid was filtered under vacuum, washed with ether, and dried, which afforded 20 (37 mg, 66% yield) as a light yellow solid. ¹H NMR (600 MHz, DMSO-d₆) δ 9.93 (t, J=6.0 Hz, 1H), 9.59 (s, 1H), 8.67 (s, 1H), 7.44-7.35 (m, 4H), 5.12 (s, 2H), 4.57 (d, J=6.0 Hz, 2H), 4.39 (s, 2H), 4.09-3.99 (m, 4H), 3.96 (s, 2H), 3.69-3.63 (m, 2H), 0.73-0.62 (m, 4H). LC/MS: Eluent system A (retention time: 5.69 min); ESI-MS 580 [M+H]⁺, 578 [M−H]⁻.

### Synthesis of N-(4-chlorobenzyl)-2-(3-(2-(dimethylamino)-2-oxoethyl)-2-oxoimidazolidin-1-yl)-5-(2-(3-fluoro-3-methylazetidin-1-yl)-2-oxoethyl)-6-oxo-5,6-dihydropyrido[2,3-b]pyrazine-7-carboxamide, (Compound 21)

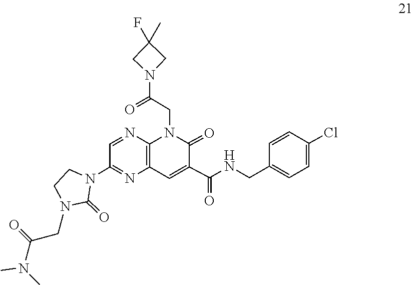 [SMILES: CN(C)C(=O)CN1CCN(c2cnc3c(cc(C(=O)NCc4ccc(Cl)cc4)c(=O)n3CC(=O)N3CC(C)(F)C3)n2)C1=O]

Preparation of N-(4-chlorobenzyl)-2-(3-(2-(dimethylamino)-2-oxoethyl)-2-oxoimidazolidin-1-yl)-5-(2-(3-fluoro-3-methylazetidin-1-yl)-2-oxoethyl)-6-oxo-5,6-dihydropyrido[2,3-b]pyrazine-7-carboxamide, 21. To a solution of [3-(7-{[(4-chlorophenyl)methyl]carbamoyl}-5-[2-(3-fluoro-3-methylazetidin-1-yl)-2-oxoethyl]-6-oxo-5,6-dihydropyrido[2,3-b]pyrazin-2-yl)-2-oxoimidazolidin-1-yl]acetic acid, 13 (80 mg, 0.14 mmol) in DMF (2 mL) was added HATU (78 mg, 0.21 mmol) at room temperature. After 30 min, a 2.0 M solution of dimethylamine in THF (0.7 mL, 1.4 mmol) was added to the reaction mixture via a syringe. After 1 h at room temperature, the solvent was removed under reduced pressure and the product was purified by column chromatography on silica gel with gradient of 0% to 10% MeOH in EtOAc, which provided 21 (6.7 mg, 85% yield) as a light yellow solid. 1H NMR (600 MHz, DMSO-d₆) δ 9.91 (t, J=6.0 Hz, 1H), 9.58 (s, 1H), 8.66 (s, 1H), 7.44-7.33 (m, 4H), 5.19-5.09 (m, 2H), 4.61-4.40 (m, 4H), 4.16 (br s, 2H), 4.08-3.97 (m, 4H), 3.63-3.59 (m, 2H), 2.98 (s, 3H), 2.85 (s, 3H), 1.63 (d, J=22.2 Hz, 3H). ¹⁹F NMR (565 MHz, DMSO-d₆) δ −138.89-−139.16 (m, 1F). LC/MS: Eluent system A (retention time: 5.45 min); ESI-MS 613 [M+H]⁺.

### Synthesis of 2-(3-{2-[acetyl(methyl)amino]ethyl}-2-oxoimidazolidin-1-yl)-N-[(4-chlorophenyl)methyl]-5-[2-(3-fluoro-3-methylazetidin-1-yl)-2-oxoethyl]-6-oxo-5,6-dihydropyrido[2,3-b]pyrazine-7-carboxamide, (Compound 22)

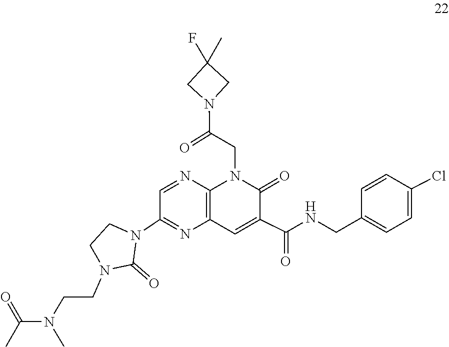 [SMILES: CC(=O)N(C)CCN1CCN(c2cnc3c(cc(C(=O)NCc4ccc(Cl)cc4)c(=O)n3CC(=O)N3CC(C)(F)C3)n2)C1=O]

22 was synthesized as in Scheme 16.

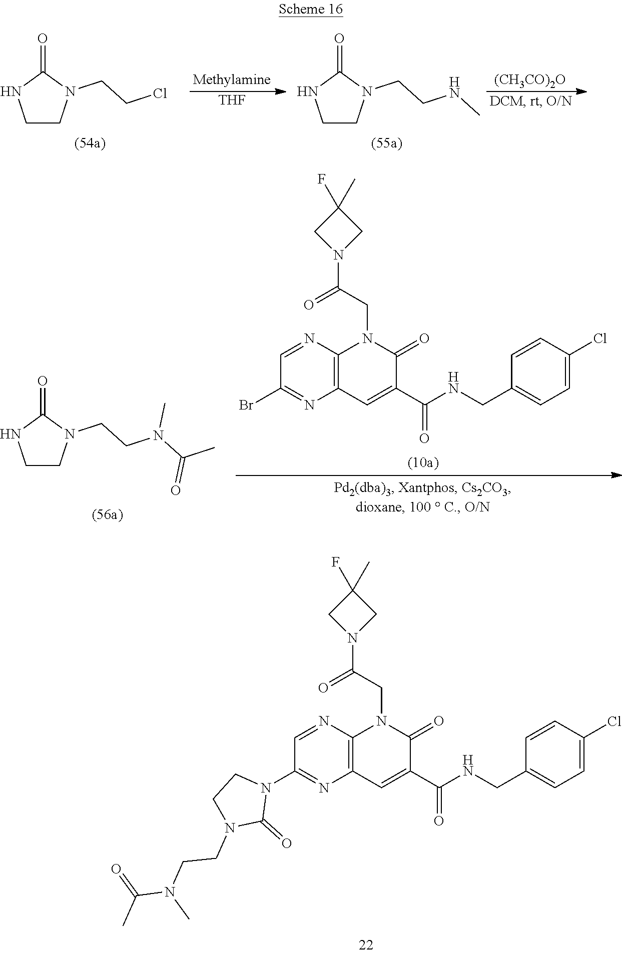

Preparation of 1-[2-(methylamino)ethyl]imidazolidin-2-one, (55a). To a sealed-tube was added 1-(2-chloroethyl)imidazolidin-2-one (54a) (350 mg, 2.4 mmol) and 2.0 M methylamine solution in THF (2 mL). The tube was then sealed with a cap and heated at 100° C. overnight. After cooling, the solid was filtered under vacuum. The filtrate was concentrated under reduced pressure and dried, which provided (55a) (310 mg, 92% yield) as a light yellow solid.

Preparation of N-methyl-N-[2-(2-oxoimidazolidin-1-yl)ethyl]acetamide, (56a). To a solution of 1-[2-(methylamino)ethyl]imidazolidin-2-one (55a) (50 mg, 0.350 mol) in DCM (10 mL) was added acetic anhydride (0.1 mL, 0.70 mmol) at room temperature. After 2 h, the solvent was removed under reduced pressure and after drying provided (56a) (65 mg, quantitative yield) as an off-white solid. This material was used in the next step without further purification.

Preparation of 2-(3-{2-[acetyl(methyl)amino]ethyl}-2-oxoimidazolidin-1-yl)-N-[(4-chlorophenyl)methyl]-5-[2-(3-fluoro-3-methylazetidin-1-yl)-2-oxoethyl]-6-oxo-5,6-dihydropyrido[2,3-b]pyrazine-7-carboxamide, 22. A similar procedure as described for compound 1 was followed: with 2-bromo-N-[(4-chlorophenyl)methyl]-5-[2-(3-fluoro-3-methylazetidin-1-yl)-2-oxoethyl]-6-oxo-5,6-dihydropyrido[2,3-b]pyrazine-7-carboxamide (10a) (70 mg, 0.13 mmol), N-methyl-N-[2-(2-oxoimidazolidin-1-yl)ethyl]acetamide (56a) (65 mg, 0.35 mmol), Pd₂(dba)₃ (6.1 mg, 0.007 mmol), Xantphos (11.6 mg, 0.02 mmol), and Cs₂CO₃ (61 mg, 0.19 mmol) in dioxane (10 mL), followed by silica gel column purification (eluted with 0% to 10% MeOH in EtOAc), which produced 22 (14 mg, 17% yield) as a light yellow solid. ¹H NMR (600 MHz, DMSO-d₆) δ 9.91 (t, J=5.8 Hz, 1H), 9.59 (s, 1H), 8.65 (s, 1H), 7.42-7.36 (m, 4H), 5.20-5.06 (m, 2H), 4.61-4.40 (m, 4H), 4.05-3.94 (m, 4H), 3.66-3.60 (m, 2H), 3.52-3.47 (m, 2H), 3.46-3.42 (m, 1H), 3.41-3.38 (m, 1H), 3.06-2.79 (m, 3H), 2.08-1.87 (m, 3H), 1.63 (d, J=22.1 Hz, 3H). LC/MS: Eluent system A (retention time: 5.61 min); ESI-MS 627 [M+H]⁺.

### Synthesis of N-(4-chlorobenzyl)-5-(2-(3-fluoro-3-methylazetidin-1-yl)-2-oxoethyl)-2-(3-(2-(methylamino)ethyl)-2-oxoimidazolidin-1-yl)-6-oxo-5,6-dihydropyrido[2,3-b]pyrazine-7-carboxamide hydrochloride Salt, (Compound 23)

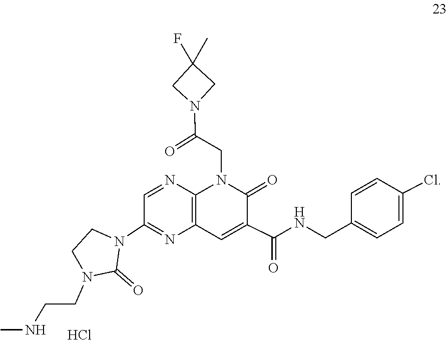 [SMILES: CNCCN1CCN(c2cnc3c(cc(C(=O)NCc4ccc(Cl)cc4)c(=O)n3CC(=O)N3CC(C)(F)C3)n2)C1=O ; Cl]

23 was synthesized as in Scheme 17.

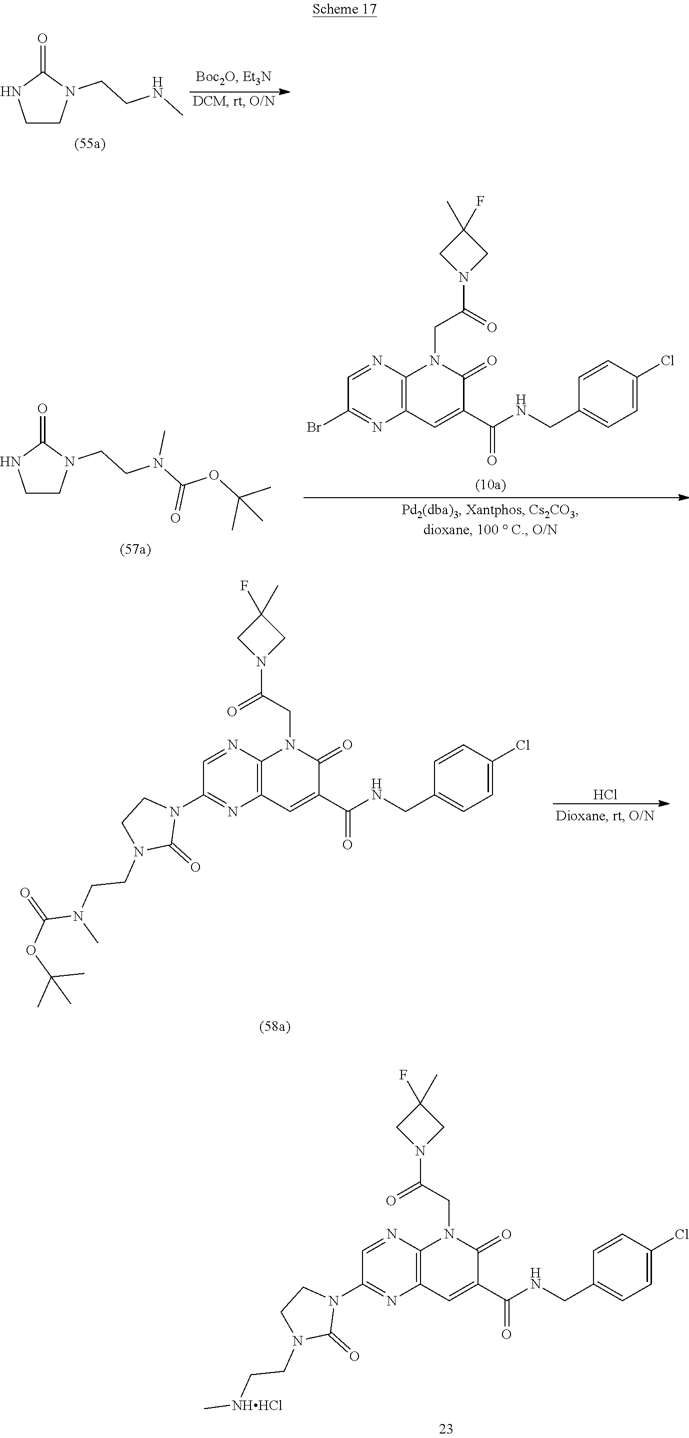

Preparation of tert-butyl methyl[2-(2-oxoimidazolidin-1-yl)ethyl]carbamate, (57a). To a solution of 1-[2-(methylamino)ethyl]imidazolidin-2-one (55a) (150 mg, 1.0 mmol) in DCM (20 mL) was added boc anhydride (0.46 mL, 2.0 mmol), followed by triethylamine (0.7 mL, 5.0 mmol) at room temperature. After stirring 2 h, the solvent was removed under reduced pressure and the product was purified by column chromotrography on silica gel with gradient of 0% to 20% MeOH in EtOAc, which provided (57a) (225 mg, 92% yield) as a white solid.

Preparation of tert-butyl {2-[3-(7-{[(4-chlorophenyl)methyl]carbamoyl}-5-[2-(3-fluoro-3-methylazetidin-1-yl)-2-oxoethyl]-6-oxo-5,6-dihydropyrido[2,3-b]pyrazin-2-yl)-2-oxoimidazolidin-1-yl]ethyl}methylcarbamate, (58a). A similar procedure as described for compound 1 was followed: with 2-bromo-N-[(4-chlorophenyl)methyl]-5-[2-(3-fluoro-3-methylazetidin-1-yl)-2-oxoethyl]-6-oxo-5,6-dihydropyrido[2,3-b]pyrazine-7-carboxamide (10a) (120 mg, 0.23 mmol), tert-butyl methyl[2-(2-oxoimidazolidin-1-yl)ethyl]carbamate (57a) (112 mg, 0.46 mmol), Pd₂(dba)₃ (10.5 mg, 0.012 mmol), Xantphos (20.0 mg, 0.035 mmol), and Cs₂CO₃ (105 mg, 0.32 mmol) in dioxane (20 mL), followed by silica gel column purification (eluted with a gradient of 0% to 5% MeOH in EtOAc) generated (58a) (131 mg, 84% yield) as a light yellow solid.

Preparation of N-(4-chlorobenzyl)-5-(2-(3-fluoro-3-methylazetidin-1-yl)-2-oxoethyl)-2-(3-(2-(methylamino)ethyl)-2-oxoimidazolidin-1-yl)-6-oxo-5,6-dihydropyrido[2,3-b]pyrazine-7-carboxamide, 23. To a solution of (58a) (50 mg, 0.073 mmol) in dioxane (20 mL) was added a 4M HCl solution in dioxane (1 mL) at room temperature. After stirring overnight, the solvent was removed under reduced pressure. The residue was co-evaporated with DCM/Ether (1:1, 3×10 mL). To the residue was added ether and solid was filtered under vacuum and dried, which afforded 23 (41 mg, 90% yield) as a light yellow solid. ¹H NMR (600 MHz, DMSO-d₆) δ 9.91 (t, J=6.0 Hz, 1H), 9.59 (s, 1H), 8.67 (s, 1H), 8.47 (br s, 2H), 7.45-7.33 (m, 4H), 5.20-5.06 (m, 2H), 4.61-4.42 (m, 4H), 4.08-3.96 (m, 4H), 3.67-3.59 (m, 2H), 3.59-3.53 (m, 2H), 3.19-3.12 (m, 2H), 2.59 (s, 3H), 1.63 (d, J=22.1 Hz, 3H). ¹⁹F NMR (565 MHz, DMSO-d₆) δ−138.86-−139.15 (m, 1F). LC/MS: Eluent system A (retention time: 4.32 min); ESI-MS 585 [M+H]⁺.

### Synthesis of N-(4-chlorobenzyl)-5-(2-(3-fluoro-3-methylazetidin-1-yl)-2-oxoethyl)-2-(3-(2-(N-methylmethylsulfonamido)ethyl)-2-oxoimidazolidin-1-yl)-6-oxo-5,6-dihydropyrido[2,3-b]pyrazine-7-carboxamide, (Compound 24)

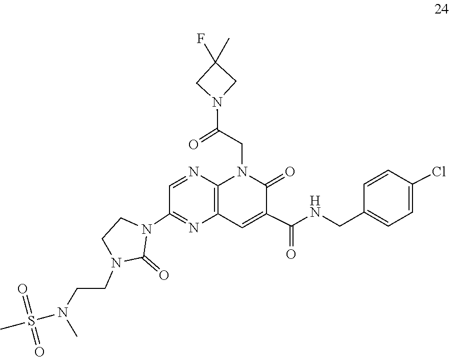 [SMILES: CN(CCN1CCN(c2cnc3c(cc(C(=O)NCc4ccc(Cl)cc4)c(=O)n3CC(=O)N3CC(C)(F)C3)n2)C1=O)S(C)(=O)=O]

Preparation of N-(4-chlorobenzyl)-5-(2-(3-fluoro-3-methylazetidin-1-yl)-2-oxoethyl)-2-(3-(2-(N-methylmethylsulfonamido)ethyl)-2-oxoimidazolidin-1-yl)-6-oxo-5,6-dihydropyrido[2,3-b]pyrazine-7-carboxamide, 24. To a solution of N-(4-chlorobenzyl)-5-(2-(3-fluoro-3-methylazetidin-1-yl)-2-oxoethyl)-2-(3-(2-(methylamino)ethyl)-2-oxoimidazolidin-1-yl)-6-oxo-5,6-dihydropyrido[2,3-b]pyrazine-7-carboxamide, 23 (45 mg, 0.072 mmol) in DCM (3 mL) was added triethylamine (49 μL, 0.350 mmol), followed by methanesulfonyl chloride (9 μL, 0.11 mmol). After stirring 1 h at room temperature, the solvent was removed under reduced pressure and the crude product was purified by column chromotrography on silica gel with a gradient of 0% to 10% MeOH in EtOAc, which provided 24 (33 mg, 69% yield) as a light yellow solid. ¹H NMR (600 MHz, DMSO-d₆) δ 9.91 (t, J=6.0 Hz, 1H), 9.60 (s, 1H), 8.65 (s, 1H), 7.43-7.35 (m, 4H), 5.18-5.08 (m, 2H), 4.58-4.42 (m, 4H), 4.06-3.96 (m, 4H), 3.67-3.62 (m, 2H), 3.49-3.45 (m, 2H), 3.29-3.25 (m, 2H), 2.89 (s, 3H), 2.82 (s, 3H), 1.63 (d, J=22.1 Hz, 3H). ¹⁹F NMR (565 MHz, DMSO-d₆) δ− 138.88-−139.16 (m, 1F). LC/MS: Eluent system A (retention time: 5.55 min); ESI-MS 663 [M+H]⁺.

### Synthesis of N-[(4-chlorophenyl)methyl]-2-{3-[2-(cyanoamino)-2-oxoethyl]-2-oxoimidazolidin-1-yl}-5-[2-(3-fluoro-3-methylazetidin-1-yl)-2-oxoethyl]-6-oxo-5,6-dihydropyrido[2,3-b]pyrazine-7-carboxamide, (Compound 26)

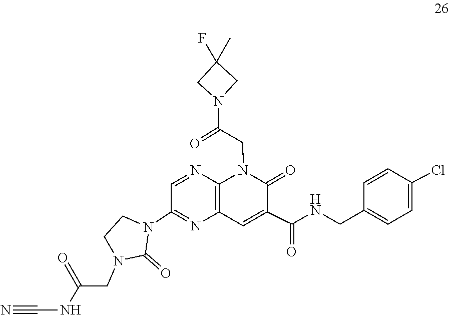 [SMILES: CC1(F)CN(C(=O)Cn2c(=O)c(C(=O)NCc3ccc(Cl)cc3)cc3nc(N4CCN(CC(=O)NC#N)C4=O)cnc32)C1]

Preparation of N-[(4-chlorophenyl)methyl]-2-{3-[2-(cyanoamino)-2-oxoethyl]-2-oxoimidazolidin-1-yl}-5-[2-(3-fluoro-3-methylazetidin-1-yl)-2-oxoethyl]-6-oxo-5,6-dihydropyrido[2,3-b]pyrazine-7-carboxamide 26. To a solution of [3-(7-{[(4-chlorophenyl)methyl]carbamoyl}-5-[2-(3-fluoro-3-methylazetidin-1-yl)-2-oxoethyl]-6-oxo-5,6-dihydropyrido[2,3-b]pyrazin-2-yl)-2-oxoimidazolidin-1-yl]acetic acid 13 (50 mg, 0.085 mmol) and cyanamide (7 mg, 0.17 mmol) in DMF (10 mL) was added HATU (65 mg, 0.17 mmol) and DIPEA (0.075 mL, 0.43 mmol). After 4 h, the volatiles were removed under reduced pressure. The resulting residue was dissolved in DCM (10 mL) and absorbed to silica gel (10 g). The resulting dried silica gel was loaded on column, and eluented with a gradient of 0 to 20% MeOH in EtOAc, which generated 26 (31 mg, 60% yield) as a yellow solid. 1H NMR (600 MHz, DMSO-d₆) δ 9.93 (t, J=6.0 Hz, 1H), 9.63 (s, 1H), 8.67 (s, 1H), 7.43-7.37 (m, 4H), 5.22-5.09 (m, 2H), 4.59-4.55 (m, 2H), 4.54-4.42 (m, 2H), 4.07-3.97 (m, 4H), 3.71-3.61 (m, 4H), 1.64 (d, J=22.2 Hz, 3H). ¹⁹F NMR (565 MHz, DMSO-d₆) δ −139.90-−139.15 (m, 1F). LC/MS: Eluent system A (retention time: 4.88 min); ESI-MS 610 [M+H]⁺.

### Synthesis of N-[(4-chlorophenyl)methyl]-5-[2-(3-fluoro-3-methylazetidin-1-yl)-2-oxoethyl]-6-oxo-2-{2-oxo-3-[(1H-tetrazol-5-yl)methyl]imidazolidin-1-yl}-5,6-dihydropyrido[2,3-b]pyrazine-7-carboxamide, (Compound 27)

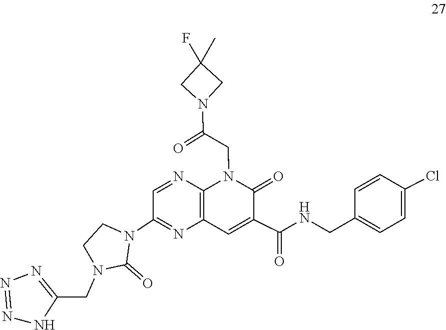 [SMILES: CC1(F)CN(C(=O)Cn2c(=O)c(C(=O)NCc3ccc(Cl)cc3)cc3nc(N4CCN(Cc5nnn[nH]5)C4=O)cnc32)C1]

27 was synthesized as in Scheme 18.

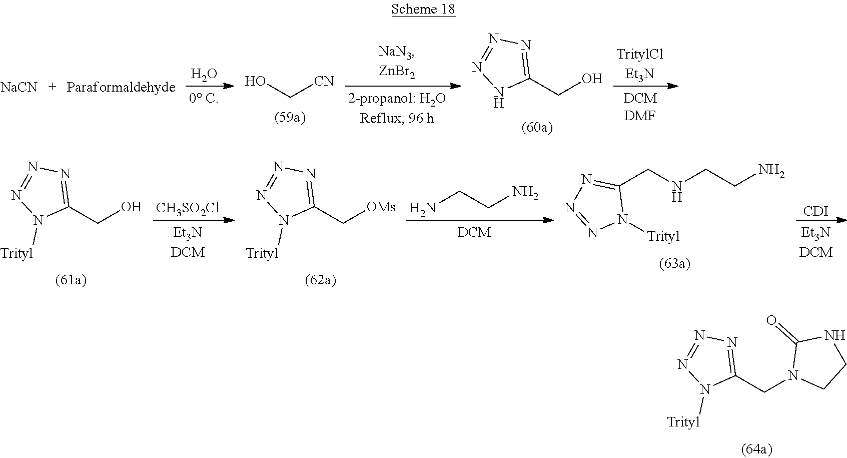

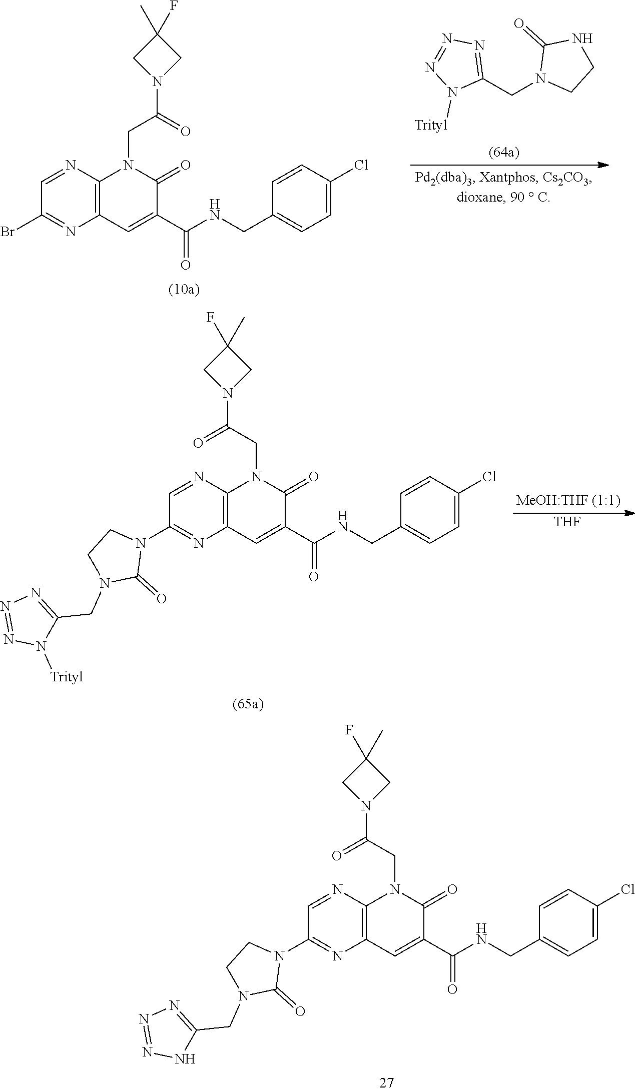

Preparation of hydroxyacetonitrile, (59a). To a solution of sodium cyanide (3.16 g, 64.49 mmol) in water (10 mL) cooled in an ice bath was added paraformaldehyde (1.26 g, 41.92 mmol). After 1 h, the pH was adjusted to 2 using conc. H₂SO₄, and the resulting mixture was extracted with Et₂O (2×100 mL). The combined organic layer was washed with water (1×50 mL), saturated brine solution (1×50 mL), dried over anhydrous Na₂SO₄, filtered, concentrated under reduced pressure, which produced (59a) as a colorless oil (1.6 g, 44% yield). This material was used in the next step without further purification.

Preparation of (1H-tetrazol-5-yl)methanol, (60a). To a solution of hydroxyacetonitrile, (59a) (1.60 g, 28.06 mmol) in 2-propanol (15 mL) and water (30 mL) were added sodium azide (3.65 g, 56.1 mmol) and zinc bromide (3.16 g, 14.0 mmol). The resulting mixture was then heated to reflux. After 96 h, the mixture was cooled to room temperature, and 30 mL of 3N HCl and 30 mL of EtOAc were added. After stirring for 10 min, the organic layer was separated and the aqueous layer was extracted with EtOAc (2×30 mL). The combined organic layer was concentrated under reduced pressure, which generated (60a) as a white solid (1.9 g. 68% yield). This material was used in the next step without further purification.

Preparation of [1-(triphenylmethyl)-1H-tetrazol-5-yl]methanol, (61a). To a solution of (60a) (1.90 g, 18.98 mmol) in DCM (20 mL) and DMF (5 mL) were added Et₃N (4.0 mL, 28.5 mmol) and trityl chloride (5.29 g, 18.98 mmol). After 1 h, the mixture was diluted with water (25 mL), and extracted with DCM (2×25 mL). The combined organic layer was washed with saturated brine solution (1×25 mL), dried over anhydrous Na₂SO₄, filtered, concentrated under reduced pressure. The resulting residue was dissolved in CHCl₃ (10 mL) and loaded on a 40 g silica gel column (Silicycle) and the product was purified by Biotage® with a gradient of 0 to 100% EtOAc in hexanes, which generated (61a) (2.55 g, 39% yield) as a white solid.

Preparation of [1-(triphenylmethyl)-1H-tetrazol-5-yl]methyl methanesulfonate, (62a). To a solution of [1-(triphenylmethyl)-1H-tetrazol-5-yl]methanol, (61a) (1.0 g, 2.9 mmol) in DCM (25 mL) was added Et₃N (2.0 mL, 14.6 mmol), and the mixture was then cooled in an ice bath and then methanesulfonyl chloride (0.25 mL, 3.2 mmol) was added dropwise over 15 min under nitrogen atmosphere. After 15 min, the ice bath removed and after 1 h at ambient temperature, ice-chilled water (25 mL) was added. After stirring for 10 min, the layers were separated and the aqueous layer was extracted with DCM (2×25 mL). The combine organic layer was washed with saturated NaHCO₃ (1×25 mL), saturated brine solution (1×25 mL), dried over anhydrous Na₂SO₄, filtered, and concentrated under reduced pressure. This generated (62a) (1.23 g) as an off-white solid. This material was used in the next step without further purification.

Preparation of N¹-{[1-(triphenylmethyl)-1H-tetrazol-5-yl]methyl}ethane-1,2-diamine, (63a). A solution of ethylenediamine (9.8 mL, 14.626 mmol) in DCM (10 mL) was cooled in an ice bath and added dropwise over 15 min to a solution of (62a) (1.23 g, 2.925 mmol) in DCM (5 mL). After 15 min, allowed to warm to room temperature. After 1 h, the mixture was concentrated under reduced pressure, co-evaporated with hexanes (2×10 mL), which produced (63a) (1.2 g) as a gummy white solid. This material was used in the next step without further purification.

Preparation of 1-{[1-(triphenylmethyl)-1H-tetrazol-5-yl]methyl}imidazolidin-2-one, (64a). To a solution of N¹-{[1-(triphenylmethyl)-1H-tetrazol-5-yl]methyl}ethane-1,2-diamine, (63a) (1.2 g, 3.12 mmol) in DCM (15 mL) was added Et₃N (0.87 mL, 6.24 mmol) followed by CDI (0.51 g, 3.12 mmol) portion wise. After overnight at room temperature, quenched the reaction mixture with water (25 mL), extracted with DCM (3×25 mL), dried over anhydrous Na₂SO₄, filtered, and concentrated under reduced pressure. The resulting residue was dissolved in CHCl₃ (5 mL) and loaded on 12 g silica gel column (Silicycle) and the product was purified by Biotage® with a gradient of 0 to 100% EtOAc in hexanes, which generated (64a) (0.3 g, 23% yield) as an off-white solid. ¹H NMR (600 MHz, DMSO-d₆) δ 7.43-7.38 (m, 9H), 7.05-7.00 (m, 6H), 6.55 (s, 1H), 4.55 (s, 2H), 3.32-3.28 (m, 2H), 3.25-3.20 (m, 2H).

Preparation of N-[(4-chlorophenyl)methyl]-5-[2-(3-fluoro-3-methylazetidin-1-yl)-2-oxoethyl]-6-oxo-2-(2-oxo-3-{[1-(triphenylmethyl)-1H-tetrazol-5-yl]methyl}imidazolidin-1-yl)-5,6-dihydropyrido[2,3-b]pyrazine-7-carboxamide, (65a). To a solution of 2-bromo-N-[(4-chlorophenyl)methyl]-5-[2-(3-fluoro-3-methylazetidin-1-yl)-2-oxoethyl]-6-oxo-5,6-dihydropyrido[2,3-b]pyrazine-7-carboxamide, (10a) (100 mg, 0.19 mmol) and 1-{[1-(triphenylmethyl)-1H-tetrazol-5-yl]methyl}imidazolidin-2-one, (64a) (157 mg, 0.38 mmol) in anhydrous 1,4-dioxane (10 mL) was added Pd₂(dba)₃ (8.8 mg, 0.0096 mmol), Xantphos (16.6 mg, 0.0287 mmol) and the resulting mixture was degassed by bubbling nitrogen for 5 min. Then, Cs₂CO₃ (125 mg, 0.38 mmol) was added and the reaction mixture was heated on oil bath at 100° C. After overnight, the oil bath was removed and after cooling to ambient temperature, the mixture was vacuum filtered, and the collected solid was washed with CHCl₃ (3×5 mL). The combined filtrates were concentrated under reduced pressure. The resulting residue was dissolved in CHCl₃ (10 mL) and loaded on 12 g silica gel column (Silicycle) and the product was purified by Biotage© with a gradient of 0 to 100% EtOAc in hexanes, which produced (65a) (122 mg, 75% yield) as a yellow solid.

Preparation of N-[(4-chlorophenyl)methyl]-5-[2-(3-fluoro-3-methylazetidin-1-yl)-2-oxoethyl]-6-oxo-2-{2-oxo-3-[(1H-tetrazol-5-yl)methyl]imidazolidin-1-yl}-5,6-dihydropyrido[2,3-b]pyrazine-7-carboxamide, 27. To a solution of N-[(4-chlorophenyl)methyl]-5-[2-(3-fluoro-3-methylazetidin-1-yl)-2-oxoethyl]-6-oxo-2-(2-oxo-3-{[1-(triphenylmethyl)-1H-tetrazol-5-yl]methyl}imidazolidin-1-yl)-5,6-dihydropyrido[2,3-b]pyrazine-7-carboxamide, (65a) (100 mg, 0.117 mmol) in MeOH:THF (1:1, 5 mL) was added trifluoroacetic acid (1 mL). After overnight, the mixture was concentrated under reduced pressure, then the residue treated with CHCl₃ (3 mL) and concentrate under reduce pressure twice. The resulting residue was dissolved in CHCl₃ (5 mL) and loaded on 12 g silica gel column (Silicycle) and the product was purified by Biotage® with a gradient of 0 to 100% EtOAc in hexanes, followed by trituration with Et₂O and hexanes, which produced 27 (52.3 mg, 73% yield) as a yellow solid. ¹H NMR (600 MHz, DMSO-d₆) δ 9.91 (t, J=6.0 Hz, 1H), 9.62 (s, 1H), 8.67 (s, 1H), 7.43-7.37 (m, 4H), 5.21-5.09 (m, 2H), 4.73 (s, 2H), 4.57 (br d, J=6.6 Hz, 2H), 4.55-4.42 (m, 2H), 4.08-3.98 (m, 4H), 3.68-3.58 (m, 2H), 1.65 (d, J=22.1 Hz, 3H). ¹⁹F NMR (565 MHz, DMSO-d₆) δ −138.89-−139.15 (m, 1F). LC/MS: Eluent system A (retention time: 5.23 min); ESI-MS: 610 [M+H]⁺.

### Synthesis of 2-{3-[2-(azetidin-1-yl)-2-oxoethyl]-2-oxoimidazolidin-1-yl}-N-[(4-chlorophenyl)methyl]-5-[2-(3-fluoro-3-methylazetidin-1-yl)-2-oxoethyl]-6-oxo-5,6-dihydropyrido[2,3-b]pyrazine-7-carboxamide, (Compound 28)

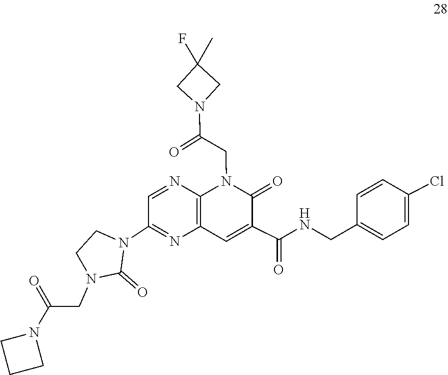 [SMILES: CC1(F)CN(C(=O)Cn2c(=O)c(C(=O)NCc3ccc(Cl)cc3)cc3nc(N4CCN(CC(=O)N5CCC5)C4=O)cnc32)C1]

Preparation of 2-{3-[2-(azetidin-1-yl)-2-oxoethyl]-2-oxoimidazolidin-1-yl}-N-[(4-chlorophenyl)methyl]-5-[2-(3-fluoro-3-methylazetidin-1-yl)-2-oxoethyl]-6-oxo-5,6-dihydropyrido[2,3-b]pyrazine-7-carboxamide, 28. To a solution of [3-(7-{[(4-chlorophenyl)methyl]carbamoyl}-5-[2-(3-fluoro-3-methylazetidin-1-yl)-2-oxoethyl]-6-oxo-5,6-dihydropyrido[2,3-b]pyrazin-2-yl)-2-oxoimidazolidin-1-yl]acetic acid 13 (50 mg, 0.085 mmol) and azetidine hydrochloride (16 mg, 0.17 mmol) in DMF (5 mL) was added HATU (65 mg, 0.17 mmol) and Et₃N (0.060 mL, 0.43 mmol). After 2 h, the mixture was concentrated under reduced pressure. The residue was dissolved in DCM (10 mL) and absorbed to silica gel (10 g). The resulting dried silica gel was loaded on column, eluted with a gradient of 0 to 15% MeOH in EtOAc, which generated 28 (36 mg, 68% yield) as a yellow solid. ¹H NMR (600 MHz, DMSO-d₆) δ 9.92 (br t, J=6.0 Hz, 1H), 9.59 (s, 1H), 8.67 (s, 1H), 7.45-7.37 (m, 4H), 5.20-5.09 (m, 2H), 4.59-4.56 (m, 2H), 4.55-4.43 (m, 2H), 4.22-4.17 (m, 2H), 4.07-3.99 (m, 4H), 3.95-3.88 (m, 4H), 3.64-3.60 (m, 2H), 2.28-2.22 (m, 2H), 1.64 (d, J=22.2 Hz, 3H). ¹⁹F NMR (565 MHz, DMSO-d₆) δ−138.93-−139.12 (m, 1F). LC/MS: Eluent system A (retention time: 5.55 min); ESI-MS 625 [M+H]⁺.

### Synthesis of 2-[3-(2-aminoethyl)-2-oxoimidazolidin-1-yl]-5-[2-(5-azaspiro[2.3]hexan-5-yl)-2-oxoethyl]-N-[(4-chlorophenyl)methyl]-6-oxo-5,6-dihydropyrido[2,3-b]pyrazine-7-carboxamide, (Compound 36)

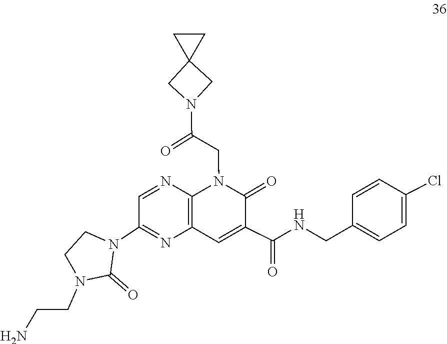 [SMILES: NCCN1CCN(c2cnc3c(cc(C(=O)NCc4ccc(Cl)cc4)c(=O)n3CC(=O)N3CC4(CC4)C3)n2)C1=O]

36 was synthesized as in Scheme 19.

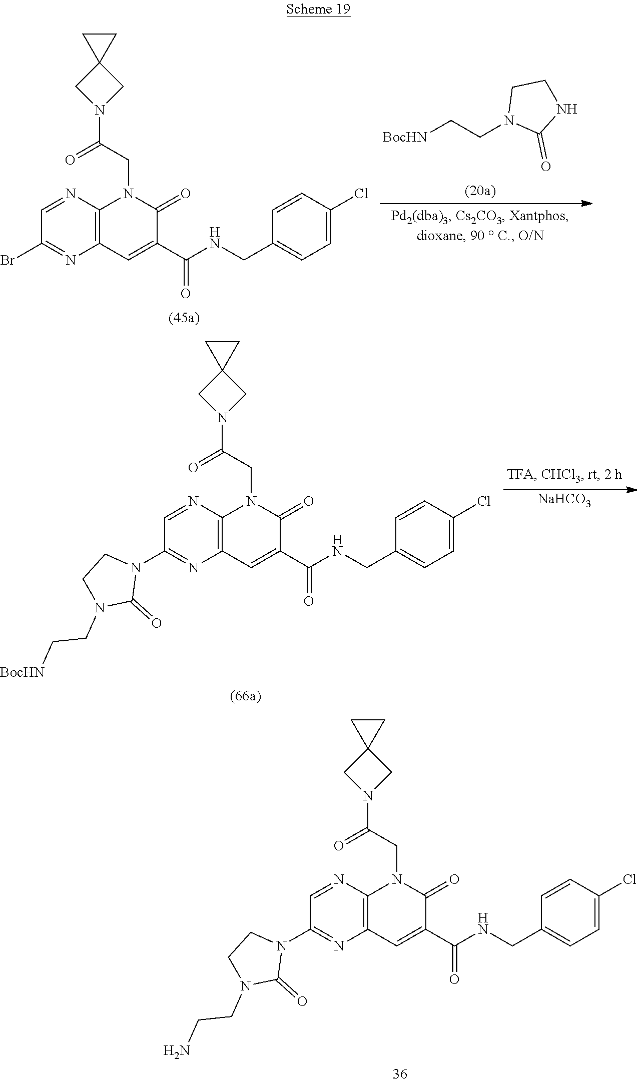

Preparation of tert-butyl {2-[3-(5-[2-(5-azaspiro[2.3]hexan-5-yl)-2-oxoethyl]-7-{[(4-chlorophenyl)methyl]carbamoyl}-6-oxo-5,6-dihydropyrido[2,3-b]pyrazin-2-yl)-2-oxoimidazolidin-1-yl]ethyl}carbamate, (66a). A mixture of 5-[2-(5-azaspiro[2.3]hexan-5-yl)-2-oxoethyl]-2-bromo-N-[(4-chlorophenyl)methyl]-6-oxo-5,6-dihydropyrido[2,3-b]pyrazine-7-carboxamide (45a) (65 mg, 0.126 mmol), tert-butyl [2-(2-oxoimidazolidin-1-yl)ethyl]carbamate (20a) (86 mg, 0.377 mmol), Pd₂(dba)₃ (7 mg, 0.008 mmol), Xantphos (15 mg, 0.026 mmol), cesium carbonate (50 mg, 0.154 mmol) and dioxane (3 mL) was heated overnight at 90° C. After cooling to ambient temperature, it was concentrated under reduced pressure. The product was purified by column chromatography on silica gel (eluted with a gradient of 0% to 5% MeOH-EtOAc), which produced (66a) (68 mg, 81% yield) as a light yellow solid.

Preparation of 2-[3-(2-aminoethyl)-2-oxoimidazolidin-1-yl]-5-[2-(5-azaspiro[2.3]hexan-5-yl)-2-oxoethyl]-N-[(4-chlorophenyl)methyl]-6-oxo-5,6-dihydropyrido[2,3-b]pyrazine-7-carboxamide, 36. To a solution of tert-butyl {2-[3-(5-[2-(5-azaspiro[2.3]hexan-5-yl)-2-oxoethyl]-7-{[(4-chlorophenyl)methyl]carbamoyl}-6-oxo-5,6-dihydropyrido[2,3-b]pyrazin-2-yl)-2-oxoimidazolidin-1-yl]ethyl}carbamate (66a) (68 mg, 0.102 mmol) in chloroform (5 mL) was added TFA (1 mL). After 2 h, the mixture was concentrated under reduced pressure. The resulting gum was dissolved in chloroform (25 mL) and concentrated under reduced pressure. The residue was suspended in ether (10 mL) and filtered. The resulting solid was mixed with saturated aqueous NaHCO₃ (0.2 mL), filtered and the collected solid washed with water (2 mL), which generated 36 (36 mg, 62% yield) as a light yellow solid. ¹H NMR (600 MHz, DMSO-d₆) δ 9.95 (t, J=6.0 Hz, 1H), 9.63 (s, 1H), 8.67 (s, 1H), 7.46-7.35 (m, 4H), 5.13 (s, 2H), 4.58 (d, J=6.0 Hz, 2H), 4.41 (br. s, 2H), 4.16-3.99 (m, 2H), 3.97 (s, 2H), 3.66-3.59 (m, 2H), 3.36-3.32 (m, 4H), 2.85 (t, J=6.2 Hz, 2H), 0.76-0.64 (m, 4H). LC/MS: Eluent system A (retention time: 4.88 min); ESI-MS: 565 [M+H]⁺.

### Synthesis of N-[(4-chlorophenyl)methyl]-5-[2-(3-fluoro-3-methylazetidin-1-yl)-2-oxoethyl]-6-oxo-2-{2-oxo-3-[2-(L-valylamino)ethyl]imidazolidin-1-yl}-5,6-dihydropyrido[2,3-b]pyrazine-7-carboxamide—hydrochloride Salt, (Compound 37)

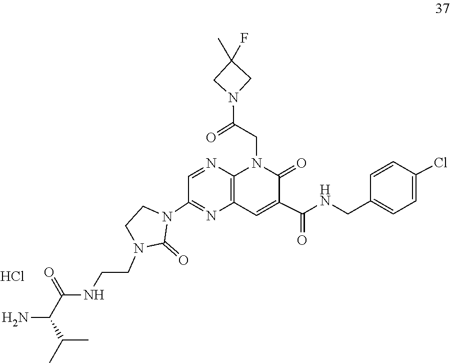 [SMILES: Cl ; CC(C)[C@H](N)C(=O)NCCN1CCN(c2cnc3c(cc(C(=O)NCc4ccc(Cl)cc4)c(=O)n3CC(=O)N3CC(C)(F)C3)n2)C1=O]

37 was synthesized as in Scheme 20.

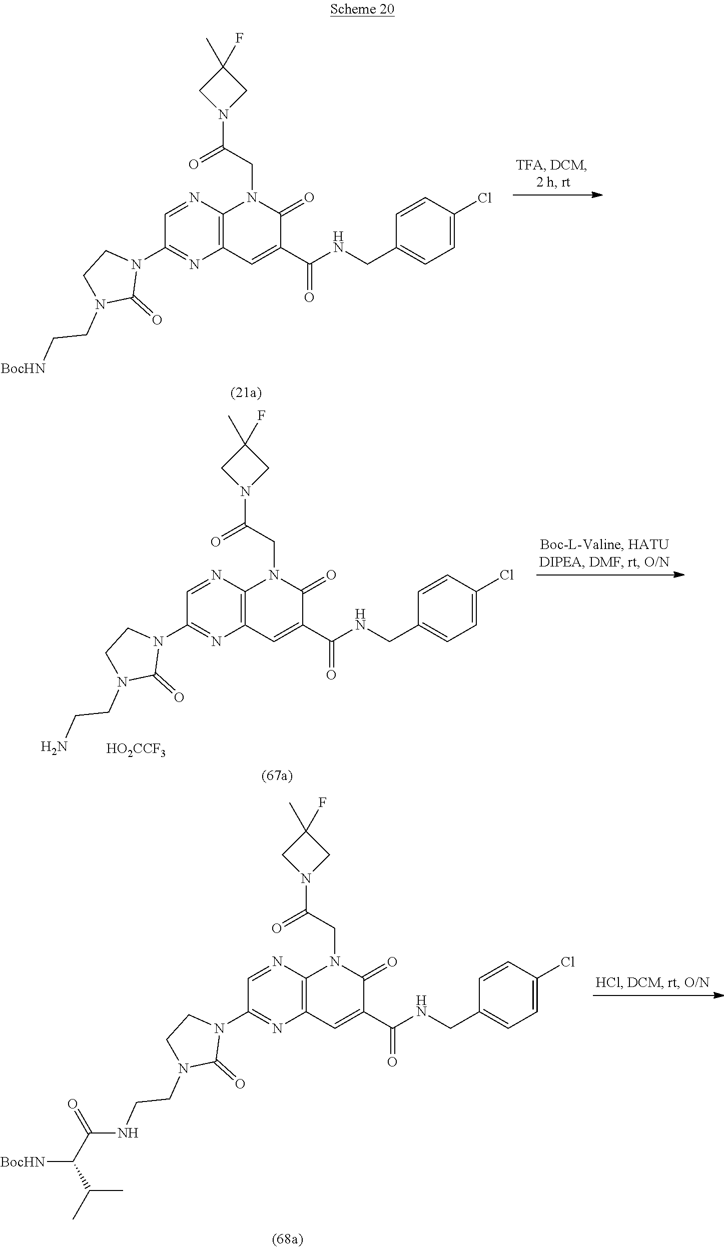

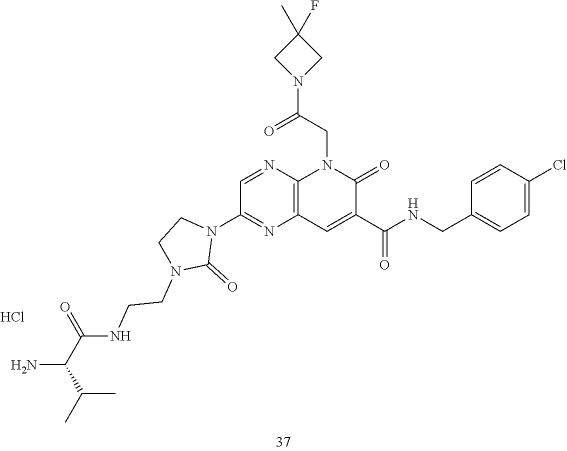 [SMILES: Cl ; CC(C)[C@H](N)C(=O)NCCN1CCN(c2cnc3c(cc(C(=O)NCc4ccc(Cl)cc4)c(=O)n3CC(=O)N3CC(C)(F)C3)n2)C1=O]

Preparation of 2-[3-(2-aminoethyl)-2-oxoimidazolidin-1-yl]-N-[(4-chlorophenyl)methyl]-5-[2-(3-fluoro-3-methylazetidin-1-yl)-2-oxoethyl]-6-oxo-5,6-dihydropyrido[2,3-b]pyrazine-7-carboxamide-TFA salt, (67a) [TFA salt of 6]. To a solution of tert-butyl {2-[3-(7-{[(4-chlorophenyl)methyl]carbamoyl}-5-[2-(3-fluoro-3-methylazetidin-1-yl)-2-oxoethyl]-6-oxo-5,6-dihydropyrido[2,3-b]pyrazin-2-yl)-2-oxoimidazolidin-1-yl]ethyl}carbamate (21a) (584 mg, 0.871 mmol) in DCM (10 mL) was added TFA (5 mL). After 2 h, the mixture was concentrated under reduced pressure. The resulting gum was suspended in ether (5 mL) and filtered, which after drying generated (67a) (390 mg, 67%) as a light yellow solid.

Preparation of tert-butyl [(2S)-1-({2-[3-(7-{[(4-chlorophenyl)methyl]carbamoyl}-5-[2-(3-fluoro-3-methylazetidin-1-yl)-2-oxoethyl]-6-oxo-5,6-dihydropyrido[2,3-b]pyrazin-2-yl)-2-oxoimidazolidin-1-yl]ethyl}amino)-3-methyl-1-oxobutan-2-yl]carbamate, (68a). To a solution of Boc-L-valine (36 mg, 0.165 mmol) and HATU (63 mg, 0.165 mmol) in DMF (3 mL) was added DIPEA (97 mg, 0.75 mmol). After 10 min, 2-[3-(2-aminoethyl)-2-oxoimidazolidin-1-yl]-N-[(4-chlorophenyl)methyl]-5-[2-(3-fluoro-3-methylazetidin-1-yl)-2-oxoethyl]-6-oxo-5,6-dihydropyrido[2,3-b]pyrazine-7-carboxamide-TFA salt (67a) (100 mg, 0.146 mmol) was added. After overnight, the mixture was concentrated under reduced pressure. The product was purified by column chromatography on silica (eluted with a gradient of 0% to 5% MeOH in CHCl₃) producing (68a) (100 mg, 63% yield) as a gum.

Preparation of N-[(4-chlorophenyl)methyl]-5-[2-(3-fluoro-3-methylazetidin-1-yl)-2-oxoethyl]-6-oxo-2-{2-oxo-3-[2-(L-valylamino)ethyl]imidazolidin-1-yl}-5,6-dihydropyrido[2,3-b]pyrazine-7-carboxamide—hydrochloride salt, 37. To the solution of tert-butyl [(2S)-1-({2-[3-(7-{[(4-chlorophenyl)methyl]carbamoyl}-5-[2-(3-fluoro-3-methylazetidin-1-yl)-2-oxoethyl]-6-oxo-5,6-dihydropyrido[2,3-b]pyrazin-2-yl)-2-oxoimidazolidin-1-yl]ethyl}amino)-3-methyl-1-oxobutan-2-yl]carbamate (68a) (100 mg, 0.13 mmol) in DCM (10 mL) was added HCl (1 mL, 4.0 mmol; 4M solution in dioxane).

After overnight, the mixture was concentrated under reduced pressure. The residue was suspended in ether (10 mL), filtered, washed with saturated NaHCO₃ (0.5 mL, aqueous) and water (3×1 mL) and dried under vacuum, generating 37 (58 mg, 66% yield) as yellow solid. ¹H NMR (600 MHz, DMSO-d₆) δ 9.90 (t, J=6.0 Hz, 1H), 9.58 (s, 1H), 8.65 (s, 1H), 8.61-8.54 (m, 1H), 8.08 (br s, 3H), 7.44-7.32 (m, 4H), 5.18-5.07 (m, 2H), 4.55 (d, J=6.8 Hz, 2H), 4.53-4.41 (m, 2H), 4.06-3.92 (m, 4H), 3.68-3.59 (m, 2H), 3.57-3.49 (m, 2H), 3.46-3.35 (m, 1H), 3.35-3.32 (m, 1H), 3.25-3.22 (m, 1H), 2.09-1.98 (m, 1H), 1.62 (d, J=21.8 Hz, 3H), 0.87 (d, J=6.8 Hz, 3H), 0.83 (d, J=6.8 Hz, 3H). ¹⁹F NMR (565 MHz, DMSO-d₆) δ−137.84-−130.42 (m, 1F). LC/MS: Eluent system A (retention time: 4.84 min); ESI-MS: 670 [M+H]⁺.

### Synthesis of N-[(4-chlorophenyl)methyl]-5-[2-(3-fluoro-3-methylazetidin-1-yl)-2-oxoethyl]-2-{3-[2-(glycylamino)ethyl]-2-oxoimidazolidin-1-yl}-6-oxo-5,6-dihydropyrido[2,3-b]pyrazine-7-carboxamide—hydrochloride Salt, (Compound 38)

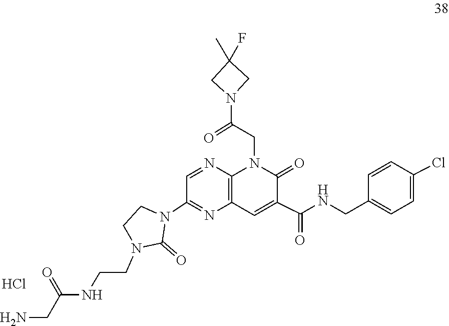 [SMILES: Cl ; CC1(F)CN(C(=O)Cn2c(=O)c(C(=O)NCc3ccc(Cl)cc3)cc3nc(N4CCN(CCNC(=O)CN)C4=O)cnc32)C1]

38 was synthesized as in Scheme 21.

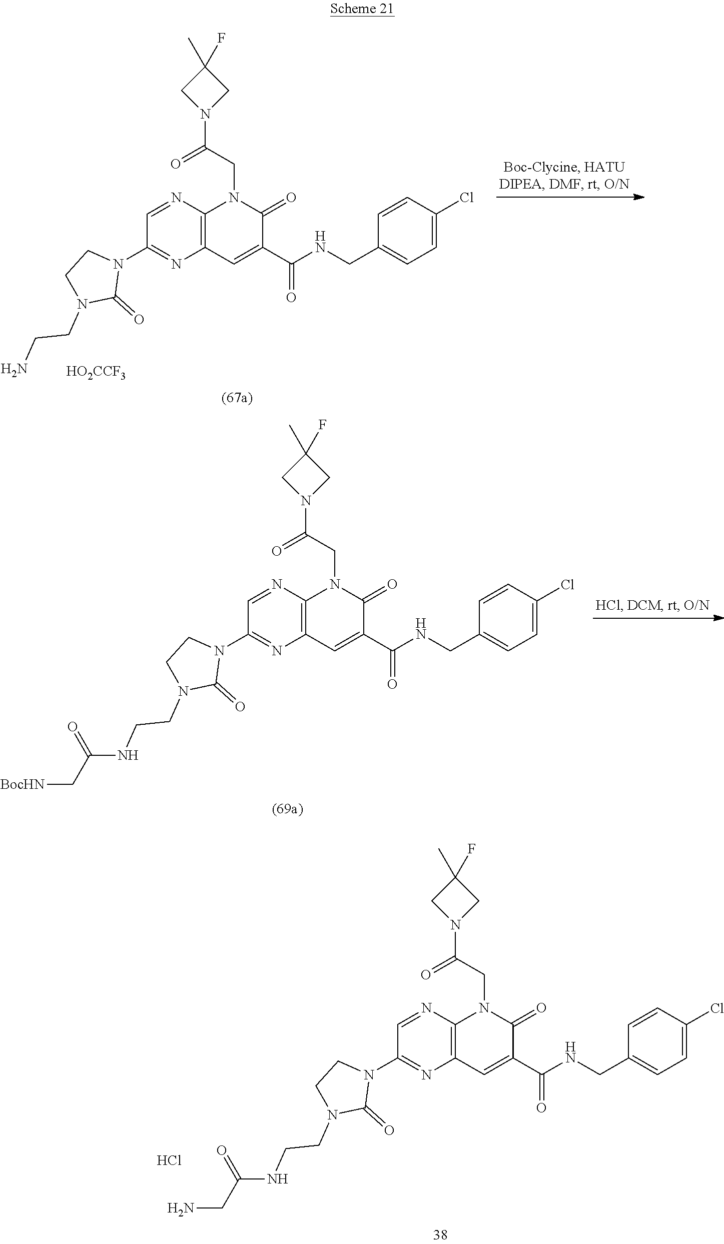

Preparation of tert-butyl [2-({2-[3-(7-{[(4-chlorophenyl)methyl]carbamoyl}-5-[2-(3-fluoro-3-methylazetidin-1-yl)-2-oxoethyl]-6-oxo-5,6-dihydropyrido[2,3-b]pyrazin-2-yl)-2-oxoimidazolidin-1-yl]ethyl}amino)-2-oxoethyl]carbamate, (69a). To a solution of Boc-glycine (47 mg, 0.199 mmol) and HATU (76 mg, 0.199 mmol) in DMF (3 mL) was added DIPEA (129 mg, 0.99 mmol). After 10 min, 2-[3-(2-aminoethyl)-2-oxoimidazolidin-1-yl]-N-[(4-chlorophenyl)methyl]-5-[2-(3-fluoro-3-methylazetidin-1-yl)-2-oxoethyl]-6-oxo-5,6-dihydropyrido[2,3-b]pyrazine-7-carboxamide-TFA salt (67a) (100 mg, 0.146 mmol) was added. After overnight, the mixture was concentrated under reduced pressure. The product was purified by column chromatography on silica (eluted with a gradient of 0% to 5% MeOH—CHCl₃) producing (69a) (76 mg, 71% yield) as a gum.

Preparation of N-[(4-chlorophenyl)methyl]-5-[2-(3-fluoro-3-methylazetidin-1-yl)-2-oxoethyl]-2-{3-[2-(glycylamino)ethyl]-2-oxoimidazolidin-1-yl}-6-oxo-5,6-dihydropyrido[2,3-b]pyrazine-7-carboxamide—hydrochloride salt, 38. To a solution of tert-butyl [2-({2-[3-(7-{[(4-chlorophenyl)methyl]carbamoyl}-5-[2-(3-fluoro-3-methylazetidin-1-yl)-2-oxoethyl]-6-oxo-5,6-dihydropyrido[2,3-b]pyrazin-2-yl)-2-oxoimidazolidin-1-yl]ethyl}amino)-2-oxoethyl]carbamate (69a) (76 mg, 0.11 mmol) in DCM (10 mL) was added HCl (0.5 mL, 2.0 mmol, 4M solution in dioxane). After stirring overnight, the mixture was concentrated under reduced pressure. The residue was suspended in ether (10 mL), filtered, washed with saturated NaHCO₃ (0.5 mL, aqueous) and water (3×1 mL) and dried under vacuum generating 38 (60 mg, 86% yield) as yellow solid. ¹H NMR (600 MHz, DMSO-d₆) δ 9.91 (t, J=6.2 Hz, 1H), 9.59 (s, 1H), 8.65 (s, 1H), 8.47 (br s, 1H), 8.00 (br.s, 3H), 7.45-7.32 (m, 4H), 5.20-5.02 (m, 2H), 4.55 (d, J=6.8 Hz, 2H), 4.54-4.41 (m, 2H), 4.07-3.93 (m, 4H), 3.62 (t, J=7.9 Hz, 2H), 3.49 (s, 2H), 3.38-3.30 (m, 4H), 1.63 (d, J=21.8 Hz, 3H). ¹⁹F NMR (565 MHz, DMSO-d₆) δ 134.46-−134.04 (m, 1F). LC/MS: Eluent system A (retention time: 4.25 min); ESI-MS: 628 [M+H]⁺.

### Synthesis of N-[(4-chlorophenyl)methyl]-5-[2-(3-fluoro-3-methylazetidin-1-yl)-2-oxoethyl]-2-{3-[2-(morpholin-4-yl)ethyl]-2-oxoimidazolidin-1-yl}-6-oxo-5,6-dihydropyrido[2,3-b]pyrazine-7-carboxamide, (Compound 39)

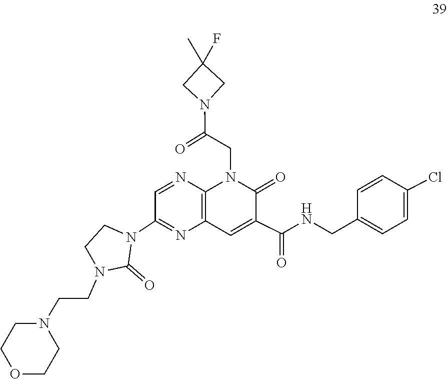 [SMILES: CC1(F)CN(C(=O)Cn2c(=O)c(C(=O)NCc3ccc(Cl)cc3)cc3nc(N4CCN(CCN5CCOCC5)C4=O)cnc32)C1]

39 was synthesized as in Scheme 22.

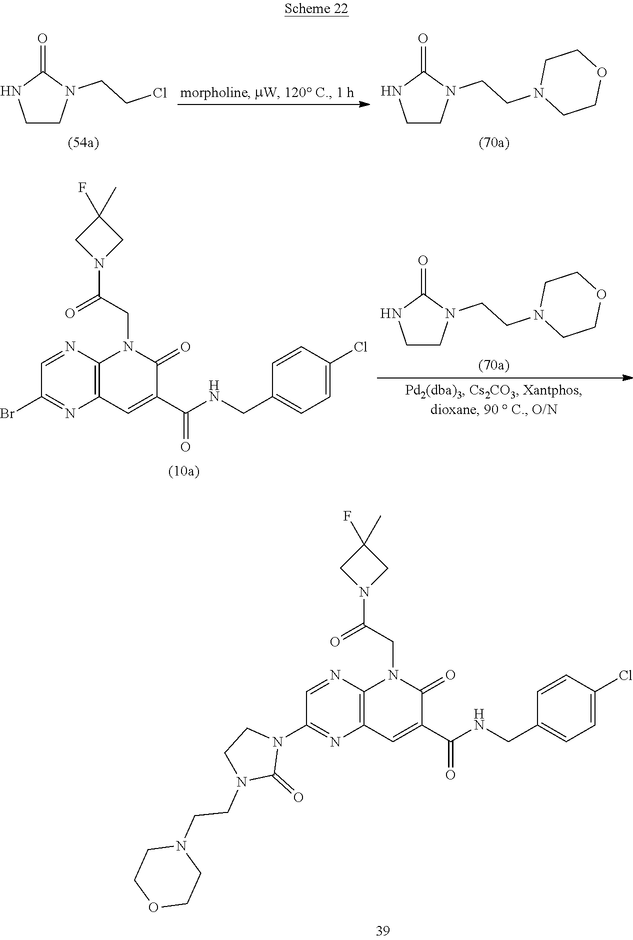 [SMILES: O=C1NCCN1CCCl ; O=C1NCCN1CCN1CCOCC1 ; CC1(F)CN(C(=O)Cn2c(=O)c(C(=O)NCc3ccc(Cl)cc3)cc3nc(Br)cnc32)C1 ; O=C1NCCN1CCN1CCOCC1 ; CC1(F)CN(C(=O)Cn2c(=O)c(C(=O)NCc3ccc(Cl)cc3)cc3nc(N4CCN(CCN5CCOCC5)C4=O)cnc32)C1]

Preparation of 1-[2-(morpholin-4-yl)ethyl]imidazolidin-2-one, (70a). A mixture of 1-(2-chloroethyl)imidazolidin-2-one (54a) (100 mg, 0.68 mmol) and morpholine (1 mL) in a sealed tube was irradiated in a microwave reactor for 1 h at 120° C. After cooling to ambient temperature, the mixture was concentrated under reduced pressure, which generated 1-[2-(morpholin-4-yl)ethyl]imidazolidin-2-one (70a) (158 mg) as a colorless, viscous liquid. This material was used in the next step without further purification.

Preparation of N-[(4-chlorophenyl)methyl]-5-[2-(3-fluoro-3-methylazetidin-1-yl)-2-oxoethyl]-2-{3-[2-(morpholin-4-yl)ethyl]-2-oxoimidazolidin-1-yl}-6-oxo-5,6-dihydropyrido[2,3-b]pyrazine-7-carboxamide, 39. A mixture of 2-bromo-N-[(4-chlorophenyl)methyl]-5-[2-(3-fluoro-3-methylazetidin-1-yl)-2-oxoethyl]-6-oxo-5,6-dihydropyrido[2,3-b]pyrazine-7-carboxamide (10a) (68 mg, 0.129 mmol), 1-[2-(morpholin-4-yl)ethyl]imidazolidin-2-one (70a) (129 mg, 0.65 mmol), Pd₂(dba)₃ (6 mg, 0.007 mmol), Xantphos (11 mg, 0.019 mmol), cesium carbonate (63 mg, 0.195 mmol) in dioxane (5 mL) was heated at 90° C. After overnight the mixture was cooled to ambient temperature and was concentrated under reduced pressure. The product was purified by column chromatography on silica gel (eluted with a gradient of 0% to 5% MeOH in CHCl₃), which produced 39 (57 mg, 69% yield) as a light yellow solid. ¹H NMR (600 MHz, DMSO-d₆) δ 9.92 (t, J=6.0 Hz, 1H), 9.61 (s, 1H), 8.66 (s, 1H), 7.43-7.37 (m, 4H), 5.22-5.03 (m, 2H), 4.57 (d, J=7.1 Hz, 2H), 4.55-4.43 (m, 2H), 4.08-3.95 (m, 4H), 3.72-3.61 (m, 2H), 3.61-3.56 (m, 4H), 3.41 (t, J=6.4 Hz, 2H), 2.52-2.48 (m, 2H), 2.44-2.41 (m, 4H), 1.64 (d, J=21.8 Hz, 3H). ¹⁹F NMR (565 MHz, DMSO-d₆) δ −139.12-138.93 (m, 1F). LC/MS: Eluent system A (retention time: 4.13 min); ESI-MS: 641 [M+H]⁺.

### Synthesis of 2-[3-(2-amino-2-oxoethyl)-2-oxoimidazolidin-1-yl]-N-[(4-chlorophenyl)methyl]-5-[2-(3-fluoro-3-methylazetidin-1-yl)-2-oxoethyl]-6-oxo-5,6-dihydropyrido[2,3-b]pyrazine-7-carboxamide, (Compound 40)

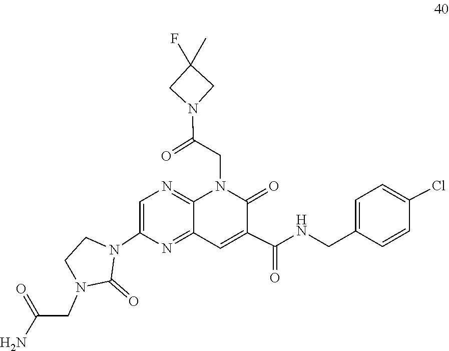 [SMILES: CC1(F)CN(C(=O)Cn2c(=O)c(C(=O)NCc3ccc(Cl)cc3)cc3nc(N4CCN(CC(N)=O)C4=O)cnc32)C1]

Preparation of 2-[3-(2-amino-2-oxoethyl)-2-oxoimidazolidin-1-yl]-N-[(4-chlorophenyl)methyl]-5-[2-(3-fluoro-3-methylazetidin-1-yl)-2-oxoethyl]-6-oxo-5,6-dihydropyrido[2,3-b]pyrazine-7-carboxamide, 40. To a solution of [3-(7-{[(4-chlorophenyl)methyl]carbamoyl}-5-[2-(3-fluoro-3-methylazetidin-1-yl)-2-oxoethyl]-6-oxo-5,6-dihydropyrido[2,3-b]pyrazin-2-yl)-2-oxoimidazolidin-1-yl]acetic acid, 13 (150 mg, 0.256 mmol) in DMF (5 mL) in a sealable tube, was added HATU (136.3 mg, 0.358 mmol) followed by 0.4 M ammonia in THF (3 mL, 1.20 mmol) and the tube was sealed. After overnight, saturated brine solution (15 mL) was added and the mixture was extracted with CHCl₃ (3×15 mL). The combine organic layer was washed with saturated NaHCO₃ (1×15 mL), saturated brine solution (1×15 mL), dried over anhydrous Na₂SO₄, filtered, and concentrated under reduced pressure. The residue was dissolved in CHCl₃ (10 mL) and loaded on 12 g silica gel column (Silicycle) and the product was purified by Biotage© with a gradient of 0 to 6% MeOH in CHCl₃, followed by trituration with Et₂O and hexanes and drying, produced 40 (125.2 mg, 84% yield) as a yellow solid. ¹H NMR (600 MHz, DMSO-d₆) δ 9.92 (t, J=6.0 Hz, 1H), 9.61 (s, 1H), 8.67 (s, 1H), 7.56 (br.s, 1H), 7.44-7.36 (m, 4H), 7.20 (br.s, 1H), 5.21-5.09 (m, 2H), 4.57 (br. d, J=6.9 Hz, 2H), 4.55-4.40 (m, 2H), 4.09-3.97 (m, 4H), 3.85 (s, 2H), 3.67-3.60 (m, 2H), 1.65 (d, J=22.1 Hz, 3H). ¹⁹F NMR (565 MHz, DMSO-d₆) δ −138.88-−139.19 (m, 1F). LC/MS: Eluent system A (retention time: 5.05 min); ESI-MS: 585 [M+H]⁺.

### Synthesis of N-[(4-chlorophenyl)methyl]-2-(2,4-dioxoimidazolidin-1-yl)-5-[2-(3-fluoro-3-methylazetidin-1-yl)-2-oxoethyl]-6-oxo-5,6-dihydropyrido[2,3-b]pyrazine-7-carboxamide, (Compound 41)

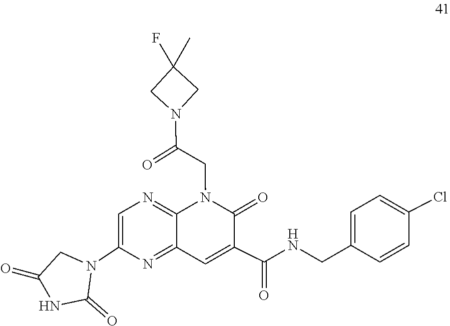 [SMILES: CC1(F)CN(C(=O)Cn2c(=O)c(C(=O)NCc3ccc(Cl)cc3)cc3nc(N4CC(=O)NC4=O)cnc32)C1]

41 was synthesized as in Scheme 23.

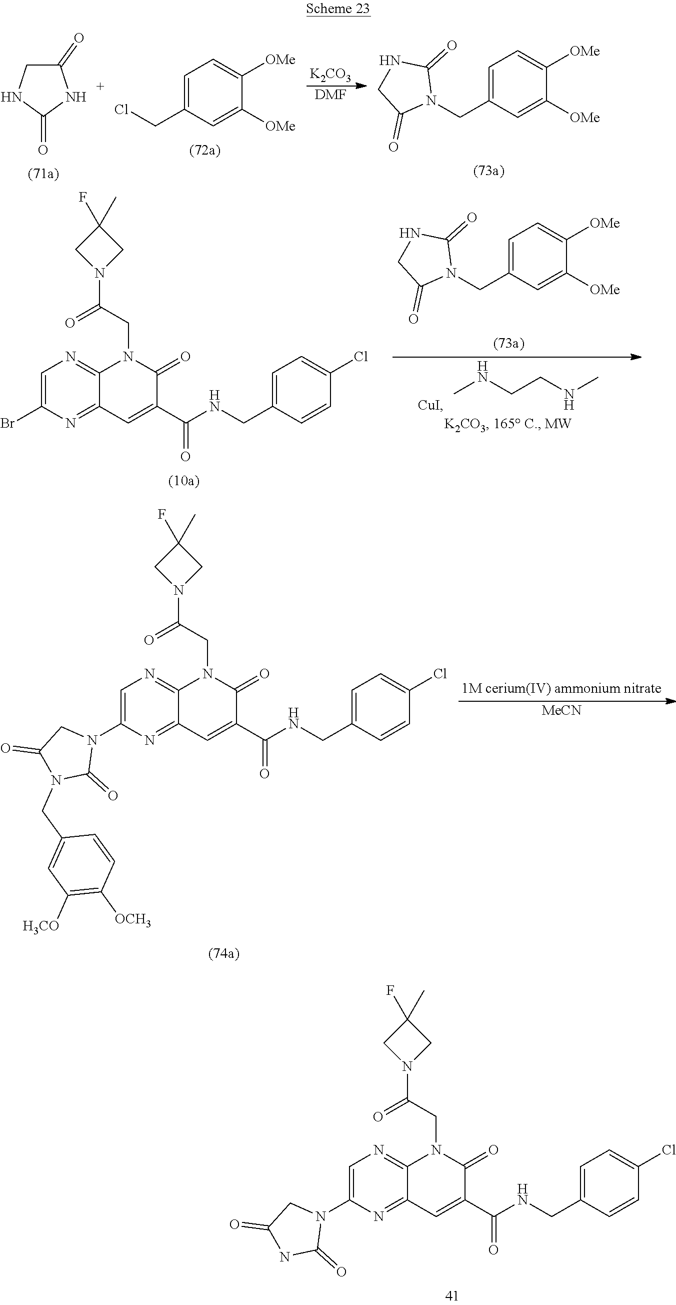

Preparation of 3-[(3,4-dimethoxyphenyl)methyl]imidazolidine-2,4-dione, (73a). A mixture of hydantoin (71a) (300 mg, 3.00 mmol), 3,4-dimethoxybenzyl chloride (72a) (560 mg, 3.00 mmol), and K₂CO₃ (1.24 g, 8.993 mmol) in DMF (10 mL) was stirred at room temperature. After overnight, water (25 mL) was added and the mixture was extracted with CHCl₃ (3×25 mL). The combined organic layer was washed with saturated brine solution (1×25 mL), dried over anhydrous Na₂SO₄, filtered, and concentrated under reduced pressure. The residue was dissolved in CHCl₃ (10 mL) and loaded on 12 g silica gel column (Silicycle) and the product was purified by Biotage© with a gradient of 0 to 100% EtOAC in hexanes, which produced (73a) (380 mg, 51% yield) as an off-white solid. ¹H NMR (600 MHz, DMSO-d₆) δ 8.09 (s, 1H), 6.90-6.88 (m, 2H), 6.78 (dd, J=2.0, 8.2 Hz, 1H), 4.45 (s, 2H), 3.96 (d, J=0.9 Hz, 2H), 3.72 (s, 6H).

Preparation of N-[(4-chlorophenyl)methyl]-2-{3-[(3,4-dimethoxyphenyl)methyl]-2,4-dioxoimidazolidin-1-yl}-5-[2-(3-fluoro-3-methylazetidin-1-yl)-2-oxoethyl]-6-oxo-5,6-dihydropyrido[2,3-b]pyrazine-7-carboxamide, (74a). A mixture of 2-bromo-N-[(4-chlorophenyl)methyl]-5-[2-(3-fluoro-3-methylazetidin-1-yl)-2-oxoethyl]-6-oxo-5,6-dihydropyrido[2,3-b]pyrazine-7-carboxamide (10a) (100 mg, 0.19 mmol), 3-[(3,4-dimethoxyphenyl)methyl]imidazolidine-2,4-dione, (73a) (96 mg, 0.38 mmol), N,N′-dimethylethylenediamine (1.7 mg, 0.019 mmol), copper(I) iodide (3.64 mg, 0.019 mmol), potassium carbonate (79.3 mg, 0.574 mmol) in 1,4-dioxane (5 mL) was placed in a microwave reactor vial that heated in a microwave to 165° C. for 1 h. After cooling to ambient temperature, the mixture was concentrated under reduced pressure. The resulting residue was dissolved in CHCl₃ (10 mL) and filtered, the collected solid was washed with CHCl₃ (3×3 mL). The filtrates were combined and concentrated under reduced pressure. The residue was dissolved in CHCl₃ (10 mL) and loaded on 25 g silica gel column (Silicycle) and the product was purified by Biotage® with a gradient of 0 to 100% EtOAc in hexanes, which generated (74a) (130 mg, 98% yield) as a yellow solid.

Preparation of N-[(4-chlorophenyl)methyl]-2-(2,4-dioxoimidazolidin-1-yl)-5-[2-(3-fluoro-3-methylazetidin-1-yl)-2-oxoethyl]-6-oxo-5,6-dihydropyrido[2,3-b]pyrazine-7-carboxamide, 41. To a solution of (74a) (60 mg, 0.087 mmol) in acetonitrile (5 mL) was added 1M cerium(IV) ammonium nitrate (CAN) (172.8 μL, 0.173 mmol). After 18 h, another portion of 1 M cerium(IV) ammonium nitrate (0.26 mL, 0.26 mmol) was added and stirred additional 72 h at room temperature. To the mixture was added saturated NaHCO₃ (10 mL) and the mixture was extracted with EtOAc (3×10 mL). The combined organic layer was washed with saturated brine solution (1×10 mL), dried over anhydrous Na₂SO₄, filtered, and concentrated under reduced pressure. The residue was dissolved in CHCl₃ (10 mL) and loaded on 25 g silica gel column (Silicycle) and the product was purified by Biotage® with a gradient of 0 to 2% MeOH in CHCl₃, followed by trituration with Et₂O and hexanes that after drying produced 41 (9.3 mg, 20% yield) as a yellow solid. 1H NMR (600 MHz, DMSO-d₆) δ 11.62 (br s, 1H), 9.86 (t, J=6.0 Hz, 1H), 9.52 (s, 1H), 8.67 (s, 1H), 7.43-7.37 (m, 4H), 5.19-5.10 (m, 2H), 4.57 (br d, J=6.4 Hz, 2H), 4.55-4.43 (m, 4H), 4.07-3.97 (m, 2H), 1.66 (d, J=22.1 Hz, 3H). ¹⁹F NMR (565 MHz, DMSO-d₆) δ −138.89-−139.16 (m, 1F). LC/MS: Eluent system A (retention time: 5.19 min); ESI-MS: 542 [M+H]⁺.

### Synthesis of N-[(4-chlorophenyl)methyl]-2-{3-[2-(dimethylamino)ethyl]-2-oxoimidazolidin-1-yl}-5-[2-(3-fluoro-3-methylazetidin-1-yl)-2-oxoethyl]-6-oxo-5,6-dihydropyrido[2,3-b]pyrazine-7-carboxamide, (Compound 42)

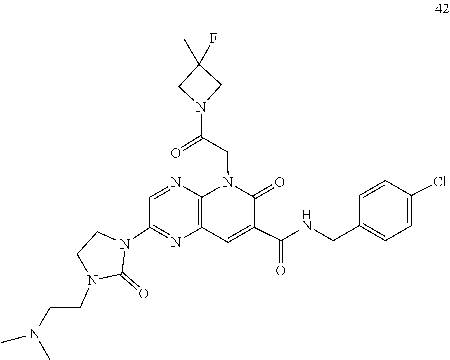 [SMILES: CN(C)CCN1CCN(c2cnc3c(cc(C(=O)NCc4ccc(Cl)cc4)c(=O)n3CC(=O)N3CC(C)(F)C3)n2)C1=O]

42 was synthesized as in Scheme 24.

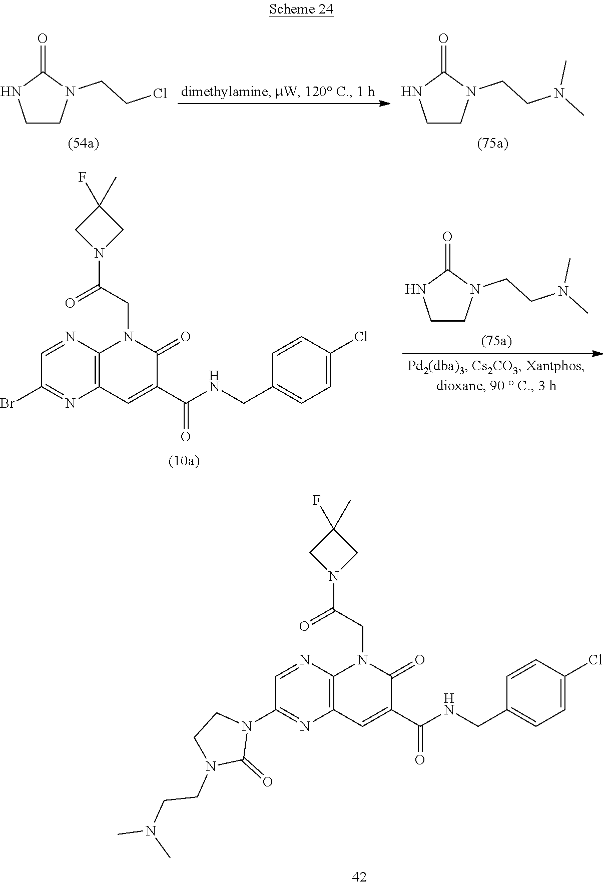 [SMILES: O=C1NCCN1CCCl ; CN(C)CCN1CCNC1=O ; CC1(F)CN(C(=O)Cn2c(=O)c(C(=O)NCc3ccc(Cl)cc3)cc3nc(Br)cnc32)C1 ; CN(C)CCN1CCNC1=O ; CN(C)CCN1CCN(c2cnc3c(cc(C(=O)NCc4ccc(Cl)cc4)c(=O)n3CC(=O)N3CC(C)(F)C3)n2)C1=O]

Preparation of 1-[2-(dimethylamino)ethyl]imidazolidin-2-one, (75a). A mixture of 1-(2-chloroethyl)imidazolidin-2-one (54a) (100 mg, 0.68 mmol) and dimethylamine (2 mL, 4 mmol, 2 M in THF) in a sealed tube was irradiated in a microwave reactor for 1 h at 120° C. The mixture was concentrated under reduced pressure generated (75a) (100 mg) as a colorless gum. This material was used in the next step without further purification.

Preparation of N-[(4-chlorophenyl)methyl]-2-{3-[2-(dimethylamino)ethyl]-2-oxoimidazolidin-1-yl}-5-[2-(3-fluoro-3-methylazetidin-1-yl)-2-oxoethyl]-6-oxo-5,6-dihydropyrido[2,3-b]pyrazine-7-carboxamide, 42. A mixture of 2-bromo-N-[(4-chlorophenyl)methyl]-5-[2-(3-fluoro-3-methylazetidin-1-yl)-2-oxoethyl]-6-oxo-5,6-dihydropyrido[2,3-b]pyrazine-7-carboxamide (10a) (65 mg, 0.125 mmol), 1-[2-(dimethylamino)ethyl]imidazolidin-2-one (75a) (100 mg, 0.63 mmol), Pd₂(dba)₃ (6 mg, 0.007 mmol), Xantphos (11 mg, 0.019 mmol), cesium carbonate (49 mg, 0.149 mmol) and dioxane (5 mL) was heated overnight at 90° C. After cooling to ambient temperature, the mixture was concentrated under reduced pressure. The product was purified by column chromatography on silica gel (eluted with a gradient of 0% to 5% MeOH in CHCl₃), which produced 42 (41 mg, 54% yield) as a light yellow solid. ¹H NMR (600 MHz, DMSO-d₆) δ 9.92 (t, J=6.0 Hz, 1H), 9.62 (s, 1H), 8.66 (s, 1H), 7.43-7.35 (m, 4H), 5.19-5.06 (m, 2H), 4.57 (d, J=7.15 Hz, 2H), 4.55-4.43- (m, 2H), 4.10-3.94 (m, 4H), 3.72-3.58 (m, 2H), 3.43-3.35 (m, 2H), 2.47-2.41 (m, 2H), 2.19 (s, 6H), 1.63 (d, J=21.8 Hz, 3H). ¹⁹F NMR (565 MHz, DMSO-d₆) δ −139.12-−138.93 (m, 1F). LC/MS: Eluent system A (retention time: 4.39 min); ESI-MS: 599 [M+H]⁺.

### Synthesis of 2-[3-(7-{[(4-chlorophenyl)methyl]carbamoyl}-5-[2-(3,3-dimethylazetidin-1-yl)-2-oxoethyl]-6-oxo-5,6-dihydropyrido[2,3-b]pyrazin-2-yl)-2-oxoimidazolidin-1-yl]-2-methylpropanoic Acid, (Compound 43)

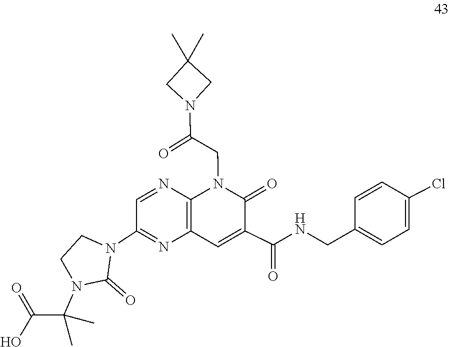 [SMILES: CC1(C)CN(C(=O)Cn2c(=O)c(C(=O)NCc3ccc(Cl)cc3)cc3nc(N4CCN(C(C)(C)C(=O)O)C4=O)cnc32)C1]

43 was synthesized as in Scheme 25.

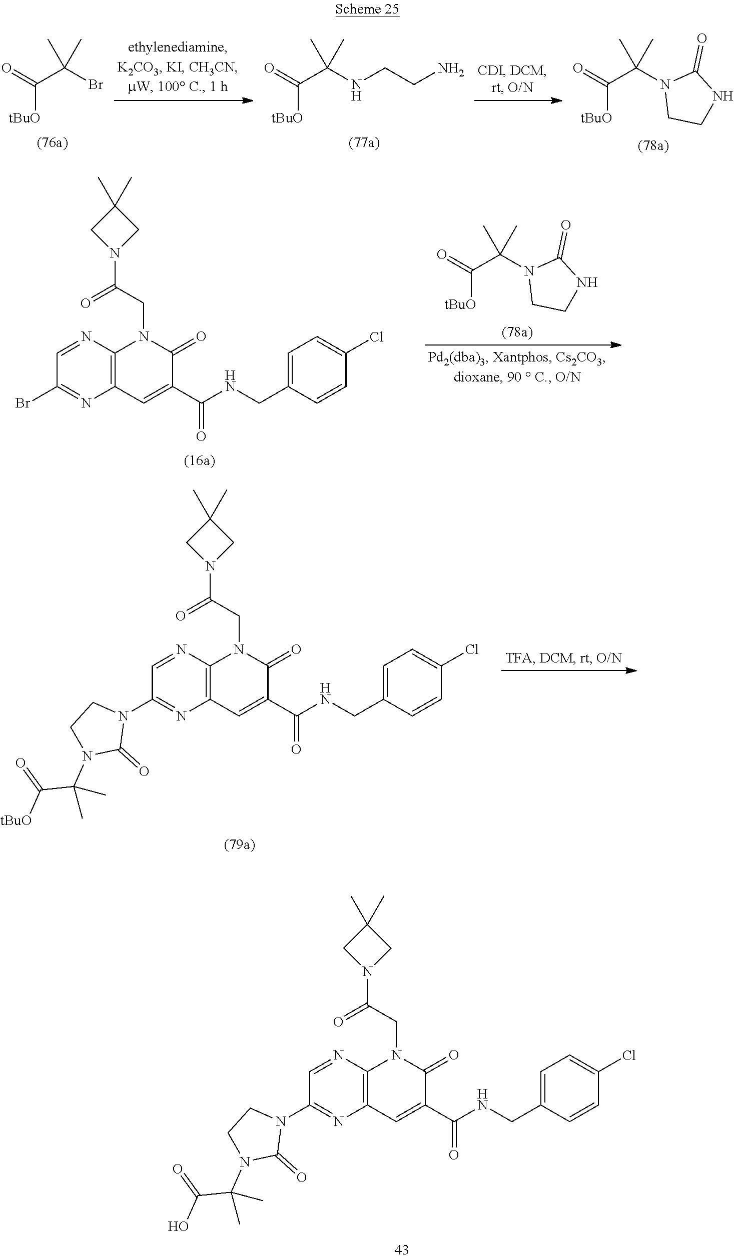

Preparation of tert-butyl N-(2-aminoethyl)-2-methylalaninate (77a). A mixture of tert-butyl 2-bromo-2-methylpropanoate (76a) (1.0 g, 4.48 mmol), ethylenediamine (2.7 g, 44.8 mmol), K₂CO₃ (0.68 g, 4.93 mmol), KI (75 mg, 0.45 mmol) and CH₃CN (6 mL) in a sealed tube was irradiated in a microwave reactor for 1 h at 100° C. After cooling to ambient temperature, the mixture was concentrated under reduced pressure. The residue was then mixed with ethyl acetate (100 mL), and the organic layer was washed with water (2×25 mL) and brine (25 mL). The organic layer was dried over MgSO₄ (anhydrous), filtered and concentrated under reduced pressure which generated (77a) (0.52 g) as a colorless liquid. This material was used in the next step without further purification.

Preparation of tert-butyl 2-methyl-2-(2-oxoimidazolidin-1-yl)propanoate, (78a). To a solution of tert-butyl N-(2-aminoethyl)-2-methylalaninate (77a) (0.52 g, 2.57 mmol) in DCM (25 mL) was added CDI (1.3 g, 7.71 mmol) in three portions over 30 min. After overnight, the mixture was diluted with CHCl₃ (75 mL), and washed with HCl (2×25 mL, 1 M aqueous) and water (3×25 mL), dried over Na₂SO₄, filtered and concentrated under reduced pressure, which provided (78a) (0.33 g, 55% yield) as a white solid.

Preparation of tert-butyl 2-[3-(7-{[(4-chlorophenyl)methyl]carbamoyl}-5-[2-(3,3-dimethylazetidin-1-yl)-2-oxoethyl]-6-oxo-5,6-dihydropyrido[2,3-b]pyrazin-2-yl)-2-oxoimidazolidin-1-yl]-2-methylpropanoate, (79a). A mixture of 2-bromo-N-[(4-chlorophenyl)methyl]-5-[2-(3,3-dimethylazetidin-1-yl)-2-oxoethyl]-6-oxo-5,6-dihydropyrido[2,3-b]pyrazine-7-carboxamide (16a) (70 mg, 0.135 mmol), tert-butyl 2-methyl-2-(2-oxoimidazolidin-1-yl)propanoate (78a) (62 mg, 0.269 mmol), Pd₂(dba)₃ (7 mg, 0.008 mmol), Xantphos (12 mg, 0.021 mmol), cesium carbonate (66 mg, 0.202 mmol) and dioxane (3 mL) was heated at 90° C. After overnight and cooling to ambient temperature, the mixture was concentrated under reduced pressure. The product was purified by column chromatography on silica gel (eluted with a gradient of 0% to 5% MeOH in CHCl₃), which produced (79a) (71 mg, 79% yield) as a light yellow solid.

Preparation of 2-[3-(7-{[(4-chlorophenyl)methyl]carbamoyl}-5-[2-(3,3-dimethylazetidin-1-yl)-2-oxoethyl]-6-oxo-5,6-dihydropyrido[2,3-b]pyrazin-2-yl)-2-oxoimidazolidin-1-yl]-2-methylpropanoic acid, 43. To a solution of tert-butyl 2-[3-(7-{[(4-chlorophenyl)methyl]carbamoyl}-5-[2-(3,3-dimethylazetidin-1-yl)-2-oxoethyl]-6-oxo-5,6-dihydropyrido[2,3-b]pyrazin-2-yl)-2-oxoimidazolidin-1-yl]-2-methylpropanoate (79a) (71 mg, 0.107 mmol) in DCM (2 mL) was added TFA (2 mL). After overnight, the mixture was concentrated under reduced pressure, then the resulting gum was dissolved in chloroform (25 mL) and concentrated again under reduced pressure. The residue was suspended in ether (10 mL), filtered and dried under vacuum which generated 43 (28 mg, 42% yield) as a light yellow solid. ¹H NMR (600 MHz, DMSO-d₆) δ 12.62 (br s, 1H), 9.93 (t, J=6.0 Hz, 1H), 9.54 (s, 1H), 8.66 (s, 1H), 7.46-7.34 (m, 4H), 5.08 (s, 2H), 4.57 (d, J=6.0 Hz, 2H), 4.10-3.95 (m, 4H), 3.71 (t, J=7.9 Hz, 2H), 3.59 (br s, 2H), 1.50 (s, 6H), 1.29 (s, 6H). LC/MS: Eluent system A (retention time: 6.31 min); ESI-MS: 610 [M+H]⁺, ESI-MS: 608 [M−H]⁻.

### Synthesis of 2-[3-(5-[2-(5-azaspiro[2.3]hexan-5-yl)-2-oxoethyl]-7-{[(4-chlorophenyl)methyl]carbamoyl}-6-oxo-5,6-dihydropyrido[2,3-b]pyrazin-2-yl)-2-oxoimidazolidin-1-yl]-2-methylpropanoic Acid, (Compound 44)

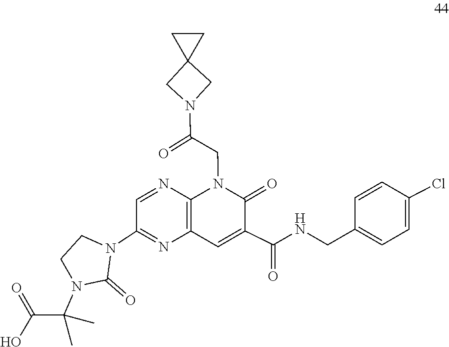 [SMILES: CC(C)(C(=O)O)N1CCN(c2cnc3c(cc(C(=O)NCc4ccc(Cl)cc4)c(=O)n3CC(=O)N3CC4(CC4)C3)n2)C1=O]

44 was synthesized as in Scheme 26.

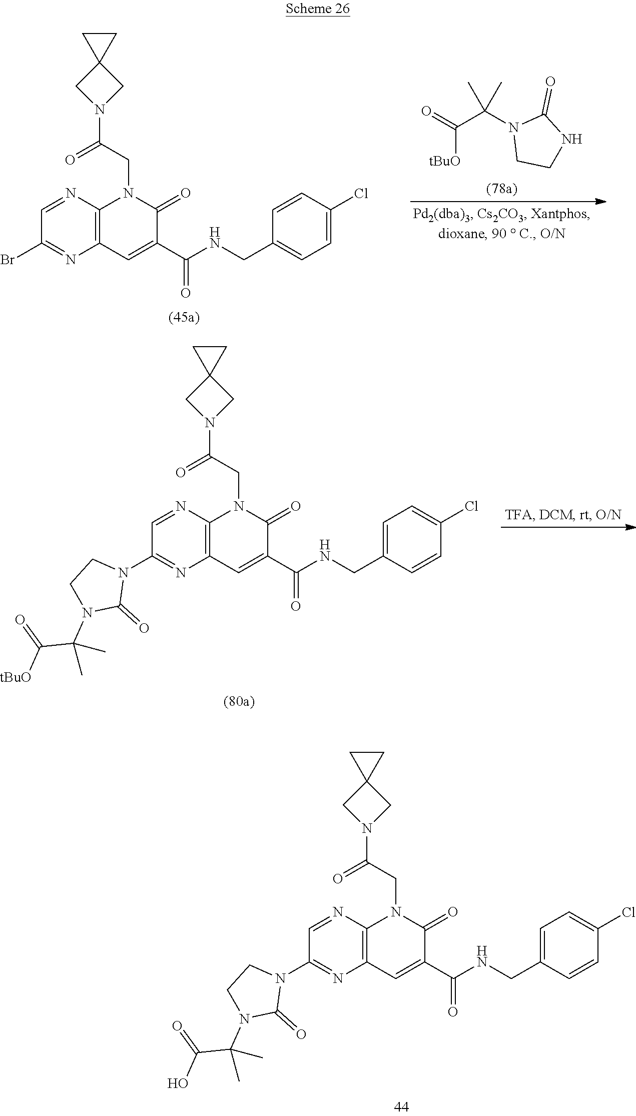 [SMILES: O=C(NCc1ccc(Cl)cc1)c1cc2nc(Br)cnc2n(CC(=O)N2CC3(CC3)C2)c1=O ; CC(C)(C)OC(=O)C(C)(C)N1CCNC1=O ; CC(C)(C)OC(=O)C(C)(C)N1CCN(c2cnc3c(cc(C(=O)NCc4ccc(Cl)cc4)c(=O)n3CC(=O)N3CC4(CC4)C3)n2)C1=O ; CC(C)(C(=O)O)N1CCN(c2cnc3c(cc(C(=O)NCc4ccc(Cl)cc4)c(=O)n3CC(=O)N3CC4(CC4)C3)n2)C1=O]

Preparation of tert-butyl 2-[3-(5-[2-(5-azaspiro[2.3]hexan-5-yl)-2-oxoethyl]-7-{[(4-chlorophenyl)methyl]carbamoyl}-6-oxo-5,6-dihydropyrido[2,3-b]pyrazin-2-yl)-2-oxoimidazolidin-1-yl]-2-methylpropanoate, (80a). A mixture of 5-[2-(5-azaspiro[2.3]hexan-5-yl)-2-oxoethyl]-2-bromo-N-[(4-chlorophenyl)methyl]-6-oxo-5,6-dihydropyrido[2,3-b]pyrazine-7-carboxamide (45a) (71 mg, 0.137 mmol), tert-butyl 2-methyl-2-(2-oxoimidazolidin-1-yl)propanoate (78a) (62 mg, 0.269 mmol), Pd₂(dba)₃ (7 mg, 0.008 mmol), Xantphos (12 mg, 0.021 mmol), cesium carbonate (66 mg, 0.202 mmol) and dioxane (3 mL) was heated at 90° C. After overnight the mixture was cooled to ambient temperature and then concentrated under reduced pressure. The product was purified by column chromatography on silica gel (eluted with a gradient of 0% to 5% MeOH in CHCl₃), which produced (80a) (62 mg, 68% yield) as a light yellow solid.

Preparation of 2-[3-(5-[2-(5-azaspiro[2.3]hexan-5-yl)-2-oxoethyl]-7-{[(4-chlorophenyl)methyl]carbamoyl}-6-oxo-5,6-dihydropyrido[2,3-b]pyrazin-2-yl)-2-oxoimidazolidin-1-yl]-2-methylpropanoic acid, 44. To a solution of tert-butyl 2-[3-(5-[2-(5-azaspiro[2.3]hexan-5-yl)-2-oxoethyl]-7-{[(4-chlorophenyl)methyl]carbamoyl}-6-oxo-5,6-dihydropyrido[2,3-b]pyrazin-2-yl)-2-oxoimidazolidin-1-yl]-2-methylpropanoate (80a) (62 mg, 0.093 mmol) in DCM (2 mL) was added TFA (2 mL). After overnight, the mixture was concentrated under reduced pressure, then the resulting gum was dissolved in chloroform (25 mL) and concentrated again under reduced pressure. The residue was suspended in ether (10 mL) and filtered to generate 2-[3-(5-[2-(5-azaspiro[2.3]hexan-5-yl)-2-oxoethyl]-7-{[(4-chlorophenyl)methyl]carbamoyl}-6-oxo-5,6-dihydropyrido[2,3-b]pyrazin-2-yl)-2-oxoimidazolidin-1-yl]-2-methylpropanoic acid 44 (41 mg, 72% yield) as a light yellow solid. ¹H NMR (600 MHz, DMSO-d₆) δ 12.62 (br s, 1H), 9.94 (t, J=6.0 Hz, 1H), 9.55 (s, 1H), 8.67 (s, 1H), 7.45-7.35 (m, 4H), 5.12 (s, 2H), 4.58 (d, J=6.0 Hz, 2H), 4.40 (s, 2H), 4.03 (t, J=7.9 Hz, 2H), 3.97 (s, 2H), 3.71 (t, J=7.9 Hz, 2H), 1.50 (s, 6H), 0.75-0.64 (m, 4H). LC/MS: Eluent system A (retention time: 6.14 min); ESI-MS: 608 [M+H]⁺, ESI-MS: 606 [M−H]⁻.

### Synthesis of 2-(3-(6-{[(4-chlorophenyl)methyl]carbamoyl}-8-(2-(3,3-dimethylazetidin-1-yl)-2-oxoethyl)-7-oxo-7,8-dihydro-1,8-naphthyridin-3-yl)-2-oxoimidazolidin-1-yl)-2-methylpropanoic Acid, (Compound 45)

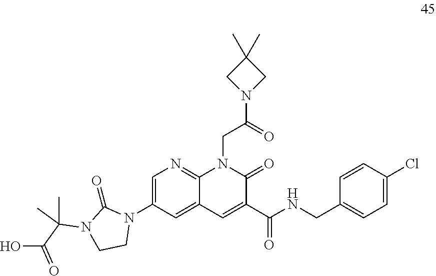 [SMILES: CC1(C)CN(C(=O)Cn2c(=O)c(C(=O)NCc3ccc(Cl)cc3)cc3cc(N4CCN(C(C)(C)C(=O)O)C4=O)cnc32)C1]

45 was synthesized as in Scheme 27.

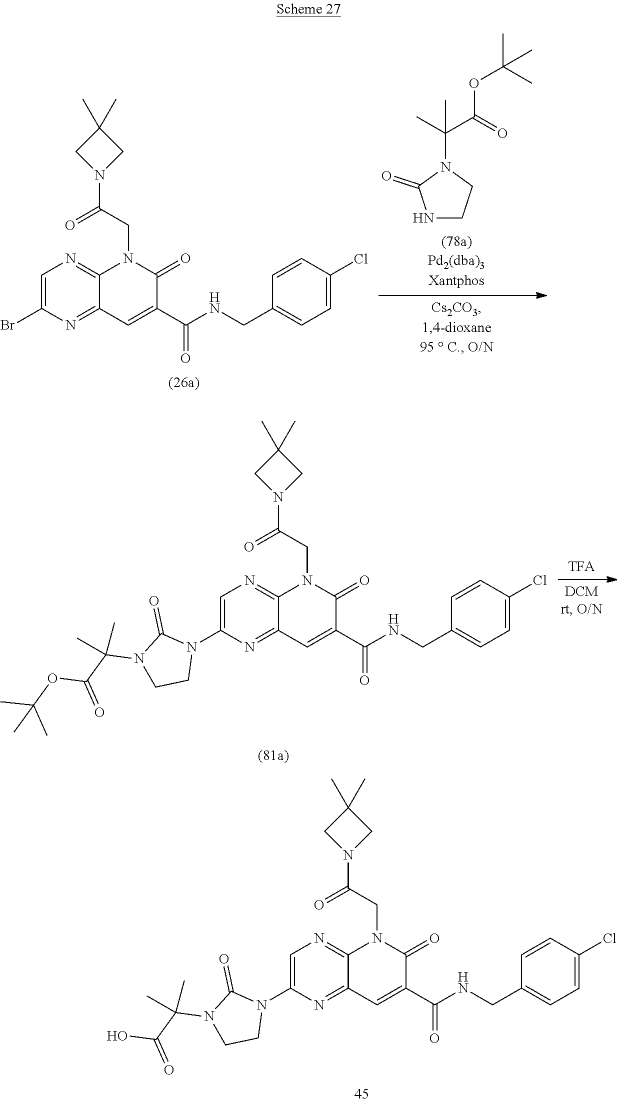 [SMILES: CC1(C)CN(C(=O)Cn2c(=O)c(C(=O)NCc3ccc(Cl)cc3)cc3nc(Br)cnc32)C1 ; CC(C)(C)OC(=O)C(C)(C)N1CCNC1=O ; CC1(C)CN(C(=O)Cn2c(=O)c(C(=O)NCc3ccc(Cl)cc3)cc3nc(N4CCN(C(C)(C)C(=O)OC(C)(C)C)C4=O)cnc32)C1 ; CC1(C)CN(C(=O)Cn2c(=O)c(C(=O)NCc3ccc(Cl)cc3)cc3nc(N4CCN(C(C)(C)C(=O)O)C4=O)cnc32)C1]

Preparation of tert-butyl 2-(3-(6-((4-chlorobenzyl)carbamoyl)-8-(2-(3,3-dimethylazetidin-1-yl)-2-oxoethyl)-7-oxo-7,8-dihydro-1,8-naphthyridin-3-yl)-2-oxoimidazolidin-1-yl)-2-methylpropanoate, (81a). A mixture of 6-bromo-N-(4-chlorobenzyl)-1-(2-(3,3-dimethylazetidin-1-yl)-2-oxoethyl)-2-oxo-1,2-dihydro-1,8-naphthyridine-3-carboxamide (26a) (45 mg, 0.087 mmol), tert-butyl 2-methyl-2-(2-oxoimidazolidin-1-yl)propanoate (78a) (45 mg, 0.197 mmol), bis(dibenzylideneacetone)palladium(0) (21 mg, 0.023 mmol), Xantphos (43 mg, 0.075 mmol), and caesium carbonate (130 mg, 0.40 mmol) in anhydrous 1,4-dioxane (8 mL) was heated to 95° C. After overnight, the reaction mixture was filtered through Celite® and the solid washed with chloroform (20 mL) and the combined filtrate concentrated under reduced pressure. The product was purified by column chromatography on silica (eluted with 0% to 100% ethyl acetate in DCM) followed by recrystallization from methanol provided (81a) (42 mg, 72% yield) as yellow crystalline powder.

Preparation of 2-(3-(6-{[(4-chlorophenyl)methyl]carbamoyl}-8-(2-(3,3-dimethylazetidin-1-yl)-2-oxoethyl)-7-oxo-7,8-dihydro-1,8-naphthyridin-3-yl)-2-oxoimidazolidin-1-yl)-2-methylpropanoic acid, 45. A solution of tert-butyl 2-(3-(6-((4-chlorobenzyl)carbamoyl)-8-(2-(3,3-dimethylazetidin-1-yl)-2-oxoethyl)-7-oxo-7,8-dihydro-1,8-naphthyridin-3-yl)-2-oxoimidazolidin-1-yl)-2-methylpropanoate (81a) (42 mg, 0.063 mmol) in DCM (5 mL) was mixed with TFA (3 mL). After overnight, then mixture was concentrated under reduced pressure and the resulting residue triturated with ether and dried, which provided 45 (34 mg, 89% yield) as yellow powder. ¹H NMR (600 MHz, DMSO-d6) δ 12.29 (br.s, 1H), 9.97 (t, J=6.1 Hz, 1H), 9.09 (d, J=2.7 Hz, 1H), 8.92 (s, 1H), 8.50 (d, J=2.7 Hz, 1H), 7.42-7.36 (m, 4H), 5.09 (s, 2H), 4.57 (d, J=6.1 Hz, 2H), 4.01 (s, 2H), 3.93-3.89 (m, 2H), 3.69-3.65 (m, 2H), 3.58 (s, 2H), 1.48 (s, 6H), 1.29 (s, 6H). LC/MS: Eluent system A (retention time: 6.10 min); ESI-MS 609 [M+H]⁺, 607 [M−H]⁻

### Synthesis of 2-[3-(7-{[(4-chlorophenyl)methyl]carbamoyl}-5-[2-(3,3-dimethylazetidin-1-yl)-2-oxoethyl]-6-oxo-5,6-dihydropyrido[2,3-b]pyrazin-2-yl)-2-oxoimidazolidin-1-yl]propanoic Acid, (Compound 46)

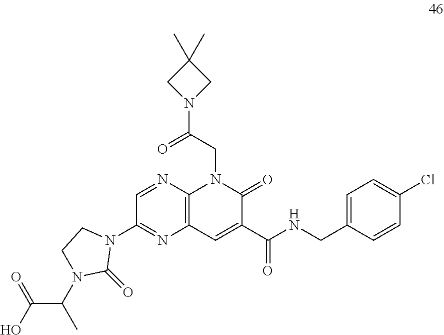 [SMILES: CC(C(=O)O)N1CCN(c2cnc3c(cc(C(=O)NCc4ccc(Cl)cc4)c(=O)n3CC(=O)N3CC(C)(C)C3)n2)C1=O]

46 was synthesized as in Scheme 28.

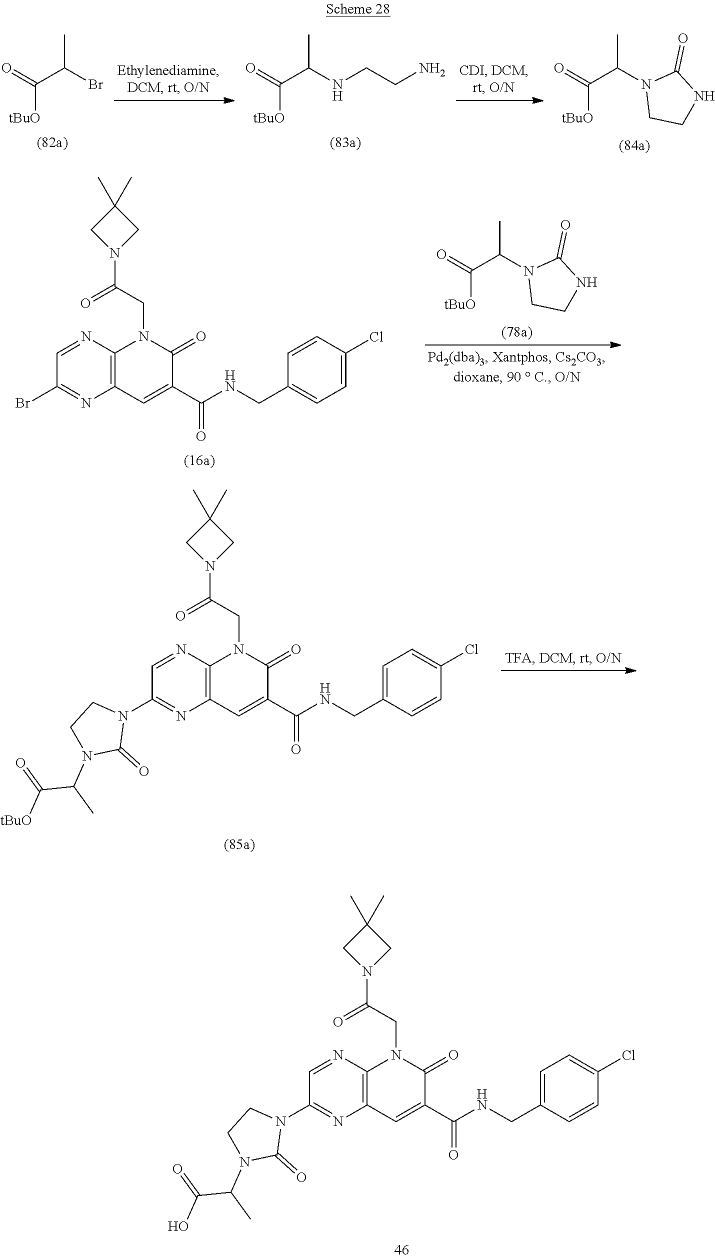

Preparation of tert-butyl N-(2-aminoethyl)alaninate, (83a). To the solution of ethylendiamine (2.86 g, 47.8 mmol) in DCM (100 mL) was slowly added tert-butyl 2-bromopropanoate (82a) (1.0 g, 4.78 mmol). After overnight, the mixture was concentrated under reduced pressure and the residue was dissolved in ethyl acetate (100 mL) and washed with water (25 mL) and brine (25 mL), dried over Na₂SO₄ and concentrated under reduced pressure, which provided (83a) (0.40 g) as a colorless, viscous oil. This material was used in the next step without further purification.

Preparation of tert-butyl 2-(2-oxoimidazolidin-1-yl)propanoate, (84a). To a solution of tert-butyl N-(2-aminoethyl)alaninate (83a) (0.40 g, 2.12 mmol) in DCM (25 mL) was added CDI (1.03 g, 6.37 mmol) in three portions over 30 min. After overnight, CHCl₃ (75 mL) was added, and the resulting mixture washed with HCl (2×25 mL, 1 N aqueous) and water (3×25 mL), dried over Na₂SO₄ and concentrated under reduced pressure, which provided (84a) (0.34 g, 75% yield) as a white solid.

Preparation of tert-butyl 2-[3-(7-{[(4-chlorophenyl)methyl]carbamoyl}-5-[2-(3,3-dimethylazetidin-1-yl)-2-oxoethyl]-6-oxo-5,6-dihydropyrido[2,3-b]pyrazin-2-yl)-2-oxoimidazolidin-1-yl]propanoate, (85a). A mixture of 2-bromo-N-[(4-chlorophenyl)methyl]-5-[2-(3,3-dimethylazetidin-1-yl)-2-oxoethyl]-6-oxo-5,6-dihydropyrido[2,3-b]pyrazine-7-carboxamide (16a) (65 mg, 0.125 mmol), tert-butyl 2-(2-oxoimidazolidin-1-yl)propanoate (84a) (54 mg, 0.249 mmol), Pd₂(dba)₃ (7 mg, 0.008 mmol), Xantphos (12 mg, 0.021 mmol), cesium carbonate (66 mg, 0.202 mmol) and dioxane (3 mL) was heated 90° C. After overnight, and cooling to ambient temperature, the mixture was concentrated under reduced pressure. The product was purified by column chromatography on silica gel (eluted with a gradient of 0% to 5% MeOH in CHCl₃), which generated (85a) (69 mg, 84% yield) as a light yellow solid.

Preparation of 2-[3-(7-{[(4-chlorophenyl)methyl]carbamoyl}-5-[2-(3,3-dimethylazetidin-1-yl)-2-oxoethyl]-6-oxo-5,6-dihydropyrido[2,3-b]pyrazin-2-yl)-2-oxoimidazolidin-1-yl]propanoic acid, 46. To a solution of tert-butyl 2-[3-(7-{[(4-chlorophenyl)methyl]carbamoyl}-5-[2-(3,3-dimethylazetidin-1-yl)-2-oxoethyl]-6-oxo-5,6-dihydropyrido[2,3-b]pyrazin-2-yl)-2-oxoimidazolidin-1-yl]propanoate (85a) (69 mg, 0.105 mmol) in DCM (5 mL) was added TFA (2 mL). After overnight the mixture was concentrated under reduced pressure, and the resulting material was dissolved in chloroform (25 mL) and concentrated again under reduced pressure. The residue was suspended in ether (10 mL), filtered and dried which generated 46 (30 mg, 48% yield) as a light yellow solid. 1H NMR (600 MHz, DMSO-d₆) δ 12.98 (br s, 1H), 9.93 (t, J=6.0 Hz, 1H), 9.59 (s, 1H), 8.67 (s, 1H), 7.44-7.36 (m, 4H), 5.09 (br s, 2H), 4.57 (d, J=6.0 Hz, 2H), 4.51 (q, J=7.3 Hz, 1H), 4.14-4.10 (m, 1H), 4.07-3.96 (m, 3H), 3.69-3.60 (m, 2H), 3.59 (br.s, 2H), 1.43 (d, J=7.3 Hz, 3H), 1.29 (s, 6H). LC/MS: Eluent system A (retention time: 6.11 min); ESI-MS: 596 [M+H]⁺, ESI-MS: 594 [M−H]⁻.

### Example 2: Protein Expression and Purification

Steps Performed:

1. Codon optimization and DNA synthesis of HCMV DNA polymerase (UL54) encoding gene (Accession #P08546);

2. Subclone synthesized DNA encoding for HCMV DNA pol into pFastBac vector with a 6His tag at the C-terminus and validate the insert fragment by DNA sequencing analysis;

3. Use of DH10Bac cells to generate bacmid DNA;

4. Transfection of the bacmid DNA into Sf9 cells;

5. Amplifying primary baculovirus and validation of protein expression by Western blot;

6. Express up to 1 L of Sf9 cells infected with UL54-containing virus;

7. Purify over-expressed HCMV DNA pol by Ni-affinity column followed by conventional/FPLC chromatography methods until the final product is >90% homogeneity at the concentration of >1 mg/mL;

8. Formulate the purified protein in a proper buffer system by dialysis;

9. Measure protein concentration and validate by SDS-PAGE and Western blot.

Materials:

pFastBac vector was originally purchased from Invitrogen. Anti-6Hismonoclonal antibody was purchased from Sigma Aldrich. Ni-sepharose resin was purchased from GE Healthcare.

Methods:

1. Subcloning

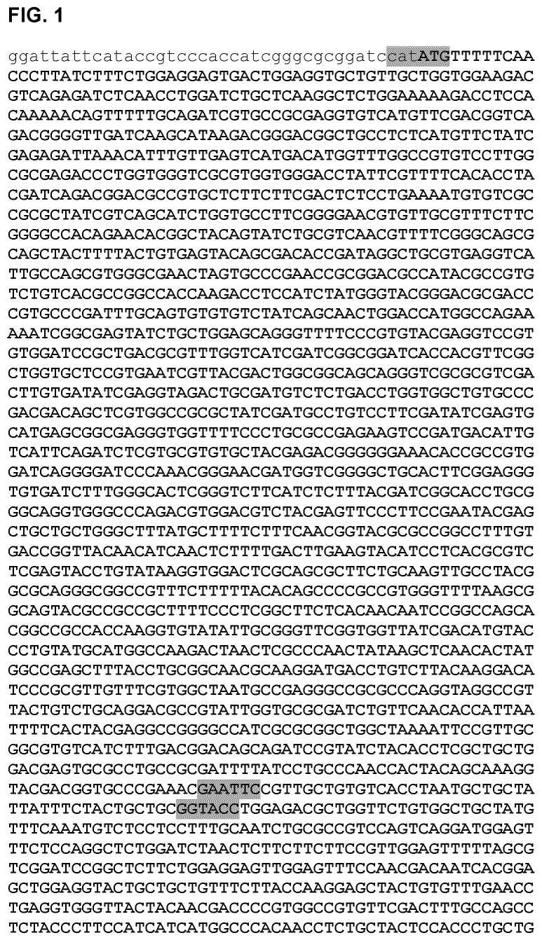

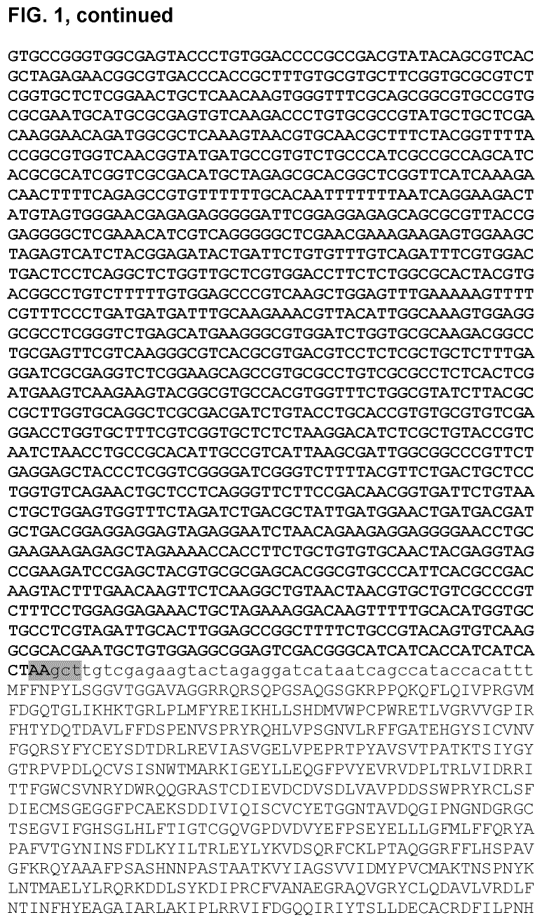

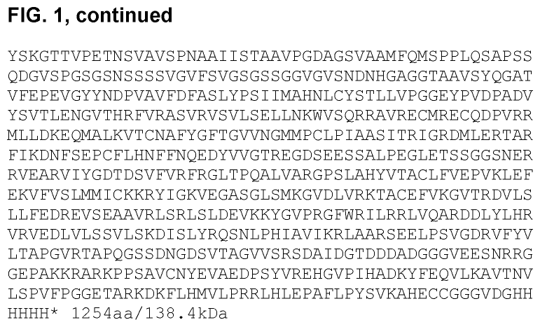

Synthesized DNA encoding for codon optimized HCMV DNA polymerase gene (UL54) was cloned into the pFastBac baculovirus vector between NdeI and HindIII sites with a 6His tag at the C-terminus. Positive clones were validated by DNA sequencing analysis (see FIG. 1).

2. Expression

Sequence-validated pFB-UL54-6H (c2) was transformed into DH10b cells. The bacmid DNA was isolated and transfected into Sf9 cells. Expression of UL54 was determined by Western blot using the monoclonal antibody against 6His tag. The primary baculoirus was collected and further amplified. For production of UL54, 1 L of Sf9 cells were infected with amplified virus (MOI=1.5) and the cell pellet was harvested 96 hrs post-infection.

3. Purification

Cell lysate was prepared. Over-expressed HCMV DNA polymerase (UL54) was purified by Ni-affinity column followed by other conventional/FPLC chromatographic methods.

4. Formulation

Peak fractions were pooled, concentrated and dialyzed in the formulation buffer containing 20 mM Tris-Cl, pH7.5, 20% glycerol, 150 mM NaCl, 2 mM DTT and 2 mM EDTA.

5. Validation

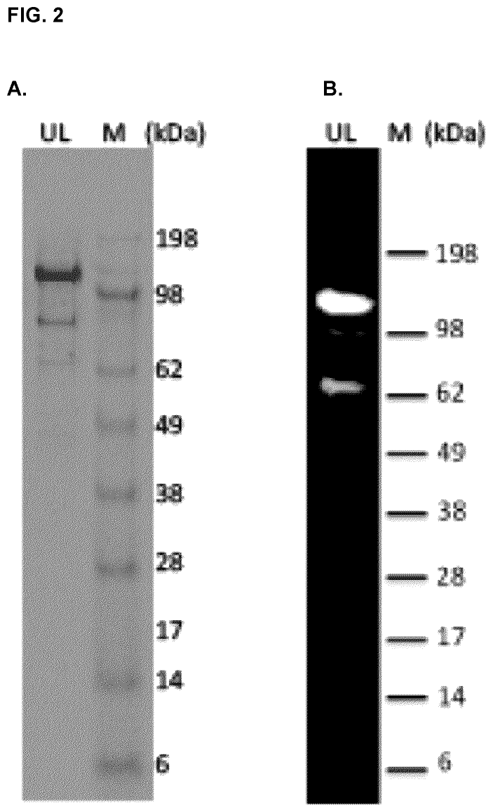

Purified and formulated HCMV DNA polymerase was validated by SDSPAGE (FIG. 2, panel A) and Western blot (FIG. 2, panel B) using the monoclonal antibody against 6His tag.

### Example 3: Determination of Enzyme Inhibition in HCMV DNA Polymerase Assay

The HCMV DNA polymerase assay was developed by ProFoldin. The assay detects the formation of DNA from the DNA polymerase reaction in the presence of a DNA template, dATP and dGTP. The formation of the DNA is detected by using a fluorescence method. The fluorescence intensity increases when DNA is generated by the enzyme reaction.

A time-course experiment was performed aside the enzyme inhibition assay to ensure the assay was sensitive to the enzyme inhibition. The wells for the time-course experiment contained 1 μL of DMSO and the same reaction mixture as that in the enzyme inhibition assay. The time-course data varied between plates. The reactions were stopped when the fluorescence reached a value of about 3700 for each plate.

Exemplary solid compounds (e.g., as described herein) were dissolved in DMSO to make 10 μM stock solutions. PFA (phosphonoformic acid or phosphonomethanoic acid) was used as a positive control. The compound mother plates were prepared at 50× concentrated compound concentrations with a 2- fold serial dilution. All the exemplary compounds except PFA were diluted in DMSO. PFA was diluted in water. The maximum compound concentration was 2 mM that gave the final concentration of 40 μM in the assay. One microliter of the 50× stock solutions was transferred into the assay plates. Three copies of assay plates were prepared, two for HCMV DNA polymerase assay and one for compound background control.

The following is the protocol for enzyme inhibition performed at 37° C.:

1. Prepared a premix composed of 35347 μL of H₂O, 5220 μL of 10× assay buffer and 149 μL of 350×HCMV DNA polymerase.

2. Added 39 μL of the premix into the wells with 1 μL of the compound. Incubated the mixture for at least 5 min.

3. Added 10 μL of 5×dNTP (0.1 mM), 5×DNA template solutions into each well and incubated the solution until the fluorescence value reached a value about 3700 for the DMSO-only control wells.

4. Added 50 μL of the 1× dye into the wells.

5. Read the fluorescence at emission wavelength of 535 nm and excitation wavelength of 485 nm.

For the compound background reading, added 99 μL of water in the wells with 1 μL of compounds and read the fluorescence. The 100% inhibition value Fₜ₀ was from the wells with zero reaction time. The 0% inhibition value F₀ was from the wells with DMSO (no inhibitor).

The background values were subtracted from the fluorescence reading to get the net fluorescence values. The net fluorescence values were used for calculation of the percentage inhibition.

% Inhibition=100%−[(Fᵢ−Fₜ₀)×100%/(F₀−Fₜ₀)]

Where

Fᵢ=Fluorescence with inhibitor

F₀=Fluorescence without inhibitor but DMSO (0% inhibition):

Fₜ₀=Fluorescence without enzyme activity (100% inhibition)

The assay was performed in duplicate for the test article at 40, 20, 10, 2.5, 1.25, 0.625, 0.313, 0.156 and 0.078 μM. The IC₅₀ curves were obtained by fitting the percentage inhibition values with the compound concentrations using Prism GraphPad software. The results are presented in Table 2.

TABLE 2
HCMV polymerase activity
Activity IC₅₀ > 1 microM = + and IC₅₀ < 1 microM ++

| | HCMV |
|---|---|
| Compound | Polymerase |
| Number | Activity |
| 1 | ++ |
| 2 | ++ |
| 3 | ++ |
| 4 | ++ |
| 6 | ++ |
| 10 | ++ |
| 11 | ++ |
| 12 | ++ |
| 13 | ++ |
| 14 | ++ |
| 15 | ++ |
| 16 | ++ |
| 17 | ++ |
| 18 | ++ |
| 19 | ++ |
| 21 | ++ |
| 22 | ++ |
| 23 | ++ |
| 24 | + |
| 26 | ++ |
| 27 | ++ |
| 28 | ++ |
| 36 | ++ |
| 37 | + |
| 38 | ++ |
| 39 | + |
| 40 | ++ |
| 41 | ++ |
| 42 | + |
| 43 | ++ |
| 44 | ++ |
| 45 | ++ |
| 46 | ++ |

### Example 4: Evaluation of In Vitro Inhibitory Activity of Exemplary Compounds Against HCMV

### Abbreviation

ATCC American Type Culture
   Collection
CAS Chinese Academy
   Sciences
CC Cell Control
CC50 50% Cytotoxic
   Concentration
CCK8 Cell Counting Kit 8
CPD Compound
DMSO Dimethylsulfoxide
EC50 50% Effective
   Concentration
EMEM Eagle's Minimal
   Essential Medium
FBS Fetal Bovine Serum
GFP Green Fluorescent
   Protein
HCMV Human
   Cytomegalovirus
MC Medium Control
MOI Multiplicity of
   Infection
NA Not Available
NEAA Non-Essential Amino
   Acid
OD Optical Density
PS Penicillin-Streptomycin
SI Selectivity Index
VC Virus Control

Exemplary compounds (e.g., as described herein) were prepared as 20 mM stock solutions with DMSO. Reference compound was ganciclovir (Sigma). Compounds were tested at 8 concentrations, 3-fold serial dilutions, in duplicate for EC₅₀ and CC₅₀ determinations. The highest testing concentrations of the exemplary compounds and reference compound were 40 μM and 100 μM, respectively. The final concentration of DMSO in the cell culture was 0.5%.

Virus:

HCMV US3-6-EGFP-HCMV-AD169 strain was obtained from Institute Pasteur of Shanghai, CAS. The virus contains GFP as a reporter of the viral replication.

Cell Lines:

MRC5 cells (ATCC CCL-171) was obtained from the ATCC and maintained in the EMEM (Sigma) supplemented with 10% FBS (HyClone), 1% L-glutamine (Gibco), 1% NEAA (Gibco), and 1% PS (Hyclone). Reagent:

The major reagent used in the study was CCK8 (Shanghai Life iLab).

Instruments:

The major instruments used in the study were Acumen Cellista (TTP LabTech) and SpectraMax340PC384 (Molecular Device).

Study Protocol:

| | | Compound | | |
|---|---|---|---|---|
| | | treatment | | |
| | | duration (day)/ | Reference | Detection |
| Virus | Cells | Endpoint | compound | reagent |
| HCMV | 20,000 | 4 Fluorometry | Ganciclovir | GFP. CCK8 |
| US3-6-EGFP- | MRC5 | | | |
| HCMV-AD169 | cells per | | | |
| | °6-well | | | |

In 96-well plates, MRC5 cells were seeded at 20,000 cells per well and cultured at 37° C. and 5% C02 overnight. Next day, compounds (8 concentrations, 3-fold dilutions, in duplicate) and US3-6-EGFP-HCMV-AD169 (MOI=0.1) were added to the cells. The resulting cultures were incubated at 37° C. and 5% C02 for 4 days. Fluorescence intensity was determined by Acumen Cellista (TTP LabTech). The antiviral activity of each compound was calculated based on the inhibition of expression of GFP at each concentration normalized by the cell control (cells without virus infection or compound treatment).

Cytotoxicity of the compounds was assessed under the same conditions, but without virus infection, in parallel. Cell viability was measured with CCK8 following the manufacturer's manual. CC₅₀ values were then calculated based on cytotoxicity at the test concentrations normalized by the medium control (medium only).

Antiviral activity and cytotoxicity of the compounds were expressed as % Inhibition and % Viability, respectively, and calculated with the formulas below:

Inhibition (%)=100−(Raw data_{CPD}−Average_{CC})/(Average_{VC}−Average_{CC})*100

Viability (%)=(Raw data_{CPD}−Average_{MC})/(Average_{CC}−Average_{MC})*100

Raw data_{CPD} indicates the values of the compound-treated wells; Average_{VC}, Average_{CC} and Average_{MC} indicate the average values of the virus control (cells infected with virus, without compound treatment), cell control and medium control wells, respectively.

EC₅₀ and CC₅₀ values were calculated using the GraphPad Prism software. The results are presented in Table 3.

TABLE 3
Inhibitory activity against HCMV
Activity EC₅₀ > 1 microM = + and EC₅₀ < 1 microM ++

| | MCR5 | HCMV |
|---|---|---|
| Compound | CC₅₀ | Antiviral |
| Number | (μM) | EC₅₀ range |
| 1 | <20 | ++ |
| 2 | <40 | ++ |
| 3 | >40 | ++ |
| 4 | >40 | ++ |
| 5 | >40 | + |
| 6 | >40 | ++ |
| 7 | >40 | + |
| 12 | >40 | ++ |
| 13 | >40 | + |
| 14 | >40 | + |
| 15 | >40 | ++ |
| 16 | >40 | + |
| 18 | >40 | |
| 19 | >40 | + |
| 20 | >40 | + |
| 21 | >40 | ++ |
| 22 | >40 | + |
| 26 | >40 | + |
| 27 | >40 | + |
| 28 | >40 | ++ |
| 36 | 18 | ++ |
| 37 | >40 | ++ |
| 40 | >40 | + |
| 41 | 23 | + |
| 42 | >40 | ++ |
| 43 | >40 | ++ |
| 44 | >40 | ++ |
| 45 | >40 | ++ |
| 46 | >40 | ++ |

### Example 5: Inhibition of hERG Potassium Channel

This automated patch clamp method (SyncroPatch 384PE) CHO cells stably expressing hERG potassium channels from Sophion Biosciences were used for this test.

Cell Culture:

The cells were cultured in a humidified and air-controlled (5% C02) incubator at 37° C.

TABLE 4
CHO hERG Culture Medium

| | | Catalog | Volume |
|---|---|---|---|
| Reagent | Supplier | Number | (mL) |
| Ham's F12 | Invitrogen | 31765-035 | 500 |
| FBS | HyClone | SV30087.03 | 50 |
| G418 Geneticin | Invitrogen | 10131-027 | 1 |
| Hygromycin B | Invitrogen | 1068-010 | 1 |

Preparation of Cells:

The CHO cells which were at least two days after plating and more than 75% confluent would be used for experiments. Before testing, cells were harvested using TrypLE and resuspended in the physiological solution at the room temperature.

Recording Solutions:

For the electrophysiological recordings the following solutions were used (Table 5). The physiological solution and external solution were prepared at least one month. The intracellular solution was prepared in batches aliquoted, and stored at 4° C. until used.

TABLE 5
Composition of Physiological, External and Internal Solutions

| | Physiological | External | Internal |
|---|---|---|---|
| Reagent | Solution (mM) | Solution(mM) | Solution(mM) |
| NaCl | 140 | 80 | 10 |
| KCl | 4 | 4 | 10 |
| KF | — | — | 110 |
| CaCl₂ | 2 | 2 | — |
| MgCl₂ | 1 | 1 | — |
| Glucose | 5 | 5 | — |
| NMDG | — | 60 | — |
| HEPES | 10 | 10 | 10 |
| EGTA | — | — | 10 |
| pH | 7.4 with NaOH | 7.4 with NaOH | 7.2 with KOH |
| Osmolarity | ~298 mOsm | ~289 mOsm | ~280 mOsm |

Preparation of Compounds:

Exemplary compounds (e.g., as described herein) were dissolved in 100% DMSO to obtain stock solutions for different test concentrations. Then the stock solutions were further diluted into external solution to achieve final concentrations for testing. Visual check for precipitation was conducted before testing. Final DMSO concentration in external solution was not more than 0.30% for the compounds.

Voltage command Protocol:

From this holding potential of −80 mV, the voltage was first stepped to −50 mV for 80 ms for leak subtraction, and then stepped to +20 mV for 4800 ms to open hERG channels. After that, the voltage was stepped back down to −50 mV for 5000 ms, causing a “rebound” or tail current, which was measured and collected for data analysis. Finally, the voltage was stepped back to the holding potential (−80 mV, 1000 ms). This voltage command protocol was repeated every 20000 msec. This command protocol was performed continuously during the test (vehicle control and test compound).

SyncroPatch Whole-Cell Recording:

hERG SyncroPatch assay was conducted at room temperature. The Setup, Prime Chip, Catch and Seal Cells, Amplifier Settings, Voltage and Application Protocols were established with Biomek Software (Nanion).

One addition of 40 μL of the vehicle was applied, followed by 300s for a baseline period. Then the doses of exemplary test compounds were added with 40 μL. The exposure of test compound at each concentration was no less than 300s. The recording for the whole process had to pass the quality control or the well was abandoned and the compound was retested, all automatically set by PatchControl. Five concentrations (0.494 μM, 1.48 μM, 4.44 μM, 13.33 μM and 40.00 μM) were tested for each compound. Minimum 2 replicates per concentration were obtained.

Data Analysis:

Data analysis was carried out using DataControl, Excel 2013 (Microsoft) and GraphPad Prism 5.0.

Within each well recording, percent of control values were calculated for each test compound concentration current response based on peak current in presence of reference control (current response/peak current)×100%. The Dose-Response curves were fit to the standard Hill equation as shown below:

Ipost cpd/Ipre cpd=Bottom+(Top−Bottom)/(1+10\hat{ }((Log IC₅₀−X)*HillSlope))

Where X is the logarithm of concentration, Ipost cpd/Ipre cpd is the normalized peak current amplitude, Top is 1 and Bottom is equal to 0.

Curve-fitting and IC₅₀ calculations were performed by GraphPad Prism 5.0. If the inhibition obtained at the lowest concentration tested was over 50%, or at the highest concentration tested was less than 50%, we reported the IC₅₀ as less than lowest concentration, or higher than highest concentration, respectively. The results are presented in Table 6.

For comparison, HCMV DNA polymerase inhibitors reported in the literature were used to compare to the hERG inhibition of the compounds disclosed herein. The following are the reported HCMV DNA polymerase inhibitors that were evaluated for hERG inhibition.

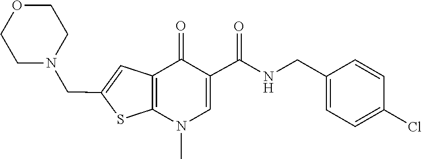 [SMILES: Cn1cc(C(=O)NCc2ccc(Cl)cc2)c(=O)c2cc(CN3CCOCC3)sc21]

PNU-243672 (Mark E. Schnute, Michele M. Cudahy, Roger J. Brideau, Fred L. Homa, Todd A. Hopkins, Mary L. Knechtel, Nancee L. Oien, Thomas W. Pitts, Roger A. Poorman, Michael W. Wathen, and Janet L. Wieber, J. Med. Chem. 2005, 48, 5794-5804)

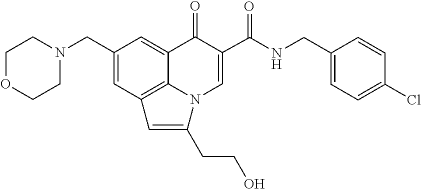 [SMILES: O=C(NCc1ccc(Cl)cc1)c1cn2c(CCO)cc3cc(CN4CCOCC4)cc(c1=O)c32]

PHA-529311 (Caroll B. Hartline, Emma A. Harden, Stephanie L. Williams-Aziz, Nicole L. Kushner, Roger J. Brideau, Earl R. Kern, Antiviral Research 65 (2005) 97-105 and R. L. Dorow,* Paul M. Herrinton,* Richard A. Hohler, Mark T. Maloney, Michael A. Mauragis, William E. McGhee, Jeffery A. Moeslein, Joseph W. Strohbach, and Michael F. VeleyOrganic Process Research & Development 2006, 10, 493-499).

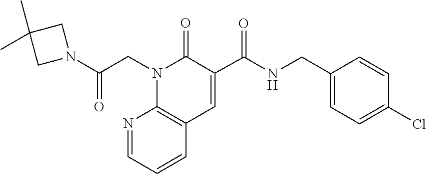 [SMILES: CC1(C)CN(C(=O)Cn2c(=O)c(C(=O)NCc3ccc(Cl)cc3)cc3cccnc32)C1]

1007 (Lee Fader et al., Cytomegalovirus Inhibitor Compounds; U.S. Pat. No. 9,315,499).

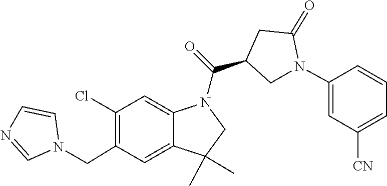 [SMILES: CC1(C)CN(C(=O)[C@H]2CC(=O)N(c3cccc(C#N)c3)C2)c2cc(Cl)c(Cn3ccnc3)cc21]

13b1 (Thibeault, Carl et al., Inhibitors of Cytomegalovirus; International Publication No. WO2014/070976 (PCT/US2013/067670)).

TABLE 6
Inhibition of hERG potassium channel
Activity IC₅₀ > 10 microM = + and IC₅₀ < 10 microM = ++

| Compound | Reference | CHO-hERG |
|---|---|---|
| Number | Compound | Activity Range |
| | PHA-529311 | ++ |
| | PNU-243672 | ++ |
| | 1007 | ++ |
| | 13b1 | ++ |
| 1 | | + |
| 2 | | ++ |
| 3 | | + |
| 4 | | ++ |
| 5 | | + |
| 6 | | + |
| 7 | | + |
| 12 | | + |
| 13 | | + |
| 14 | | + |
| 15 | | ++ |
| 16 | | + |
| 18 | | + |
| 19 | | + |
| 20 | | + |
| 21 | | + |
| 22 | | + |
| 26 | | + |
| 27 | | + |
| 28 | | + |
| 36 | | + |
| 37 | | + |
| 40 | | + |
| 41 | | + |
| 42 | | + |
| 43 | | + |
| 44 | | + |
| 45 | | + |
| 46 | | + |

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. Moreover, nothing disclosed herein is intended to be dedicated to the public regardless of whether such disclosure is explicitly recited in the claims.

The scope of the present invention, therefore, is not intended to be limited to the exemplary embodiments shown and described herein. Rather, the scope and spirit of present invention is embodied by the appended claims. In the claims, 35 U.S.C. § 112(f) or 35 U.S.C. § 112(6) is expressly defined as being invoked for a limitation in the claim only when the exact phrase “means for” or the exact phrase “step for” is recited at the beginning of such limitation in the claim; if such exact phrase is not used in a limitation in the claim, then 35 U.S.C. § 112 (f) or 35 U.S.C. § 112(6) is not invoked.

What is claimed is:

1. A compound of formula (I): 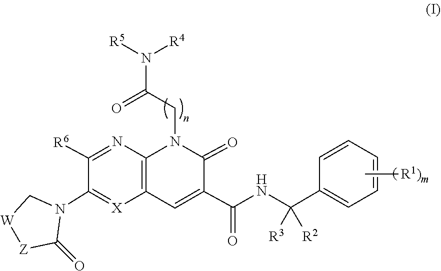
wherein:
each R¹ is independently selected from H, alkyl, substituted alkyl, halogen, nitrile, nitro, trifluoromethyl, hydroxyl, amino, and substituted amino;
R² and R³ are each independently selected from H, alkyl and substituted alkyl;
R⁴ and R⁵ are each independently selected from H, alkyl, substituted alkyl, cycloalkyl, substituted cycloalkyl, cycloalkenyl, substituted cycloalkenyl, aryl, substituted aryl, heterocycle, substituted heterocycle, heteroaryl, and substituted heteroaryl, or
R⁴ and R⁵ together with the nitrogen to which they are attached form a cycle selected from heteroaryl, substituted heteroaryl, heterocycle, and substituted heterocycle, (e.g., azetidine, spiroazetidine);
X is CR⁷ or N;
R⁶ and R⁷ are each independently selected from H, alkyl, substituted alkyl, alkoxy, substituted alkoxy, halogen, nitrile, nitro, trifluoromethyl, hydroxyl, amino, substituted amino, cycloalkyl, substituted cycloalkyl, cycloalkenyl, substituted cycloalkenyl, aryl, substituted aryl, heterocycle, substituted heterocycle, heteroaryl, and substituted heteroaryl;
Z is selected from NR⁸, O, and C(R⁹)₂;
W is selected from C(R^{9a})₂, and C(O);
R⁸ is selected from H, alkyl, substituted alkyl, cycloalkyl, substituted cycloalkyl, cycloalkenyl, substituted cycloalkenyl, aryl, substituted aryl, heterocycle, substituted heterocycle, heteroaryl, and substituted heteroaryl;
each R⁹ and R^{9a} is independently selected from H, alkyl, substituted alkyl, alkoxy, substituted alkoxy, halogen, nitrile, nitro, trifluoromethyl, hydroxyl, amino, substituted amino, cycloalkyl, substituted cycloalkyl, cycloalkenyl, substituted cycloalkenyl, aryl, substituted aryl, heterocycle, substituted heterocycle, heteroaryl, and substituted heteroaryl;
n is an integer from 1 to 6; and
m is an integer from 1 to 5;
or a pharmaceutically acceptable salt, solvate, or hydrate thereof.

2. The compound of claim 1, wherein R⁴ and R⁵ together with the nitrogen to which they are attached form a cycle selected from azetidine, and spiroazetidine, wherein the azetidine and the spiroazetidine are optionally substituted.

3. The compound of claim 2, wherein the cycle is selected from one of the following structures: 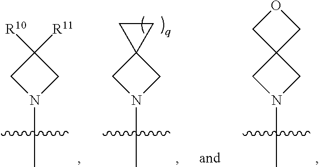
wherein:
R¹⁰ and R¹¹ are each independently selected from C₁₋₆ alkyl, hydroxyl, alkoxy, —(CH₂)ₚOH, and halogen;
p is an integer from 1 to 6; and
q is an integer from 1 to 4.

4. The compound of claim 1, of the formula (II): 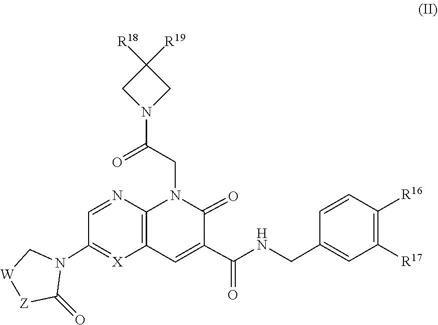
wherein:
X is CR⁷ or N;
R⁷ is selected from H, alkyl, substituted alkyl, alkoxy, substituted alkoxy, halogen, nitrile, nitro, trifluoromethyl, hydroxyl, amino, substituted amino, cycloalkyl, substituted cycloalkyl, cycloalkenyl, substituted cycloalkenyl, aryl, substituted aryl, heterocycle, substituted heterocycle, heteroaryl, and substituted heteroaryl;
Z is selected from NR⁸, O, and C(R⁹)₂;
W is selected from C(R^{9a})₂, and C(O);
R⁸ is selected from H, alkyl, substituted alkyl, cycloalkyl, substituted cycloalkyl, cycloalkenyl, substituted cycloalkenyl, aryl, substituted aryl, heterocycle, substituted heterocycle, heteroaryl, and substituted heteroaryl;
each R⁹ and R^{9a} is independently selected from H, alkyl, substituted alkyl, alkoxy, substituted alkoxy, halogen, nitrile, nitro, trifluoromethyl, hydroxyl, amino, substituted amino, cycloalkyl, substituted cycloalkyl, cycloalkenyl, substituted cycloalkenyl, aryl, substituted aryl, heterocycle, substituted heterocycle, heteroaryl, and substituted heteroaryl;
R¹⁶ is selected from C₁₋₆ alkyl, halogen, nitrile, and trifluoromethyl;
R¹⁷ is selected from hydrogen, and halogen;
R¹⁸ and R¹⁹ are each independently selected from C₁₋₆ alkyl, hydroxyl, alkoxy, —(CH₂)ₚOH, and halogen, or
R¹⁸ and R¹⁹ together with the carbon to which they are attached form a 3-6 membered cycle selected from cycloalkyl, substituted cycloalkyl, cycloalkenyl, substituted cycloalkenyl, aryl, substituted aryl, heteroaryl, substituted heteroaryl, heterocycle, and substituted heterocycle; and
p is an integer from 1 to 6.

5. The compound of any one of the preceding claims, wherein X is N.

6. The compound of claim 4 or 5, wherein R¹⁸ and R¹⁹ are independently selected from C₁₋₆ alkyl, and halogen, or
R¹⁸ and R¹⁹ together with the carbon to which they are attached form a cycle selected from C₃₋₆cycloalkyl, substituted C₃₋₆cycloalkyl, C₃₋₆heterocycle, and substituted C₃₋₆heterocycle.

7. The compound of claim 6, wherein R¹⁸ and R¹⁹ are both methyl.

8. The compound of claim 6, wherein R¹⁸ is F, and R¹⁹ is methyl.

9. The compound of claim 6, wherein R¹⁸ and R¹⁹ are both F.

10. The compound of claim 6, wherein R¹⁸ and R¹⁹ together with the carbon to which they are attached forms a cycle selected from cyclopropyl, substituted cyclopropyl, oxetane, and substituted oxetane.

11. The compound of any one of the preceding claims, wherein Z is C(R⁹)H, wherein R⁹ is selected from H, C₁₋₆ alkyl, C₃₋₆cycloalkyl, substituted C₃₋₆cycloalkyl, hydroxyl, alkoxy, —(CH₂)ₚOR¹², —(CH₂)ₚCO₂R¹², and —(CH₂)ₚN(R¹³)₂;
R¹² is selected from H, alkyl and substituted alkyl;
each R¹³ is independently selected from H, C₁₋₆ alkyl, substituted C₁₋₆ alkyl, S(O)₂R¹⁴, and C(O)R¹⁴, or
two R¹³ together with the nitrogen to which they are attached form a cycle selected from heteroaryl, substituted heteroaryl, heterocycle, and substituted heterocycle;
R¹⁴ is selected from C₁₋₆ alkyl, and substituted C₁₋₆ alkyl; and
p is an integer from 1 to 6.

12. The compound of claim 11, wherein two R¹³ together with the nitrogen to which they are attached forms a cycle selected from, azetidine, pyrrolidine, pyrazolidine, imidazolidine, pyrrole, morpholine, piperidine, piperazine, thiomorpholine, tetrazole, triazole, imidazole, pyrazole, pyridine, pyrimidine, pyrazine, and trizine, wherein the cycle is optionally substituted (e.g., with one or more substituents).

13. The compound of claim 11, wherein R¹⁴ is substituted C₁₋₆ alkyl, wherein one or more substituents is selected from halogen, C₃₋₆cycloalkyl, substituted C₃₋₆cycloalkyl, C₃₋₆heterocycle, and substituted C₃₋₆heterocycle (e.g., F, cyclopropyl, oxetane).

14. The compound of claim 11, wherein R⁹ is H, or C₁₋₃alkyl.

15. The compound of any one of claims 1 to 10, wherein Z is NR⁸, wherein R⁸ is selected from H, C₁₋₆ alkyl, substituted C₁₋₆ alkyl, —C(O)(CH₂)ₚN(R¹³)₂, C₃₋₆cycloalkyl, substituted C₃₋₆cycloalkyl, C₃₋₆heterocycle, and substituted C₃₋₆heterocycle;
each R¹³ is independently selected from H, C₁₋₆ alkyl, substituted C₁₋₆ alkyl, S(O)₂R¹⁴, and C(O)R¹⁴, or
two R¹³ together with the nitrogen to which they are attached form a cycle selected from heteroaryl, substituted heteroaryl, heterocycle, and substituted heterocycle;
R¹⁴ is selected from C1-6 alkyl, and substituted C1-6 alkyl; and
p is an integer from 1 to 6.

16. The compound of claim 15, wherein R⁸ is C₁₋₆ alkyl substituted with one or two R¹⁵ groups, wherein each R¹⁵ is independently selected from heterocycle, substituted heterocycle, CH₃, CH₂F, CHF₂, CF₃, CN, C(NH)NH₂, OH, CH₂OH, CO₂H, N(R¹³)₂, CH₂N(R¹³)₂, C(O)N(R¹³)₂, C(O)NHCN, and S(O)₂N(R¹³)₂.

17. The compound of claim 15 or 16, wherein two R¹³ together with the nitrogen to which they are attached forms a cycle selected from, azetidine, pyrrolidine, pyrazolidine, imidazolidine, pyrrole, morpholine, piperidine, piperazine, thiomorpholine, tetrazole, triazole, imidazole, pyrazole, pyridine, pyrimidine, pyrazine, and trizine, wherein the cycle is optionally substituted (e.g., with one or more substituents).

18. The compound of claim 15, wherein R¹⁴ is substituted C₁₋₆ alkyl, wherein one or more substituents is selected from halogen, C₃₋₆cycloalkyl, substituted C₃₋₆cycloalkyl, C₃₋₆heterocycle, and substituted C₃₋₆heterocycle (e.g., F, cyclopropyl, oxetane).

19. The compound of claim 15, wherein R⁸ is selected from H, C₁₋₃ alkyl, —CH₂(R¹⁵), —CH(R¹⁵)₂, —C(O)(CH₂)ₚN(R¹³)₂, and cyclopropyl;
each R¹⁵ is independently selected from heterocycle, substituted heterocycle, CH₃, CH₂F, CHF₂, CF₃, CN, C(NH)NH₂, OH, CH₂OH, CO₂H, N(R¹³)₂, CH₂N(R¹³)₂, C(O)N(R¹³)₂, C(O)NHCN, S(O)₂R¹⁴ and S(O)₂N(R¹³)₂; and
each R¹³ is independently selected from H, C₁₋₆ alkyl, substituted C₁₋₆ alkyl, S(O)₂R¹⁴, and C(O)R¹⁴, or
two R¹³ together with the nitrogen to which they are attached form a cycle selected from heteroaryl, substituted heteroaryl, heterocycle, and substituted heterocycle;

20. The compound of claim 19, wherein R⁸ is selected from H, methyl, and ethyl.

21. The compound of claim 19, wherein R⁸ is selected from —CH₂CO₂H, —C(CH₂NH₂)HCO₂H, —CH₂C(═O)N(CH₃)₂, —CH₂C(═O)N(CH₃)H, —CH₂C(═O)NH₂, —CH₂CH₂NH₂, —CH₂CH₂N(CH₃)₂, —CH₂CH₂NHC(═O)CH₂NH₂, —CH₂CH₂NHC(═O)CH(CH(CH₃)₂)(NH₂), —CH(CH₃)(CO₂H), —C(CH₃)₂(CO₂H), 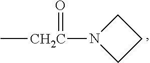
—CH₂CH₂N(CH₃)H,

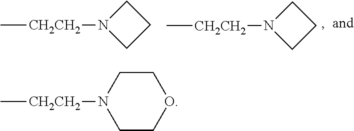

22. The compound of claim 15, wherein each R¹³ is independently selected from H, methyl and ethyl.

23. The compound of claim 16, wherein R¹⁵ is selected from tetrazole, —CO₂H, N(R¹³)₂, —CH₂N(R¹³)₂, —C(O)N(R¹³)₂, C(O)NHCN, and S(O)₂N(R¹³)₂.

24. The compound of claim 23, wherein R¹⁵ is selected from CO₂H, N(R¹³)₂, —CH₂N(R¹³)₂, and —C(O)N(R¹³)₂.

25. The compound of claim 15, wherein R¹⁴ is selected from methyl, ethyl, and CHF₂.

26. The compound of any one of claims 1 to 10, wherein Z is O.

27. The compound of any one of the preceding claims, wherein W is C(R^{9a})H, wherein:
R^{9a} is selected from H, C₁₋₆ alkyl, C₃₋₆cycloalkyl, substituted C₃₋₆cycloalkyl, hydroxyl, alkoxy, —(CH₂)ₚCO₂R¹², —(CH₂)ₚN(R¹³)₂, and —(CH₂)ₚOR^{9b};
R^{9b} is selected from H, —C(O)CH₃, —(CH₂)ₚN(R¹³)₂, and —(CH₂)ₚCO₂R¹²;
R¹² is selected from H, alkyl and substituted alkyl;
each R¹³ is independently selected from H, C₁₋₆ alkyl, substituted C₁₋₆ alkyl, S(O)₂R¹⁴, and C(O)R¹⁴, or
two R¹³ together with the nitrogen to which they are attached form a cycle selected from heteroaryl, substituted heteroaryl, heterocycle, and substituted heterocycle;
R¹⁴ is selected from C₁₋₆ alkyl, and substituted C₁₋₆ alkyl; and
p is an integer from 1 to 6.

28. The compound of claim 27, wherein W is CH₂.

29. The compound of any one of claims 1 to 26, wherein W is C(O).

30. The compound of any one of claims 4 to 29, where R¹⁶ is selected from F, Cl, methyl and nitrile.

31. The compound of claim 30, wherein R¹⁶ is Cl.

32. The compound of claim 4, wherein the compound of formula (II) is of any one of formulae (IIIA) to (IIIC): 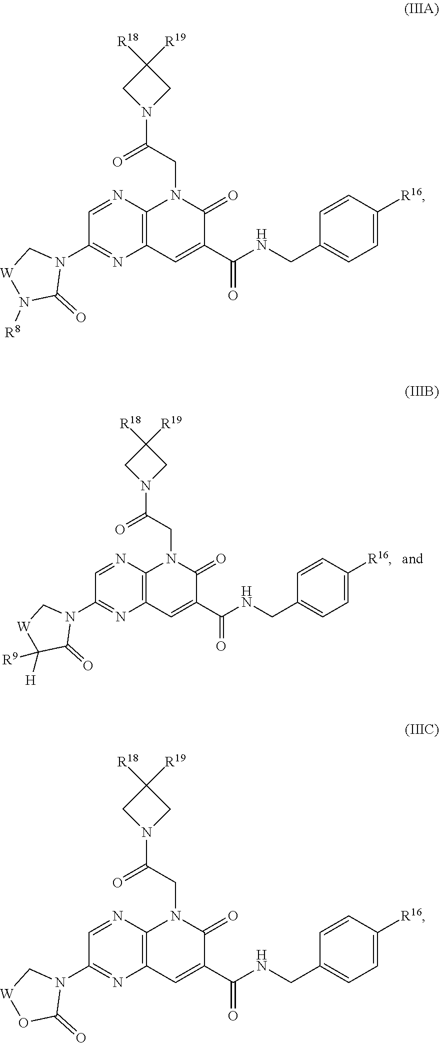
wherein:
each W is independently selected from C(R^{9a})₂, and C(O);
R⁸ is selected from H, C₁₋₆ alkyl, substituted C₁₋₆ alkyl, —C(O)(CH₂)ₚN(R¹³)₂, C₃₋₆cycloalkyl, substituted C₃₋₆cycloalkyl, C₃₋₆heterocycle, and substituted C₃₋₆heterocycle;
each R⁹ and R^{9a} is independently selected from H, C₁₋₆ alkyl, C₃₋₆ cycloalkyl, substituted C₃₋₆heterocycle, hydroxyl, alkoxy, —(CH₂)ₚOR¹², —(CH₂)ₚCO₂R¹², and —(CH₂)ₚN(R¹³)₂;
R¹² is selected from H, alkyl and substituted alkyl;
each R¹³ is independently selected from H, C₁₋₆ alkyl, substituted C₁₋₆ alkyl, S(O)₂R¹⁴, and C(O)R¹⁴, or
two R¹³ together with the nitrogen to which they are attached form a cycle selected from heteroaryl, substituted heteroaryl, heterocycle, and substituted heterocycle;
R¹⁴ is selected from C₁₋₆ alkyl, and substituted C₁₋₆ alkyl;
R¹⁶ is selected from C₁₋₆ alkyl, halogen, nitrile, and trifluoromethyl;
R¹⁸ and R¹⁹ are each independently selected from C₁₋₆ alkyl, hydroxyl, alkoxy, —(CH₂)ₚOH, and halogen, or
R¹⁸ and R¹⁹ together with the carbon to which they are attached form a 3-6 membered cycle selected from, cycloalkyl, substituted cycloalkyl, cycloalkenyl, substituted cycloalkenyl, aryl, substituted aryl, heteroaryl, substituted heteroaryl, heterocycle, and substituted heterocycle; and
each p is independently an integer from 1 to 6.

33. The compound of claim 32, wherein the compound is of any one of formulae (IVA) to (IVC):

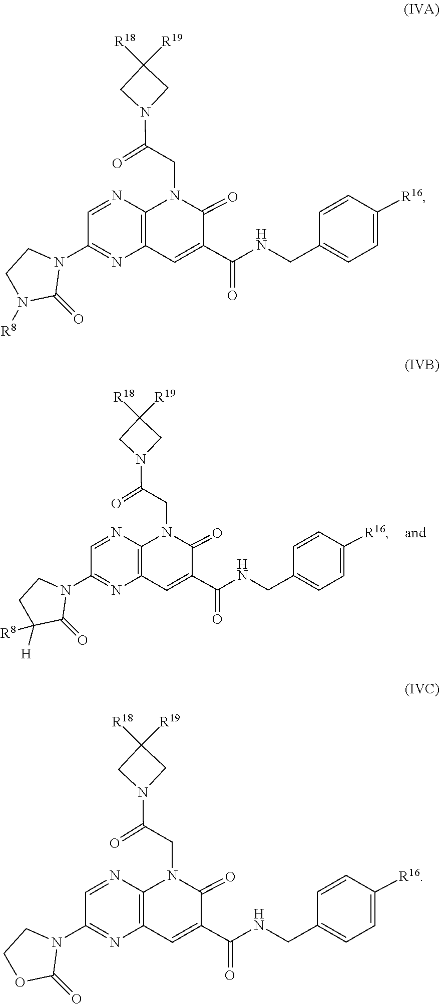

34. The compound of claim 11, wherein the compound of formula (II) is of any one of (IIID) to (IIIF): 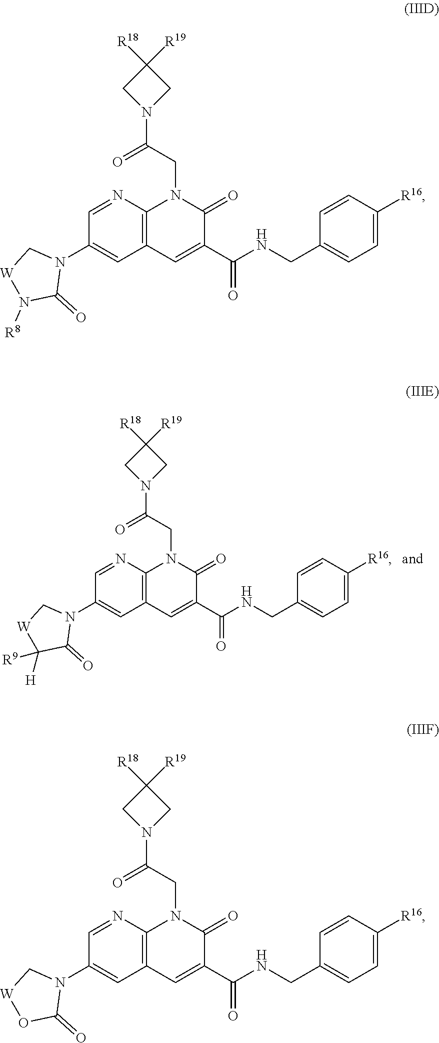
wherein:
each W is independently selected from C(R^{9a})₂, and C(O);
R⁸ is selected from H, C₁₋₆ alkyl, substituted C₁₋₆ alkyl, —C(O)(CH₂)ₚN(R¹³)₂, C₃₋₆cycloalkyl, substituted C₃₋₆cycloalkyl, C₃₋₆heterocycle, and substituted C₃₋₆heterocycle;
each R⁹ and R^{9a} is independently selected from H, C₁₋₆ alkyl, C₃₋₆ cycloalkyl, substituted C₃₋₆heterocycle, hydroxyl, alkoxy, —(CH₂)ₚOR¹², —(CH₂)ₚCO₂R¹², and —(CH₂)ₚN(R¹³)₂;
R¹² is selected from H, alkyl and substituted alkyl;
each R¹³ is independently selected from H, C₁₋₆ alkyl, substituted C₁₋₆ alkyl, S(O)₂R¹⁴, and C(O)R¹⁴, or
two R¹³ together with the nitrogen to which they are attached form a cycle selected from heteroaryl, substituted heteroaryl, heterocycle, and substituted heterocycle;
R¹⁴ is selected from C₁₋₆ alkyl, and substituted C₁₋₆ alkyl;
R¹⁶ is selected from C₁₋₆ alkyl, halogen, nitrile, and trifluoromethyl;
R¹⁸ and R¹⁹ are each independently selected from C₁₋₆ alkyl, hydroxyl, alkoxy, —(CH₂)ₚOH, and halogen, or
R¹⁸ and R¹⁹ together with the carbon to which they are attached form a 3-6 membered cycle selected from, cycloalkyl, substituted cycloalkyl, cycloalkenyl, substituted cycloalkenyl, aryl, substituted aryl, heteroaryl, substituted heteroaryl, heterocycle, and substituted heterocycle; and
each p is independently an integer from 1 to 6.

35. A compound of formula (V): 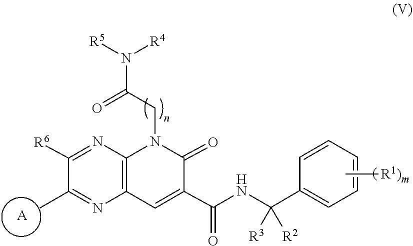
wherein:
each R¹ is independently selected from H, alkyl, substituted alkyl, halogen, nitrile, nitro, trifluoromethyl, hydroxyl, amino, and substituted amino;
R² and R³ are each independently selected from H, alkyl and substituted alkyl;
R⁴ and R⁵ are each independently selected from H, alkyl, substituted alkyl, cycloalkyl, substituted cycloalkyl, cycloalkenyl, substituted cycloalkenyl, aryl, substituted aryl, heterocycle, substituted heterocycle, heteroaryl, and substituted heteroaryl, or
R⁴ and R⁵ together with the nitrogen to which they are attached form a cycle selected from, heteroaryl, substituted heteroaryl, heterocycle, and substituted heterocycle, (e.g., azetidine, spiroazetidine);
R⁶ is selected from hydrogen, alkyl, substituted alkyl, alkoxy, substituted alkoxy, halogen, nitrile, nitro, trifluoromethyl, hydroxyl, amino, substituted amino, cycloalkyl, substituted cycloalkyl, cycloalkenyl, substituted cycloalkenyl, aryl, substituted aryl, heterocycle, substituted heterocycle, heteroaryl, and substituted heteroaryl;
Ring A is selected from cycloalkyl, substituted cycloalkyl, cycloalkenyl, substituted cycloalkenyl, aryl, substituted aryl, heterocycle, substituted heterocycle, heteroaryl, and substituted heteroaryl;
n is an integer from 1 to 6; and
m is an integer from 1 to 5;
or a pharmaceutically acceptable salt, solvate, or hydrate thereof.

36. The compound of claim 1, selected from one of the following structures:

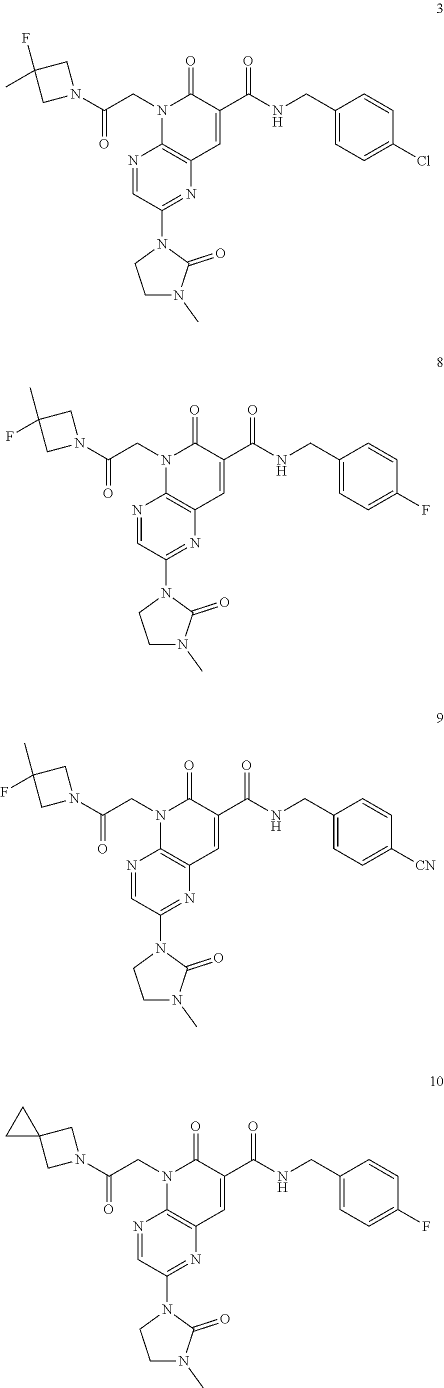 [SMILES: CN1CCN(c2cnc3c(cc(C(=O)NCc4ccc(Cl)cc4)c(=O)n3CC(=O)N3CC(C)(F)C3)n2)C1=O ; CN1CCN(c2cnc3c(cc(C(=O)NCc4ccc(F)cc4)c(=O)n3CC(=O)N3CC(C)(F)C3)n2)C1=O ; CN1CCN(c2cnc3c(cc(C(=O)NCc4ccc(C#N)cc4)c(=O)n3CC(=O)N3CC(C)(F)C3)n2)C1=O ; CN1CCN(c2cnc3c(cc(C(=O)NCc4ccc(F)cc4)c(=O)n3CC(=O)N3CC4(CC4)C3)n2)C1=O]

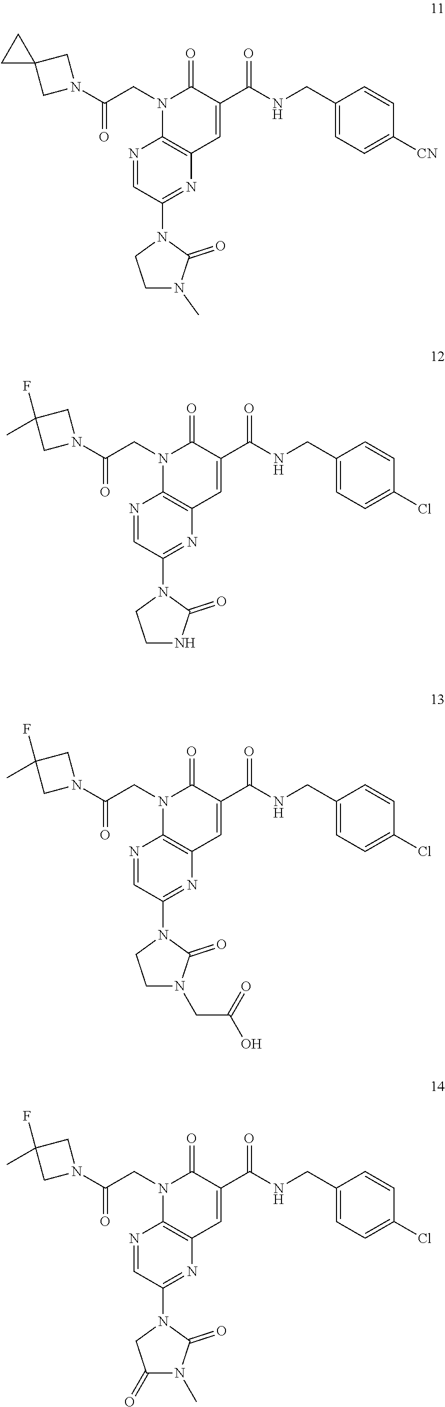 [SMILES: CN1CCN(c2cnc3c(cc(C(=O)NCc4ccc(C#N)cc4)c(=O)n3CC(=O)N3CC4(CC4)C3)n2)C1=O ; CC1(F)CN(C(=O)Cn2c(=O)c(C(=O)NCc3ccc(Cl)cc3)cc3nc(N4CCNC4=O)cnc32)C1 ; CC1(F)CN(C(=O)Cn2c(=O)c(C(=O)NCc3ccc(Cl)cc3)cc3nc(N4CCN(CC(=O)O)C4=O)cnc32)C1 ; CN1C(=O)CN(c2cnc3c(cc(C(=O)NCc4ccc(Cl)cc4)c(=O)n3CC(=O)N3CC(C)(F)C3)n2)C1=O]

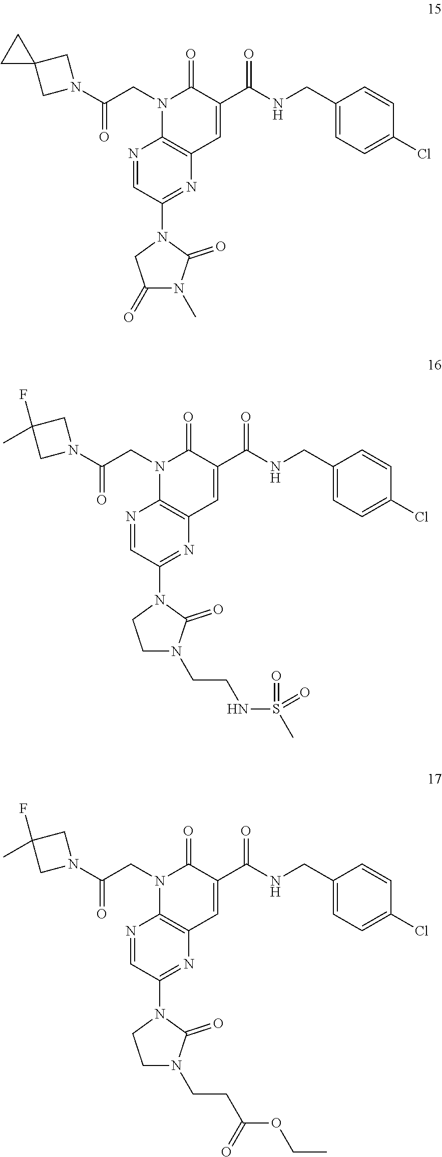 [SMILES: CN1C(=O)CN(c2cnc3c(cc(C(=O)NCc4ccc(Cl)cc4)c(=O)n3CC(=O)N3CC4(CC4)C3)n2)C1=O ; CC1(F)CN(C(=O)Cn2c(=O)c(C(=O)NCc3ccc(Cl)cc3)cc3nc(N4CCN(CCNS(C)(=O)=O)C4=O)cnc32)C1 ; CCOC(=O)CCN1CCN(c2cnc3c(cc(C(=O)NCc4ccc(Cl)cc4)c(=O)n3CC(=O)N3CC(C)(F)C3)n2)C1=O]

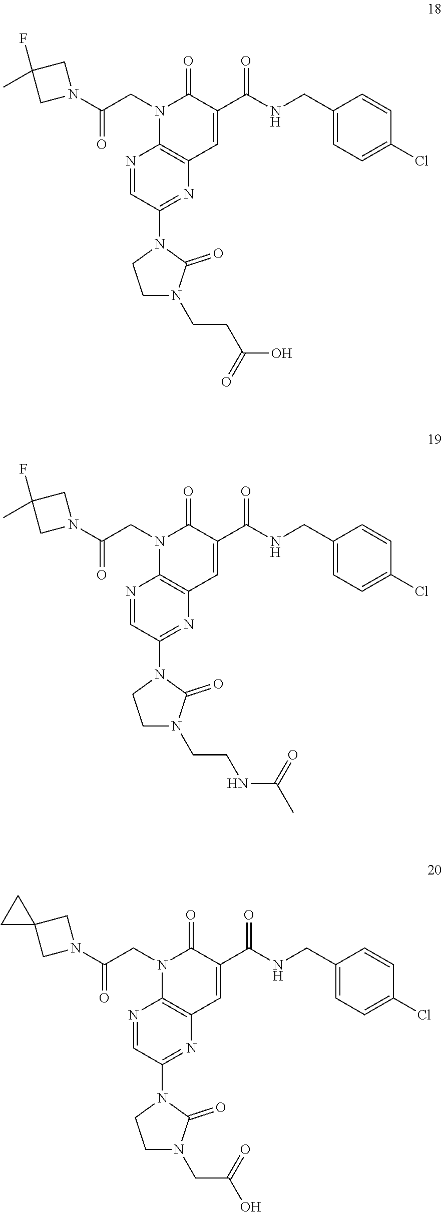 [SMILES: CC1(F)CN(C(=O)Cn2c(=O)c(C(=O)NCc3ccc(Cl)cc3)cc3nc(N4CCN(CCC(=O)O)C4=O)cnc32)C1 ; CC(=O)NCCN1CCN(c2cnc3c(cc(C(=O)NCc4ccc(Cl)cc4)c(=O)n3CC(=O)N3CC(C)(F)C3)n2)C1=O ; O=C(O)CN1CCN(c2cnc3c(cc(C(=O)NCc4ccc(Cl)cc4)c(=O)n3CC(=O)N3CC4(CC4)C3)n2)C1=O]

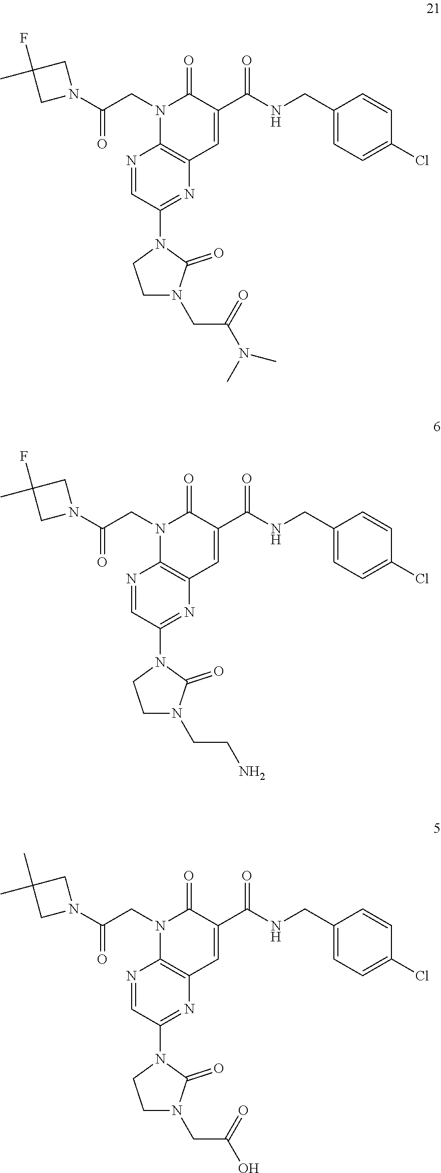 [SMILES: CN(C)C(=O)CN1CCN(c2cnc3c(cc(C(=O)NCc4ccc(Cl)cc4)c(=O)n3CC(=O)N3CC(C)(F)C3)n2)C1=O ; CC1(F)CN(C(=O)Cn2c(=O)c(C(=O)NCc3ccc(Cl)cc3)cc3nc(N4CCN(CCN)C4=O)cnc32)C1 ; CC1(C)CN(C(=O)Cn2c(=O)c(C(=O)NCc3ccc(Cl)cc3)cc3nc(N4CCN(CC(=O)O)C4=O)cnc32)C1]

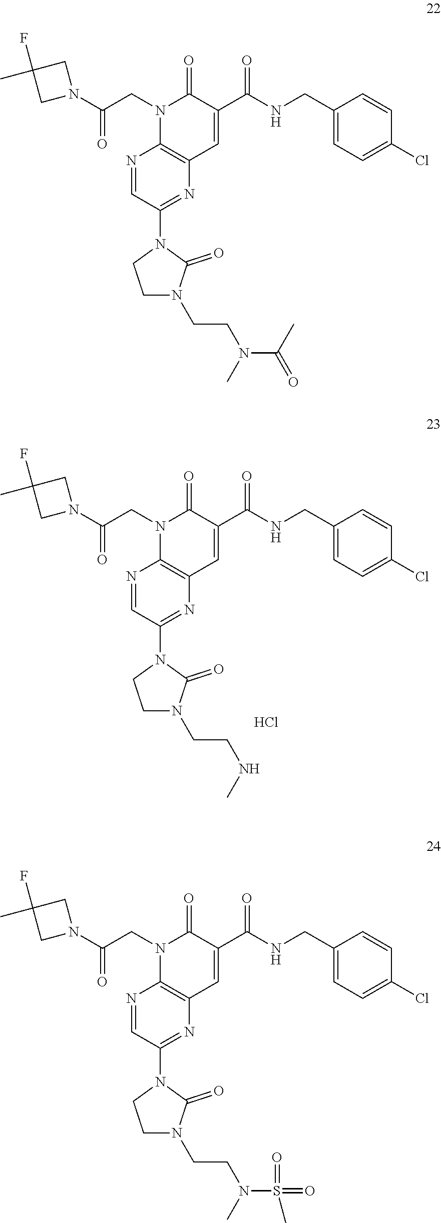 [SMILES: CC(=O)N(C)CCN1CCN(c2cnc3c(cc(C(=O)NCc4ccc(Cl)cc4)c(=O)n3CC(=O)N3CC(C)(F)C3)n2)C1=O ; CNCCN1CCN(c2cnc3c(cc(C(=O)NCc4ccc(Cl)cc4)c(=O)n3CC(=O)N3CC(C)(F)C3)n2)C1=O ; Cl ; CN(CCN1CCN(c2cnc3c(cc(C(=O)NCc4ccc(Cl)cc4)c(=O)n3CC(=O)N3CC(C)(F)C3)n2)C1=O)S(C)(=O)=O]

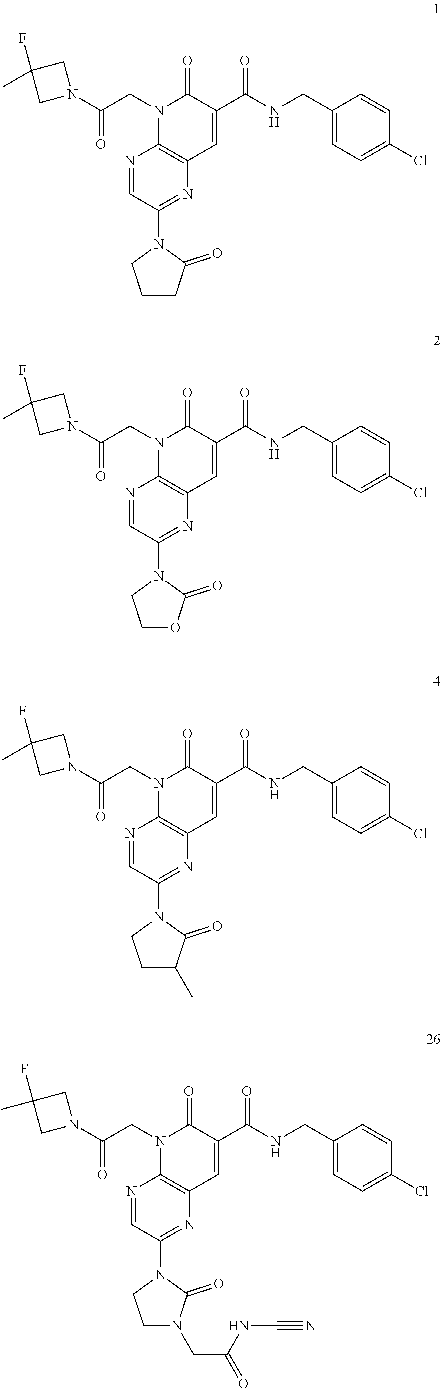 [SMILES: CC1(F)CN(C(=O)Cn2c(=O)c(C(=O)NCc3ccc(Cl)cc3)cc3nc(N4CCCC4=O)cnc32)C1 ; CC1(F)CN(C(=O)Cn2c(=O)c(C(=O)NCc3ccc(Cl)cc3)cc3nc(N4CCOC4=O)cnc32)C1 ; CC1CCN(c2cnc3c(cc(C(=O)NCc4ccc(Cl)cc4)c(=O)n3CC(=O)N3CC(C)(F)C3)n2)C1=O ; CC1(F)CN(C(=O)Cn2c(=O)c(C(=O)NCc3ccc(Cl)cc3)cc3nc(N4CCN(CC(=O)NC#N)C4=O)cnc32)C1]

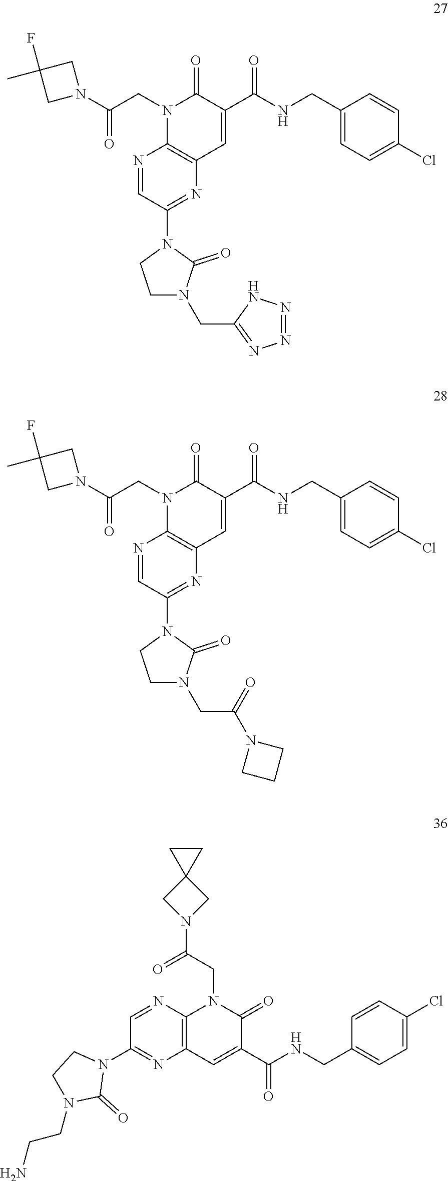 [SMILES: CC1(F)CN(C(=O)Cn2c(=O)c(C(=O)NCc3ccc(Cl)cc3)cc3nc(N4CCN(Cc5nnn[nH]5)C4=O)cnc32)C1 ; CC1(F)CN(C(=O)Cn2c(=O)c(C(=O)NCc3ccc(Cl)cc3)cc3nc(N4CCN(CC(=O)N5CCC5)C4=O)cnc32)C1 ; NCCN1CCN(c2cnc3c(cc(C(=O)NCc4ccc(Cl)cc4)c(=O)n3CC(=O)N3CC4(CC4)C3)n2)C1=O]

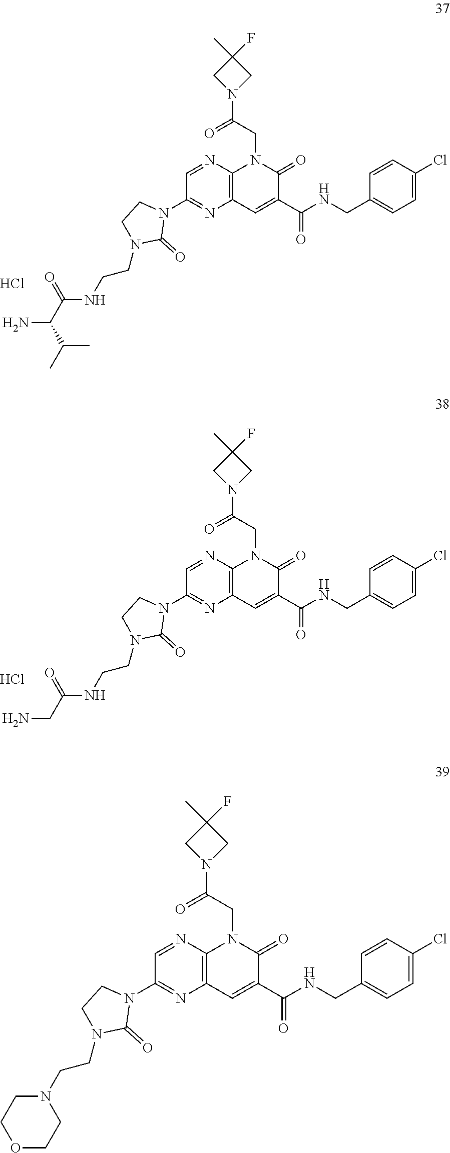 [SMILES: Cl ; CC(C)[C@H](N)C(=O)NCCN1CCN(c2cnc3c(cc(C(=O)NCc4ccc(Cl)cc4)c(=O)n3CC(=O)N3CC(C)(F)C3)n2)C1=O ; Cl ; CC1(F)CN(C(=O)Cn2c(=O)c(C(=O)NCc3ccc(Cl)cc3)cc3nc(N4CCN(CCNC(=O)CN)C4=O)cnc32)C1 ; CC1(F)CN(C(=O)Cn2c(=O)c(C(=O)NCc3ccc(Cl)cc3)cc3nc(N4CCN(CCN5CCOCC5)C4=O)cnc32)C1]

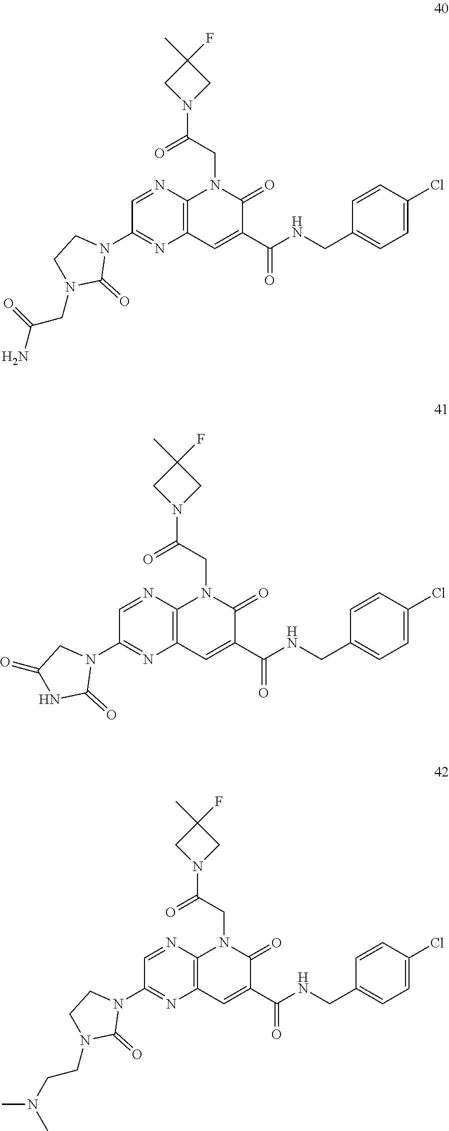 [SMILES: CC1(F)CN(C(=O)Cn2c(=O)c(C(=O)NCc3ccc(Cl)cc3)cc3nc(N4CCN(CC(N)=O)C4=O)cnc32)C1 ; CC1(F)CN(C(=O)Cn2c(=O)c(C(=O)NCc3ccc(Cl)cc3)cc3nc(N4CC(=O)NC4=O)cnc32)C1 ; CN(C)CCN1CCN(c2cnc3c(cc(C(=O)NCc4ccc(Cl)cc4)c(=O)n3CC(=O)N3CC(C)(F)C3)n2)C1=O]

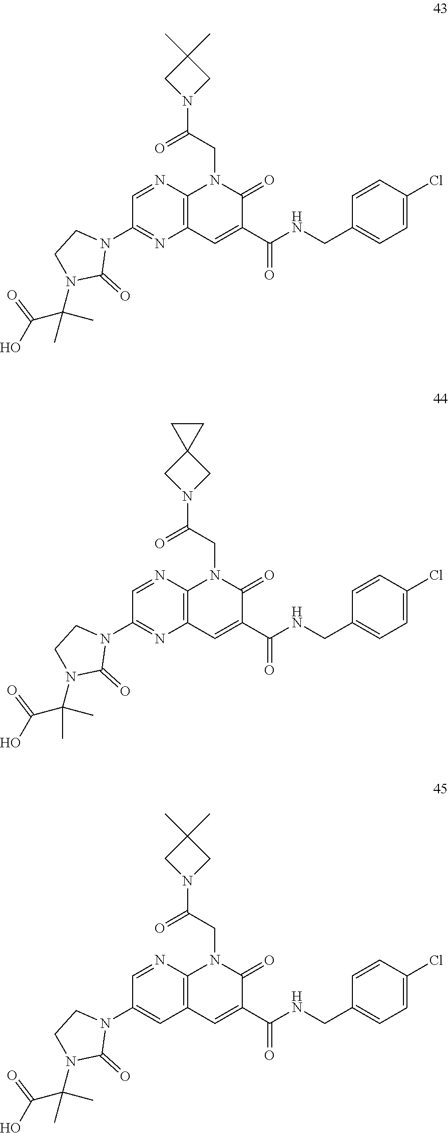 [SMILES: CC1(C)CN(C(=O)Cn2c(=O)c(C(=O)NCc3ccc(Cl)cc3)cc3nc(N4CCN(C(C)(C)C(=O)O)C4=O)cnc32)C1 ; CC(C)(C(=O)O)N1CCN(c2cnc3c(cc(C(=O)NCc4ccc(Cl)cc4)c(=O)n3CC(=O)N3CC4(CC4)C3)n2)C1=O ; CC1(C)CN(C(=O)Cn2c(=O)c(C(=O)NCc3ccc(Cl)cc3)cc3cc(N4CCN(C(C)(C)C(=O)O)C4=O)cnc32)C1]

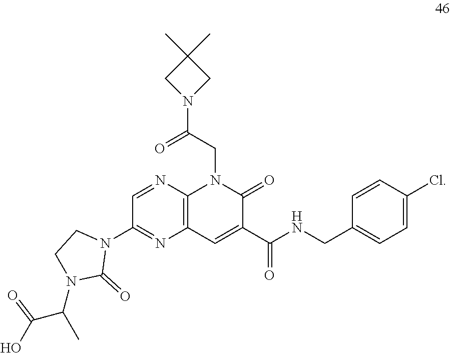 [SMILES: CC(C(=O)O)N1CCN(c2cnc3c(cc(C(=O)NCc4ccc(Cl)cc4)c(=O)n3CC(=O)N3CC(C)(C)C3)n2)C1=O]

37. The compound of claim 1, of the structure:

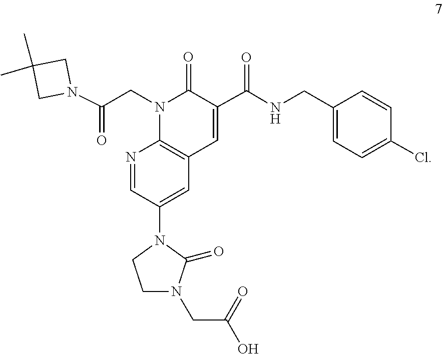 [SMILES: CC1(C)CN(C(=O)Cn2c(=O)c(C(=O)NCc3ccc(Cl)cc3)cc3cc(N4CCN(CC(=O)O)C4=O)cnc32)C1]

38. The compound of claim 1, selected from one of the following structures:

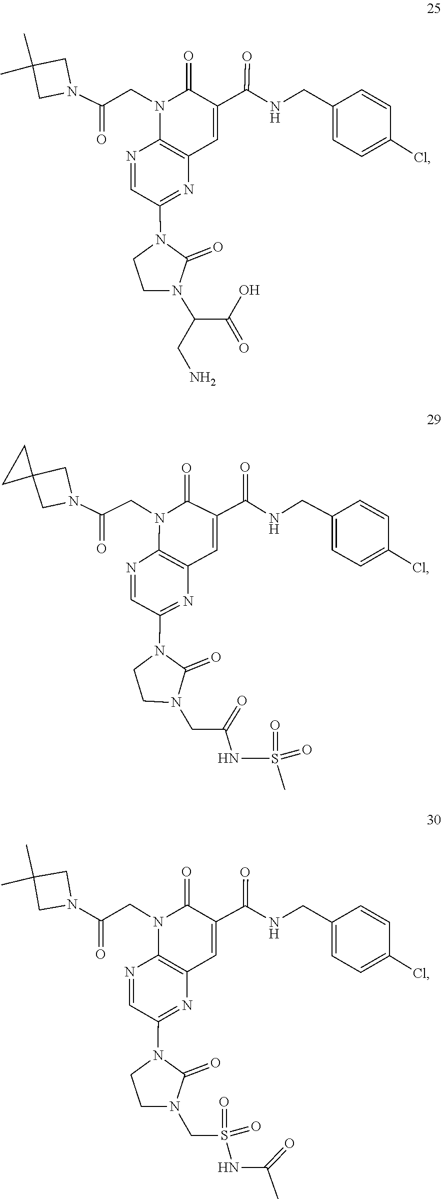 [SMILES: CC1(C)CN(C(=O)Cn2c(=O)c(C(=O)NCc3ccc(Cl)cc3)cc3nc(N4CCN(C(CN)C(=O)O)C4=O)cnc32)C1 ; CS(=O)(=O)NC(=O)CN1CCN(c2cnc3c(cc(C(=O)NCc4ccc(Cl)cc4)c(=O)n3CC(=O)N3CC4(CC4)C3)n2)C1=O ; CC(=O)NS(=O)(=O)CN1CCN(c2cnc3c(cc(C(=O)NCc4ccc(Cl)cc4)c(=O)n3CC(=O)N3CC(C)(C)C3)n2)C1=O]

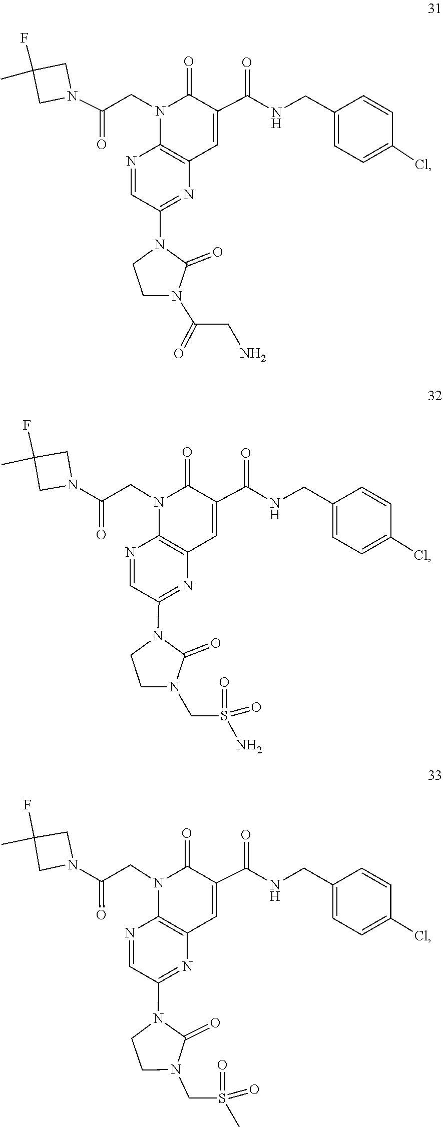 [SMILES: CC1(F)CN(C(=O)Cn2c(=O)c(C(=O)NCc3ccc(Cl)cc3)cc3nc(N4CCN(C(=O)CN)C4=O)cnc32)C1 ; CC1(F)CN(C(=O)Cn2c(=O)c(C(=O)NCc3ccc(Cl)cc3)cc3nc(N4CCN(CS(N)(=O)=O)C4=O)cnc32)C1 ; CC1(F)CN(C(=O)Cn2c(=O)c(C(=O)NCc3ccc(Cl)cc3)cc3nc(N4CCN(CS(C)(=O)=O)C4=O)cnc32)C1]

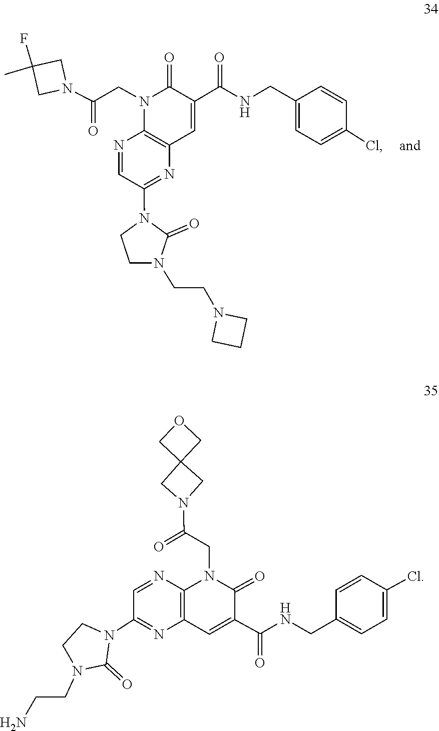 [SMILES: CC1(F)CN(C(=O)Cn2c(=O)c(C(=O)NCc3ccc(Cl)cc3)cc3nc(N4CCN(CCN5CCC5)C4=O)cnc32)C1 ; NCCN1CCN(c2cnc3c(cc(C(=O)NCc4ccc(Cl)cc4)c(=O)n3CC(=O)N3CC4(COC4)C3)n2)C1=O]

39. A method of inhibiting a herpesvirus in a cell infected with a herpesvirus, the method comprising contacting the cell with a compound of any one of claims 1 to 38.

40. The method of claim 39, wherein the herpesvirus is selected from cytomegalovirus (CMV), herpes simplex virus-1 (HSV-1), herpes simplex virus-2 (HSV-2), varicella-zoster virus (VZV), Epstein-Barr virus (EBV), and Kaposi's sarcoma-associated herpesvirus (KSHV).

41. The method of claim 40, wherein the herpesvirus is cytomegalovirus (CMV).

42. The method of claim 41, wherein the herpesvirus is human cytomegalovirus (HCMV).

43. A method of treating or preventing a herpesvirus infection in an individual, the method comprising administering to the individual an effective amount of a compound according to any one of claims 1 to 38.

44. The method of claim 43, wherein the herpesvirus is selected from cytomegalovirus (CMV), herpes simplex virus-1 (HSV-1), herpes simplex virus-2 (HSV-2), varicella-zoster virus (VZV), Epstein-Barr virus (EBV), and Kaposi's sarcoma-associated herpesvirus (KSHV).

45. The method of claim 44, wherein the herpesvirus is cytomegalovirus (CMV).

46. The method of claim 45, wherein the herpesvirus is human cytomegalovirus (HCMV).

47. The method of any one of claims 43-46, wherein the compound is administered in combination with one or more additional active agents.

48. The method of claim 47, wherein the additional active agent is an antiviral agent (e.g., an additional herpesvirus inhibitor).

49. The method of claim 48, wherein the antiviral agent is a herpesvirus inhibitor selected from acyclovir, ganciclovir, valacylovir, valganciclovir, foscarnet, and letermovir.
